SEQUENCE LISTING (309 sequences)

SEQ ID NO: 1 - DNA, 87 bp, synthetic construct
  misc_feature 1..87: note = Description of sequence: Artificial sequence
  source 1..87: mol_type = other DNA
  variation 21..70: note = n is a, c, g, or t
ggaaggaatcgtacgattccnnnnnnnnnnnnnnnnnnnnnnnnnnnnnnnnnnnnnnnnnnnnnnnnnncgtgatgcctctacctc

SEQ ID NO: 2 - DNA, 110 bp, synthetic construct
  misc_feature 1..110: note = Description of sequence: Artificial sequence
  source 1..110: mol_type = other DNA
  variation 44..93: note = n is a, c, g, or t
  misc_feature 1..22: note = DNA
  misc_feature 23: note = RNA
  misc_feature 24..110: note = DNA
gactggtatcaatctcacgtataggaaggaatcgtacgattccnnnnnnnnnnnnnnnnnnnnnnnnnnnnnnnnnnnnnnnnnnnnnnnnnncgtgatgcctctacctc

SEQ ID NO: 3 - DNA, 32 bp, synthetic construct
  misc_feature 1..32: note = Description of sequence: Artificial sequence
  source 1..32: mol_type = other DNA
gtatcaatctcacgtataggaaggaatcgtac

SEQ ID NO: 4 - DNA, 17 bp, synthetic construct
  misc_feature 1..17: note = Description of sequence: Artificial sequence
  source 1..17: mol_type = other DNA
cgtgatgcctctacctc

SEQ ID NO: 5 - DNA, 31 bp, synthetic construct
  misc_feature 1..31: note = Description of sequence: Artificial sequence
  source 1..31: mol_type = other DNA
  modified_base 15: mod_base = OTHER; note = cytosine 5' carbon bonded to C18-3'-cytosine
aacaacaacaacaacgtgatgcctctacctc

SEQ ID NO: 6 - DNA, 23 bp, synthetic construct
  misc_feature 1..23: note = Description of sequence: Artificial sequence
  source 1..23: mol_type = other DNA
  misc_feature 1..22: note = DNA
  misc_feature 23: note = RNA
gactggtatcaatctcacgtata

SEQ ID NO: 7 - DNA, 96 bp, synthetic construct
  misc_feature 1..96: note = Description of sequence: Artificial sequence
  source 1..96: mol_type = other DNA
  variation 17..66: note = n is a, c, g, or t
  variation 51..66: note = these postions may absent
  modified_base 54: mod_base = OTHER; note = 3' carbon modifed with propyl group
ccggacctccttcagnnnnnnnnnnnnnnnnnnnnnnnnnnnnnnnnnnnnnnnnnnnnnnnnnngactcgtgcgagtctccctaactgaagtaag

SEQ ID NO: 8 - DNA, 110 bp, synthetic construct
  misc_feature 1..110: note = Description of sequence: Artificial sequence
  source 1..110: mol_type = other DNA
  variation 44..93: note = n is a, c, g, or t
  variation 78..93: note = these postions may absent
  misc_feature 1..25: note = DNA
  misc_feature 26: note = RNA
  misc_feature 27..110: note = DNA
gatacatagcatcttacttcagttagggagactcgcacgagtcnnnnnnnnnnnnnnnnnnnnnnnnnnnnnnnnnnnnnnnnnnnnnnnnnnctgaaggaggtccggtc

SEQ ID NO: 9 - DNA, 31 bp, synthetic construct
  misc_feature 1..31: note = Description of sequence: Artificial sequence
  source 1..31: mol_type = other DNA
gcatcttacttcagttagggagactcgcacg

SEQ ID NO: 10 - DNA, 17 bp, synthetic construct
  misc_feature 1..17: note = Description of sequence: Artificial sequence
  source 1..17: mol_type = other DNA
gaccggacctccttcag

SEQ ID NO: 11 - DNA, 34 bp, synthetic construct
  misc_feature 1..34: note = Description of sequence: Artificial sequence
  source 1..34: mol_type = other DNA
  modified_base 18: mod_base = OTHER; note = guanine 5' carbon bonded to C18-3'-cytosine
gacaacaacaacaacaagaccggacctccttcag

SEQ ID NO: 12 - DNA, 26 bp, synthetic construct
  misc_feature 1..26: note = Description of sequence: Artificial sequence
  source 1..26: mol_type = other DNA
  misc_feature 1..25: note = DNA
  misc_feature 26: note = RNA
gatacatagcatcttacttcagttag

SEQ ID NO: 13 - DNA, 109 bp, synthetic construct
  misc_feature 1..109: note = Description of sequence: Artificial sequence
  source 1..109: mol_type = other DNA
gactggtatcaatctcacgtataggaaggaatcgtacgttccgttatggttctttctcctagccagactgttatgtgtgatacggcaaacttcgtgatgcctctacctc

SEQ ID NO: 14 - DNA, 110 bp, synthetic construct
  misc_feature 1..110: note = Description of sequence: Artificial sequence
  source 1..110: mol_type = other DNA
gatacatagcatcttacttcagttagggagactcgcacgagtcccttatcggggaattcaatgtgcgcgtttgcggcacctaaacgctcttagctgaaggaggtccggtc

SEQ ID NO: 15 - DNA, 41 bp, synthetic construct
  misc_feature 1..41: note = Description of sequence: Artificial sequence
  source 1..41: mol_type = other DNA
  modified_base 41: mod_base = OTHER; note = nucleotide bonded to quencher, quencher name: 3' Iowa Black RQ
gcggcatgcgcgtttgcggcacctaaacgctcctaatagag

SEQ ID NO: 16 - DNA, 41 bp, synthetic construct
  misc_feature 1..41: note = Description of sequence: Artificial sequence
  source 1..41: mol_type = other DNA
  modified_base 41: mod_base = OTHER; note = nucleotide bonded to quencher, quencher name: 3' Iowa Black RQ
gcggcatgcgcgtttgcggcacctaaacgcccctaatagag

SEQ ID NO: 17 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 26: mod_base = OTHER; note = nucleotide bonded to quencher, quencher name: 3' Iowa Black RQ
  modified_base 1: mod_base = OTHER; note = nucleotide bonded to fluorescent dye, dye name: 5' Alexa Fluor 647
ctctattagggagactcgcatgccgc

SEQ ID NO: 18 - DNA, 65 bp, synthetic construct
  misc_feature 1..65: note = Description of sequence: Artificial sequence
  source 1..65: mol_type = other DNA
  modified_base 1: mod_base = OTHER; note = nucleotide bonded to fluorescent dye, dye name: 5' Iowa Black FQ
tggatatctcctagccagactgttatgtgtgatacggcaaacttcgtgatgcctctacgggtccg

SEQ ID NO: 19 - DNA, 65 bp, synthetic construct
  misc_feature 1..65: note = Description of sequence: Artificial sequence
  source 1..65: mol_type = other DNA
  modified_base 1: mod_base = OTHER; note = nucleotide bonded to fluorescent dye, dye name: 5' Iowa Black FQ
tggatatctcctggccagactgttatgtgtgatacggcaaacttcgtgatgcctctacgggtccg

SEQ ID NO: 20 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 34: mod_base = OTHER; note = nucleotide bonded to fluorescent dye, dye name: 3' Alexa Fluor 488
  modified_base 1: mod_base = OTHER; note = nucleotide bonded to fluorescent dye, dye name: 5' Iowa Black FQ
cggacccgtatcaatctcacgtataggatatcca

SEQ ID NO: 21 - DNA, 41 bp, synthetic construct; identical to SEQ ID NO: 15
  misc_feature 1..41: note = Description of sequence: Artificial sequence
  source 1..41: mol_type = other DNA

SEQ ID NO: 22 - DNA, 41 bp, synthetic construct; identical to SEQ ID NO: 16
  misc_feature 1..41: note = Description of sequence: Artificial sequence
  source 1..41: mol_type = other DNA

SEQ ID NO: 23 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
ctctattagggagactcgcatgccgc

SEQ ID NO: 24 - DNA, 65 bp, synthetic construct; identical to SEQ ID NO: 18
  misc_feature 1..65: note = Description of sequence: Artificial sequence
  source 1..65: mol_type = other DNA

SEQ ID NO: 25 - DNA, 65 bp, synthetic construct; identical to SEQ ID NO: 19
  misc_feature 1..65: note = Description of sequence: Artificial sequence
  source 1..65: mol_type = other DNA

SEQ ID NO: 26 - DNA, 34 bp, synthetic construct; identical to SEQ ID NO: 20
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA

SEQ ID NO: 27 - DNA, 15 bp, synthetic construct
  misc_feature 1..15: note = Description of sequence: Artificial sequence
  source 1..15: mol_type = other DNA
aacaacaacaacaac

SEQ ID NO: 28 - DNA, 65 bp, synthetic construct; identical to SEQ ID NO: 18
  misc_feature 1..65: note = Description of sequence: Artificial sequence
  source 1..65: mol_type = other DNA
  modified_base 1: mod_base = OTHER; note = nucleotide bonded to quencher, quencher name: 5' Iowa Black RQ

SEQ ID NO: 29 - DNA, 65 bp, synthetic construct
  misc_feature 1..65: note = Description of sequence: Artificial sequence
  source 1..65: mol_type = other DNA
  modified_base 1: mod_base = OTHER; note = nucleotide bonded to quencher, quencher name: 5' Iowa Black RQ
tggatatctcctagtcagactgttatgtgtgatacggcaaacttcgtgatgcctctacgggtccg

SEQ ID NO: 30 - DNA, 34 bp, synthetic construct; identical to SEQ ID NO: 20
  misc_feature 1..34: note = Description of sequence: Artificial sequence
  source 1..34: mol_type = other DNA
  modified_base 34: mod_base = OTHER; note = nucleotide bonded to fluorescent dye, dye name: 3' Alexa Fluor 546
  misc_feature 1..24: note = DNA
  misc_feature 25: note = RNA
  misc_feature 26..34: note = DNA
  modified_base 1: mod_base = OTHER; note = nucleotide bonded to quencher, quencher name: 5' Iowa Black RQ

SEQ ID NO: 31 - DNA, 65 bp, synthetic construct; identical to SEQ ID NO: 29
  misc_feature 1..65: note = Description of sequence: Artificial sequence
  source 1..65: mol_type = other DNA

SEQ ID NO: 32 - DNA, 41 bp, synthetic construct
  misc_feature 1..41: note = Description of sequence: Artificial sequence
  source 1..41: mol_type = other DNA
gcggcatgcgcgtttgcggtacctaaacgctcctaatagag

SEQ ID NO: 33 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
ctctattagggaggctcgcatgccgc

SEQ ID NO: 34 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
ctctattagggagaatcgcatgccgc

SEQ ID NO: 35 - DNA, 65 bp, synthetic construct
  misc_feature 1..65: note = Description of sequence: Artificial sequence
  source 1..65: mol_type = other DNA
tggatatctcctagccagactgttatgtgtgatacggctaacttcgtgatgcctctacgggtccg

SEQ ID NO: 36 - DNA, 65 bp, synthetic construct
  misc_feature 1..65: note = Description of sequence: Artificial sequence
  source 1..65: mol_type = other DNA
tggatatctcctagccagactgttatgtgtgatacggcgaacttcgtgatgcctctacgggtccg

SEQ ID NO: 37 - DNA, 65 bp, synthetic construct
  misc_feature 1..65: note = Description of sequence: Artificial sequence
  source 1..65: mol_type = other DNA
tggatatctcctagccagactgttatgtgtgatacggcacacttcgtgatgcctctacgggtccg

SEQ ID NO: 38 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatccctctacgggtccg

SEQ ID NO: 39 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatggctctacgggtccg

SEQ ID NO: 40 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgcccctacgggtccg

SEQ ID NO: 41 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
cggacccgtaccaatctcacgtataggatatcca

SEQ ID NO: 42 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
cggacccgtatccatctcacgtataggatatcca

SEQ ID NO: 43 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
cggacccgtatcattctcacgtataggatatcca

SEQ ID NO: 44 - DNA, 26 bp, synthetic construct
  misc_feature 1..26: note = Description of sequence: Artificial sequence
  source 1..26: mol_type = other DNA
agcttctttctaatacggcttaccac

SEQ ID NO: 45 - DNA, 37 bp, synthetic construct
  misc_feature 1..37: note = Description of sequence: Artificial sequence
  source 1..37: mol_type = other DNA
gtggtaagcctgggcctctttctttttaagaaagaac

SEQ ID NO: 46 - DNA, 37 bp, synthetic construct
  misc_feature 1..37: note = Description of sequence: Artificial sequence
  source 1..37: mol_type = other DNA
gtggtaagccacccggtctttctttttaagaaagatg

SEQ ID NO: 47 - DNA, 24 bp, synthetic construct
  misc_feature 1..24: note = Description of sequence: Artificial sequence
  source 1..24: mol_type = other DNA
  misc_feature 1..9: note = DNA
  misc_feature 10: note = RNA
  misc_feature 11..24: note = DNA
  misc_feature 11^12: note = phosphorothioate linkage
agtcactataggaagatggcgaaa

SEQ ID NO: 48 - DNA, 30 bp, synthetic construct
  misc_feature 1..30: note = Description of sequence: Artificial sequence
  source 1..30: mol_type = other DNA
tttcgccatcttcaccaggaaatagtgact

SEQ ID NO: 49 - DNA, 30 bp, synthetic construct
  misc_feature 1..30: note = Description of sequence: Artificial sequence
  source 1..30: mol_type = other DNA
tttcgccatcttctggtccttatagtgact

SEQ ID NO: 50 - DNA, 30 bp, synthetic construct
  misc_feature 1..30: note = Description of sequence: Artificial sequence
  source 1..30: mol_type = other DNA
tttcgccatcttcaaaaaaaaatagtgact

SEQ ID NO: 51 - DNA, 30 bp, synthetic construct
  misc_feature 1..30: note = Description of sequence: Artificial sequence
  source 1..30: mol_type = other DNA
tttcgccatcttcttttttttatagtgact

SEQ ID NO: 52 - DNA, 30 bp, synthetic construct
  misc_feature 1..30: note = Description of sequence: Artificial sequence
  source 1..30: mol_type = other DNA
  variation 14..22: note = n is a, c, g, or t
tttcgccatcttcnnnnnnnnatagtgact

SEQ ID NO: 53 - DNA, 24 bp, synthetic construct
  misc_feature 1..24: note = Description of sequence: Artificial sequence
  source 1..24: mol_type = other DNA
tttcgccatcttcctatagtgact

SEQ ID NO: 54 - DNA, 27 bp, synthetic construct
  misc_feature 1..27: note = Description of sequence: Artificial sequence
  source 1..27: mol_type = other DNA
  modified_base 27: mod_base = OTHER; note = N is cyanine 5-Aminoallyluridine-5'-Triphosphate
agcttctttctaatacggcttaccacn

SEQ ID NO: 55 - DNA, 27 bp, synthetic construct
  misc_feature 1..27: note = Description of sequence: Artificial sequence
  source 1..27: mol_type = other DNA
  modified_base 1: mod_base = OTHER; note = nucleotide bonded to quencher, quencher name: 5' Iowa Black RQ
  modified_base 27: mod_base = OTHER; note = N is cyanine 5-Aminoallyluridine-5'-Triphosphate
agcttctttctaatacggcttaccacn

SEQ ID NO: 56 - DNA, 37 bp, synthetic construct; identical to SEQ ID NO: 45
  misc_feature 1..37: note = Description of sequence: Artificial sequence
  source 1..37: mol_type = other DNA
  modified_base 1: mod_base = OTHER; note = nucleotide bonded to quencher, quencher name: 5' Iowa Black RQ

SEQ ID NO: 57 - DNA, 37 bp, synthetic construct; identical to SEQ ID NO: 46
  misc_feature 1..37: note = Description of sequence: Artificial sequence
  source 1..37: mol_type = other DNA
  modified_base 1: mod_base = OTHER; note = nucleotide bonded to quencher, quencher name: 5' Iowa Black RQ

SEQ ID NO: 58 - DNA, 24 bp, synthetic construct
  misc_feature 1..24: note = Description of sequence: Artificial sequence
  source 1..24: mol_type = other DNA
  misc_feature 1..9: note = DNA
  misc_feature 10: note = RNA
  misc_feature 11..24: note = DNA
  misc_feature 11^12: note = phosphorothioate linkage
  modified_base 1: mod_base = OTHER; note = nucleotide bonded to fluorescent dye, dye name: 5'6-FAM
agtcactataggaagatggcgaaa

SEQ ID NO: 59 - DNA, 24 bp, synthetic construct
  misc_feature 1..24: note = Description of sequence: Artificial sequence
  source 1..24: mol_type = other DNA
  misc_feature 1..9: note = DNA
  misc_feature 10: note = RNA
  misc_feature 11..24: note = DNA
  misc_feature 11^12: note = phosphorothioate linkage
  modified_base 1: mod_base = OTHER; note = nucleotide bonded to fluorescent dye, dye name: 5'6-FAM
  modified_base 24: mod_base = OTHER; note = nucleotide bonded to quencher, quencher name: 3' Iowa Black FQ
agtcactataggaagatggcgaaa

SEQ ID NO: 60 - DNA, 30 bp, synthetic construct; identical to SEQ ID NO: 48
  misc_feature 1..30: note = Description of sequence: Artificial sequence
  source 1..30: mol_type = other DNA
  modified_base 30: mod_base = OTHER; note = nucleotide bonded to quencher, quencher name: 3' Iowa Black FQ

SEQ ID NO: 61 - DNA, 30 bp, synthetic construct; identical to SEQ ID NO: 51
  misc_feature 1..30: note = Description of sequence: Artificial sequence
  source 1..30: mol_type = other DNA
  modified_base 30: mod_base = OTHER; note = nucleotide bonded to quencher, quencher name: 3' Iowa Black FQ

SEQ ID NO: 62 - DNA, 30 bp, synthetic construct; identical to SEQ ID NO: 49
  misc_feature 1..30: note = Description of sequence: Artificial sequence
  source 1..30: mol_type = other DNA
  modified_base 30: mod_base = OTHER; note = nucleotide bonded to quencher, quencher name: 3' Iowa Black FQ

SEQ ID NO: 63 - DNA, 29 bp, synthetic construct
  misc_feature 1..29: note = Description of sequence: Artificial sequence
  source 1..29: mol_type = other DNA
  modified_base 1: mod_base = OTHER; note = nucleotide bonded to quencher, quencher name: 5' Iowa Black RQ
  modified_base 29: mod_base = OTHER; note = N is cyanine 5-Aminoallyluridine-5'-Triphosphate
acttcctttctaatacggcttaccacatn

SEQ ID NO: 64 - DNA, 41 bp, synthetic construct
  misc_feature 1..41: note = Description of sequence: Artificial sequence
  source 1..41: mol_type = other DNA
  modified_base 1: mod_base = OTHER; note = nucleotide bonded to quencher, quencher name: 5' Iowa Black RQ
atgtggtaagcctgggcctctttcctttttaaggaaagaac

SEQ ID NO: 65 - DNA, 41 bp, synthetic construct
  misc_feature 1..41: note = Description of sequence: Artificial sequence
  source 1..41: mol_type = other DNA
  modified_base 1: mod_base = OTHER; note = nucleotide bonded to quencher, quencher name: 5' Iowa Black RQ
atgtggtaagccacccggtctttcctttttaaggaaagatg

SEQ ID NO: 66 - DNA, 24 bp, synthetic construct
  misc_feature 1..24: note = Description of sequence: Artificial sequence
  source 1..24: mol_type = other DNA
  misc_feature 1..8: note = DNA
  misc_feature 9: note = RNA
  misc_feature 10..24: note = DNA
  misc_feature 10^11: note = phosphorothioate linkage
  modified_base 1: mod_base = OTHER; note = nucleotide bonded to fluorescent dye, dye name: 5'6-FAM
  modified_base 24: mod_base = OTHER; note = nucleotide bonded to quencher, quencher name: 3' Iowa Black FQ
agcactataggaagcatggcgacg

SEQ ID NO: 67 - DNA, 30 bp, synthetic construct
  misc_feature 1..30: note = Description of sequence: Artificial sequence
  source 1..30: mol_type = other DNA
  modified_base 30: mod_base = OTHER; note = nucleotide bonded to quencher, quencher name: 3' Iowa Black FQ
cgtcgccatgcttcaccaggaaatagtgct

SEQ ID NO: 68 - DNA, 30 bp, synthetic construct
  misc_feature 1..30: note = Description of sequence: Artificial sequence
  source 1..30: mol_type = other DNA
  modified_base 30: mod_base = OTHER; note = nucleotide bonded to quencher, quencher name: 3' Iowa Black FQ
cgtcgccatgcttcttttttttatagtgct

SEQ ID NO: 69 - DNA, 28 bp, synthetic construct
  misc_feature 1..28: note = Description of sequence: Artificial sequence
  source 1..28: mol_type = other DNA
  modified_base 1: mod_base = OTHER; note = nucleotide bonded to quencher, quencher name: 5' Iowa Black RQ
  modified_base 28: mod_base = OTHER; note = N is cyanine 5-Aminoallyluridine-5'-Triphosphate
agcttctttctaatacggcttaccactn

SEQ ID NO: 70 - DNA, 37 bp, synthetic construct
  misc_feature 1..37: note = Description of sequence: Artificial sequence
  source 1..37: mol_type = other DNA
  modified_base 1: mod_base = OTHER; note = nucleotide bonded to quencher, quencher name: 5' Iowa Black RQ
gtggtaagccacccggtctttctttttaagaaagaac

SEQ ID NO: 71 - DNA, 24 bp, synthetic construct
  misc_feature 1..24: note = Description of sequence: Artificial sequence
  source 1..24: mol_type = other DNA
  misc_feature 1..9: note = DNA
  misc_feature 10: note = RNA
  misc_feature 11..24: note = DNA
  misc_feature 11^12: note = phosphorothioate linkage
  modified_base 1: mod_base = OTHER; note = nucleotide bonded to fluorescent dye, dye name: 5'6-FAM
  modified_base 24: mod_base = OTHER; note = nucleotide is bonded to a quencher, quencher name: 3' Dabcyl
agtcactataggaagatggcgaaa

SEQ ID NO: 72 - DNA, 24 bp, synthetic construct
  misc_feature 1..24: note = Description of sequence: Artificial sequence
  source 1..24: mol_type = other DNA
  modified_base 1: mod_base = OTHER; note = nucleotide bonded to fluorescent dye, dye name: 5'6-FAM
  modified_base 24: mod_base = OTHER; note = nucleotide is bonded to a quencher, quencher name: 3' Dabcyl
agtcactataggaagatggcgaaa

SEQ ID NO: 73 - DNA, 30 bp, synthetic construct; identical to SEQ ID NO: 48
  misc_feature 1..30: note = Description of sequence: Artificial sequence
  source 1..30: mol_type = other DNA
  modified_base 30: mod_base = OTHER; note = nucleotide is bonded to a quencher, quencher name: 3' Dabcyl

SEQ ID NO: 74 - DNA, 34 bp, synthetic construct; identical to SEQ ID NO: 20
  misc_feature 1..34: note = Description of sequence: Artificial sequence
  source 1..34: mol_type = other DNA

SEQ ID NO: 75 - DNA, 34 bp, synthetic construct; identical to SEQ ID NO: 20
  misc_feature 1..34: note = Description of sequence: Artificial sequence
  source 1..34: mol_type = other DNA
  modified_base 25: mod_base = OTHER; note = 2'O-methyl-adenosine

SEQ ID NO: 76 - DNA, 26 bp, synthetic construct
  misc_feature 1..26: note = Description of sequence: Artificial sequence
  source 1..26: mol_type = other DNA
ctctattagggagactcgcatgccgc

SEQ ID NO: 77 - DNA, 26 bp, synthetic construct
  misc_feature 1..26: note = Description of sequence: Artificial sequence
  source 1..26: mol_type = other DNA
  modified_base 9: mod_base = gm
ctctattagggagactcgcatgccgc

SEQ ID NO: 78 - DNA, 34 bp, synthetic construct; identical to SEQ ID NO: 20
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM

SEQ ID NO: 79 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtaacaatctcacgtataggatatcca

SEQ ID NO: 80 - DNA, 34 bp, synthetic construct; identical to SEQ ID NO: 41
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM

SEQ ID NO: 81 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtagcaatctcacgtataggatatcca

SEQ ID NO: 82 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtataaatctcacgtataggatatcca

SEQ ID NO: 83 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtattaatctcacgtataggatatcca

SEQ ID NO: 84 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatgaatctcacgtataggatatcca

SEQ ID NO: 85 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatctatctcacgtataggatatcca

SEQ ID NO: 86 - DNA, 34 bp, synthetic construct; identical to SEQ ID NO: 42
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM

SEQ ID NO: 87 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcgatctcacgtataggatatcca

SEQ ID NO: 88 - DNA, 34 bp, synthetic construct; identical to SEQ ID NO: 43
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM

SEQ ID NO: 89 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcactctcacgtataggatatcca

SEQ ID NO: 90 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcagtctcacgtataggatatcca

SEQ ID NO: 91 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaaactcacgtataggatatcca

SEQ ID NO: 92 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaacctcacgtataggatatcca

SEQ ID NO: 93 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaagctcacgtataggatatcca

SEQ ID NO: 94 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatatcacgtataggatatcca

SEQ ID NO: 95 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatttcacgtataggatatcca

SEQ ID NO: 96 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatgtcacgtataggatatcca

SEQ ID NO: 97 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgaataggatatcca

SEQ ID NO: 98 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgcataggatatcca

SEQ ID NO: 99 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacggataggatatcca

SEQ ID NO: 100 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtttaggatatcca

SEQ ID NO: 101 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtctaggatatcca

SEQ ID NO: 102 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtgtaggatatcca

SEQ ID NO: 103 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtaaaggatatcca

SEQ ID NO: 104 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtacaggatatcca

SEQ ID NO: 105 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtagaggatatcca

SEQ ID NO: 106 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 25: note = t is uracil at this position
  misc_feature 26..34: note = DNA
  modified_base 1: mod_base = OTHER; note = nucleotide bonded to fluorescent dye, dye name: 5'6-FAM
cggacccgtatcaatctcacgtattggatatcca

SEQ ID NO: 107 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtatcggatatcca

SEQ ID NO: 108 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtatgggatatcca

SEQ ID NO: 109 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtataagatatcca

SEQ ID NO: 110 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtatatgatatcca

SEQ ID NO: 111 - DNA, 34 bp, synthetic construct
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 1: mod_base = OTHER; note = nucleotide bonded to fluorescent dye, dye name: 5'6-FAM
cggacccgtatcaatctcacgtatacgatatcca

SEQ ID NO: 112 - DNA, 65 bp, synthetic construct
  misc_feature 1..65: note = Description of sequence: Artificial sequence
  source 1..65: mol_type = other DNA
tggatatcacctagccagactgttatgtgtgatacggcaaacttcgtgatgcctctacgggtccg

SEQ ID NO: 113 - DNA, 65 bp, synthetic construct
  misc_feature 1..65: note = Description of sequence: Artificial sequence
  source 1..65: mol_type = other DNA
tggatatcccctagccagactgttatgtgtgatacggcaaacttcgtgatgcctctacgggtccg

SEQ ID NO: 114 - DNA, 65 bp, synthetic construct
  misc_feature 1..65: note = Description of sequence: Artificial sequence
  source 1..65: mol_type = other DNA
tggatatcgcctagccagactgttatgtgtgatacggcaaacttcgtgatgcctctacgggtccg

SEQ ID NO: 115 - DNA, 65 bp, synthetic construct
  misc_feature 1..65: note = Description of sequence: Artificial sequence
  source 1..65: mol_type = other DNA
tggatatctgctagccagactgttatgtgtgatacggcaaacttcgtgatgcctctacgggtccg

SEQ ID NO: 116 - DNA, 65 bp, synthetic construct
  misc_feature 1..65: note = Description of sequence: Artificial sequence
  source 1..65: mol_type = other DNA
tggatatcttctagccagactgttatgtgtgatacggcaaacttcgtgatgcctctacgggtccg

SEQ ID NO: 117 - DNA, 65 bp, synthetic construct
  misc_feature 1..65: note = Description of sequence: Artificial sequence
  source 1..65: mol_type = other DNA
tggatatctactagccagactgttatgtgtgatacggcaaacttcgtgatgcctctacgggtccg

SEQ ID NO: 118 - DNA, 65 bp, synthetic construct
  misc_feature 1..65: note = Description of sequence: Artificial sequence
  source 1..65: mol_type = other DNA
tggatatctccttgccagactgttatgtgtgatacggcaaacttcgtgatgcctctacgggtccg

SEQ ID NO: 119 - DNA, 65 bp, synthetic construct
  misc_feature 1..65: note = Description of sequence: Artificial sequence
  source 1..65: mol_type = other DNA
tggatatctcctcgccagactgttatgtgtgatacggcaaacttcgtgatgcctctacgggtccg

SEQ ID NO: 120 - DNA, 65 bp, synthetic construct
  misc_feature 1..65: note = Description of sequence: Artificial sequence
  source 1..65: mol_type = other DNA
tggatatctcctaaccagactgttatgtgtgatacggcaaacttcgtgatgcctctacgggtccg

SEQ ID NO: 121 - DNA, 65 bp, synthetic construct
  misc_feature 1..65: note = Description of sequence: Artificial sequence
  source 1..65: mol_type = other DNA
tggatatctcctacccagactgttatgtgtgatacggcaaacttcgtgatgcctctacgggtccg

SEQ ID NO: 122 - DNA, 65 bp, synthetic construct
  misc_feature 1..65: note = Description of sequence: Artificial sequence
  source 1..65: mol_type = other DNA
tggatatctcctatccagactgttatgtgtgatacggcaaacttcgtgatgcctctacgggtccg

SEQ ID NO: 123 - DNA, 65 bp, synthetic construct
  misc_feature 1..65: note = Description of sequence: Artificial sequence
  source 1..65: mol_type = other DNA
tggatatctcctaggcagactgttatgtgtgatacggcaaacttcgtgatgcctctacgggtccg

SEQ ID NO: 124 - DNA, 65 bp, synthetic construct; identical to SEQ ID NO: 29
  misc_feature 1..65: note = Description of sequence: Artificial sequence
  source 1..65: mol_type = other DNA

SEQ ID NO: 125 - DNA, 65 bp, synthetic construct
  misc_feature 1..65: note = Description of sequence: Artificial sequence
  source 1..65: mol_type = other DNA
tggatatctcctagacagactgttatgtgtgatacggcaaacttcgtgatgcctctacgggtccg

SEQ ID NO: 126 - DNA, 65 bp, synthetic construct
  misc_feature 1..65: note = Description of sequence: Artificial sequence
  source 1..65: mol_type = other DNA
tggatatctcctagcgagactgttatgtgtgatacggcaaacttcgtgatgcctctacgggtccg

SEQ ID NO: 127 - DNA, 65 bp, synthetic construct
  misc_feature 1..65: note = Description of sequence: Artificial sequence
  source 1..65: mol_type = other DNA
tggatatctcctagctagactgttatgtgtgatacggcaaacttcgtgatgcctctacgggtccg

SEQ ID NO: 128 - DNA, 65 bp, synthetic construct
  misc_feature 1..65: note = Description of sequence: Artificial sequence
  source 1..65: mol_type = other DNA
tggatatctcctagcaagactgttatgtgtgatacggcaaacttcgtgatgcctctacgggtccg

SEQ ID NO: 129 - DNA, 65 bp, synthetic construct
  misc_feature 1..65: note = Description of sequence: Artificial sequence
  source 1..65: mol_type = other DNA
tggatatctcctagccaggctgttatgtgtgatacggcaaacttcgtgatgcctctacgggtccg

SEQ ID NO: 130 - DNA, 65 bp, synthetic construct
  misc_feature 1..65: note = Description of sequence: Artificial sequence
  source 1..65: mol_type = other DNA
tggatatctcctagccagtctgttatgtgtgatacggcaaacttcgtgatgcctctacgggtccg

SEQ ID NO: 131 - DNA, 65 bp, synthetic construct
  misc_feature 1..65: note = Description of sequence: Artificial sequence
  source 1..65: mol_type = other DNA
tggatatctcctagccagcctgttatgtgtgatacggcaaacttcgtgatgcctctacgggtccg

SEQ ID NO: 132 - DNA, 65 bp, synthetic construct
  misc_feature 1..65: note = Description of sequence: Artificial sequence
  source 1..65: mol_type = other DNA
tggatatctcctagccagagtgttatgtgtgatacggcaaacttcgtgatgcctctacgggtccg

SEQ ID NO: 133 - DNA, 65 bp, synthetic construct
  misc_feature 1..65: note = Description of sequence: Artificial sequence
  source 1..65: mol_type = other DNA
tggatatctcctagccagattgttatgtgtgatacggcaaacttcgtgatgcctctacgggtccg

SEQ ID NO: 134 - DNA, 65 bp, synthetic construct
  misc_feature 1..65: note = Description of sequence: Artificial sequence
  source 1..65: mol_type = other DNA
tggatatctcctagccagaatgttatgtgtgatacggcaaacttcgtgatgcctctacgggtccg

SEQ ID NO: 135 - DNA, 65 bp, synthetic construct
  misc_feature 1..65: note = Description of sequence: Artificial sequence
  source 1..65: mol_type = other DNA
tggatatctcctagccagacagttatgtgtgatacggcaaacttcgtgatgcctctacgggtccg

SEQ ID NO: 136 - DNA, 65 bp, synthetic construct
  misc_feature 1..65: note = Description of sequence: Artificial sequence
  source 1..65: mol_type = other DNA
tggatatctcctagccagaccgttatgtgtgatacggcaaacttcgtgatgcctctacgggtccg

SEQ ID NO: 137 - DNA, 65 bp, synthetic construct
  misc_feature 1..65: note = Description of sequence: Artificial sequence
  source 1..65: mol_type = other DNA
tggatatctcctagccagacggttatgtgtgatacggcaaacttcgtgatgcctctacgggtccg

SEQ ID NO: 138 - DNA, 65 bp, synthetic construct
  misc_feature 1..65: note = Description of sequence: Artificial sequence
  source 1..65: mol_type = other DNA
tggatatctcctagccagactgttgtgtgtgatacggcaaacttcgtgatgcctctacgggtccg

SEQ ID NO: 139 - DNA, 65 bp, synthetic construct
  misc_feature 1..65: note = Description of sequence: Artificial sequence
  source 1..65: mol_type = other DNA
tggatatctcctagccagactgttttgtgtgatacggcaaacttcgtgatgcctctacgggtccg

SEQ ID NO: 140 - DNA, 65 bp, synthetic construct
  misc_feature 1..65: note = Description of sequence: Artificial sequence
  source 1..65: mol_type = other DNA
tggatatctcctagccagactgttctgtgtgatacggcaaacttcgtgatgcctctacgggtccg

SEQ ID NO: 141 - DNA, 65 bp, synthetic construct
  misc_feature 1..65: note = Description of sequence: Artificial sequence
  source 1..65: mol_type = other DNA
tggatatctcctagccagactgttaagtgtgatacggcaaacttcgtgatgcctctacgggtccg

SEQ ID NO: 142 - DNA, 65 bp, synthetic construct
  misc_feature 1..65: note = Description of sequence: Artificial sequence
  source 1..65: mol_type = other DNA
tggatatctcctagccagactgttacgtgtgatacggcaaacttcgtgatgcctctacgggtccg

SEQ ID NO: 143 - DNA, 65 bp, synthetic construct
  misc_feature 1..65: note = Description of sequence: Artificial sequence
  source 1..65: mol_type = other DNA
tggatatctcctagccagactgttaggtgtgatacggcaaacttcgtgatgcctctacgggtccg

SEQ ID NO: 144 - DNA, 65 bp, synthetic construct
  misc_feature 1..65: note = Description of sequence: Artificial sequence
  source 1..65: mol_type = other DNA
tggatatctcctagccagactgttatgtatgatacggcaaacttcgtgatgcctctacgggtccg

SEQ ID NO: 145 - DNA, 65 bp, synthetic construct
  misc_feature 1..65: note = Description of sequence: Artificial sequence
  source 1..65: mol_type = other DNA
tggatatctcctagccagactgttatgtctgatacggcaaacttcgtgatgcctctacgggtccg

SEQ ID NO: 146 - DNA, 65 bp, synthetic construct
  misc_feature 1..65: note = Description of sequence: Artificial sequence
  source 1..65: mol_type = other DNA
tggatatctcctagccagactgttatgtttgatacggcaaacttcgtgatgcctctacgggtccg

SEQ ID NO: 147 - DNA, 65 bp, synthetic construct
  misc_feature 1..65: note = Description of sequence: Artificial sequence
  source 1..65: mol_type = other DNA
tggatatctcctagccagactgttatgtgagatacggcaaacttcgtgatgcctctacgggtccg

SEQ ID NO: 148 - DNA, 65 bp, synthetic construct
  misc_feature 1..65: note = Description of sequence: Artificial sequence
  source 1..65: mol_type = other DNA
tggatatctcctagccagactgttatgtgcgatacggcaaacttcgtgatgcctctacgggtccg

SEQ ID NO: 149 - DNA, 65 bp, synthetic construct
  misc_feature 1..65: note = Description of sequence: Artificial sequence
  source 1..65: mol_type = other DNA
tggatatctcctagccagactgttatgtgggatacggcaaacttcgtgatgcctctacgggtccg

SEQ ID NO: 150 - DNA, 65 bp, synthetic construct
  misc_feature 1..65: note = Description of sequence: Artificial sequence
  source 1..65: mol_type = other DNA
tggatatctcctagccagactgttatgtgtaatacggcaaacttcgtgatgcctctacgggtccg

SEQ ID NO: 151 - DNA, 65 bp, synthetic construct
  misc_feature 1..65: note = Description of sequence: Artificial sequence
  source 1..65: mol_type = other DNA
tggatatctcctagccagactgttatgtgtcatacggcaaacttcgtgatgcctctacgggtccg

SEQ ID NO: 152 - DNA, 65 bp, synthetic construct
  misc_feature 1..65: note = Description of sequence: Artificial sequence
  source 1..65: mol_type = other DNA
tggatatctcctagccagactgttatgtgttatacggcaaacttcgtgatgcctctacgggtccg

SEQ ID NO: 153 - DNA, 65 bp, synthetic construct
  misc_feature 1..65: note = Description of sequence: Artificial sequence
  source 1..65: mol_type = other DNA
tggatatctcctagccagactgttatgtgtggtacggcaaacttcgtgatgcctctacgggtccg

SEQ ID NO: 154 - DNA, 65 bp, synthetic construct
  misc_feature 1..65: note = Description of sequence: Artificial sequence
  source 1..65: mol_type = other DNA
tggatatctcctagccagactgttatgtgtgttacggcaaacttcgtgatgcctctacgggtccg

SEQ ID NO: 155 - DNA, 65 bp, synthetic construct
  misc_feature 1..65: note = Description of sequence: Artificial sequence
  source 1..65: mol_type = other DNA
tggatatctcctagccagactgttatgtgtgctacggcaaacttcgtgatgcctctacgggtccg

SEQ ID NO: 156 - DNA, 65 bp, synthetic construct
  misc_feature 1..65: note = Description of sequence: Artificial sequence
  source 1..65: mol_type = other DNA
tggatatctcctagccagactgttatgtgtgaaacggcaaacttcgtgatgcctctacgggtccg

SEQ ID NO: 157 - DNA, 65 bp, synthetic construct
  misc_feature 1..65: note = Description of sequence: Artificial sequence
  source 1..65: mol_type = other DNA
tggatatctcctagccagactgttatgtgtgacacggcaaacttcgtgatgcctctacgggtccg

SEQ ID NO: 158 - DNA, 65 bp, synthetic construct
  misc_feature 1..65: note = Description of sequence: Artificial sequence
  source 1..65: mol_type = other DNA
tggatatctcctagccagactgttatgtgtgagacggcaaacttcgtgatgcctctacgggtccg

SEQ ID NO: 159 - DNA, 65 bp, synthetic construct
  misc_feature 1..65: note = Description of sequence: Artificial sequence
  source 1..65: mol_type = other DNA
tggatatctcctagccagactgttatgtgtgatacagcaaacttcgtgatgcctctacgggtccg

SEQ ID NO: 160 - DNA, 65 bp, synthetic construct
  misc_feature 1..65: note = Description of sequence: Artificial sequence
  source 1..65: mol_type = other DNA
tggatatctcctagccagactgttatgtgtgataccgcaaacttcgtgatgcctctacgggtccg

SEQ ID NO: 161 - DNA, 65 bp, synthetic construct
  misc_feature 1..65: note = Description of sequence: Artificial sequence
  source 1..65: mol_type = other DNA
tggatatctcctagccagactgttatgtgtgatactgcaaacttcgtgatgcctctacgggtccg

SEQ ID NO: 162 - DNA, 65 bp, synthetic construct
  misc_feature 1..65: note = Description of sequence: Artificial sequence
  source 1..65: mol_type = other DNA
tggatatctcctagccagactgttatgtgtgatacgacaaacttcgtgatgcctctacgggtccg

SEQ ID NO: 163 - DNA, 65 bp, synthetic construct
  misc_feature 1..65: note = Description of sequence: Artificial sequence
  source 1..65: mol_type = other DNA
tggatatctcctagccagactgttatgtgtgatacgccaaacttcgtgatgcctctacgggtccg

SEQ ID NO: 164 - DNA, 65 bp, synthetic construct
  misc_feature 1..65: note = Description of sequence: Artificial sequence
  source 1..65: mol_type = other DNA
tggatatctcctagccagactgttatgtgtgatacgtcaaacttcgtgatgcctctacgggtccg

SEQ ID NO: 165 - DNA, 65 bp, synthetic construct
  misc_feature 1..65: note = Description of sequence: Artificial sequence
  source 1..65: mol_type = other DNA
tggatatctcctagccagactgttatgtgtgatacgggaaacttcgtgatgcctctacgggtccg

SEQ ID NO: 166 - DNA, 65 bp, synthetic construct
  misc_feature 1..65: note = Description of sequence: Artificial sequence
  source 1..65: mol_type = other DNA
tggatatctcctagccagactgttatgtgtgatacggtaaacttcgtgatgcctctacgggtccg

SEQ ID NO: 167 - DNA, 65 bp, synthetic construct
  misc_feature 1..65: note = Description of sequence: Artificial sequence
  source 1..65: mol_type = other DNA
tggatatctcctagccagactgttatgtgtgatacggaaaacttcgtgatgcctctacgggtccg

SEQ ID NO: 168 - DNA, 65 bp, synthetic construct
  misc_feature 1..65: note = Description of sequence: Artificial sequence
  source 1..65: mol_type = other DNA
tggatatctcctagccagactgttatgtgtgatacggccaacttcgtgatgcctctacgggtccg

SEQ ID NO: 169 - DNA, 65 bp, synthetic construct
  misc_feature 1..65: note = Description of sequence: Artificial sequence
  source 1..65: mol_type = other DNA
tggatatctcctagccagactgttatgtgtgatacggcagacttcgtgatgcctctacgggtccg

SEQ ID NO: 170 - DNA, 65 bp, synthetic construct
  misc_feature 1..65: note = Description of sequence: Artificial sequence
  source 1..65: mol_type = other DNA
tggatatctcctagccagactgttatgtgtgatacggcatacttcgtgatgcctctacgggtccg

SEQ ID NO: 171 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacatcgtgatgcctctacgggtccg

SEQ ID NO: 172 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacctcgtgatgcctctacgggtccg

SEQ ID NO: 173 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacgtcgtgatgcctctacgggtccg

SEQ ID NO: 174 - DNA, 65 bp, synthetic construct
  misc_feature 1..65: note = Description of sequence: Artificial sequence
  source 1..65: mol_type = other DNA
tggatatctcctagccagactgttatgtgtgatacggcaaactacgtgatgcctctacgggtccg

SEQ ID NO: 175 - DNA, 65 bp, synthetic construct
  misc_feature 1..65: note = Description of sequence: Artificial sequence
  source 1..65: mol_type = other DNA
tggatatctcctagccagactgttatgtgtgatacggcaaactccgtgatgcctctacgggtccg

SEQ ID NO: 176 - DNA, 65 bp, synthetic construct
  misc_feature 1..65: note = Description of sequence: Artificial sequence
  source 1..65: mol_type = other DNA
tggatatctcctagccagactgttatgtgtgatacggcaaactgcgtgatgcctctacgggtccg

SEQ ID NO: 177 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgaagcctctacgggtccg

SEQ ID NO: 178 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgacgcctctacgggtccg

SEQ ID NO: 179 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgaggcctctacgggtccg

SEQ ID NO: 180 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatacctctacgggtccg

SEQ ID NO: 181 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgattcctctacgggtccg

SEQ ID NO: 182 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgtctctacgggtccg

SEQ ID NO: 183 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgactctacgggtccg

SEQ ID NO: 184 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgcgtctacgggtccg

SEQ ID NO: 185 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgcttctacgggtccg

SEQ ID NO: 186 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgcatctacgggtccg

SEQ ID NO: 187 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgccgctacgggtccg

SEQ ID NO: 188 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgccactacgggtccg

SEQ ID NO: 189 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgcctgtacgggtccg

SEQ ID NO: 190 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgcctttacgggtccg

SEQ ID NO: 191 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatgcctatacgggtccg

SEQ ID NO: 192 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggagactcgcatgccgc

SEQ ID NO: 193 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattaggaagactcgcatgccgc

SEQ ID NO: 194 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattaggtagactcgcatgccgc

SEQ ID NO: 195 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattaggcagactcgcatgccgc

SEQ ID NO: 196 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggtgactcgcatgccgc

SEQ ID NO: 197 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggcgactcgcatgccgc

SEQ ID NO: 198 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggggactcgcatgccgc

SEQ ID NO: 199 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggaaactcgcatgccgc

SEQ ID NO: 200 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggatactcgcatgccgc

SEQ ID NO: 201 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggacactcgcatgccgc

SEQ ID NO: 202 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggagtctcgcatgccgc

SEQ ID NO: 203 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggagcctcgcatgccgc

SEQ ID NO: 204 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggaggctcgcatgccgc

SEQ ID NO: 205 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggagaatcgcatgccgc

SEQ ID NO: 206 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggagattcgcatgccgc

SEQ ID NO: 207 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggagagtcgcatgccgc

SEQ ID NO: 208 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggagacacgcatgccgc

SEQ ID NO: 209 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggagacccgcatgccgc

SEQ ID NO: 210 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 1: mod_base = OTHER; note = nucleotide bonded to fluorescent dye, dye name: 5'6-FAM
ctctattagggagacgcgcatgccgc

SEQ ID NO: 211 - DNA, 41 bp, synthetic construct
  misc_feature 1..41: note = Description of sequence: Artificial sequence
  source 1..41: mol_type = other DNA
gcggcatgcgcgtttacggcacctaaacgctcctaatagag

SEQ ID NO: 212 - DNA, 41 bp, synthetic construct
  misc_feature 1..41: note = Description of sequence: Artificial sequence
  source 1..41: mol_type = other DNA
gcggcatgcgcgtttccggcacctaaacgctcctaatagag

SEQ ID NO: 213 - DNA, 41 bp, synthetic construct
  misc_feature 1..41: note = Description of sequence: Artificial sequence
  source 1..41: mol_type = other DNA
gcggcatgcgcgttttcggcacctaaacgctcctaatagag

SEQ ID NO: 214 - DNA, 41 bp, synthetic construct
  misc_feature 1..41: note = Description of sequence: Artificial sequence
  source 1..41: mol_type = other DNA
gcggcatgcgcgtttggggcacctaaacgctcctaatagag

SEQ ID NO: 215 - DNA, 41 bp, synthetic construct
  misc_feature 1..41: note = Description of sequence: Artificial sequence
  source 1..41: mol_type = other DNA
gcggcatgcgcgtttgtggcacctaaacgctcctaatagag

SEQ ID NO: 216 - DNA, 41 bp, synthetic construct
  misc_feature 1..41: note = Description of sequence: Artificial sequence
  source 1..41: mol_type = other DNA
gcggcatgcgcgtttgaggcacctaaacgctcctaatagag

SEQ ID NO: 217 - DNA, 41 bp, synthetic construct
  misc_feature 1..41: note = Description of sequence: Artificial sequence
  source 1..41: mol_type = other DNA
gcggcatgcgcgtttgcagcacctaaacgctcctaatagag

SEQ ID NO: 218 - DNA, 41 bp, synthetic construct
  misc_feature 1..41: note = Description of sequence: Artificial sequence
  source 1..41: mol_type = other DNA
gcggcatgcgcgtttgctgcacctaaacgctcctaatagag

SEQ ID NO: 219 - DNA, 41 bp, synthetic construct
  misc_feature 1..41: note = Description of sequence: Artificial sequence
  source 1..41: mol_type = other DNA
gcggcatgcgcgtttgccgcacctaaacgctcctaatagag

SEQ ID NO: 220 - DNA, 41 bp, synthetic construct
  misc_feature 1..41: note = Description of sequence: Artificial sequence
  source 1..41: mol_type = other DNA
gcggcatgcgcgtttgcgacacctaaacgctcctaatagag

SEQ ID NO: 221 - DNA, 41 bp, synthetic construct
  misc_feature 1..41: note = Description of sequence: Artificial sequence
  source 1..41: mol_type = other DNA
gcggcatgcgcgtttgcgccacctaaacgctcctaatagag

SEQ ID NO: 222 - DNA, 41 bp, synthetic construct
  misc_feature 1..41: note = Description of sequence: Artificial sequence
  source 1..41: mol_type = other DNA
gcggcatgcgcgtttgcgtcacctaaacgctcctaatagag

SEQ ID NO: 223 - DNA, 41 bp, synthetic construct
  misc_feature 1..41: note = Description of sequence: Artificial sequence
  source 1..41: mol_type = other DNA
gcggcatgcgcgtttgcgggacctaaacgctcctaatagag

SEQ ID NO: 224 - DNA, 41 bp, synthetic construct
  misc_feature 1..41: note = Description of sequence: Artificial sequence
  source 1..41: mol_type = other DNA
gcggcatgcgcgtttgcggaacctaaacgctcctaatagag

SEQ ID NO: 225 - DNA, 41 bp, synthetic construct
  misc_feature 1..41: note = Description of sequence: Artificial sequence
  source 1..41: mol_type = other DNA
gcggcatgcgcgtttgcggcgcctaaacgctcctaatagag

SEQ ID NO: 226 - DNA, 41 bp, synthetic construct
  misc_feature 1..41: note = Description of sequence: Artificial sequence
  source 1..41: mol_type = other DNA
gcggcatgcgcgtttgcggcccctaaacgctcctaatagag

SEQ ID NO: 227 - DNA, 41 bp, synthetic construct
  misc_feature 1..41: note = Description of sequence: Artificial sequence
  source 1..41: mol_type = other DNA
gcggcatgcgcgtttgcggctcctaaacgctcctaatagag

SEQ ID NO: 228 - DNA, 41 bp, synthetic construct
  misc_feature 1..41: note = Description of sequence: Artificial sequence
  source 1..41: mol_type = other DNA
gcggcatgcgcgtttgcggcagctaaacgctcctaatagag

SEQ ID NO: 229 - DNA, 41 bp, synthetic construct
  misc_feature 1..41: note = Description of sequence: Artificial sequence
  source 1..41: mol_type = other DNA
gcggcatgcgcgtttgcggcatctaaacgctcctaatagag

SEQ ID NO: 230 - DNA, 41 bp, synthetic construct
  misc_feature 1..41: note = Description of sequence: Artificial sequence
  source 1..41: mol_type = other DNA
gcggcatgcgcgtttgcggcaactaaacgctcctaatagag

SEQ ID NO: 231 - DNA, 41 bp, synthetic construct
  misc_feature 1..41: note = Description of sequence: Artificial sequence
  source 1..41: mol_type = other DNA
gcggcatgcgcgtttgcggcacgtaaacgctcctaatagag

SEQ ID NO: 232 - DNA, 41 bp, synthetic construct
  misc_feature 1..41: note = Description of sequence: Artificial sequence
  source 1..41: mol_type = other DNA
gcggcatgcgcgtttgcggcacttaaacgctcctaatagag

SEQ ID NO: 233 - DNA, 41 bp, synthetic construct
  misc_feature 1..41: note = Description of sequence: Artificial sequence
  source 1..41: mol_type = other DNA
gcggcatgcgcgtttgcggcacataaacgctcctaatagag

SEQ ID NO: 234 - DNA, 41 bp, synthetic construct
  misc_feature 1..41: note = Description of sequence: Artificial sequence
  source 1..41: mol_type = other DNA
gcggcatgcgcgtttgcggcaccgaaacgctcctaatagag

SEQ ID NO: 235 - DNA, 41 bp, synthetic construct
  misc_feature 1..41: note = Description of sequence: Artificial sequence
  source 1..41: mol_type = other DNA
gcggcatgcgcgtttgcggcacccaaacgctcctaatagag

SEQ ID NO: 236 - DNA, 41 bp, synthetic construct
  misc_feature 1..41: note = Description of sequence: Artificial sequence
  source 1..41: mol_type = other DNA
gcggcatgcgcgtttgcggcaccaaaacgctcctaatagag

SEQ ID NO: 237 - DNA, 41 bp, synthetic construct
  misc_feature 1..41: note = Description of sequence: Artificial sequence
  source 1..41: mol_type = other DNA
gcggcatgcgcgtttgcggcacctaaacggtcctaatagag

SEQ ID NO: 238 - DNA, 41 bp, synthetic construct
  misc_feature 1..41: note = Description of sequence: Artificial sequence
  source 1..41: mol_type = other DNA
gcggcatgcgcgtttgcggcacctaaacgttcctaatagag

SEQ ID NO: 239 - DNA, 41 bp, synthetic construct
  misc_feature 1..41: note = Description of sequence: Artificial sequence
  source 1..41: mol_type = other DNA
gcggcatgcgcgtttgcggcacctaaacgatcctaatagag

SEQ ID NO: 240 - DNA, 41 bp, synthetic construct
  misc_feature 1..41: note = Description of sequence: Artificial sequence
  source 1..41: mol_type = other DNA
gcggcatgcgcgtttgcggcacctaaacgcacctaatagag

SEQ ID NO: 241 - DNA, 41 bp, synthetic construct
  misc_feature 1..41: note = Description of sequence: Artificial sequence
  source 1..41: mol_type = other DNA
gcggcatgcgcgtttgcggcacctaaacgcgcctaatagag

SEQ ID NO: 242 - DNA, 64 bp, synthetic construct
  misc_feature 1..64: note = Description of sequence: Artificial sequence
  source 1..64: mol_type = other DNA
ggatatctcctagccagactgttatgtgtgatacggcaaacttcgtgatgcctctacgggtccg

SEQ ID NO: 243 - DNA, 63 bp, synthetic construct
  misc_feature 1..63: note = Description of sequence: Artificial sequence
  source 1..63: mol_type = other DNA
gatatctcctagccagactgttatgtgtgatacggcaaacttcgtgatgcctctacgggtccg

SEQ ID NO: 244 - DNA, 64 bp, synthetic construct
  misc_feature 1..64: note = Description of sequence: Artificial sequence
  source 1..64: mol_type = other DNA
tggatatctcctagccagactgttatgtgtgatacggcaaacttcgtgatgcctctacgggtcc

SEQ ID NO: 245 - DNA, 62 bp, synthetic construct
  misc_feature 1..62: note = Description of sequence: Artificial sequence
  source 1..62: mol_type = other DNA
tggatatctcctagccagactgttatgtgtgatacggcaaacttcgtgatgcctctacggtc

SEQ ID NO: 246 - DNA, 62 bp, synthetic construct
  misc_feature 1..62: note = Description of sequence: Artificial sequence
  source 1..62: mol_type = other DNA
tggatatctcctagccagactgttatgtgtgatacggcaaacttcgtgatgcctctacgggt

SEQ ID NO: 247 - DNA, 61 bp, synthetic construct
  misc_feature 1..61: note = Description of sequence: Artificial sequence
  source 1..61: mol_type = other DNA
tggatatctcctagccagactgttatgtgtgatacggcaaacttcgtgatgcctctacggg

SEQ ID NO: 248 - DNA, 40 bp, synthetic construct
  misc_feature 1..40: note = Description of sequence: Artificial sequence
  source 1..40: mol_type = other DNA
cggcatgcgcgtttgcggcacctaaacgctcctaatagag

SEQ ID NO: 249 - DNA, 39 bp, synthetic construct
  misc_feature 1..39: note = Description of sequence: Artificial sequence
  source 1..39: mol_type = other DNA
ggcatgcgcgtttgcggcacctaaacgctcctaatagag

SEQ ID NO: 250 - DNA, 38 bp, synthetic construct
  misc_feature 1..38: note = Description of sequence: Artificial sequence
  source 1..38: mol_type = other DNA
gcatgcgcgtttgcggcacctaaacgctcctaatagag

SEQ ID NO: 251 - DNA, 37 bp, synthetic construct
  misc_feature 1..37: note = Description of sequence: Artificial sequence
  source 1..37: mol_type = other DNA
catgcgcgtttgcggcacctaaacgctcctaatagag

SEQ ID NO: 252 - DNA, 36 bp, synthetic construct
  misc_feature 1..36: note = Description of sequence: Artificial sequence
  source 1..36: mol_type = other DNA
atgcgcgtttgcggcacctaaacgctcctaatagag

SEQ ID NO: 253 - DNA, 40 bp, synthetic construct
  misc_feature 1..40: note = Description of sequence: Artificial sequence
  source 1..40: mol_type = other DNA
gcggcatgcgcgtttgcggcacctaaacgctcctaataga

SEQ ID NO: 254 - DNA, 39 bp, synthetic construct
  misc_feature 1..39: note = Description of sequence: Artificial sequence
  source 1..39: mol_type = other DNA
gcggcatgcgcgtttgcggcacctaaacgctcctaatag

SEQ ID NO: 255 - DNA, 38 bp, synthetic construct
  misc_feature 1..38: note = Description of sequence: Artificial sequence
  source 1..38: mol_type = other DNA
gcggcatgcgcgtttgcggcacctaaacgctcctaata

SEQ ID NO: 256 - DNA, 65 bp, synthetic construct
  misc_feature 1..65: note = Description of sequence: Artificial sequence
  source 1..65: mol_type = other DNA
tggatatctcctagccagactgttatgtgtgatacggctaacttcgtgatggctctacgggtccg

SEQ ID NO: 257 - DNA, 65 bp, synthetic construct
  misc_feature 1..65: note = Description of sequence: Artificial sequence
  source 1..65: mol_type = other DNA
tggatatctcctagccagactgttatgtgtgatacggcacacttcgtgatggctctacgggtccg

SEQ ID NO: 258 - DNA, 65 bp, synthetic construct
  misc_feature 1..65: note = Description of sequence: Artificial sequence
  source 1..65: mol_type = other DNA
tggatatctcctagccagactgttatgtgtgatacggcacacttcgtgatgcccctacgggtccg

SEQ ID NO: 259 - DNA, 65 bp, synthetic construct
  misc_feature 1..65: note = Description of sequence: Artificial sequence
  source 1..65: mol_type = other DNA
tggatatctcctagccagactgttatgtgtgatacggctaacttcgtgatccctctacgggtccg

SEQ ID NO: 260 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatccccctacgggtccg

SEQ ID NO: 261 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatggccctacgggtccg

SEQ ID NO: 262 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatcgctctacgggtccg

SEQ ID NO: 263 - DNA, 65 bp, synthetic construct
  misc_feature 1..65: note = Description of sequence: Artificial sequence
  source 1..65: mol_type = other DNA
tggatatctcctagccagactgttatgtgtgatacggcacacttcgtgatccctctacgggtccg

SEQ ID NO: 264 - DNA, 65 bp, synthetic construct
  misc_feature 1..65: note = Description of sequence: Artificial sequence
  source 1..65: mol_type = other DNA
tggatatctcctagccagactgttatgtgtgatacggcgaacttcgtgatggctctacgggtccg

SEQ ID NO: 265 - DNA, 65 bp, synthetic construct
  misc_feature 1..65: note = Description of sequence: Artificial sequence
  source 1..65: mol_type = other DNA
tggatatctcctagccagactgttatgtgtgatacggctaacttcgtgatgcccctacgggtccg

SEQ ID NO: 266 - DNA, 65 bp, synthetic construct
  misc_feature 1..65: note = Description of sequence: Artificial sequence
  source 1..65: mol_type = other DNA
tggatatctcctagccagactgttatgtgtgatacggcgaacttcgtgatgcccctacgggtccg

SEQ ID NO: 267 - DNA, 65 bp, synthetic construct
  misc_feature 1..65: note = Description of sequence: Artificial sequence
  source 1..65: mol_type = other DNA
tggatatctcctagccagactgttatgtgtgatacggctcacttcgtgatgcctctacgggtccg

SEQ ID NO: 268 - DNA, 65 bp, synthetic construct
  misc_feature 1..65: note = Description of sequence: Artificial sequence
  source 1..65: mol_type = other DNA
tggatatctcctagccagactgttatgtgtgatacggcgcacttcgtgatgcctctacgggtccg

SEQ ID NO: 269 - DNA, 65 bp, synthetic construct
  misc_feature 1..65: note = Description of sequence: Artificial sequence
  source 1..65: mol_type = other DNA
tggatatctcctagccagactgttatgtgtgatacggcgaacttcgtgatccctctacgggtccg

SEQ ID NO: 270 - DNA, 65 bp, synthetic construct
  misc_feature 1..65: note = Description of sequence: Artificial sequence
  source 1..65: mol_type = other DNA
tggatatctcctagccagactgttatgtgtgatacggctaacttcgtgatcgctctacgggtccg

SEQ ID NO: 271 - DNA, 65 bp, synthetic construct
  misc_feature 1..65: note = Description of sequence: Artificial sequence
  source 1..65: mol_type = other DNA
tggatatctcctagccagactgttatgtgtgatacggctaacttcgtgatccccctacgggtccg

SEQ ID NO: 272 - DNA, 65 bp, synthetic construct
  misc_feature 1..65: note = Description of sequence: Artificial sequence
  source 1..65: mol_type = other DNA
tggatatctcctagccagactgttatgtgtgatacggcacacttcgtgatcgctctacgggtccg

SEQ ID NO: 273 - DNA, 65 bp, synthetic construct
  misc_feature 1..65: note = Description of sequence: Artificial sequence
  source 1..65: mol_type = other DNA
tggatatctcctagccagactgttatgtgtgatacggctaacttcgtgatggccctacgggtccg

SEQ ID NO: 274 - DNA, 65 bp, synthetic construct
  misc_feature 1..65: note = Description of sequence: Artificial sequence
  source 1..65: mol_type = other DNA
tggatatctcctagccagactgttatgtgtgatacggcgaacttcgtgatccccctacgggtccg

SEQ ID NO: 275 - DNA, 65 bp, synthetic construct
  misc_feature 1..65: note = Description of sequence: Artificial sequence
  source 1..65: mol_type = other DNA
tggatatctcctagccagactgttatgtgtgatacggcacacttcgtgatccccctacgggtccg

SEQ ID NO: 276 - DNA, 65 bp, synthetic construct
  misc_feature 1..65: note = Description of sequence: Artificial sequence
  source 1..65: mol_type = other DNA
tggatatctcctagccagactgttatgtgtgatacggcacacttcgtgatggccctacgggtccg

SEQ ID NO: 277 - DNA, 65 bp, synthetic construct
  misc_feature 1..65: note = Description of sequence: Artificial sequence
  source 1..65: mol_type = other DNA
tggatatctcctagccagactgttatgtgtgatacggcgcacttcgtgatggctctacgggtccg

SEQ ID NO: 278 - DNA, 65 bp, synthetic construct
  misc_feature 1..65: note = Description of sequence: Artificial sequence
  source 1..65: mol_type = other DNA
tggatatctcctagccagactgttatgtgtgatacggctcacttcgtgatgcccctacgggtccg

SEQ ID NO: 279 - DNA, 65 bp, synthetic construct
  misc_feature 1..65: note = Description of sequence: Artificial sequence
  source 1..65: mol_type = other DNA
tggatatctcctagccagactgttatgtgtgatacggcgcacttcgtgatccctctacgggtccg

SEQ ID NO: 280 - DNA, 65 bp, synthetic construct
  misc_feature 1..65: note = Description of sequence: Artificial sequence
  source 1..65: mol_type = other DNA
tggatatctcctagccagactgttatgtgtgatacggctcacttcgtgatccctctacgggtccg

SEQ ID NO: 281 - DNA, 65 bp, synthetic construct
  misc_feature 1..65: note = Description of sequence: Artificial sequence
  source 1..65: mol_type = other DNA
tggatatctcctagccagactgttatgtgtgatacggcgaacttcgtgatggccctacgggtccg

SEQ ID NO: 282 - DNA, 65 bp, synthetic construct
  misc_feature 1..65: note = Description of sequence: Artificial sequence
  source 1..65: mol_type = other DNA
tggatatctcctagccagactgttatgtgtgatacggctcacttcgtgatggctctacgggtccg

SEQ ID NO: 283 - DNA, 65 bp, synthetic construct
  misc_feature 1..65: note = Description of sequence: Artificial sequence
  source 1..65: mol_type = other DNA
tggatatctcctagccagactgttatgtgtgatacggcaaacttcgtgatcgccctacgggtccg

SEQ ID NO: 284 - DNA, 65 bp, synthetic construct
  misc_feature 1..65: note = Description of sequence: Artificial sequence
  source 1..65: mol_type = other DNA
tggatatctcctagccagactgttatgtgtgatacggcgaacttcgtgatcgctctacgggtccg

SEQ ID NO: 285 - DNA, 65 bp, synthetic construct
  misc_feature 1..65: note = Description of sequence: Artificial sequence
  source 1..65: mol_type = other DNA
tggatatctcctagccagactgttatgtgtgatacggcgcacttcgtgatgcccctacgggtccg

SEQ ID NO: 286 - DNA, 65 bp, synthetic construct
  misc_feature 1..65: note = Description of sequence: Artificial sequence
  source 1..65: mol_type = other DNA
tggatatctcctagccagactgttatgtgtgatacggcgcacttcgtgatcgctctacgggtccg

SEQ ID NO: 287 - DNA, 65 bp, synthetic construct
  misc_feature 1..65: note = Description of sequence: Artificial sequence
  source 1..65: mol_type = other DNA
tggatatctcctagccagactgttatgtgtgatacggctaacttcgtgatcgccctacgggtccg

SEQ ID NO: 288 - DNA, 65 bp, synthetic construct
  misc_feature 1..65: note = Description of sequence: Artificial sequence
  source 1..65: mol_type = other DNA
tggatatctcctagccagactgttatgtgtgatacggctcacttcgtgatccccctacgggtccg

SEQ ID NO: 289 - DNA, 65 bp, synthetic construct
  misc_feature 1..65: note = Description of sequence: Artificial sequence
  source 1..65: mol_type = other DNA
tggatatctcctagccagactgttatgtgtgatacggctcacttcgtgatcgctctacgggtccg

SEQ ID NO: 290 - DNA, 65 bp, synthetic construct
  misc_feature 1..65: note = Description of sequence: Artificial sequence
  source 1..65: mol_type = other DNA
tggatatctcctagccagactgttatgtgtgatacggctcacttcgtgatggccctacgggtccg

SEQ ID NO: 291 - DNA, 65 bp, synthetic construct
  misc_feature 1..65: note = Description of sequence: Artificial sequence
  source 1..65: mol_type = other DNA
tggatatctcctagccagactgttatgtgtgatacggcacacttcgtgatcgccctacgggtccg

SEQ ID NO: 292 - DNA, 65 bp, synthetic construct
  misc_feature 1..65: note = Description of sequence: Artificial sequence
  source 1..65: mol_type = other DNA
tggatatctcctagccagactgttatgtgtgatacggcgcacttcgtgatccccctacgggtccg

SEQ ID NO: 293 - DNA, 65 bp, synthetic construct
  misc_feature 1..65: note = Description of sequence: Artificial sequence
  source 1..65: mol_type = other DNA
tggatatctcctagccagactgttatgtgtgatacggcgaacttcgtgatcgccctacgggtccg

SEQ ID NO: 294 - DNA, 65 bp, synthetic construct
  misc_feature 1..65: note = Description of sequence: Artificial sequence
  source 1..65: mol_type = other DNA
tggatatctcctagccagactgttatgtgtgatacggcgcacttcgtgatggccctacgggtccg

SEQ ID NO: 295 - DNA, 65 bp, synthetic construct
  misc_feature 1..65: note = Description of sequence: Artificial sequence
  source 1..65: mol_type = other DNA
tggatatctcctagccagactgttatgtgtgatacggctcacttcgtgatcgccctacgggtccg

SEQ ID NO: 296 - DNA, 65 bp, synthetic construct
  misc_feature 1..65: note = Description of sequence: Artificial sequence
  source 1..65: mol_type = other DNA
tggatatctcctagccagactgttatgtgtgatacggcgcacttcgtgatcgccctacgggtccg

SEQ ID NO: 297 - DNA, 65 bp, synthetic construct; identical to SEQ ID NO: 36
  misc_feature 1..65: note = Description of sequence: Artificial sequence
  source 1..65: mol_type = other DNA
  modified_base 1: mod_base = OTHER; note = nucleotide bonded to fluorescent dye, dye name: 5' Iowa Black FQ

SEQ ID NO: 298 - DNA, 65 bp, synthetic construct; identical to SEQ ID NO: 37
  misc_feature 1..65: note = Description of sequence: Artificial sequence
  source 1..65: mol_type = other DNA
  modified_base 1: mod_base = OTHER; note = nucleotide bonded to fluorescent dye, dye name: 5' Iowa Black FQ

SEQ ID NO: 299 - DNA, 65 bp, synthetic construct; identical to SEQ ID NO: 40
  misc_feature 1..65: note = Description of sequence: Artificial sequence
  source 1..65: mol_type = other DNA
  modified_base 1: mod_base = OTHER; note = nucleotide bonded to fluorescent dye, dye name: 5' Iowa Black FQ

SEQ ID NO: 300 - DNA, 34 bp, synthetic construct; identical to SEQ ID NO: 41
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 34: mod_base = OTHER; note = nucleotide bonded to fluorescent dye, dye name: 3' Alexa Fluor 488
  modified_base 1: mod_base = OTHER; note = nucleotide bonded to fluorescent dye, dye name: 5' Iowa Black FQ

SEQ ID NO: 301 - DNA, 34 bp, synthetic construct; identical to SEQ ID NO: 42
  misc_feature 1..34: note = Description of sequence: Artificial sequence
  source 1..34: mol_type = other DNA
  misc_feature 1..24: note = DNA
  misc_feature 25: note = RNA
  misc_feature 26..34: note = DNA
  modified_base 34: mod_base = OTHER; note = nucleotide bonded to fluorescent dye, dye name: 3' Alexa Fluor 488
  modified_base 1: mod_base = OTHER; note = nucleotide bonded to fluorescent dye, dye name: 5' Iowa Black FQ

SEQ ID NO: 302 - DNA, 41 bp, synthetic construct
  misc_feature 1..41: note = Description of sequence: Artificial sequence
  source 1..41: mol_type = other DNA
  modified_base 41: mod_base = OTHER; note = nucleotide bonded to quencher, quencher name: 3' Iowa Black RQ
gcggcatgcgcgtttgtggtacctaaacgctcctaatagag

SEQ ID NO: 303 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 26: mod_base = OTHER; note = nucleotide bonded to quencher, quencher name: 3' Iowa Black RQ
  modified_base 1: mod_base = OTHER; note = nucleotide bonded to fluorescent dye, dye name: 5' Alexa Fluor 647
ctctattagggaggctcgcatgccgc

SEQ ID NO: 304 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 26: mod_base = OTHER; note = nucleotide bonded to quencher, quencher name: 3' Iowa Black RQ
  modified_base 1: mod_base = OTHER; note = nucleotide bonded to fluorescent dye, dye name: 5' Alexa Fluor 647
ctctattagggagaatcgcatgccgc

SEQ ID NO: 305 - DNA, 26 bp, synthetic construct
  misc_feature 1..26: note = Description of sequence: Artificial sequence
  source 1..26: mol_type = other DNA
  misc_feature 1..8: note = DNA
  misc_feature 9: note = RNA
  misc_feature 10..26: note = DNA
  modified_base 26: mod_base = OTHER; note = nucleotide bonded to quencher, quencher name: 3' Iowa Black RQ
  modified_base 1: mod_base = OTHER; note = nucleotide bonded to fluorescent dye, dye name: 5' Alexa Fluor 647
ctctattagggagattcgcatgccgc

SEQ ID NO: 306 - DNA, 21 bp, synthetic construct
  misc_feature 1..21: note = Description of sequence: Artificial sequence
  source 1..21: mol_type = other DNA
tcctagccagactgttatgtg

SEQ ID NO: 307-309 - skipped (3 entries)

(19) United States
(12) Patent Application Publication
(10) Pub. No.: US 2025/0314668 A1
(43) Pub. Date: Oct. 9, 2025

(54) DNAZYMES FOR SENSING AND IMAGING TARGET MOLECULES

(71) Applicants: BOARD OF REGENTS, THE UNIVERSITY OF TEXAS SYSTEM, Austin, TX (US); THE BOARD OF TRUSTEES OF THE UNIVERSITY OF ILLINOIS, Urbana, IL (US)

(72) Inventors: Yi LU, Austin, TX (US); Seyed-Fakhreddin TORABI, Urbana, IL (US); Yuting WU, Austin, TX (US); Ryan J. LAKE, Urbana, IL (US); Zhenglin YANG, Austin, TX (US); Whitney BESETZNY, Austin, TX (US); Jacqueline VAN STAPPEN, Austin, TX (US); Xiangli SHAO, Austin, TX (US)

(21) Appl. No.: 19/171,976

(22) Filed: Apr. 7, 2025

Related U.S. Application Data
(60) Provisional application No. 63/575,447, filed on Apr. 5, 2024.

(51) Int. Cl. G01N 33/84; C12N 15/113; G01N 21/64

(52) U.S. Cl. CPC G01N 33/84; C12N 15/113; G01N 21/6428; C12N 2310/127; G01N 2021/6432; G01N 2021/6439

(57) ABSTRACT

Various DNAzyme-based fluorescence sensors can be used to detect metal ions of various oxidation states. They are made of two different DNA strands, one called the substrate strand, and the other called the enzyme strand (E) which can catalyze the cleavage of the RNA base in the presence of specific target molecule. DNAzymes can be applied for high special and temporal resolution imaging of target ions in living cells and tissues with high specificity.

### CROSS-REFERENCE TO RELATED APPLICATIONS

This application claims the benefit of priority to U.S. Provisional Application No. 63/575,447, filed Apr. 5, 2024, which is incorporated by reference herein in its entirety.

### GOVERNMENT SUPPORT CLAUSE

This invention was made with government support under Grant No. R35 GM141931 awarded by the National Institutes of Health. The Government has certain rights in the invention.

### REFERENCE TO SEQUENCE LISTING

The sequence listing submitted on Apr. 7, 2025, as an .XML file entitled “10046-605US1_ST26.xml” created on Apr. 4, 2025, and having a file size of 546,847 bytes is hereby incorporated by reference pursuant to 37 C.F.R. § 1.52(e) (5).

### BACKGROUND

The current technology for highly selective and simultaneous sensing of Fe²⁺ and Fe³⁺ (or other metal ions with multiple oxidation states) in living cells or in vivo has had limited success. Current technology is mainly based on laboratory techniques, such as inductively coupled plasma mass spectrometry, electron paramagnetic resonance, x-ray fluorescence, and magnetic resonance imaging, which cannot readily provide spatial or temporal information in vivo because of their restrictive requirements for sample pretreatment or excessive time needed for data collection. They also focus on the total iron pool instead of the labile iron pool, which is an important portion of iron that contributes to biological events, such as ferroptosis. To visualize these labile iron pools, histochemical methods based on potassium ferricyanide or potassium ferrocyanide were developed, to distinguish Fe²⁺ and Fe³⁺ and acquire spatial information, but this method can only detect Fe²⁺ and Fe³⁺ separately on fixed tissue slices, not in living cells or in vivo.

Fluorescence sensors have also been developed to visualize labile Fe²⁺ and Fe³⁺ simultaneously in vivo and provide spatiotemporal information in living cells. However, most of these methods either have low selectivity for Fe²⁺ and Fe³⁺ over other metal ions, require organic solvents, or cannot be adapted readily for in vivo sensing applications. Recently, some Fe²⁺ sensors based on organic molecules and fluorophores have achieved sufficient selectivity and sensitivity for imaging in cells and mouse models. To image two different oxidation states of the same metal ions, such as Fe²⁺ and Fe³⁺ simultaneously, two sensors that are not only specific for the respective Fe²⁺ and Fe³⁺ but also two fluorophores that do not have much overlapping excitation and emission spectra to avoid interference in the detection are needed.

Because the target recognition and fluorescent readouts of the organic molecule sensors are coupled together, it is difficult to replace the fluorophore with one that has a different fluorescence emission spectrum to avoid overlap of fluorescent signals. Changing fluorophore moieties for these sensors normally requires a redesign of the sensors, which can adversely affect their other properties, such as loss of brightness of fluorescence, reduced selectivity, or change of subcellular localization of the sensor. Therefore, what is needed in the art is the simultaneous monitoring of two oxidation states of the same metal ion in living cells or in vivo.

### SUMMARY

Disclosed herein are DNAzyme sensors and methods of using the same to detect target molecules in cells and/or tissues. DNAzyme sensors have been previously used to detect metal ions in living cells or in vivo, however, conventional DNAzyme sensors tend to have low specificity for their intended target molecule when in the presence of multiple metal ions. Furthermore, conventional DNAzyme sensors often cannot accurately distinguish between different oxidation states of the same metal ion and are thus more limited to metal ions with single oxidation states (e.g., Zn²⁺). The DNAzyme sensors described herein are able to detect target molecules with high specificity in the presence of multiple metal ions. These DNAzyme sensors can further distinguish between different oxidation states of the same metal ion, even when multiple oxidation states of said metal ion are simultaneously present. As such, these DNAzyme sensors allow for accurate detection of target molecules, particularly metal ions with varying oxidation states, which can enable simultaneous detection of multiple oxidation states of multiple metal ions. These DNAzyme sensors can further be used to monitor disease progression, the physiological impact of a therapeutic agent, or to monitor disease treatment.

In an aspect, provided is a composition for simultaneously detecting a target ion in multiple oxidation states, the composition comprising: i) a first DNAzyme sensor comprising: a first substrate strand comprising a first cleavage site, wherein the first cleavage site is uncleaved when the target ion in a first oxidation state is not present; and a first enzyme strand at least partially complementary to the first substrate strand and comprising a first catalytic loop; wherein the first catalytic loop is capable of cleaving the first substrate strand at the first cleavage site in the presence of the target ion in the first oxidation state, wherein said cleavage provides a first detectable signal; and ii) a second DNAzyme sensor comprising: a second substrate strand comprising a second cleavage site, wherein the second cleavage site is uncleaved when the target ion in a second oxidation state is not present; and a second enzyme strand at least partially complementary to the second substrate strand and comprising a second catalytic loop; wherein the second catalytic loop is capable of cleaving the second substrate strand at the second cleavage site in the presence of the target ion in the second oxidation state, wherein said cleavage provides a second detectable signal.

In another aspect, provided is a DNAzyme sensor comprising: a substrate strand comprising a cleavage site, wherein the cleavage site is uncleaved when Fe²⁺ is not present; and an enzyme strand at least partially complementary to the substrate strand and comprising a catalytic loop; wherein the catalytic loop is capable of cleaving the substrate strand at the cleavage site in the presence of Fe²⁺, wherein said cleavage provides a detectable signal; and wherein the catalytic loop comprises SEQ ID NO: 306 or a variant thereof.

In yet another aspect, provided is a DNAzyme sensor comprising: a substrate strand comprising a cleavage site, wherein the cleavage site is uncleaved when Fe³⁺ is not present; and an enzyme strand at least partially complementary to the substrate strand and comprising a catalytic loop; wherein the catalytic loop is capable of cleaving the substrate strand at the cleavage site in the presence of Fe³⁺, wherein said cleavage provides a detectable signal; and wherein the catalytic loop comprises SEQ ID NO: 307 or a variant thereof.

In yet still another aspect, provided is a method of using any of the disclosed DNAzymes or compositions to detect a target molecule in a cell or tissue, the method comprising: a) providing the DNAzyme or composition to the cell or tissue; and b) detecting the first detectable signal and the second detectable signal.

In yet still another aspect, provided is a method of spatially identifying a target molecule in a cell or tissue, the method comprising: a) providing to the cell or tissue any of the disclosed DNAzyme sensors or compositions; and b) imaging the cell or tissue, thereby allowing for spatial identification of the target molecule.

In yet still another aspect, provided is a kit comprising: i) a DNAzyme sensor comprising: a substrate strand comprising a cleavage site, wherein the cleavage site is uncleaved when a target molecule is not present; and an enzyme strand at least partially complementary to the substrate strand and comprising a catalytic loop; wherein the catalytic loop is capable of cleaving the substrate strand at the cleavage site in the presence of the target molecule, wherein said cleavage provides a detectable signal; and ii) an inactive DNAzyme sensor comprising: the substrate strand; and an inactive enzyme strand at least partially complementary to the substrate strand and comprising at least one mutation, wherein the at least one mutation prevents the inactive enzyme strand from cleaving the substrate strand.

In yet still another aspect, provided is a method of using any of the disclosed kits to detect a target molecule in a cell or tissue by: a) providing the inactive DNAzyme sensor to the cell or tissue; b) detecting the detectable signal, thereby providing a reference level of the detectable signal; c) providing the DNAzyme sensor to the cell or tissue; and d) detecting the detectable signal; wherein the reference level is used to eliminate background noise in step d).

In yet still another aspect, provided is a method of determining an effect of a therapeutic agent on a target molecule, the method comprising: a) administering the therapeutic agent to a cell or tissue; b) exposing the cell or tissue to any of the disclosed DNAzyme sensors or compositions; and c) detecting the detectable signal; and d) using said detectable signal to determine the effect of the therapeutic agent on iron.

In yet still another aspect, provided is a method of determining an effect of a therapeutic agent on a target molecule in a cell or tissue, the method comprising: a) administering the therapeutic agent to the cell or tissue; b) spatially identifying the target molecule in the cell or tissue according to any of the disclosed methods of spatially identifying a target molecule in a cell or tissue; and c) comparing the spatial identification of step b) to a spatial identification of the target molecule in a control cell or control tissue.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIG. 1 depicts two predicted secondary structures of the cis-acting Fe(III)-B12 DNAzyme (SEQ ID NO: 14) that represent different catalytic core conformations were selected for truncation studies. In each secondary structure, potential enzyme-substrate binding arms (boxed in green and blue) were identified. Regions outside the binding arms were removed by truncation. Removed regions include 1) 3′- and 5′-tails below binding arms boxed in green and 2) loop nucleotides on the left side of the binding arm boxed in blue. Binding arms in trans-acting DNAzymes were extended if their melting temperature was below 20° C. to support proper enzyme-substrate hybridization at room temperature. The intended cleavage site is a single guanosine ribonucleotide (rG), which is boxed in red. Activity of trans-acting DNAzymes were tested in the presence of Fe³⁺ to identify the minimal catalytic sequence that efficiently cleaves the substrate strand. Both versions of truncation-2 were equally active. Therefore, truncation-2a, which is the minimal catalytic region, was used to design the Fe(III)-B12 fluorescent sensor.

FIG. 2 depicts Kobs of the Fe(III)-B12 sensor based on one-phase decay fitting with the fluorescence increase towards different concentrations of Fe³⁺.

FIGS. 3A-3B depict selectivity of Fe(II)-H5 and Fe(III)-B12 catalytic beacon sensors for alternative metal ions, specifically selectivity of the fluorescent catalytic beacon sensors of Fe(II)-H5 (FIG. 3A) and Fe(III)-B12 (FIG. 3B) against 200 UM of the relevant metal ion after 30 min incubation. Mn³⁺ was tested both with and without 5 mM pyruvate (w/pyro) to help stabilize it in its oxidized form. Measurements were performed in 20 mM sodium acetate, 5 mM Bis-Tris, 200 mM NaCl at pH 6.0. The fluorescence intensity in the presence of different metal ions was normalized to the fluorescent signal without addition of divalent or trivalent metal ions (i.e., just buffer) at 30-minute time point, for a direct comparison. As a result, fluorescence quenching by the paramagnetic Fe³⁺, Cu²⁺ and Ni²⁺ resulted in a negative response. The concentration (200 μM) used in this study is higher than that of Fe³⁺ under most physiological conditions.

FIGS. 4A-4B depict cleavage activity in the presence of Fe²⁺, Fe³⁺, or a mixture of both for the Fe(II)-H5 and Fe(III)-B12 DNAzymes. FIG. 4A shows DNAzyme activity for the Fe(II)-H5 DNAzyme was monitored with 100 μM Fe²⁺ in 20 mM acetate buffer pH 6.0, 5 mM Bis-Tris and 200 mM NaCl in the presence or absence of 20 μM Fe³⁺. No activity was observed with only 20 μM Fe³⁺ (green triangles). FIG. 4B shows DNAzyme activity for the Fe(III)-B12 DNAzyme was monitored with 20 μM Fe³⁺ in 20 mM acetate buffer pH 6.0, 5 mM Bis-Tris and 200 mM NaCl in the presence or absence of 100 μM Fe²⁺. No cleavage was noticed with 100 μM Fe²⁺ (blue circles).

FIG. 5 depicts a heat map illustrating the impact of point mutations within the catalytic core of the Fe³⁺ B12 DNAzyme, against the unaltered Fe³⁺ B12 substrate strand for comparative analysis with 5 mM Bis Tris, 40 mM Sodium Acetate, 200 mM NaCl at pH 5.5 with 100 μM FeCl₃. The reaction occurred overnight at room temperature and analyzed by polyacrylamide gel electrophoresis.

FIG. 6 depicts a heat map illustrating the impact of point mutations within the catalytic core of the Fe³⁺ B12 Substrate strand, against the unaltered Fe³⁺ B12 DNAzyme strand for comparative analysis with 5 mM Bis Tris, 40 mM Sodium Acetate, 200 mM NaCl at pH 5.5 with 100 μM FeCl₃. The reaction occurred overnight at room temperature and analyzed by polyacrylamide gel electrophoresis.

FIG. 7 depicts a heat map illustrating the impact of point mutations within the catalytic core of the Fe²⁺ H5 DNAzyme, against the unaltered Fe²⁺ H5 substrate strand for comparative analysis with 25 mM Bis Tris, 200 mM NaCl at pH 6.5 with 100 μM FeCl₂ in anaerobic conditions. The reaction occurred overnight at room temperature and analyzed by polyacrylamide gel electrophoresis.

FIG. 8 depicts a heat map illustrating the impact of point mutations within the catalytic core of the Fe²⁺ H5 Substrate strand, against the unaltered Fe²⁺ H5 DNAzyme strand for comparative analysis with 25 mM Bis Tris, 200 mM NaCl at pH 6.5 with 100 μM FeCl₂ in anaerobic conditions. The reaction occurred overnight at room temperature and analyzed by polyacrylamide gel electrophoresis.

FIGS. 9A-9B depict the sequences of Cu⁺ and Cu²⁺ DNAzyme. FIG. 9A shows a substrate strand of SEQ ID NO: 44 (with a cleavage site at residue 18) and an enzyme strand of SEQ ID NO: 45. FIG. 9B shows a substrate strand of SEQ ID NO: 47 (with a cleavage site at linkage *) and an enzyme strand of SEQ ID NO: 48.

FIG. 10 depicts gel and fluorescence-based activity assay for Cu⁺ and Cu²⁺ DNAzyme. PAGE Gel images of Cu⁺ DNAzyme cleaving the substrate in the presence of various concentrations of Cut. Lane 1: Cu⁺ aS, Lane 2: Cu⁺ aE, Lane 3-10: the concentration of Cu²⁺ is 0 nM, 125 nM, 250 nM, 500 nM, 1 μM, 2 μM, 4 μM, and 8 μM, respectively, the concentration of ascorbate is 50 μM. Quantification of Cu⁺. Inset is the linear response at low Cu⁺ concentrations. denaturing gel images of Cu²⁺ DNAzyme cleaving the substrate in the presence of various concentrations of Cu²⁺. Lane1-8: the concentration of Cu²⁺ is 0 μM, 1 μM, 2 μM, 4 μM, 6 μM, 8 μM, 10 μM, and 20 μM, respectively. Quantification of Cu²⁺. Inset: the linear response at low Cu²⁺ concentrations.

FIG. 11 depicts the sequences of active (substrate strand is SEQ ID NO: 47, enzyme strand is SEQ ID NO: 48) and inactive Cu²⁺ DNAzyme. For inactive Cu²⁺ DNAzyme, the catalytic core sequence is converted into complement sequence (SEQ ID NO: 49), polyA (SEQ ID NO: 50), polyN (SEQ ID NO: 51), polyT (SEQ ID NO: 52) of the original active sequences, or substrate strand without RNA modification (substrate strand is SEQ ID NO: 72, enzyme strand is SEQ ID NO: 53). F: fluorophore labels.

FIGS. 12A-12B depict different Cu sensors performance at different ionic strength conditions. Before Cu DNAzyme transfection, anneal 12 uM Cu⁺ DNAzyme in 50 mM MOPS buffer with 25 mM NaCl, 50 mM NaCl, and 75 mM NaCl, pH7.4. After transfection, the final Cut DNAzyme concentration is 400 nM.

FIG. 13 depicts the sequences of the upgraded Cu DNAzyme version. F: fluorophore labels; Q: quencher labels. Cu1 aE v2 has a substrate strand of SEQ ID NO: 63 and an enzyme strand of SEQ ID NO: 64. Cu1 iE v2 has a substrate strand of SEQ ID NO: 63 and an inactive enzyme strand of SEQ ID NO: 65. Cu2 aE v2 has a substrate strand of SEQ ID NO: 66 and an enzyme strand of SEQ ID NO: 67. Cu2 iE v2 has a substrate strand of SEQ ID NO: 66 and an inactive enzyme strand of SEQ ID NO: 68.

FIGS. 14A-14B depict fluorescence resulting of upgraded Cu DNAzyme. The concentration of Cu²⁺ is 8 μM, the concentration of ascorbate is 50 μM.

FIGS. 15A-15B depict upgraded Cu DNAzyme sensors activity in Hela cells. Cu⁺ and Cu²⁺ DNAzyme transfected individually or co-delivered into Hela cells.

FIGS. 16A-16F depict gel and fluorescence-based activity assay for Cu⁺ and Cu²⁺ DNAzyme. FIG. 16A shows the secondary structure of the Cu⁺ DNAzyme (substrate strand is SEQ ID NO: 44, enzyme strand is SEQ ID NO: 45). F and Q denote fluorophore and quencher, respectively. FIG. 16B shows PAGE Gel images of Cu⁺ DNAzyme cleaving the substrate in the presence of various concentrations of Cut. Lane 1: Cu1 aS, Lane 2: Cu1 aE, Lane 3-10: the concentration of Cu²⁺ is 0 nM, 125 nM, 250 nM, 500 nM, 1μ, 2μ, 4μ, and 8 μM, respectively, the concentration of ascorbate is 50 μM. FIG. 16C shows quantification of Cu⁺. Inset: the linear response at low Cu⁺ concentrations. FIG. 16D shows the secondary structure of the Cu²⁺ DNAzyme (substrate strand is SEQ ID NO: 47, enzyme strand is SEQ ID NO: 48). FIG. 16E shows dPAGE gel images of Cu²⁺ DNAzyme cleaving the substrate in the presence of various concentrations of Cu²⁺. Lane1-8: the concentration of Cu²⁺ is 0 μM, 1 μM, 2 μM, 4 μM, 6 μM, 8 μM, 10 μM, and 20 μM, respectively. FIG. 16F shows quantification of Cu²⁺. Inset: the linear response at low Cu²⁺ concentrations.

FIGS. 17A-17E depict confocal microscopy images of SH-SY5Y cells treated with PDTC. FIG. 17A shows SH-SY5Y cells transfected with Cu⁺ and Cu²⁺ DNAzyme. FIGS. 17B-7D show SH-SY5Y cells transfected with Cu⁺ and Cu²⁺ DNAzyme along with various Cu²⁺ and PDTC concentration, the concentration of Cu²⁺ and PDTC is 10 μM, 50 μM, and 100 μM. After DNAzyme transfection, the cells were incubated with 10 μM, 50 μM, and 100 μM of Cu²⁺ and PDTC in Opti-MEM for 30 min, cells were washed with 1×HBSS and the medium was replaced by Opti-MEM for images. FIG. 17E shows the distribution of FAM and Cy5 intensity of confocal images of SH-SY5Y cells treated with Cu²⁺ and PDTC (****p<0.0001). The green channel is from FAM fluorescence. The red channel is from Cy5 fluorescence. Scale bar 20 μm.

FIGS. 18A-18E depict confocal microscopy images of SH-SY5Y cells treated with BCS. FIG. 18A shows SH-SY5Y cells transfected with Cu⁺ and Cu²⁺ DNAzyme. FIGS. 18B-18D show SH-SY5Y cells transfected with Cu⁺ and Cu²⁺ DNAzyme along with various BCS concentration. The cells were incubated with 150 μM, 200 μM, and 250 μM BCS in DMEM/10% FBS medium overnight and washed with and HBSS, followed by DNAzyme transfection and images. FIG. 18E shows the distribution of FAM and Cy5 intensity of confocal images of SH-SY5Y cells treated with BCS (****p<0.0001). The green channel is from FAM fluorescence. The red channel is from Cy5 fluorescence. Scale bar 20 μm.

FIGS. 19A-19E depict confocal microscopy images of SH-SY5Y cells treated with Aβ. FIG. 19A shows SH-SY5Y cells transfected with Cu⁺ and Cu²⁺ DNAzyme. FIGS. 19B-19D show SH-SY5Y cells transfected with Cu⁺ and Cu²⁺ DNAzyme along with different Aβ aggregation state. FIG. 19E shows the distribution of FAM and Cy5 intensity of confocal images of SH-SY5Y cells treated with Aβ (****p<0.0001, ***p=0.0001). The green channel is from FAM fluorescence. The red channel is from Cy5 fluorescence. Scale bar 20 μm.

FIGS. 20A-20D depict confocal microscopy images of Cu⁺ and Cu²⁺ in iPSCs-derived neurons treated with Aβ40. FIG. 20A shows iPSCs-derived neurons transfected with Cu⁺ and Cu²⁺ DNAzyme. FIGS. 20B-20D show iPSCs-derived neurons transfected with Cu⁺ and Cu²⁺ DNAzyme along with different Aβ aggregation state. Scale bar 25 μm.

FIGS. 21A-21C depict cell death in FDX1 regulated SH-SY5Y cells with Aβ-Cu treatment. FIG. 21A shows wild type and FDX1 knock down SH-SY5Y cells treated with 10 μM Aβ and 100 μM, 50 μM, 10 μM, 2 μM, 1 μM, and 0.5 μM CuCl₂. FIG. 21B shows Aβ-Cu (20:1) treatment FDX1 knock down SH-SY5Y cells incubated with 25 UM Tempol or 100 μM Trolox. FIG. 21C shows ROS level in FDX1 knock down SH-SY5Y cells. For all graphs, ****p<0.0001, ***p<0.001, **p<0.01, and ns is p>0.05.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single-dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, the term “DNAzymes,” also called “deoxyribozymes,” are DNA molecules that display enzymatic activities, such as protein enzymes and ribozymes, in the presence of a cofactor such as metal ions or another target molecule.

### DNAzymes, Compositions, and Kits

In an aspect, provided is a DNAzyme sensor comprising: a substrate strand comprising a cleavage site and a detectable signal, wherein the detectable signal is initially deactivated; and an enzyme strand at least partially complementary to the substrate strand. The enzyme strand can cleave the substrate strand at the cleavage site in presence of a target molecule, thereby activating the detectable signal.

In some aspects, the cleavage site can include at least one RNA base. In some aspects, the cleavage site can include 1 to 5 RNA bases (e.g., 1 RNA base, 2 RNA bases, 3 RNA bases, 4 RNA bases, or 5 RNA bases).

In some aspects, the cleavage site can be interspersed between two segments of DNA. In some aspects, the two segments of DNA can have a same length. In other aspects, the two segments of DNA can have different lengths. In some aspects, each of the two segments of DNA comprises 3 to 30 DNA bases (e.g., 6 to 27 DNA bases, 9 to 24 DNA bases, 12 to 21 DNA bases, 15 to 18 DNA bases, 3 to 18 DNA bases, 6 to 15 DNA bases, 9 to 12 DNA bases, 15 to 30 DNA bases, 18 to 27 DNA bases, 21 to 24 DNA bases).

In some aspects, the substrate strand can further include at least one non-natural nucleic acid. In some aspects, the at least one non-natural nucleic acid can be a locked nucleic acid (LNA).

In some aspects, the enzyme strand can include at least one loop region.

In some aspects, the enzyme strand can include a target molecule binding region. In some aspects, the target molecule can be a metal ion having two or more oxidation states. In some aspects, the metal ion can be Fe²⁺ and the catalytic loop can comprise SEQ ID NO: 306 (TCCTAGCCAGACTGTTATGTG) or a variant thereof. In some aspects, the metal ion can be Fe³⁺ and the catalytic loop can comprise SEQ ID NO: 307 (CGGCAC) or a variant thereof. In some aspects, the metal ion can be Cu⁺ and the catalytic loop can comprise SEQ ID NO: 308 (TGGGCC) or a variant thereof. In some aspects, the metal ion can be Cu²⁺ and the catalytic loop can comprise SEQ ID NO: 309 (ACCAGGAA) or a variant thereof. In some aspects, the metal ion can be Mn²⁺ or Mn⁴⁺. In some aspects, the metal ion can be Cr³⁺ or Cr⁶⁺. In some aspects, the metal ion can be Co²⁺ or Co³⁺. In some aspects, the metal ion can be Pb²⁺ or Pb⁴⁺. In some aspects, the metal ion can be Ag⁺ or Ag²⁺. In some aspects, particularly if the enzyme strand is at least partially complementary to both itself and the substrate strand and the DNAzyme forms a triplex structure (i.e., with the substrate strand and two portions of the enzyme strand), the interaction between the catalytic loop and the target molecule may further be strengthened by nucleotides on the tail end of the enzyme strand positioned near the catalytic loop (e.g., an AC dinucleotide sequence on the tail end of the enzyme strand can further strengthen the interaction between the catalytic loop and Cu⁺).

In other aspects, the target molecule can be a metal ion with only one oxidation state. In some aspects, the metal ion can be Mg²⁺, Na⁺, Li⁺, Zn²⁺, K⁺, Cd²⁺, or Ca²⁺. In yet other aspects, the target molecule can be UO₂²⁺. In yet other aspects, the target molecule can be a protein or a small molecule.

In some aspects, the catalytic loop can have a Michaelis constant (Kₘ) for Fe²⁺ of about 1×10⁻⁴ min⁻¹ μM⁻¹ or more (e.g., about 1.05×10⁻⁴ min⁻¹ μM⁻¹ or more, about 1.1×10⁻⁴ min⁻¹ μM⁻¹ or more, about 1.15×10⁻⁴ min⁻¹ μM⁻¹ or more, about 1.2×10⁻⁴ min⁻¹ μM⁻¹ or more, about 1.25×10⁻⁴ min⁻¹ μM⁻¹ or more, about 1.3×10⁻⁴ min⁻¹ μM⁻¹ or more, about 1.35×10⁻⁴ min⁻¹ μM⁻¹ or more, about 1.4×10⁻⁴ min⁻¹ μM⁻¹ or more, about 1.45×10⁻⁴ min⁻¹ μM⁻¹ or more, about 1.5×10⁻⁴ min⁻¹ μM⁻¹ or more). In some aspects, the catalytic loop can have a Michaelis constant (Kₘ) for Fe²⁺ of about 1.5×10⁻⁴ min⁻¹ μM⁻¹ or less (e.g., about 1.45×10⁻⁴ min⁻¹ μM⁻¹ or less, about 1.4×10⁻⁴ min⁻¹ μM⁻¹ or less, about 1.35×10⁻⁴ min⁻¹ μM⁻¹ or less, about 1.3×10⁻⁴ min⁻¹ μM⁻¹ or less, about 1.25×10⁻⁴ min⁻¹ μM⁻¹ or less, about 1.2×10⁻⁴ min⁻¹ μM⁻¹ or less, about 1.15×10⁻⁴ min⁻¹ μM⁻¹ or less, about 1.1×10⁻⁴ min⁻¹ μM⁻¹ or less, about 1.05× 10⁻⁴ min⁻¹ μM⁻¹ or less, about 1×10⁻⁴ min⁻¹ μM⁻¹ or less). The catalytic loop can have a Michaelis constant (Kₘ) ranging from any of the minimum values described above to any of the maximum values described above. For example, in some aspects, the catalytic loop can have a Michaelis constant (Kₘ) or from about 1×10⁻⁴ min⁻¹ μM⁻¹ to about 1.5×10⁻⁴ min⁻¹ μM⁻¹ (e.g., from about 1.05× 10⁻⁴ min⁻¹ μM⁻¹ to about 1.45×10⁻⁴ min⁻¹ μM⁻¹, from about 1.1×10⁻⁴ min⁻¹ μM⁻¹ to about 1.4×10⁻⁴ min⁻¹ μM⁻¹, from about 1.15×10⁻⁴ min⁻¹ μM⁻¹ to about 1.35× 10⁻⁴ min⁻¹ μM⁻¹, from about 1.2×10⁻⁴ min⁻¹ μM⁻¹ to about 1.3×10⁻⁴ min⁻¹ μM⁻¹, from about 1× 10⁻⁴ min⁻¹ μM⁻¹ to about 1.25×10⁻⁴ min⁻¹ μM⁻¹, from about 1.05×10⁻⁴ min⁻¹ μM⁻¹ to about 1.2× 10⁻⁴ min⁻¹ μM⁻¹, from about 1.1×10⁻⁴ min⁻¹ μM⁻¹ to about 1.15×10⁻⁴ min⁻¹ μM⁻¹, from about 1.25×10⁻⁴ min⁻¹ μM⁻¹ to about 1.5×10⁻⁴ min⁻¹ μM⁻¹, from about 1.3× 10⁻⁴ min⁻¹ μM⁻¹ to about 1.45×10⁻⁴ min⁻¹ μM⁻¹, from about 1.35×10⁻⁴ min⁻¹ μM⁻¹ to about 1.4×10⁻⁴ min⁻¹ μM⁻¹).

In some aspects, the catalytic loop can have a Michaelis constant (Kₘ) for Fe³⁺ of about 1×10⁻² min⁻¹ μM⁻¹ or more (e.g., about 1.05×10⁻² min⁻¹ μM⁻¹ or more, about 1.1×10⁻² min⁻¹ μM⁻¹ or more, about 1.15× 10⁻² min⁻¹ μM⁻¹ or more, about 1.2× 10⁻² min⁻¹ μM⁻¹ or more, about 1.25× 10⁻² min⁻¹ μM⁻¹ or more, about 1.3×10⁻² min⁻¹ μM⁻¹ or more, about 1.35× 10⁻² min⁻¹ μM⁻¹ or more, about 1.4×10⁻² min⁻¹ μM⁻¹ or more, about 1.45×10⁻² min⁻¹ μM⁻¹ or more, about 1.5×10 2 min⁻¹ μM⁻¹ or more). In some aspects, the catalytic loop can have a Michaelis constant (Kₘ) for Fe³⁺ of about 1.5× 10⁻² min⁻¹ μM⁻¹ or less (e.g., about 1.45×10⁻² min⁻¹ μM⁻¹ or less, about 1.4×10⁻² min⁻¹ μM⁻¹ or less, about 1.35× 10⁻² min⁻¹ μM⁻¹ or less, about 1.3×10⁻² min⁻¹ μM⁻¹ or less, about 1.25× 10⁻² min⁻¹ μM⁻¹ or less, about 1.2× 10⁻² min⁻¹ μM⁻¹ or less, about 1.15× 10⁻² min⁻¹ μM⁻¹ or less, about 1.1×10⁻² min⁻¹ μM⁻¹ or less, about 1.05× 10⁻² min⁻¹ μM⁻¹ or less, about 1×10⁻² min⁻¹ μM⁻¹ or less). The catalytic loop can have a Michaelis constant (Kₘ) for Fe³⁺ ranging from any of the minimum values described above to any of the maximum values described above. For example, in some aspects, the catalytic loop can have a Michaelis constant (Kₘ) for Fe³⁺ of from about 1× 10⁻² min⁻¹ μM⁻¹ to about 1.5×10⁻² min⁻¹ μM⁻¹ (e.g., from about 1.05× 10⁻² min⁻¹ μM⁻¹ to about 1.45× 10⁻² min⁻¹ μM⁻¹, from about 1.1×10⁻² min⁻¹ μM⁻¹ to about 1.4× 10⁻² min⁻¹ μM⁻¹, from about 1.15×10⁻² min⁻¹ μM⁻¹ to about 1.35×10⁻² min⁻¹ μM⁻¹, from about 1.2× 10⁻² min⁻¹ μM⁻¹ to about 1.3× 10⁻² min⁻¹ μM⁻¹, from about 1× 10⁻² min⁻¹ μM⁻¹ to about 1.25×10⁻² min⁻¹ μM⁻¹, from about 1.05× 10⁻² min⁻¹ μM⁻¹ to about 1.2× 10⁻² min⁻¹ μM⁻¹, from about 1.1×10⁻² min⁻¹ μM⁻¹ to about 1.15×10⁻² min⁻¹ μM⁻¹, from about 1.25×10⁻² min⁻¹ μM⁻¹ to about 1.5×10⁻² min⁻¹ μM⁻¹, from about 1.3×10⁻² min⁻¹ μM⁻¹ to about 1.45×10⁻² min⁻¹ μM⁻¹, from about 1.35×10⁻² min⁻¹ μM⁻¹ to about 1.4× 10⁻² min⁻¹ μM⁻¹).

In some aspects, the detectable signal can be a fluorophore or a fluorescent dye. In some aspects, the detectable signal can be a photoacoustic dye; and, when the substrate strand is cleaved, the detectable signal can be activated upon exposure to an acoustic signal. In some aspects, the detectable signal can be indocyanine green, methylene blue, or Evans blue.

In some aspects, the detectable signal can be conjugated to a first end of the substrate strand, and a quencher can be conjugated to a complementary end of the enzyme strand. In some aspects, the detectable signal can be conjugated to a first end of the substrate strand, and a quencher can be conjugated to a second end of the substrate strand. In some aspects, the detectable signal can be conjugated to a first end of the substrate strand, a first quencher can be conjugated to a complementary end of the enzyme strand, and a second quencher can be conjugated to a second end of the substrate strand.

The fluorescence properties of the sensors can be changed by changing the fluorophore and quencher pairs. Thus, the sensors can be applied to provide spatial-temporal information of the metal ion, such as Fe²⁺ and Fe³⁺, simultaneously with other biomarkers or sensors. Moreover, the signaling readout can be changed according to the sensing needs. By changing the fluorophore-quencher pairs into other signaling-out put pairs, such as photoacoustic, non-invasive sensing with a different form of signaling output can be achieved. Moreover, by delivering the sensors to specific locations inside the cells, imaging of Fe²⁺ and Fe³⁺ in different subcellular localizations can be accomplished.

Examples of fluorophores include, but are not limited to, Hydroxycoumarin, Alexa fluor, Aminocoumarin, Methoxycoumarin, Cascade Blue, Pacific Blue, Pacific Orange, Lucifer yellow, Alexa fluor 430, NBD, R-Phycoerythrin (PE), PE-Cy5 conjugates, PE-Cy7 conjugates, Red 613, PerCP, Cy2, TruRed, FluorX, Fluorescein, FAM, BODIPY-FL, TET, Alexa fluor 532, HEX, TRITC, Cy3, TMR, Alexa fluor 546, Alexa fluor 555, Tamara, X-Rhodamine, Lissamine Rhodamine B, ROX, Alexa fluor 568, Cy3.5 581, Texas Red, Alexa fluor 594, Alexa fluor 633, LC red 640, Allophycocyanin (APC), Alexa fluor 633, APC-Cy7 conjugates, Cy5, Cy5.5, LC red 705, Cy7, IRDye 800 CW, IRDye 700, Cy7.5, Dy780, Dy781, DyLight 800, IRDye 800 CW, Alexa Fluor 647, Alexa Fluor 488, Alexa Fluor 660, Alexa Fluor 680, Alexa Fluor 750, Alexa Fluor 790, JOE, and MAX.

Examples of quenchers include, but are not limited to, DQ-I, Dabcyl, Eclipse, Iowa Black FQ, BHQ-1, QSY-7, BHQ-2, DDQ-II, Iowa Black RQ, QSY-21, BHQ-3, IRDye QC-1, and ZEN.

In some aspects, the substrate strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 1; and the enzyme strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 2.

In some aspects, the substrate strand can include any one of the sequences in TABLE 1; and the enzyme strand can include any one of the sequences in TABLE 2.

In some aspects, the substrate strand comprises 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 3; and the enzyme strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 4.

In some aspects, the substrate strand can include any one of the sequences in TABLE 3; and the enzyme strand can include any one of the sequences in TABLE 4.

In some aspects, the substrate strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 5; and the enzyme strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 6.

In some aspects, the substrate strand can include any one of the sequences in TABLE 5; and the enzyme strand can include any one of the sequences in TABLE 6.

In some aspects, the substrate strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 7; and the enzyme strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 8.

In some aspects, the substrate strand can include any one of the sequences in TABLE 7; and the enzyme strand can include any one of the sequences in TABLE 8.

TABLE 1
Fe²⁺ substrate strands.

| | | SEQ |
|---|---|---|
| Strand | Sequence | ID NO |
| Fe(II)-H5-rS | /5IABkFQ/CGGACCCGTATCAATCTCACGTATrAGGATAT | 20 |
| | CCA/3AlexF488N/ | |
| Fe(II)-H5-rS | CGGACCCGTATCAATCTCACGTATrAGGATATCCA | 26 |
| Iron sensor | /5IAbRQ/CGGACCCGTATCAATCTCACGTATrAGGATATC | 30 |
| (Fe²⁺) | CA/3AlexF546N/ | |
| Fe²⁺ H5 | CGGACCCGTACCAATCTCACGTATrAGGATATCCA | 41 |
| Substrate T11C | | |
| Fe²⁺ H5 | CGGACCCGTATCCATCTCACGTATrAGGATATCCA | 42 |
| Substrate A13C | | |
| Fe²⁺ H5 | CGGACCCGTATCATTCTCACGTATrAGGATATCCA | 43 |
| Substrate A14T | | |
| Fe²⁺ H5 | CGGACCCGTATCAATCTCACGTATAGGATATCCA | 74 |
| Original | | |
| Substrate (2′H) | | |
| Fe²⁺ H5 | CGGACCCGTATCAATCTCACGTATmAGGATATCCA | 75 |
| Original | | |
| Substrate | | |
| (2′OMe) | | |
| Fe²⁺ H5 | /56- | 78 |
| Original | FAM/CGGACCCGTATCAATCTCACGTATrAGGATATCCA | |
| Substrate FAM | | |
| Fe²⁺ H5 | /56- | 79 |
| Substrate T11A | FAM/CGGACCCGTAACAATCTCACGTATrAGGATATCCA | |
| FAM | | |
| Fe²⁺ H5 | /56- | 80 |
| Substrate T11C | FAM/CGGACCCGTACCAATCTCACGTATrAGGATATCCA | |
| FAM | | |
| Fe²⁺ H5 | /56- | 81 |
| Substrate T11G | FAM/CGGACCCGTAGCAATCTCACGTATrAGGATATCCA | |
| FAM | | |
| Fe²⁺ H5 | /56- | 82 |
| Substrate C12A | FAM/CGGACCCGTATAAATCTCACGTATrAGGATATCCA | |
| FAM | | |
| Fe²⁺ H5 | /56- | 83 |
| Substrate C12T | FAM/CGGACCCGTATTAATCTCACGTATrAGGATATCCA | |
| FAM | | |
| Fe²⁺ H5 | /56- | 84 |
| Substrate C12G | FAM/CGGACCCGTATGAATCTCACGTATrAGGATATCCA | |
| FAM | | |
| Fe²⁺ H5 | /56- | 85 |
| Substrate A13T | FAM/CGGACCCGTATCTATCTCACGTATrAGGATATCCA | |
| FAM | | |
| Fe²⁺ H5 | /56- | 86 |
| Substrate A13C | FAM/CGGACCCGTATCCATCTCACGTATrAGGATATCCA | |
| FAM | | |
| Fe²⁺ H5 | /56- | 87 |
| Substrate A13G | FAM/CGGACCCGTATCGATCTCACGTATrAGGATATCCA | |
| FAM | | |
| Fe²⁺ H5 | /56- | 88 |
| Substrate A14T | FAM/CGGACCCGTATCATTCTCACGTATrAGGATATCCA | |
| FAM | | |
| Fe²⁺ H5 | /56- | 89 |
| Substrate A14C | FAM/CGGACCCGTATCACTCTCACGTATrAGGATATCCA | |
| FAM | | |
| Fe²⁺ H5 | /56- | 90 |
| Substrate A14G | FAM/CGGACCCGTATCAGTCTCACGTATrAGGATATCCA | |
| FAM | | |
| Fe²⁺ H5 | /56- | 91 |
| Substrate T15A | FAM/CGGACCCGTATCAAACTCACGTATrAGGATATCCA | |
| FAM | | |
| Fe²⁺ H5 | /56- | 92 |
| Substrate T15C | FAM/CGGACCCGTATCAACCTCACGTATrAGGATATCCA | |
| FAM | | |
| Fe²⁺ H5 | /56- | 93 |
| Substrate T15G | FAM/CGGACCCGTATCAAGCTCACGTATrAGGATATCCA | |
| FAM | | |
| Fe²⁺ H5 | /56- | 94 |
| Substrate C16A | FAM/CGGACCCGTATCAATATCACGTATrAGGATATCCA | |
| FAM | | |
| Fe²⁺ H5 | /56- | 95 |
| Substrate C16T | FAM/CGGACCCGTATCAATTTCACGTATrAGGATATCCA | |
| FAM | | |
| Fe²⁺ H5 | /56- | 96 |
| Substrate C16G | FAM/CGGACCCGTATCAATGTCACGTATrAGGATATCCA | |
| FAM | | |
| Fe²⁺ H5 | /56- | 97 |
| Substrate T22A | FAM/CGGACCCGTATCAATCTCACGAATrAGGATATCCA | |
| FAM | | |
| Fe²⁺ H5 | /56- | 98 |
| Substrate T22C | FAM/CGGACCCGTATCAATCTCACGCATrAGGATATCCA | |
| FAM | | |
| Fe²⁺ H5 | /56- | 99 |
| Substrate T22G | FAM/CGGACCCGTATCAATCTCACGGATrAGGATATCCA | |
| FAM | | |
| Fe²⁺ H5 | /56- | 100 |
| Substrate A23T | FAM/CGGACCCGTATCAATCTCACGTTTrAGGATATCCA | |
| FAM | | |
| Fe²⁺ H5 | /56- | 101 |
| Substrate A23C | FAM/CGGACCCGTATCAATCTCACGTCTrAGGATATCCA | |
| FAM | | |
| Fe²⁺ H5 | /56- | 102 |
| Substrate A23G | FAM/CGGACCCGTATCAATCTCACGTGTrAGGATATCCA | |
| FAM | | |
| Fe²⁺ H5 | /56- | 103 |
| Substrate T24A | FAM/CGGACCCGTATCAATCTCACGTAArAGGATATCCA | |
| FAM | | |
| Fe²⁺ H5 | /56- | 104 |
| Substrate T24C | FAM/CGGACCCGTATCAATCTCACGTACrAGGATATCCA | |
| FAM | | |
| Fe²⁺ H5 | /56- | 105 |
| Substrate T24G | FAM/CGGACCCGTATCAATCTCACGTAGrAGGATATCCA | |
| FAM | | |
| Fe²⁺ H5 | /56- | 106 |
| Substrate | FAM/CGGACCCGTATCAATCTCACGTATrUGGATATCCA | |
| rA25rU FAM | | |
| Fe²⁺ H5 | /56- | 107 |
| Substrate | FAM/CGGACCCGTATCAATCTCACGTATrCGGATATCCA | |
| rA25rC FAM | | |
| Fe²⁺ H5 | /56- | 108 |
| Substrate | FAM/CGGACCCGTATCAATCTCACGTATrGGGATATCCA | |
| rA25rG FAM | | |
| Fe²⁺ H5 | /56- | 109 |
| Substrate G26A | FAM/CGGACCCGTATCAATCTCACGTATrAAGATATCCA | |
| FAM | | |
| Fe²⁺ H5 | /56- | 110 |
| Substrate G26T | FAM/CGGACCCGTATCAATCTCACGTATrATGATATCCA | |
| FAM | | |
| Fe²⁺ H5 | /56- | 111 |
| Substrate G26C | FAM/CGGACCCGTATCAATCTCACGTATrACGATATCCA | |
| FAM | | |
| Fe²⁺ H5 | /5IABkFQ/CGGACCCGTACCAATCTCACGTATrAGGATAT | 300 |
| Substrate T11C | CCA/3AlexF488N/ | |
| 3′ Alexa | | |
| Fluor ™ 488 | | |
| (NHS Ester) | | |
| Fe²⁺ H5 | /5IABkFQ/CGGACCCGTATCCATCTCACGTATrAGGATAT | 301 |
| Substrate A13C | CCA/3AlexF488N/ | |
| 3′ Alexa | | |
| Fluor ™ 488 | | |
| (NHS Ester) | | |

TABLE 2
Fe²⁺ enzyme strands.

| | | SEQ |
|---|---|---|
| Strand | Sequence | ID NO |
| Fe(II)-H5-E | /5IABkFQ/TGGATATCTCCTAGCCAGACTGTTATGTGTGA | 18 |
| | TACGGCAAACTTCGTGATGCCTCTACGGGTCCG | |
| Fe(II)-H5-E | TGGATATCTCCTAGCCAGACTGTTATGTGTGATACGGCA | 24 |
| | AACTTCGTGATGCCTCTACGGGTCCG | |
| Iron sensor | /5IAbRQ/TGGATATCTCCTAGCCAGACTGTTATGTGTGAT | 28 |
| (Fe²⁺) | ACGGCAAACTTCGTGATGCCTCTACGGGTCCG | |
| Fe²⁺ H5 | TGGATATCTCCTAGCCAGACTGTTATGTGTGATACGGCT | 35 |
| DNAzyme | AACTTCGTGATGCCTCTACGGGTCCG | |
| A39T | | |
| Fe²⁺ H5 | TGGATATCTCCTAGCCAGACTGTTATGTGTGATACGGCG | 36 |
| DNAzyme | AACTTCGTGATGCCTCTACGGGTCCG | |
| A39G | | |
| Fe²⁺ H5 | TGGATATCTCCTAGCCAGACTGTTATGTGTGATACGGCA | 37 |
| DNAzyme | CACTTCGTGATGCCTCTACGGGTCCG | |
| A40C | | |
| Fe²⁺ H5 | TGGATATCTCCTAGCCAGACTGTTATGTGTGATAC | 38 |
| DNAzyme | GGCAAACTTCGTGATCCCTCTACGGGTCCG | |
| G51C | | |
| Fe²⁺ H5 | TGGATATCTCCTAGCCAGACTGTTATGTGTGA | 39 |
| DNAzyme | TACGGCAAACTTCGTGATGGCTCTACGGGTCCG | |
| C52G | | |
| Fe²⁺ H5 | TGGATATCTCCTAGCCAGACTGTTATGTGTG | 40 |
| DNAzyme | ATACGGCAAACTTCGTGATGCCCCTACGGGTCCG | |
| T54C | | |
| Fe²⁺ H5 | TGGATATCACCTAGCCAGACTGTTATGTGTGATACGGC | 112 |
| DNAzyme T9A | AAACTTCGTGATGCCTCTACGGGTCCG | |
| Fe²⁺ H5 | TGGATATCCCCTAGCCAGACTGTTATGTGTGATACGGCA | 113 |
| DNAzyme T9C | AACTTCGTGATGCCTCTACGGGTCCG | |
| Fe²⁺ H5 | TGGATATCGCCTAGCCAGACTGTTATGTGTGATACGGC | 114 |
| DNAzyme T9G | AAACTTCGTGATGCCTCTACGGGTCCG | |
| Fe²⁺ H5 | TGGATATCTGCTAGCCAGACTGTTATGTGTGATACGGCA | 115 |
| DNAzyme | AACTTCGTGATGCCTCTACGGGTCCG | |
| C10G | | |
| Fe²⁺ H5 | TGGATATCTTCTAGCCAGACTGTTATGTGTGATACGGCA | 116 |
| DNAzyme | AACTTCGTGATGCCTCTACGGGTCCG | |
| C10T | | |
| Fe²⁺ H5 | TGGATATCTACTAGCCAGACTGTTATGTGTGATACGGCA | 117 |
| DNAzyme | AACTTCGTGATGCCTCTACGGGTCCG | |
| C10A | | |
| Fe²⁺ H5 | TGGATATCTCCTTGCCAGACTGTTATGTGTGATACGGCA | 118 |
| DNAzyme | AACTTCGTGATGCCTCTACGGGTCCG | |
| A13T | | |
| Fe²⁺ H5 | TGGATATCTCCTCGCCAGACTGTTATGTGTGATACGGCA | 119 |
| DNAzyme | AACTTCGTGATGCCTCTACGGGTCCG | |
| A13C | | |
| Fe²⁺ H5 | TGGATATCTCCTAACCAGACTGTTATGTGTGATACGGCA | 120 |
| DNAzyme | AACTTCGTGATGCCTCTACGGGTCCG | |
| G14A | | |
| Fe²⁺ H5 | TGGATATCTCCTACCCAGACTGTTATGTGTGATACGGCA | 121 |
| DNAzyme | AACTTCGTGATGCCTCTACGGGTCCG | |
| G14C | | |
| Fe²⁺ H5 | TGGATATCTCCTATCCAGACTGTTATGTGTGATACGGCA | 122 |
| DNAzyme | AACTTCGTGATGCCTCTACGGGTCCG | |
| G14T | | |
| Fe²⁺ H5 | TGGATATCTCCTAGGCAGACTGTTATGTGTGATACGGCA | 123 |
| DNAzyme | AACTTCGTGATGCCTCTACGGGTCCG | |
| C15G | | |
| Fe²⁺ H5 | TGGATATCTCCTAGTCAGACTGTTATGTGTGATACGGCA | 124 |
| DNAzyme | AACTTCGTGATGCCTCTACGGGTCCG | |
| C15T | | |
| Fe²⁺ H5 | TGGATATCTCCTAGACAGACTGTTATGTGTGATACGGCA | 125 |
| DNAzyme | AACTTCGTGATGCCTCTACGGGTCCG | |
| C15A | | |
| Fe²⁺ H5 | TGGATATCTCCTAGCGAGACTGTTATGTGTGATACGGCA | 126 |
| DNAzyme | AACTTCGTGATGCCTCTACGGGTCCG | |
| C16G | | |
| Fe²⁺ H5 | TGGATATCTCCTAGCTAGACTGTTATGTGTGATACGGCA | 127 |
| DNAzyme | AACTTCGTGATGCCTCTACGGGTCCG | |
| C16T | | |
| Fe²⁺ H5 | TGGATATCTCCTAGCAAGACTGTTATGTGTGATACGGCA | 128 |
| DNAzyme | AACTTCGTGATGCCTCTACGGGTCCG | |
| C16A | | |
| Fe²⁺ H5 | TGGATATCTCCTAGCCAGGCTGTTATGTGTGATACGGCA | 129 |
| DNAzyme | AACTTCGTGATGCCTCTACGGGTCCG | |
| A19G | | |
| Fe²⁺ H5 | TGGATATCTCCTAGCCAGTCTGTTATGTGTGATACGGCA | 130 |
| DNAzyme | AACTTCGTGATGCCTCTACGGGTCCG | |
| A19T | | |
| Fe²⁺ H5 | TGGATATCTCCTAGCCAGCCTGTTATGTGTGATACGGCA | 131 |
| DNAzyme | AACTTCGTGATGCCTCTACGGGTCCG | |
| A19C | | |
| Fe²⁺ H5 | TGGATATCTCCTAGCCAGAGTGTTATGTGTGATACGGCA | 132 |
| DNAzyme | AACTTCGTGATGCCTCTACGGGTCCG | |
| C20G | | |
| Fe²⁺ H5 | TGGATATCTCCTAGCCAGATTGTTATGTGTGATACGGCA | 133 |
| DNAzyme | AACTTCGTGATGCCTCTACGGGTCCG | |
| C20T | | |
| Fe²⁺ H5 | TGGATATCTCCTAGCCAGAATGTTATGTGTGATACGGCA | 134 |
| DNAzyme | AACTTCGTGATGCCTCTACGGGTCCG | |
| C20A | | |
| Fe²⁺ H5 | TGGATATCTCCTAGCCAGACAGTTATGTGTGATACGGC | 135 |
| DNAzyme | AAACTTCGTGATGCCTCTACGGGTCCG | |
| T21A | | |
| Fe²⁺ H5 | TGGATATCTCCTAGCCAGACCGTTATGTGTGATACGGCA | 136 |
| DNAzyme | AACTTCGTGATGCCTCTACGGGTCCG | |
| T21C | | |
| Fe²⁺ H5 | TGGATATCTCCTAGCCAGACGGTTATGTGTGATACGGC | 137 |
| DNAzyme | AAACTTCGTGATGCCTCTACGGGTCCG | |
| T21G | | |
| Fe²⁺ H5 | TGGATATCTCCTAGCCAGACTGTTGTGTGTGATACGGCA | 138 |
| DNAzyme | AACTTCGTGATGCCTCTACGGGTCCG | |
| A25G | | |
| Fe²⁺ H5 | TGGATATCTCCTAGCCAGACTGTTTTGTGTGATACGGCA | 139 |
| DNAzyme | AACTTCGTGATGCCTCTACGGGTCCG | |
| A25T | | |
| Fe²⁺ H5 | TGGATATCTCCTAGCCAGACTGTTCTGTGTGATACGGCA | 140 |
| DNAzyme | AACTTCGTGATGCCTCTACGGGTCCG | |
| A25C | | |
| Fe²⁺ H5 | TGGATATCTCCTAGCCAGACTGTTAAGTGTGATACGGC | 141 |
| DNAzyme | AAACTTCGTGATGCCTCTACGGGTCCG | |
| T26A | | |
| Fe²⁺ H5 | TGGATATCTCCTAGCCAGACTGTTACGTGTGATACGGCA | 142 |
| DNAzyme | AACTTCGTGATGCCTCTACGGGTCCG | |
| T26C | | |
| Fe²⁺ H5 | TGGATATCTCCTAGCCAGACTGTTAGGTGTGATACGGC | 143 |
| DNAzyme | AAACTTCGTGATGCCTCTACGGGTCCG | |
| T26G | | |
| Fe²⁺ H5 | TGGATATCTCCTAGCCAGACTGTTATGTATGATACGGCA | 144 |
| DNAzyme | AACTTCGTGATGCCTCTACGGGTCCG | |
| G29A | | |
| Fe²⁺ H5 | TGGATATCTCCTAGCCAGACTGTTATGTCTGATACGGCA | 145 |
| DNAzyme | AACTTCGTGATGCCTCTACGGGTCCG | |
| G29C | | |
| Fe²⁺ H5 | TGGATATCTCCTAGCCAGACTGTTATGTTTGATACGGCA | 146 |
| DNAzyme | AACTTCGTGATGCCTCTACGGGTCCG | |
| G29T | | |
| Fe²⁺ H5 | TGGATATCTCCTAGCCAGACTGTTATGTGAGATACGGC | 147 |
| DNAzyme | AAACTTCGTGATGCCTCTACGGGTCCG | |
| T30A | | |
| Fe²⁺ H5 | TGGATATCTCCTAGCCAGACTGTTATGTGCGATACGGCA | 148 |
| DNAzyme | AACTTCGTGATGCCTCTACGGGTCCG | |
| T30C | | |
| Fe²⁺ H5 | TGGATATCTCCTAGCCAGACTGTTATGTGGGATACGGC | 149 |
| DNAzyme | AAACTTCGTGATGCCTCTACGGGTCCG | |
| T30G | | |
| Fe²⁺ H5 | TGGATATCTCCTAGCCAGACTGTTATGTGTAATACGGCA | 150 |
| DNAzyme | AACTTCGTGATGCCTCTACGGGTCCG | |
| G31A | | |
| Fe²⁺ H5 | TGGATATCTCCTAGCCAGACTGTTATGTGTCATACGGCA | 151 |
| DNAzyme | AACTTCGTGATGCCTCTACGGGTCCG | |
| G31C | | |
| Fe²⁺ H5 | TGGATATCTCCTAGCCAGACTGTTATGTGTTATACGGCA | 152 |
| DNAzyme | AACTTCGTGATGCCTCTACGGGTCCG | |
| G31T | | |
| Fe²⁺ H5 | TGGATATCTCCTAGCCAGACTGTTATGTGTGGTACGGCA | 153 |
| DNAzyme | AACTTCGTGATGCCTCTACGGGTCCG | |
| A32G | | |
| Fe²⁺ H5 | TGGATATCTCCTAGCCAGACTGTTATGTGTGTTACGGCA | 154 |
| DNAzyme | AACTTCGTGATGCCTCTACGGGTCCG | |
| A32T | | |
| Fe²⁺ H5 | TGGATATCTCCTAGCCAGACTGTTATGTGTGCTACGGCA | 155 |
| DNAzyme | AACTTCGTGATGCCTCTACGGGTCCG | |
| A32C | | |
| Fe²⁺ H5 | TGGATATCTCCTAGCCAGACTGTTATGTGTGAAACGGC | 156 |
| DNAzyme | AAACTTCGTGATGCCTCTACGGGTCCG | |
| T33A | | |
| Fe²⁺ H5 | TGGATATCTCCTAGCCAGACTGTTATGTGTGACACGGCA | 157 |
| DNAzyme | AACTTCGTGATGCCTCTACGGGTCCG | |
| T33C | | |
| Fe²⁺ H5 | TGGATATCTCCTAGCCAGACTGTTATGTGTGAGACGGC | 158 |
| DNAzyme | AAACTTCGTGATGCCTCTACGGGTCCG | |
| T33G | | |
| Fe²⁺ H5 | TGGATATCTCCTAGCCAGACTGTTATGTGTGATACAGCA | 159 |
| DNAzyme | AACTTCGTGATGCCTCTACGGGTCCG | |
| G36A | | |
| Fe²⁺ H5 | TGGATATCTCCTAGCCAGACTGTTATGTGTGATACCGCA | 160 |
| DNAzyme | AACTTCGTGATGCCTCTACGGGTCCG | |
| G36C | | |
| Fe²⁺ H5 | TGGATATCTCCTAGCCAGACTGTTATGTGTGATACTGCA | 161 |
| DNAzyme | AACTTCGTGATGCCTCTACGGGTCCG | |
| G36T | | |
| Fe²⁺ H5 | TGGATATCTCCTAGCCAGACTGTTATGTGTGATACGACA | 162 |
| DNAzyme | AACTTCGTGATGCCTCTACGGGTCCG | |
| G37A | | |
| Fe²⁺ H5 | TGGATATCTCCTAGCCAGACTGTTATGTGTGATACGCCA | 163 |
| DNAzyme | AACTTCGTGATGCCTCTACGGGTCCG | |
| G37C | | |
| Fe²⁺ H5 | TGGATATCTCCTAGCCAGACTGTTATGTGTGATACGTCA | 164 |
| DNAzyme | AACTTCGTGATGCCTCTACGGGTCCG | |
| G37T | | |
| Fe²⁺ H5 | TGGATATCTCCTAGCCAGACTGTTATGTGTGATACGGGA | 165 |
| DNAzyme | AACTTCGTGATGCCTCTACGGGTCCG | |
| C38G | | |
| Fe²⁺ H5 | TGGATATCTCCTAGCCAGACTGTTATGTGTGATACGGTA | 166 |
| DNAzyme | AACTTCGTGATGCCTCTACGGGTCCG | |
| C38T | | |
| Fe²⁺ H5 | TGGATATCTCCTAGCCAGACTGTTATGTGTGATACGGAA | 167 |
| DNAzyme | AACTTCGTGATGCCTCTACGGGTCCG | |
| C38A | | |
| Fe²⁺ H5 | TGGATATCTCCTAGCCAGACTGTTATGTGTGATACGGCC | 168 |
| DNAzyme | AACTTCGTGATGCCTCTACGGGTCCG | |
| A39C | | |
| Fe²⁺ H5 | TGGATATCTCCTAGCCAGACTGTTATGTGTGATACGGCA | 169 |
| DNAzyme | GACTTCGTGATGCCTCTACGGGTCCG | |
| A40G | | |
| Fe²⁺ H5 | TGGATATCTCCTAGCCAGACTGTTATGTGTGATACGGCA | 170 |
| DNAzyme | TACTTCGTGATGCCTCTACGGGTCCG | |
| A40T | | |
| Fe²⁺ H5 | TGGATATCTCCTAGCCAGACTGTTATGTGTGATACGGCA | 171 |
| DNAzyme | AACATCGTGATGCCTCTACGGGTCCG | |
| T43A | | |
| Fe²⁺ H5 | TGGATATCTCCTAGCCAGACTGTTATGTGTGATACGGCA | 172 |
| DNAzyme | AACCTCGTGATGCCTCTACGGGTCCG | |
| T43C | | |
| Fe²⁺ H5 | TGGATATCTCCTAGCCAGACTGTTATGTGTGATACGGCA | 173 |
| DNAzyme | AACGTCGTGATGCCTCTACGGGTCCG | |
| T43G | | |
| Fe²⁺ H5 | TGGATATCTCCTAGCCAGACTGTTATGTGTGATACGGCA | 174 |
| DNAzyme | AACTACGTGATGCCTCTACGGGTCCG | |
| T44A | | |
| Fe²⁺ H5 | TGGATATCTCCTAGCCAGACTGTTATGTGTGATACGGCA | 175 |
| DNAzyme | AACTCCGTGATGCCTCTACGGGTCCG | |
| T44C | | |
| Fe²⁺ H5 | TGGATATCTCCTAGCCAGACTGTTATGTGTGATACGGCA | 176 |
| DNAzyme | AACTGCGTGATGCCTCTACGGGTCCG | |
| T44G | | |
| Fe²⁺ H5 | TGGATATCTCCTAGCCAGACTGTTATGTGTGATACGGCA | 177 |
| DNAzyme | AACTTCGTGAAGCCTCTACGGGTCCG | |
| T50A | | |
| Fe²⁺ H5 | TGGATATCTCCTAGCCAGACTGTTATGTGTGATACGGCA | 178 |
| DNAzyme | AACTTCGTGACGCCTCTACGGGTCCG | |
| T50C | | |
| Fe²⁺ H5 | TGGATATCTCCTAGCCAGACTGTTATGTGTGATACGGCA | 179 |
| DNAzyme | AACTTCGTGAGGCCTCTACGGGTCCG | |
| T50G | | |
| Fe²⁺ H5 | TGGATATCTCCTAGCCAGACTGTTATGTGTGATACGGCA | 180 |
| DNAzyme | AACTTCGTGATACCTCTACGGGTCCG | |
| G51A | | |
| Fe²⁺ H5 | TGGATATCTCCTAGCCAGACTGTTATGTGTGATACGGCA | 181 |
| DNAzyme | AACTTCGTGATTCCTCTACGGGTCCG | |
| G51T | | |
| Fe²⁺ H5 | TGGATATCTCCTAGCCAGACTGTTATGTGTGATACGGCA | 182 |
| DNAzyme | AACTTCGTGATGTCTCTACGGGTCCG | |
| C52T | | |
| Fe²⁺ H5 | TGGATATCTCCTAGCCAGACTGTTATGTGTGATACGGCA | 183 |
| DNAzyme | AACTTCGTGATGACTCTACGGGTCCG | |
| C52A | | |
| Fe²⁺ H5 | TGGATATCTCCTAGCCAGACTGTTATGTGTGATACGGCA | 184 |
| DNAzyme | AACTTCGTGATGCGTCTACGGGTCCG | |
| C53G | | |
| Fe²⁺ H5 | TGGATATCTCCTAGCCAGACTGTTATGTGTGATACGGCA | 185 |
| DNAzyme | AACTTCGTGATGCTTCTACGGGTCCG | |
| C53T | | |
| Fe²⁺ H5 | TGGATATCTCCTAGCCAGACTGTTATGTGTGATACGGCA | 186 |
| DNAzyme | AACTTCGTGATGCATCTACGGGTCCG | |
| C53A | | |
| Fe²⁺ H5 | TGGATATCTCCTAGCCAGACTGTTATGTGTGATACGGCA | 187 |
| DNAzyme | AACTTCGTGATGCCGCTACGGGTCCG | |
| T54G | | |
| Fe²⁺ H5 | TGGATATCTCCTAGCCAGACTGTTATGTGTGATACGGCA | 188 |
| DNAzyme | AACTTCGTGATGCCACTACGGGTCCG | |
| T54A | | |
| Fe²⁺ H5 | TGGATATCTCCTAGCCAGACTGTTATGTGTGATACGGCA | 189 |
| DNAzyme | AACTTCGTGATGCCTGTACGGGTCCG | |
| C55G | | |
| Fe²⁺ H5 | TGGATATCTCCTAGCCAGACTGTTATGTGTGATACGGCA | 190 |
| DNAzyme | AACTTCGTGATGCCTTTACGGGTCCG | |
| C55T | | |
| Fe²⁺ H5 | TGGATATCTCCTAGCCAGACTGTTATGTGTGATACGGCA | 191 |
| DNAzyme | AACTTCGTGATGCCTATACGGGTCCG | |
| C55A | | |
| Fe²⁺ H5 | GGATATCTCCTAGCCAGACTGTTATGTGTGATACGGCA | 242 |
| DNAzyme | AACTTCGTGATGCCTCTACGGGTCCG | |
| Truncation 51 | | |
| Fe²⁺ H5 | GATATCTCCTAGCCAGACTGTTATGTGTGATACGGCAA | 243 |
| DNAzyme | ACTTCGTGATGCCTCTACGGGTCCG | |
| Truncation 52 | | |
| Fe²⁺ H5 | TGGATATCTCCTAGCCAGACTGTTATGTGTGATACGGCA | 244 |
| DNAzyme | AACTTCGTGATGCCTCTACGGGTCC | |
| Truncation 31 | | |
| Fe²⁺ H5 | TGGATATCTCCTAGCCAGACTGTTATGTGTGATACGGCA | 245 |
| DNAzyme | AACTTC GTG ATG CCTCTA CGGTC | |
| Truncation 32 | | |
| Fe²⁺ H5 | TGGATATCTCCTAGCCAGACTGTTATGTGTGATACG | 246 |
| DNAzyme | GCAAACTTCGTGATGCCTCTACGGGT | |
| Truncation 33 | | |
| Fe²⁺ H5 | TGGATATCTCCTAGCCAGACTGTTATGTGTGATACGGCA | 247 |
| DNAzyme | AACTTCGTGATGCCTCTACGGG | |
| Truncation 34 | | |
| Fe²⁺ H5 | TGGATATCTCCTAGCCAGACTGTTATGTGTGATACGGCT | 256 |
| DNAzyme | AACTTCGTGATGGCTCTACGGGTCCG | |
| Combo 1 | | |
| Fe²⁺ H5 | TGGATATCTCCTAGCCAGACTGTTATGTGTGATACGGCA | 257 |
| DNAzyme | CACTTCGTGATGGCTCTACGGGTCCG | |
| Combo 2 | | |
| Fe²⁺ H5 | TGGATATCTCCTAGCCAGACTGTTATGTGTGATACGGCA | 258 |
| DNAzyme | CACTTCGTGATGCCCCTACGGGTCCG | |
| Combo 3 | | |
| Fe²⁺ H5 | TGGATATCTCCTAGCCAGACTGTTATGTGTGATACGGCT | 259 |
| DNAzyme | AACTTCGTGATCCCTCTACGGGTCCG | |
| Combo 4 | | |
| Fe²⁺ H5 | TGGATATCTCCTAGCCAGACTGTTATGTGTGATACGGCA | 260 |
| DNAzyme | AACTTCGTGATCCCCCTACGGGTCCG | |
| Combo 5 | | |
| Fe²⁺ H5 | TGGATATCTCCTAGCCAGACTGTTATGTGTGATACGGCA | 261 |
| DNAzyme | AACTTCGTGATGGCCCTACGGGTCCG | |
| Combo 6 | | |
| Fe²⁺ H5 | TGGATATCTCCTAGCCAGACTGTTATGTGTGATACGGCA | 262 |
| DNAzyme | AACTTCGTGATCGCTCTACGGGTCCG | |
| Combo 7 | | |
| Fe²⁺ H5 | TGGATATCTCCTAGCCAGACTGTTATGTGTGATACGGCA | 263 |
| DNAzyme | CACTTCGTGATCCCTCTACGGGTCCG | |
| Combo 8 | | |
| Fe²⁺ H5 | TGGATATCTCCTAGCCAGACTGTTATGTGTGATACGGCG | 264 |
| DNAzyme | AACTTCGTGATGGCTCTACGGGTCCG | |
| Combo 9 | | |
| Fe²⁺ H5 | TGGATATCTCCTAGCCAGACTGTTATGTGTGATACGGCT | 265 |
| DNAzyme | AACTTCGTGATGCCCCTACGGGTCCG | |
| Combo 10 | | |
| Fe²⁺ H5 | TGGATATCTCCTAGCCAGACTGTTATGTGTGATACGGCG | 266 |
| DNAzyme | AACTTCGTGATGCCCCTACGGGTCCG | |
| Combo 11 | | |
| Fe²⁺ H5 | TGGATATCTCCTAGCCAGACTGTTATGTGTGATACGGCT | 267 |
| DNAzyme | CACTTCGTGATGCCTCTACGGGTCCG | |
| Combo 12 | | |
| Fe²⁺ H5 | TGGATATCTCCTAGCCAGACTGTTATGTGTGATACGGCG | 268 |
| DNAzyme | CACTTCGTGATGCCTCTACGGGTCCG | |
| Combo 13 | | |
| Fe²⁺ H5 | TGGATATCTCCTAGCCAGACTGTTATGTGTGATACGGCG | 269 |
| DNAzyme | AACTTCGTGATCCCTCTACGGGTCCG | |
| Combo 14 | | |
| Fe²⁺ H5 | TGGATATCTCCTAGCCAGACTGTTATGTGTGATACGGCT | 270 |
| DNAzyme | AACTTCGTGATCGCTCTACGGGTCCG | |
| Combo 15 | | |
| Fe²⁺ H5 | TGGATATCTCCTAGCCAGACTGTTATGTGTGATACGGCT | 271 |
| DNAzyme | AACTTCGTGATCCCCCTACGGGTCCG | |
| Combo 16 | | |
| Fe²⁺ H5 | TGGATATCTCCTAGCCAGACTGTTATGTGTGATACGGCA | 272 |
| DNAzyme | CACTTCGTGATCGCTCTACGGGTCCG | |
| Combo 17 | | |
| Fe²⁺ H5 | TGGATATCTCCTAGCCAGACTGTTATGTGTGATACGGCT | 273 |
| DNAzyme | AACTTCGTGATGGCCCTACGGGTCCG | |
| Combo 18 | | |
| Fe²⁺ H5 | TGGATATCTCCTAGCCAGACTGTTATGTGTGATACGGCG | 274 |
| DNAzyme | AACTTCGTGATCCCCCTACGGGTCCG | |
| Combo 19 | | |
| Fe²⁺ H5 | TGGATATCTCCTAGCCAGACTGTTATGTGTGATACGGCA | 275 |
| DNAzyme | CACTTCGTGATCCCCCTACGGGTCCG | |
| Combo 20 | | |
| Fe²⁺ H5 | TGGATATCTCCTAGCCAGACTGTTATGTGTGATACGGCA | 276 |
| DNAzyme | CACTTCGTGATGGCCCTACGGGTCCG | |
| Combo 21 | | |
| Fe²⁺ H5 | TGGATATCTCCTAGCCAGACTGTTATGTGTGATACGGCG | 277 |
| DNAzyme | CACTTCGTGATGGCTCTACGGGTCCG | |
| Combo 22 | | |
| Fe²⁺ H5 | TGGATATCTCCTAGCCAGACTGTTATGTGTGATACGGCT | 278 |
| DNAzyme | CACTTCGTGATGCCCCTACGGGTCCG | |
| Combo 23 | | |
| Fe²⁺ H5 | TGGATATCTCCTAGCCAGACTGTTATGTGTGATACGGCG | 279 |
| DNAzyme | CACTTCGTGATCCCTCTACGGGTCCG | |
| Combo 24 | | |
| Fe²⁺ H5 | TGGATATCTCCTAGCCAGACTGTTATGTGTGATACGGCT | 280 |
| DNAzyme | CACTTCGTGATCCCTCTACGGGTCCG | |
| Combo 25 | | |
| Fe²⁺ H5 | TGGATATCTCCTAGCCAGACTGTTATGTGTGATACGGCG | 281 |
| DNAzyme | AACTTCGTGATGGCCCTACGGGTCCG | |
| Combo 26 | | |
| Fe²⁺ H5 | TGGATATCTCCTAGCCAGACTGTTATGTGTGATACGGCT | 282 |
| DNAzyme | CACTTCGTGATGGCTCTACGGGTCCG | |
| Combo 27 | | |
| Fe²⁺ H5 | TGGATATCTCCTAGCCAGACTGTTATGTGTGATACGGCA | 283 |
| DNAzyme | AACTTCGTGATCGCCCTACGGGTCCG | |
| Combo 28 | | |
| Fe²⁺ H5 | TGGATATCTCCTAGCCAGACTGTTATGTGTGATACGGCG | 284 |
| DNAzyme | AACTTCGTGATCGCTCTACGGGTCCG | |
| Combo 29 | | |
| Fe²⁺ H5 | TGGATATCTCCTAGCCAGACTGTTATGTGTGATACGGCG | 285 |
| DNAzyme | CACTTCGTGATGCCCCTACGGGTCCG | |
| Combo 30 | | |
| Fe²⁺ H5 | TGGATATCTCCTAGCCAGACTGTTATGTGTGATACGGCG | 286 |
| DNAzyme | CACTTCGTGATCGCTCTACGGGTCCG | |
| Combo 31 | | |
| Fe²⁺ H5 | TGGATATCTCCTAGCCAGACTGTTATGTGTGATACGGCT | 287 |
| DNAzyme | AACTTCGTGATCGCCCTACGGGTCCG | |
| Combo 32 | | |
| Fe²⁺ H5 | TGGATATCTCCTAGCCAGACTGTTATGTGTGATACGGCT | 288 |
| DNAzyme | CACTTCGTGATCCCCCTACGGGTCCG | |
| Combo 33 | | |
| Fe²⁺ H5 | TGGATATCTCCTAGCCAGACTGTTATGTGTGATACGGCT | 289 |
| DNAzyme | CACTTCGTGATCGCTCTACGGGTCCG | |
| Combo 34 | | |
| Fe²⁺ H5 | TGGATATCTCCTAGCCAGACTGTTATGTGTGATACGGCT | 290 |
| DNAzyme | CACTTCGTGATGGCCCTACGGGTCCG | |
| Combo 35 | | |
| Fe²⁺ H5 | TGGATATCTCCTAGCCAGACTGTTATGTGTGATACGGCA | 291 |
| DNAzyme | CACTTCGTGATCGCCCTACGGGTCCG | |
| Combo 36 | | |
| Fe²⁺ H5 | TGGATATCTCCTAGCCAGACTGTTATGTGTGATACGGCG | 292 |
| DNAzyme | CACTTCGTGATCCCCCTACGGGTCCG | |
| Combo 37 | | |
| Fe²⁺ H5 | TGGATATCTCCTAGCCAGACTGTTATGTGTGATACGGCG | 293 |
| DNAzyme | AACTTCGTGATCGCCCTACGGGTCCG | |
| Combo 38 | | |
| Fe²⁺ H5 | TGGATATCTCCTAGCCAGACTGTTATGTGTGATACGGCG | 294 |
| DNAzyme | CACTTCGTGATGGCCCTACGGGTCCG | |
| Combo 39 | | |
| Fe²⁺ H5 | TGGATATCTCCTAGCCAGACTGTTATGTGTGATACGGCT | 295 |
| DNAzyme | CACTTCGTGATCGCCCTACGGGTCCG | |
| Combo 40 | | |
| Fe²⁺ H5 | TGGATATCTCCTAGCCAGACTGTTATGTGTGATACGGCG | 296 |
| DNAzyme | CACTTCGTGATCGCCCTACGGGTCCG | |
| Combo 41 | | |
| Fe²⁺ H5 | /5IABkFQ/TGGATATCTCCTAGCCAGACTGTTATGTGTGA | 297 |
| DNAzyme | TACGGCGAACTTCGTGATGCCTCTACGGGTCCG | |
| A39G 5′ Iowa | | |
| Black ™ FQ | | |
| Fe²⁺ H5 | /5IABkFQ/TGGATATCTCCTAGCCAGACTGTTATGTGTGA | 298 |
| DNAzyme | TACGGCACACTTCGTGATGCCTCTACGGGTCCG | |
| A40C 5′ Iowa | | |
| Black ™ FQ | | |
| Fe²⁺ H5 | /5IABkFQ/TGGATATCTCCTAGCCAGACTGTTATGTGTGA | 299 |
| DNAzyme | TACGGCAAACTTCGTGATGCCCCTACGGGTCCG | |
| T54C 5′ Iowa | | |
| Black ™ FQ | | |

TABLE 3
Fe³⁺ substrate strands.

| | | SEQ |
|---|---|---|
| Strand | Sequence | ID NO |
| Fe(III)-B12-rS | /5Alex647N/CTCTATTArGGGAGACTCGCATGCCGC/3IAbR | 17 |
| | QSp/ | |
| Fe(III)-B12-rS | CTCTATTArGGGAGACTCGCATGCCGC | 23 |
| Fe³⁺ B12 | CTCTATTArGGGAGGCTCGCATGCCGC | 33 |
| Substrate A14G | | |
| Fe³⁺ B12 | CTCTATTArGGGAGAATCGCATGCCGC | 34 |
| Substrate C15A | | |
| Fe³⁺ B12 | CTCTATTAGGGAGACTCGCATGCCGC | 76 |
| Original | | |
| Substrate (2′H) | | |
| Fe³⁺ B12 | CTCTATTAmGGGAGACTCGCATGCCGC | 77 |
| Original | | |
| Substrate | | |
| (2′OMe) | | |
| Fe³⁺ B12 | /56-FAM/CTCTATTArGGGAGACTCGCATGCCGC | 192 |
| Original | | |
| Substrate FAM | | |
| Fe³⁺ B12 | /56-FAM/CTCTATTArGGAAGACTCGCATGCCGC | 193 |
| Substrate G11A | | |
| FAM | | |
| Fe³⁺ B12 | /56-FAM/CTCTATTArGGTAGACTCGCATGCCGC | 194 |
| Substrate G11T | | |
| FAM | | |
| Fe³⁺ B12 | /56-FAM/CTCTATTArGGCAGACTCGCATGCCGC | 195 |
| Substrate G11C | | |
| FAM | | |
| Fe³⁺ B12 | /56-FAM/CTCTATTArGGGTGACTCGCATGCCGC | 196 |
| Substrate A12T | | |
| FAM | | |
| Fe³⁺ B12 | /56-FAM/CTCTATTArGGGCGACTCGCATGCCGC | 197 |
| Substrate A12C | | |
| FAM | | |
| Fe³⁺ B12 | /56-FAM/CTCTATTArGGGGGACTCGCATGCCGC | 198 |
| Substrate A12G | | |
| FAM | | |
| Fe³⁺ B12 | /56-FAM/CTCTATTArGGGAAACTCGCATGCCGC | 199 |
| Substrate G13A | | |
| FAM | | |
| Fe³⁺ B12 | /56-FAM/CTCTATTArGGGATACTCGCATGCCGC | 200 |
| Substrate G13T | | |
| FAM | | |
| Fe³⁺ B12 | /56-FAM/CTCTATTArGGGACACTCGCATGCCGC | 201 |
| Substrate G13C | | |
| FAM | | |
| Fe³⁺ B12 | /56-FAM/CTCTATTArGGGAGTCTCGCATGCCGC | 202 |
| Substrate A14T | | |
| FAM | | |
| Fe³⁺ B12 | /56-FAM/CTCTATTArGGGAGCCTCGCATGCCGC | 203 |
| Substrate A14C | | |
| FAM | | |
| Fe³⁺ B12 | /56-FAM/CTCTATTArGGGAGGCTCGCATGCCGC | 204 |
| Substrate A14G | | |
| FAM | | |
| Fe³⁺ B12 | /56-FAM/CTCTATTArGGGAGAATCGCATGCCGC | 205 |
| Substrate C15A | | |
| FAM | | |
| Fe³⁺ B12 | /56-FAM/CTCTATTArGGGAGATTCGCATGCCGC | 206 |
| Substrate C15T | | |
| FAM | | |
| Fe³⁺ B12 | /56-FAM/CTCTATTArGGGAGAGTCGCATGCCGC | 207 |
| Substrate C15G | | |
| FAM | | |
| Fe³⁺ B12 | /56-FAM/CTCTATTArGGGAGACACGCATGCCGC | 208 |
| Substrate T16A | | |
| FAM | | |
| Fe³⁺ B12 | /56-FAM/CTCTATTArGGGAGACCCGCATGCCGC | 209 |
| Substrate T16C | | |
| FAM | | |
| Fe³⁺ B12 | /56-FAM/CTCTATTArGGGAGACGCGCATGCCGC | 210 |
| Substrate T16G | | |
| FAM | | |
| Fe³⁺ B12 | /5Alex647N/CTCTATTArGGGAGGCTCGCATGCCGC/3IAbR | 303 |
| Substrate A14G | QSp/ | |
| 5′ Alexa | | |
| Fluor™ 647 | | |
| (NHS Ester) | | |
| Fe³⁺ B12 | /5Alex647N/CTCTATTArGGGAGAATCGCATGCCGC/3IAbR | 304 |
| Substrate C15A | QSp/ | |
| 5′ Alexa | | |
| Fluor™ 647 | | |
| (NHS Ester) | | |
| Fe³⁺ B12 | /5Alex647N/CTCTATTArGGGAGATTCGCATGCCGC/3IAbR | 305 |
| Substrate C15T | QSp/ | |
| 5′ Alexa | | |
| Fluor™ 647 | | |
| (NHS Ester) | | |

TABLE 4
Fe³⁺ enzyme strands.

| | | SEQ |
|---|---|---|
| Strand | Sequence | ID NO |
| Fe(III)-B12-E | GCGGCATGCGCGTTTGCGGCACCTAAACGCTCCTAATA | 15 |
| | GAG/3IAbRQSp/ | |
| Fe(III)-B12-E | GCGGCATGCGCGTTTGCGGCACCTAAACGCTCCTAATA | 21 |
| | GAG | |
| Fe³⁺ B12 | GCGGCATGCGCGTTTGCGGTACCTAAACGCTCCTAATA | 32 |
| DNAzyme | GAG | |
| C20T | | |
| Fe³⁺ B12 | GCGGCATGCGCGTTTACGGCACCTAAACGCTCCTAATA | 211 |
| DNAzyme | GAG | |
| G16A | | |
| Fe³⁺ B12 | GCGGCATGCGCGTTTCCGGCACCTAAACGCTCCTAATA | 212 |
| DNAzyme | GAG | |
| G16C | | |
| Fe³⁺ B12 | GCGGCATGCGCGTTTTCGGCACCTAAACGCTCCTAATA | 213 |
| DNAzyme | GAG | |
| G16T | | |
| Fe³⁺ B12 | GCGGCATGCGCGTTTGGGGCACCTAAACGCTCCTAATA | 214 |
| DNAzyme | GAG | |
| C17G | | |
| Fe³⁺ B12 | GCGGCATGCGCGTTTGTGGCACCTAAACGCTCCTAATA | 215 |
| DNAzyme | GAG | |
| C17T | | |
| Fe³⁺ B12 | GCGGCATGCGCGTTTGAGGCACCTAAACGCTCCTAATA | 216 |
| DNAzyme | GAG | |
| C17A | | |
| Fe³⁺ B12 | GCGGCATGCGCGTTTGCAGCACCTAAACGCTCCTAATA | 217 |
| DNAzyme | GAG | |
| G18A | | |
| Fe³⁺ B12 | GCGGCATGCGCGTTTGCTGCACCTAAACGCTCCTAATA | 218 |
| DNAzyme | GAG | |
| G18T | | |
| Fe³⁺ B12 | GCGGCATGCGCGTTTGCCGCACCTAAACGCTCCTAATA | 219 |
| DNAzyme | GAG | |
| G18C | | |
| Fe³⁺ B12 | GCGGCATGCGCGTTTGCGACACCTAAACGCTCCTAATA | 220 |
| DNAzyme | GAG | |
| G19A | | |
| Fe³⁺ B12 | GCGGCATGCGCGTTTGCGCCACCTAAACGCTCCTAATA | 221 |
| DNAzyme | GAG | |
| G19C | | |
| Fe³⁺ B12 | GCGGCATGCGCGTTTGCGTCACCTAAACGCTCCTAATA | 222 |
| DNAzyme | GAG | |
| G19T | | |
| Fe³⁺ B12 | GCGGCATGCGCGTTTGCGGGACCTAAACGCTCCTAATA | 223 |
| DNAzyme | GAG | |
| C20G | | |
| Fe³⁺ B12 | GCGGCATGCGCGTTTGCGGAACCTAAACGCTCCTAATA | 224 |
| DNAzyme | GAG | |
| C20A | | |
| Fe³⁺ B12 | GCGGCATGCGCGTTTGCGGCGCCTAAACGCTCCTAATA | 225 |
| DNAzyme | GAG | |
| A21G | | |
| Fe³⁺ B12 | GCGGCATGCGCGTTTGCGGCCCCTAAACGCTCCTAATA | 226 |
| DNAzyme | GAG | |
| A21C | | |
| Fe³⁺ B12 | GCGGCATGCGCGTTTGCGGCTCCTAAACGCTCCTAATA | 227 |
| DNAzyme | GAG | |
| A21T | | |
| Fe³⁺ B12 | GCGGCATGCGCGTTTGCGGCAGCTAAACGCTCCTAATA | 228 |
| DNAzyme | GAG | |
| C22G | | |
| Fe³⁺ B12 | GCGGCATGCGCGTTTGCGGCATCTAAACGCTCCTAATA | 229 |
| DNAzyme | GAG | |
| C22T | | |
| Fe³⁺ B12 | GCGGCATGCGCGTTTGCGGCAACTAAACGCTCCTAATA | 230 |
| DNAzyme | GAG | |
| C22A | | |
| Fe³⁺ B12 | GCGGCATGCGCGTTTGCGGCACGTAAACGCTCCTAATA | 231 |
| DNAzyme | GAG | |
| C23G | | |
| Fe³⁺ B12 | GCGGCATGCGCGTTTGCGGCACTTAAACGCTCCTAATA | 232 |
| DNAzyme | GAG | |
| C23T | | |
| Fe³⁺ B12 | GCGGCATGCGCGTTTGCGGCACATAAACGCTCCTAATA | 233 |
| DNAzyme | GAG | |
| C23A | | |
| Fe³⁺ B12 | GCGGCATGCGCGTTTGCGGCACCGAAACGCTCCTAATA | 234 |
| DNAzyme | GAG | |
| T24G | | |
| Fe³⁺ B12 | GCGGCATGCGCGTTTGCGGCACCCAAACGCTCCTAATA | 235 |
| DNAzyme | GAG | |
| T24C | | |
| Fe³⁺ B12 | GCGGCATGCGCGTTTGCGGCACCAAAACGCTCCTAATA | 236 |
| DNAzyme | GAG | |
| T24A | | |
| Fe³⁺ B12 | GCGGCATGCGCGTTTGCGGCACCTAAACGGTCCTAATA | 237 |
| DNAzyme | GAG | |
| C30G | | |
| Fe³⁺ B12 | GCGGCATGCGCGTTTGCGGCACCTAAACGTTCCTAATA | 238 |
| DNAzyme | GAG | |
| C30T | | |
| Fe³⁺ B12 | GCGGCATGCGCGTTTGCGGCACCTAAACGATCCTAATA | 239 |
| DNAzyme | GAG | |
| C30A | | |
| Fe³⁺ B12 | GCGGCATGCGCGTTTGCGGCACCTAAACGCACCTAATA | 240 |
| DNAzyme | GAG | |
| T31A | | |
| Fe³⁺ B12 | GCGGCATGCGCGTTTGCGGCACCTAAACGCGCCTAATA | 241 |
| DNAzyme | GAG | |
| T31G | | |
| Fe³⁺ B12 | CGGCATGCGCGTTTGCGGCACCTAAACGCTCCTAATAG | 248 |
| DNAzyme | AG | |
| Trunctation 51 | | |
| Fe³⁺ B12 | GGCATGCGCGTTTGCGGCACCTAAACGCTCCTAATAGA | 249 |
| DNAzyme | G | |
| Trunctation 52 | | |
| Fe³⁺ B12 | GCATGCGCGTTTGCGGCACCTAAACGCTCCTAATAGAG | 250 |
| DNAzyme | | |
| Trunctation 53 | | |
| Fe³⁺ B12 | CATGCGCGTTTGCGGCACCTAAACGCTCCTAATAGAG | 251 |
| DNAzyme | | |
| Trunctation 54 | | |
| Fe³⁺ B12 | ATGCGCGTTTGCGGCACCTAAACGCTCCTAATAGAG | 252 |
| DNAzyme | | |
| Trunctation 55 | | |
| Fe³⁺ B12 | GCGGCATGCGCGTTTGCGGCACCTAAACGCTCCTAATA | 253 |
| DNAzyme | GA | |
| Trunctation 31 | | |
| Fe³⁺ B12 | GCGGCATGCGCGTTTGCGGCACCTAAACGCTCCTAATA | 254 |
| DNAzyme | G | |
| Trunctation 32 | | |
| Fe³⁺ B12 | GCGGCATGCGCGTTTGCGGCACCTAAACGCTCCTAATA | 255 |
| DNAzyme | | |
| Trunctation 33 | | |
| Fe³⁺ B12 | GCGGCATGCGCGTTTGTGGTACCTAAACGCTCCTAATA | 302 |
| DNAzyme | GAG/3IAbRQSp/ | |
| C20T 3′ Iowa | | |
| BlackTM RQ | | |

TABLE 5
Cu⁺ substrate strands.

| | | SEQ |
|---|---|---|
| Strand | Sequence | ID NO |
| Cu1 aS Cy5 | AGCTTCTTTCTAATACGGCTTACCAC | 44 |
| Cu1 aS Cy5 | AGCTTCTTTCTAATACGGCTTACCA | 54 |
| | C/3Cy5Sp/ | |
| Cu1 aS Cy5 | /5IAbRQ/AGCTTCTTTCTAATACG | 55 |
| IBRQ | GCTTACCAC/3Cy5Sp/ | |
| Cu1 aS 3Cy5 | /5IAbRQ/ACTTCCTTTCTAATAC | 63 |
| 5IBRQ v2 | GGCTTACCACAT/3Cy5Sp/ | |
| Cu1 aS Cy5 | /5IAbRQ/AGCTTCTTTCTAATAC | 69 |
| | GGCTTACCACT/3Cy59p/ | |

TABLE 6
Cu+ enzyme strands.

| | | SEQ |
|---|---|---|
| Strand | Sequence | ID NO |
| Cu1 aE IBRQ | GTGGTAAGCCTGGGCCTCT | 45 |
| | TTCTTTTTAAGAAAGAAC | |
| Cu1 aE IBRQ | /5IAbRQ/GTGGTAAGCC | 56 |
| | TGGGCCTCTTTCTTTTTA | |
| | AGAAAGAAC | |
| Cu1 aE 5IBRQ | /5IAbRQ/ATGTGGTAAG | 64 |
| v2 | CCTGGGCCTCTTTCCTTT | |
| | TTAAGGAAAGAAC | |

TABLE 7
Cu²⁺ substrate strands.

| | | SEQ |
|---|---|---|
| Strand | Sequence | ID NO |
| Cu2 PSrS | AGTCACTATrA*GGAAGATGGCGAAA | 47 |
| Cu2 PSrS | /56-FAM/AGTCACTATrA*GGAAGA | 58 |
| 5FAM | TGGCGAAA | |
| Cu2 PSrS | /56-FAM/AGTCACTATrA*GGAAG | 59 |
| 5FAM 3IBFQ | ATGGCGAAA/3IABKFQ/ | |
| Cu2 PSrS | /56-FAM/AGCACTATrA*GGAAGC | 66 |
| 5FAM 3IBFQ | ATGGCGACG/3IABKFQ/ | |
| v2 | | |
| Cu2 PSrS | /56-FAM/AGTCACTATrA*GGAAGA | 71 |
| FAM | TGGCGAAA/3Dab/ | |
| Cu2 dS | /56-FAM/AGTCACTATAGGAAGATG | 72 |
| FAM | GCGAAA/3Dab/ | |

* = phosphorothioate modification

TABLE 8
Cu²⁺ enzyme strands.

| | | SEQ |
|---|---|---|
| Strand | Sequence | ID NO |
| Cu2 aE | TTTCGCCATCTTCAC | 48 |
| | CAGGAAATAGTGACT | |
| Cu2 aE 3IBFQ | TTTCGCCATCTTCAC | 60 |
| | CAGGAAATAGTGAC | |
| | T/3IABKFQ/ | |
| Cu2 aE 3IBFQ | CGTCGCCATGCTTCA | 67 |
| v2 | CCAGGAAATAGTGC | |
| | T/3IABKFQ/ | |
| Cu2 aE Q | TTTCGCCATCTTCAC | 73 |
| | CAGGAAATAGTGAC | |
| | T/3Dab/ | |

In some aspects, the substrate strand and/or enzyme strand can include at least one point mutation (e.g., at least 2 point mutations, at least 3 point mutations, at least 4 point mutations, at least 5 point mutations). In some aspects, the point mutation can include one or more of C20T, A14G, C15A, A39T, A39G, A40C, G51C, C52G, T54C, T11C, A13C, or A14T.

In another aspect, provided is a composition for simultaneously detecting a target ion in multiple oxidation states, the composition comprising: i) a first DNAzyme sensor comprising: a first substrate strand comprising a first cleavage site, wherein the first cleavage site is uncleaved when the target ion in a first oxidation state is not present; and a first enzyme strand at least partially complementary to the first substrate strand and comprising a first catalytic loop; wherein the first catalytic loop is capable of cleaving the first substrate strand at the first cleavage site in the presence of the target ion in the first oxidation state, wherein said cleavage provides a first detectable signal; and ii) a second DNAzyme sensor comprising: a second substrate strand comprising a second cleavage site, wherein the second cleavage site is uncleaved when the target ion in a second oxidation state is not present; and a second enzyme strand at least partially complementary to the second substrate strand and comprising a second catalytic loop; wherein the second catalytic loop is capable of cleaving the second substrate strand at the second cleavage site in the presence of the target ion in the second oxidation state, wherein said cleavage provides a second detectable signal. For example, in some aspects, the first DNAzyme sensor and second DNAzyme sensor can include any of the DNAzyme sensors described above.

In yet another aspect, provided is a kit comprising: i) a DNAzyme sensor comprising: a substrate strand comprising a cleavage site, wherein the cleavage site is uncleaved when a target molecule is not present; and an enzyme strand at least partially complementary to the substrate strand and comprising a catalytic loop; wherein the catalytic loop is capable of cleaving the substrate strand at the cleavage site in the presence of the target molecule, wherein said cleavage provides a detectable signal; and ii) an inactive DNAzyme sensor comprising: the substrate strand; and an inactive enzyme strand at least partially complementary to the substrate strand and comprising at least one mutation, wherein the at least one mutation prevents the inactive enzyme strand from cleaving the substrate strand. For example, in some aspects, the DNAzyme sensor can include any of the DNAzyme sensors described above.

TABLE 9
Inactive enzyme strands.

| | | SEQ ID |
|---|---|---|
| Strand | Sequence | NO |
| Fe(III)-B12- | GCGGCATGCGCGTTTGCGGCACCTAAACGCCCCTAATAG | 22 |
| iE | AG | |
| Fe(II)-H5-iE | TGGATATCTCCTGGCCAGACTGTTATGTGTGATACGGCA | 25 |
| | AACTTCGTGATGCCTCTACGGGTCCG | |
| Iron sensor | /5IAbRQ/TGGATATCTCCTAGTCAGACTGTTATGTGTGATA | 31 |
| (Fe²⁺) | CGGCAAACTTCGTGATGCCTCTACGGGTCCG | |
| Cu1 iE IBRQ | GTGGTAAGCCACCCGGTCTTTCTTTTTAAGAAAGATG | 46 |
| Cu2 iE | TTTCGCCATCTTCTGGTCCTTATAGTGACT | 49 |
| complement | | |
| Cu2 iE polyA | TTTCGCCATCTTCAAAAAAAAATAGTGACT | 50 |
| Cu2 iE polyT | TTTCGCCATCTTCTTTTTTTTATAGTGACT | 51 |
| Cu2 iE polyN | TTTCGCCATCTTCNNNNNNNNATAGTGACT | 52 |
| Cu2 iE ds | TTTCGCCATCTTCCTATAGTGACT | 53 |
| Cu1 iE IBRQ | /5IAbRQ/GTGGTAAGCCACCCGGTCTTTCTTTTTAAGAAA | 57 |
| | GATG | |
| Cu2 iE polyT | TTTCGCCATCTTCTTTTTTTTATAGTGACT/3IABKFQ/ | 61 |
| 3IBFQ | | |
| Cu2 iE | TTTCGCCATCTTCTGGTCCTTATAGTGACT/3IABKFQ/ | 62 |
| complement | | |
| 3IBFQ | | |
| Cu1 iE | /5IAbRQ/ATGTGGTAAGCCACCCGGTCTTTCCTTTTTAAGG | 65 |
| 5IBRQ v2 | AAAGATG | |
| Cu2 polyT iE | CGTCGCCATGCTTCTTTTTTTTATAGTGCT/3IABKFQ/ | 68 |
| 3IBFQ v2 | | |
| Cu1 iE Q | /5IAbRQ/GTGGTAAGCCACCCGGTCTTTCTTTTTAAGAAA | 70 |
| | GAAC | |

In some aspects, the substrate strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 1; and the inactive enzyme strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of SEQ ID NOs: 25 or 31.

In some aspects, the substrate strand can include any one of the sequences in TABLE 1; and the inactive enzyme strand can include any one of SEQ ID NOs: 25 or 31.

In some aspects, the substrate strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 3; and the inactive enzyme strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to SEQ ID NO: 22.

In some aspects, the substrate strand can include any one of the sequences in TABLE 3; and the inactive enzyme strand can include SEQ ID NO: 22.

In some aspects, the substrate strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 5; and the inactive enzyme strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of SEQ ID NOs: 46, 57, 65, or 70.

In some aspects, the substrate strand can include any one of the sequences in TABLE 5; and the inactive enzyme strand can include any one of SEQ ID NOs: 46, 57, 65, or 70.

In some aspects, the substrate strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 7; and the inactive enzyme strand can include 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of SEQ ID NOs: 49-53, 61-62, or 68.

In some aspects, the substrate strand can include any one of the sequences in TABLE 7; and the inactive enzyme strand can include any one of SEQ ID NOs: 49-53, 61-62, or 68.

### Methods

In an aspect, provided is a method of using any of the disclosed DNAzymes or compositions to detect a target molecule in a cell or tissue, the method comprising: a) providing the DNAzyme or composition to the cell or tissue; and b) detecting the first detectable signal and the second detectable signal. In some aspects, step b) can include imaging the cell or tissue.

In another aspect, provided is a method of spatially identifying a target molecule in a cell or tissue, the method comprising: a) providing to the cell or tissue any of the disclosed DNAzyme sensors or compositions; and b) imaging the cell or tissue, thereby allowing for spatial identification of the target molecule.

In some aspects, the method can further include, after step a) and before step b), exposing the cell or tissue to an acoustic signal. The detectable signal can be a photoacoustic dye, and, when the substrate strand is cleaved, the detectable signal can be activated upon exposure to the acoustic signal. In some aspects, the acoustic signal can be high frequency ultrasound (HIFU).

In some aspects, the method can further include, before step a), annealing the substrate strand and the enzyme strand together.

In some aspects, the substrate strand and the enzyme strand can be provided in a ratio of about 0.1:1 or more (e.g., about 0.2:1 or more, about 0.3:1 or more, about 0.4:1 or more, about 0.5:1 or more, about 1:1 or more, about 1.5:1 or more, about 2:1 or more, about 2.5:1 or more, about 3:1 or more, about 3.5:1 or more, about 4:1 or more, about 4.5:1 or more, about 5:1 or more, about 6:1 or more, about 7:1 or more, about 8:1 or more, about 9:1 or more, about 10:1 or more). In some aspects, the substrate strand and the enzyme strand can be provided in a ratio of about 10:1 or less (e.g., about 9:1 or less, about 8:1 or less, about 7:1 or less, about 6:1 or less, about 5:1 or less, about 4.5:1 or less, about 4:1 or less, about 3.5:1 or less, about 3:1 or less, about 2.5:1 or less, about 2:1 or less, about 1.5:1 or less, about 1:1 or less, about 0.5:1 or less, about 0.4:1 or less, about 0.3:1 or less, about 0.2:1 or less, about 0.1:1 or less). The substrate strand and the enzyme strand can be provided in a ratio ranging from any of the minimum values described above to any of the maximum values described above. For example, in some aspects, the substrate strand and the enzyme strand can be provided in a ratio of from about 0.1:1 to about 10:1 (e.g., from about 0.2:1 to about 9:1, from about 0.3:1 to about 8:1, from about 0.4:1 to about 7:1, from about 0.5:1 to about 6:1, from about 1:1 to about 5:1, from about 1.5:1 to about 4.5:1, from about 2:1 to about 4:1, from about 2.5:1 to about 3.5:1, from about 0.1:1 to about 3:1, from about 0.2:1 to about 2.5:1, from about 0.3:1 to about 2:1, from about 0.4:1 to about 1.5:1, from about 0.5:1 to about 1:1, from about 3:1 to about 10:1, from about 3.5:1 to about 9:1, from about 4:1 to about 8:1, from about 4.5:1 to about 7:1, from about 5:1 to about 6:1).

In some aspects, the method can further include, before step a), providing a reference level of the detectable signal by: i) providing to the cell or tissue an inactive DNAzyme sensor, the inactive DNAzyme sensor comprising: the substrate strand; and an inactive enzyme strand at least partially complementary to the substrate strand including at least one mutation; wherein the at least one mutation prevents the inactive enzyme strand from cleaving the substrate strand; and ii) imaging the cell or tissue, thereby providing a reference level of the detectable signal. The reference level of the detectable signal can be used to eliminate background noise in images obtained in step c). For example, in some aspects, the inactive DNAzyme sensor can include any of the inactive enzyme strands described above. In some aspects, the method can use any of the disclosed kits.

In some aspects, the substrate strand and the inactive enzyme strand can be provided in a ratio of about 0.1:1 or more (e.g., about 0.2:1 or more, about 0.3:1 or more, about 0.4:1 or more, about 0.5:1 or more, about 1:1 or more, about 1.5:1 or more, about 2:1 or more, about 2.5:1 or more, about 3:1 or more, about 3.5:1 or more, about 4:1 or more, about 4.5:1 or more, about 5:1 or more, about 6:1 or more, about 7:1 or more, about 8:1 or more, about 9:1 or more, about 10:1 or more). In some aspects, the substrate strand and the inactive enzyme strand can be provided in a ratio of about 10:1 or less (e.g., about 9:1 or less, about 8:1 or less, about 7:1 or less, about 6:1 or less, about 5:1 or less, about 4.5:1 or less, about 4:1 or less, about 3.5:1 or less, about 3:1 or less, about 2.5:1 or less, about 2:1 or less, about 1.5:1 or less, about 1:1 or less, about 0.5:1 or less, about 0.4:1 or less, about 0.3:1 or less, about 0.2:1 or less, about 0.1:1 or less). The substrate strand and the inactive enzyme strand can be provided in a ratio ranging from any of the minimum values described above to any of the maximum values described above. For example, in some aspects, the substrate strand and the inactive enzyme strand can be provided in a ratio of from about 0.1:1 to about 10:1 (e.g., from about 0.2:1 to about 9:1, from about 0.3:1 to about 8:1, from about 0.4:1 to about 7:1, from about 0.5:1 to about 6:1, from about 1:1 to about 5:1, from about 1.5:1 to about 4.5:1, from about 2:1 to about 4:1, from about 2.5:1 to about 3.5:1, from about 0.1:1 to about 3:1, from about 0.2:1 to about 2.5:1, from about 0.3:1 to about 2:1, from about 0.4:1 to about 1.5:1, from about 0.5:1 to about 1:1, from about 3:1 to about 10:1, from about 3.5:1 to about 9:1, from about 4:1 to about 8:1, from about 4.5:1 to about 7:1, from about 5:1 to about 6:1). In some aspects, the substrate strand and the inactive enzyme strand can be provided in the same or different ratio as the substrate strand to the enzyme strand.

In some aspects, the target molecule can be a metal ion having two or more oxidation states. In some aspects, the metal ion can be Fe²⁺ or Fe³⁺. In some aspects, the metal ion can be Cu⁺ or Cu²⁺. In some aspects, the metal ion can be Mn²⁺ or Mn⁴⁺. In some aspects, the metal ion can be Cr³⁺ or Cr⁶⁺. In some aspects, the metal ion can be Co²⁺ or Co³⁺. In some aspects, the metal ion can be Pb²⁺ or Pb⁴⁺. In some aspects, the metal ion can be Ag⁺ or Ag²⁺. In other aspects, the target molecule can be a metal ion with only one oxidation state. In some aspects, the metal ion can be Mg²⁺, Na⁺, Li⁺, Zn²⁺, K⁺, Cd²⁺, or Ca²⁺. In yet other aspects, the target molecule can be UO₂²⁺. In yet other aspects, the target molecule can be a protein or a small molecule.

In some aspects, step a) can further include providing to the cell or tissue two or more DNAzyme sensors, wherein each DNAzyme sensor spatially identifies a different target molecule. In some aspects, at least one target molecule may be a metal ion and at least another target molecule may not be a metal ion. In other aspects, one target molecule can be a metal ion in a first oxidation state and another target molecule can be said metal ion in a second oxidation state. In yet other aspects, one target molecule can be a first metal ion and another target molecule can be a second metal ion. In some such aspects, one target molecule may have multiple oxidation states and another target molecule may not have multiple oxidation states. In other such aspects, a first target molecule can be a first metal ion in a first oxidation state, a second target molecule can be a second metal ion in a second oxidation state, a third target molecule can be a third metal ion in a third oxidation state, and a fourth target molecule can be a fourth metal ion in a fourth oxidation state. It is understood that the methods disclosed herein can be used to detect any combination of any number of target molecules disclosed herein.

In some aspects, a first DNAzyme sensor can spatially identify Fe³⁺ and a second DNAzyme sensor can spatially identify Fe²⁺. In some such aspects, the first DNAzyme sensor can include a first substrate strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 3 and a first enzyme strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 4; and the second DNAzyme sensor can include a second substrate strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 1 and a second enzyme strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 2.

In some such aspects, the first DNAzyme sensor can include a first substrate strand including any one of the sequences in TABLE 3 and a first enzyme strand including SEQ ID NOs: 21 or 32; and the second DNAzyme sensor can include a second substrate strand including any one of the sequences in TABLE 1 and a second enzyme strand including any one of the sequences in TABLE 2.

In some aspects, a first DNAzyme sensor can spatially identify Cu²⁺ and a second DNAzyme sensor can spatially identify Cu⁺. In some such aspects, the first DNAzyme sensor can include a first substrate strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 7 and a first enzyme strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 8; and the second DNAzyme sensor can include a second substrate strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 5 and a second enzyme strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 6.

In some such aspects, the first DNAzyme sensor can include a first substrate strand including any one of the sequences in TABLE 7 and a first enzyme strand including any one of the sequences in TABLE 8; and the second DNAzyme sensor can include a second substrate strand including any one of the sequences in TABLE 5 and a second enzyme strand including any one of the sequences in TABLE 6.

In some aspects, a first DNAzyme sensor can spatially identify Fe³⁺, a second DNAzyme sensor can spatially identify Fe²⁺, a third DNAzyme sensor can spatially identify Cu²⁺, and a fourth DNAzyme sensor can spatially identify Cu⁺. In some such aspects, the first DNAzyme sensor can include a first substrate strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 3 and a first enzyme strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 4; the second DNAzyme sensor can include a second substrate strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 1 and a second enzyme strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 2; the third DNAzyme sensor can include a third substrate strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 7 and a third enzyme strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 8; and the fourth DNAzyme sensor can include a fourth substrate strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 5 and a fourth enzyme strand including 80% similarity or more (e.g., 81% similarity or more, 82% similarity or more, 83% similarity or more, 84% similarity or more, 85% similarity or more, 86% similarity or more, 87% similarity or more, 88% similarity or more, 89% similarity or more, 90% similarity or more, 91% similarity or more, 92% similarity or more, 93% similarity or more, 94% similarity or more, 95% similarity or more, 96% similarity or more, 97% similarity or more, 98% similarity or more, 99% similarity or more) to any one of the sequences in TABLE 6.

In some such aspects, the first DNAzyme sensor can include a first substrate strand including any one of the sequences in TABLE 3 and a first enzyme strand including any one of the sequences in TABLE 4; the second DNAzyme sensor can include a second substrate strand including any one of the sequences in TABLE 1 and a second enzyme strand including any one of the sequences in TABLE 2; the third DNAzyme sensor can include a third substrate strand including any one of the sequences in TABLE 7 and a third enzyme strand including any one of the sequences in TABLE 8; and the fourth DNAzyme sensor can include a fourth substrate strand including any one of the sequences in TABLE 5 and a fourth enzyme strand including any one of the sequences in TABLE 6.

In some aspects, the two or more DNAzyme sensors can be provided to the cell or tissue simultaneously, thereby allowing simultaneous detection or spatial identification of two or more target molecules. In other aspects, the two or more DNAzyme sensors can be provided to the cell or tissue at different times.

In some aspects, the method can further include, after step a) and before step b), washing the cell or tissue with a buffer.

In some aspects, the method can be used to quantify the target molecule in the cell or tissue.

In some aspects, the cell or tissue can be mammalian. In some aspects, the cell or tissue can be human. In some aspects, the cell or tissue can be cancerous. In some aspects, the cell or tissue can exhibit qualities of or can be derived from a patient having a neurogenerative disease (e.g., Alzheimer's disease) or multiple sclerosis. In some aspects, the cell or tissue can exhibit qualities of or can be derived from a patient experiencing old age or an age-related disease or disorder.

In some aspects, the method can be repeated multiple times in the same cell or tissue or in multiple cell samples or tissue samples taken from the same patient over a period of time. In some such aspects, the method can be used to quantify the target molecule in the cell or tissue over a period of time. In some such aspects, the method can be used to monitor progression of a cancer, a neurogenerative disease (e.g., Alzheimer's disease), multiple sclerosis, old age, or an age-related disease or disorder.

In some aspects, the method can be performed on a fixed tissue sample including multiple same or different cells or tissues.

In another aspect, provided is a method of determining an effect of a therapeutic agent on a target molecule, the method comprising: a) administering the therapeutic agent to a cell or tissue; b) exposing the cell or tissue to any of the disclosed DNAzyme sensors or compositions; and c) detecting the detectable signal; and d) using said detectable signal to determine the effect of the therapeutic agent on iron. For example, in some aspects, step c) can include imaging the cell or tissue.

In yet another aspect, provided is a method of determining an effect of a therapeutic agent on a target molecule in a cell or tissue, the method comprising: a) administering the therapeutic agent to the cell or tissue; b) spatially identifying the target molecule in the cell or tissue according to any of the disclosed methods of spatially identifying a target molecule in a cell or tissue; and c) comparing the spatial identification of step b) to a spatial identification of the target molecule in a control cell or control tissue.

In some aspects, the effect of the therapeutic agent on iron can include a change in target molecule amount, concentration, activity, or spatial distribution within the cell or tissue compared to a reference signal produced by a control cell or control tissue not exposed to the therapeutic agent.

In some aspects, the therapeutic agent can be an anti-cancer agent. In some aspects, the therapeutic agent can be used to treat a neurogenerative disease (e.g., Alzheimer's disease) or multiple sclerosis. In some aspects, the therapeutic agent can be used to treat an age-related disease or disorder. In some aspects, the therapeutic agent can induce apoptosis.

In some aspects, the therapeutic agent can induce ferroptosis; and the target molecule can be Fe²⁺ and/or Fe³⁺. As used herein, the term “ferroptosis” refers to cell death caused by or dependent on iron. Ferroptosis is often characterized by the accumulation of lipid peroxides. In some aspects, step b) can further include simultaneously spatially identifying Fe²⁺ and Fe³⁺ using two or more DNAzyme sensors.

In some aspects, the therapeutic agent can induce cuproptosis; and the target molecule can be Cu⁺ and/or Cu²⁺. As used herein, the term “cuproptosis” refers to cell death caused by or dependent on copper. Cuproptosis is often characterized by disruption of cellular processes due to abnormal copper accumulation within the mitochondria. In some aspects, step b) can further include simultaneously spatially identifying Cu⁺ and Cu²⁺ using two or more DNAzyme sensors.

In some aspects, the method can further include, before step a), providing a reference level of the detectable signal by: i) providing to the cell or tissue an inactive DNAzyme sensor comprising: the substrate strand; and an inactive enzyme strand at least partially complementary to the substrate strand and comprising at least one mutation, wherein the at least one mutation prevents the inactive enzyme strand from cleaving the substrate strand; and ii) detecting the detectable signal, thereby providing a reference level of the detectable signal; wherein the reference level is used to eliminate background noise in step c). For example, in some aspects, the inactive DNAzyme sensor can include any of the inactive enzyme strands described above. In some aspects, the method can use any of the disclosed kits.

In some aspects, the cell or tissue can be mammalian. In some aspects, the cell or tissue can be human. In some aspects, the cell or tissue can be cancerous. In some aspects, the cell or tissue can exhibit qualities of or can be derived from a patient having a neurogenerative disease (e.g., Alzheimer's disease) or multiple sclerosis. In some aspects, the cell or tissue can exhibit qualities of or can be derived from a patient experiencing old age or an age-related disease or disorder.

In some aspects, the method can further include, before step a), spatially identifying the target molecule in the cell or tissue according to any of the disclosed methods of spatially identifying a target molecule in a cell or tissue, and the cell or tissue before administration of the therapeutic agent can serve as the control cell or control tissue.

In some aspects, the method can further include spatially identifying the target molecule in a second cell or second tissue according to any of the disclosed methods of spatially identifying a target molecule in a cell or tissue; wherein the therapeutic agent may have not been administered to the second cell or second tissue, and the second cell or second tissue can serve as the control cell or control tissue.

In some aspects, the cell or tissue may be diseased or abnormal and the control cell or control tissue may not be diseased or abnormal. In other aspects, both the cell or tissue and the control cell or control tissue may be diseased or abnormal.

In some aspects, the cell or tissue may be cancerous and the control cell or control tissue may not be cancerous. In other aspects, both the cell or tissue and the control cell or control tissue may be cancerous.

In some aspects, the method can be repeated multiple times in the same cell or tissue or in multiple cell samples or tissue samples taken from the same patient over a period of time. In some such aspects, the method can be used to quantify the target molecule in the cell or tissue over a period of time. In some such aspects, the method can be used to monitor treatment of a cancer, a neurogenerative disease (e.g., Alzheimer's disease), multiple sclerosis, old age, or an age-related disease or disorder.

In some aspects, the method can be performed on a fixed tissue sample including multiple same or different cells or tissues.

### EXAMPLES

### Example 1: DNAzymes for Sensing and Imaging Fe²⁺ and Fe³⁺

Disclosed herein is in vitro selection and development of DNAzyme-based fluorescence sensors. They are made of two different DNA strands, one called the substrate strand, which contains an RNA base in the middle of the DNAs (rS), and the other called the enzyme strand (E) which can catalyze the cleavage of the RNA base in the presence of specific iron species.

When using the sensors, the enzyme strand and substrate strand were annealed with rS:E=1:1.1 ratio. The sensors can be used for selective detection of Fe²⁺ and Fe³⁺ respectively.

These sensors are DNA-based sensors. It can be applied for high special and temporal resolution imaging of Fe²⁺ and Fe³⁺ in living cells and tissues with high specificity. In comparison, current technology has limited success either in living cells or in vivo. They are mainly based on laboratory techniques, such as inductively coupled plasma mass spectrometry, electron paramagnetic resonance, x-ray fluorescence, and magnetic resonance imaging, which cannot readily provide spatial or temporal information in vivo because of their restrictive requirements for sample pretreatment or excessive time needed for data collection. They also focus on the total iron pool instead of the labile iron pool, which is an important portion of iron that contributes to biological events, such as ferroptosis. To visualize these labile iron pools, histochemical methods based on potassium ferricyanide or potassium ferrocyanide were developed, to distinguish Fe²⁺ from Fe³⁺ and acquire spatial information, but this method can only detect Fe²⁺ or Fe³⁺ separately on fixed tissue slices, not in living cells or in vivo.

Fluorescence sensors have also been developed to visualize labile Fe²⁺ and Fe³⁺ simultaneously in vivo and provide spatiotemporal information in living cells. However, most of these methods either have low selectivity for Fe²⁺ and Fe³⁺ over other metal ions, require organic solvents, or cannot be adapted readily for in vivo sensing applications. Recently, some Fe²⁺ sensors based on organic molecules and fluorophores have achieved sufficient selectivity and sensitivity for imaging in cells and mouse models. To image two different oxidation states of the same metal ions, such as Fe²⁺ and Fe³⁺ simultaneously, two sensors are needed that are not only specific for the respective Fe²⁺ or Fe³⁺ but two fluorophores are also needed that do not have much overlapping excitation and emission spectra to avoid interference in the detection. Because the target recognition and fluorescent readouts of the organic molecule sensors are coupled together, it is difficult to replace the fluorophore with one that has a different fluorescence emission spectrum to avoid overlap of fluorescent signals. Changing fluorophore moieties for these sensors normally requires a redesign of the sensors, which can adversely affect their other properties, such as loss of brightness of fluorescence, reduced selectivity, or change of subcellular localization of the sensor. Therefore, the simultaneous monitoring of two oxidation states of the same metal ion in living cells or in vivo has not yet been reported.

These sensors allow simultaneous imaging of Fe²⁺ and Fe³⁺ in living cells and tissues, thus providing tools for studying how iron redox changes are involved in different biological processes including ferroptosis and Alzheimer's diseases. The DNAzyme sensors can allow imaging of Fe²⁺ and Fe³⁺ simultaneously in living cells. It can provide spatial and temporal information of Fe²⁺ and Fe³⁺ with high specificity.

The sensing is based on the cleavage of the RNA base inside of the substrate strand of DNA. Thus, the unintended digestion/degradation of the RNA base can cause background signals that interfere with the detection. To overcome this potential pitfall, point mutations were introduced to the enzyme strand to remove the activity of the DNAzyme, thus the mutated sensor shares the same chemical property, and the background noise can be evaluated. By normalizing the signal, the signal that was obtained from the inactive control was also obtained with the active sensor, thus the signal that specifically responds to Fe²⁺ or Fe³⁺ can be observed.

The fluorescence properties of the sensors can be changed by changing the fluorophore and quencher pairs. Thus, the sensors can be applied to provide spatial-temporal information of Fe²⁺ and Fe³⁺ simultaneously with other biomarkers or sensors. Moreover, the signaling readout can be changed according to the sensing needs too. By changing the fluorophore-quencher pairs into other signaling-out put pairs, such as FRET, BRET, photoacoustic, non-invasive sensing can be achieved with a different form of signaling output. Moreover, by delivering the sensors to specific locations inside the cells, the imaging of Fe²⁺ and Fe³⁺ can be achieved in different subcellular localizations.

Visualizing redox-active metal ions, such as Fe²⁺ and Fe³⁺ ions, is essential for understanding their roles in biological processes and human diseases. Despite the development of imaging probes and techniques, the success in imaging both Fe²⁺ and Fe³⁺ simultaneously in living cells with high selectivity and sensitivity is limited. The existing tools are either limited to total iron amount rather than labile iron pool, which is critical for biological processes such as ferroptosis or have restricted ability in offering spatiotemporal information of both irons due to their limitations in sample processing time, selectivity, requirement of organic solvents, or cannot change the fluorophore easily. The tools disclosed herein allow imaging of Fe²⁺ and Fe³⁺ ions simultaneously with high selectivity.

The DNAzyme-based fluorescence sensors can image Fe²⁺ or Fe³⁺ with high selectivity in living cells. Their fluorescence intensities are correlated to the concentrations of Fe²⁺ or Fe³⁺. By labeling them with different fluorophore and quencher pairs, they can achieve simultaneously imaging of both irons as well as other important biomarkers and metal ions.

The sensors recognize selectively to Fe²⁺ and Fe³⁺ respectively and can show fluorescence intensity correlated to iron concentrations. The sensors provide the ability to simultaneously image Fe²⁺ and Fe³⁺ in living cells and tissues. The sensors are highly selective and sensitive for iron detection. The fluorophore can also be changed to match the needs of different fluorescence wavelengths for co-staining with other biomarkers. Finally, the sensors are bio-compatible and can be adapted for in vivo and in vitro studies.

With the information of relative amounts, distributions, and their relationships to other critical biomarkers, these tools are powerful for understanding the role of Fe²⁺ and Fe³⁺ in biological processes and human health.

### Example 2: Develop Fe²⁺/Fe³⁺ DNAzyme Based Sensors for Simultaneous Fe²⁺/Fe³⁺ Imaging in Ferroptosis and in Alzheimer's Disease Mouse Brain

Redox-active metal ions play key roles in many biological processes such as oxygen transport, energy production, and oxidative stress-related neurodegenerative diseases (1, 2). A primary example is iron (Fe), which is mostly present in ferrous [Fe(II)/Fe²⁺] or ferric [Fe(III)/Fe³⁺] states in living organisms (3, 4). Dyshomeostasis of iron and its abnormal redox cycling can lead to ferroptosis, an iron-dependent programmed cell death pathway, which is a key process in many neurodegenerative diseases including Alzheimer's disease (AD) (5-9). In addition, ferroptosis has emerged as a promising therapeutic approach to cancers (10, 11). However, how redox equilibrium and dynamic speciation are involved in ferroptosis and related to AD or cancer remains poorly understood, partly because of a lack of highly selective sensors that allow for simultaneous monitoring of both Fe²⁺ and Fe³⁺. Such sensors require detection of either Fe²⁺ or Fe³⁺ without cross-reactivity with the opposite Fe oxidation state. Because of the chemical and physical similarities between these oxidation states, it has been quite challenging to develop sensors to detect both Fe²⁺ and Fe³⁺ simultaneously with high selectivity and sensitivity.

To achieve sensing of different redox states of iron, various methods have been explored but with limited success either in living cells or in vivo. For example, laboratory techniques, such as inductively coupled plasma mass spectrometry (12, 13), electron paramagnetic resonance (14), x-ray fluorescence (15), and magnetic resonance imaging (MRI) (16-18) have been developed but cannot readily provide spatial or temporal information in vivo because of their restrictive requirements for sample pretreatment or excessive time needed for data collection. It has been shown that “labile” iron pools, which comprise only a small portion of total iron, play critical roles in many cellular processes, including lipid oxidation during ferroptosis and generating free radicals in AD (19-21), and all of the above methods can measure only the total iron without differentiating labile iron pools. To visualize these labile iron pools, histochemical methods based on potassium ferricyanide or potassium ferrocyanide was developed to distinguish Fe²⁺ from Fe³⁺ and acquire spatial information (22), but this method can only detect Fe²⁺ or Fe³⁺ separately on fixed tissue slices, not in living cells or in vivo, because the detection is based on forming insoluble blue pigments. In addition, ferricyanide can react with many other metal ions, such as Zn²⁺ and Cu²⁺, and thus is vulnerable to interference from these metal ions.

Fluorescence sensors have also been developed to visualize labile Fe²⁺ and Fe³⁺ simultaneously in vivo and provide spatiotemporal information in living cells. However, most of these methods either have low selectivity for Fe²⁺ and Fe³⁺ over other metal ions, require organic solvents, or cannot be adapted readily for in vivo sensing applications. Recently, some Fe²⁺ sensors based on organic molecules and fluorophores have achieved sufficient selectivity and sensitivity for imaging in cells (23-26) and in mouse models (27, 28). To image two different oxidation states of the same metal ions, such as Fe²⁺ and Fe³⁺ simultaneously, two sensors are needed that are not only specific for the respective Fe²⁺ or Fe³⁺ but two fluorophores are also needed that do not have much overlapping excitation and emission spectra to avoid interference in the detection. Because the target recognition and fluorescent readouts of the organic molecule sensors are coupled together, it is difficult to replace the fluorophore with one that has a different fluorescence emission spectrum to avoid overlap of fluorescent signals. Changing fluorophore moieties for these sensors normally requires redesign of the sensors, which can adversely affect their other properties, such as loss of brightness of fluorescence, reduced selectivity, or change of subcellular localization of the sensor (29). Therefore, the simultaneous monitoring of two oxidation states of the same metal ion in living cells or in vivo has not yet been reported. As a result, the redox equilibrium and dynamic distribution of Fe²⁺ and Fe³⁺ have not been investigated, although they have been hypothesized to play important roles on many biological processes including ferroptosis in neurodegenerative diseases such as AD.

To overcome the technical barrier for simultaneous monitoring Fe²⁺ and Fe³⁺ in vivo and to fill a major knowledge gap of redox equilibrium, dynamic distribution of Fe²⁺/Fe³⁺, and their roles in neurodegenerative diseases, a study was conducted which takes advantage of DNAzyme-based “catalytic beacon” sensors. DNAzymes, also called deoxyribozymes, are DNA molecules that display enzymatic activities, such as protein enzymes and ribozymes, in the presence of a cofactor such as metal ions (30-36). DNAzymes are typically isolated from a large DNA library of up to 1015 different sequences through a combinatorial process called in vitro selection (37). Among them, RNA-cleaving DNAzymes are of particular interest for sensing metal ions because these DNAzymes are often specific for a certain metal ion cofactor (30, 32). By conjugating a fluorophore at the end of the enzyme strand, two quenchers at the opposite termini of the enzyme strand and complementary substrate strand, respectively, this study takes the advantage of melting temperature differences before and after DNAzyme-catalyzed cleavage of the substrate strand and have developed a catalytic beacon approach (38-48) that produces metal ion-specific fluorescent turn-on sensors. Because the fluorophore is physically separated from the metal-binding site and fluorescent signal arises from the release of the fluorophore-labeled substrate upon cleavage, this approach can be used to sense metal ions using any fluorophore. Therefore, it is possible to use DNAzyme beacons labeled with different fluorophores that have distinct emission wavelengths for simultaneous monitoring of two or more targets. Through efforts from many laboratories, DNAzymes highly selective for different metal ions have been obtained, including Cr³⁺ (49), Ca²⁺ (50), Cd²⁺ (51), Co²⁺ (52), Cu²⁺ (53), Mg²⁺ (54), Pb²⁺ (30), UO₂²⁺ (40), Zn²⁺ (32, 55, 56), Ag⁺ (57), Li⁺ (58), and Na⁺ (59, 60). Despite decades of success using this approach, no in cellulo or in vivo DNAzyme sensor that can differentiate different oxidization states of the same metal ion has been reported.

This study reports in vitro selection and development of DNAzyme sensors with high specificity for either Fe²⁺ or Fe³⁺, which allows visualization of both Fe²⁺ and Fe³⁺ simultaneously in living cells and brain slices of AD mice models. Correlated signal changes were observed with the regulation of iron levels by addition of transferrin (Tf), an iron transport protein (61), or deferoxamine (DFO), an iron chelator (62). The study further applies these sensors to detect iron changes during ferroptosis in living cells and observed a decrease of Fe³⁺/Fe²⁺ redox ratio over time, which suggests that iron is a potential source related to the oxidative stress accumulation in this cell death pathway (7). The sensors provided spatial distributions of Fe²⁺ and Fe³⁺, as well as iron redox ratios, revealing a statistically significant increase in the Fe³⁺/Fe²⁺ ratio surrounding amyloid plaque regions but not in other brain regions and suggesting that not only total iron but also iron redox cycling play a key role in the progression of AD. These finding also suggests a correlation between amyloid plaques and the accumulation of Fe³⁺ and/or the conversion from Fe²⁺ into Fe³⁺, which provides potential direction for further functional study to understand metal redox in AD progression. These results demonstrate that simultaneous monitoring of Fe²⁺ and Fe³⁺ using iron-specific DNAzyme-based sensors provides deeper insight into the roles of redox cycling of labile iron in neurodegenerative diseases.

### Materials and Methods

DNA sequences: All DNA was ordered from Integrated DNA Technologies (IDT). Modifications are indicated with IDT's modification codes (see TABLE 10).

TABLE 10
List of DNA sequences.

| | | |
|---|---|---|
| | | SEQ ID |
| Name | Sequence (5′-3′) | NO |

Fe²⁺ In vitro Selection

| | | |
|---|---|---|
| Fe²⁺ IDT | GGAAGGAATCGTACGATTCC-N50- | 1 |
| Template | CGTGATGCCTCTACCTC | |
| Fe²⁺ Full | GACTGGTATCAATCTCACGTATrAGGAAGGAATCGT | 2 |
| Length Pool | ACGATTCC-N50-CGTGATGCCTCTACCTC | |
| Fe²⁺ P1 | GTATCAATCTCACGTATAGGAAGGAATCGTAC | 3 |
| Fe²⁺ P2 | CGTGATGCCTCTACCTC | 4 |
| Fe²⁺ P2-iSp | (AAC)5-Sp-C18-CGTGATGCCTCTACCTC | 5 |
| Fe²⁺ P3 | GACTGGTATCAATCTCACGTATrA | 6 |
| Fe³⁺ IDT | CCGGACCTCCTTCAG-N35/50- | 7 |
| Template | GACTCGTGCGAGTCTCCCTAACTGAAGTAAG-SpC3 | |
| Fe³⁺ Full | GATACATAGCATCTTACTTCAGTTArGGGAGACTCG | 8 |
| Length Pool | CACGAGTC-N35/50-CTGAAGGAGGTCCGGTC | |
| Fe³⁺ P1 | GCATCTTACTTCAGTTAGGGAGACTCGCACG | 9 |
| Fe³⁺ P2 | GACCGGACCTCCTTCAG | 10 |
| Fe³⁺ P2-iSp | GAC(AAC)4-Sp-C18-GACCGGACCTCCTTCAG | 11 |
| Fe³⁺ P3 | GATACATAGCATCTTACTTCAGTTArG | 12 |

Full-Length “cis” DNAzymes

| | | |
|---|---|---|
| Fe(II)-H5 | GACTGGTATCAATCTCACGTATAGGAAGGAATCGT | 13 |
| | ACGTTCCGTTATGGTTCTTTCTCCTAGCCAGACTGTT | |
| | ATGTGTGATACGGCAAACTTCGTGATGCCTCTACCT | |
| | C | |
| Fe(III)-B12 | GATACATAGCATCTTACTTCAGTTAGGGAGACTCGC | 14 |
| | ACGAGTCCCTTATCGGGGAATTCAATGTGCGCGTTT | |
| | GCGGCACCTAAACGCTCTTAGCTGAAGGAGGTCCG | |
| | GTC | |

Catalytic Beacon DNAzyme Sensors

| | | |
|---|---|---|
| Fe(III)-B12-E | GCGGCATGCGCGTTTGCGGCACCTAAACGCTCCTAA | 15 |
| | TAGAG/3IAbRQSp/ | |
| Fe(III)-B12-iE | GCGGCATGCGCGTTTGCGGCACCTAAACGCCCCTA | 16 |
| | ATAGAG/3IAbRQSp/ | |
| Fe(III)-B12-rS | /5Alex647N/CTCTATTArGGGAGACTCGCATGCCG | 17 |
| | C/3IAbRQSp/ | |
| Fe(II)-H5-E | /5IABKFQ/TGGATATCTCCTAGCCAGACTGTTATGTG | 18 |
| | TGATACGGCAAACTTCGTGATGCCTCTACGGGTCCG | |
| Fe(II)-H5-iE | /5IABKFQ/TGGATATCTCCTGGCCAGACTGTTATGTG | 19 |
| | TGATACGGCAAACTTCGTGATGCCTCTACGGGTCCG | |
| Fe(II)-H5-rS | /5IABKFQ/CGGACCCGTATCAATCTCACGTATrAGG | 20 |
| | ATATCCA/3AlexF488N/ | |

Catalytic Beacon DNAzyme Sensors
(without fluorophores/quenchers)

| | | |
|---|---|---|
| Fe(III)-B12-E | GCGGCATGCGCGTTTGCGGCACCTAAACGCTCCTAA | 21 |
| | TAGAG | |
| Fe(III)-B12-iE | GCGGCATGCGCGTTTGCGGCACCTAAACGCCCCTA | 22 |
| | ATAGAG | |
| Fe(III)-B12-rS | CTCTATTArGGGAGACTCGCATGCCGC | 23 |
| Fe(II)-H5-E | TGGATATCTCCTAGCCAGACTGTTATGTGTGATACG | 24 |
| | GCAAACTTCGTGATGCCTCTACGGGTCCG | |
| Fe(II)-H5-iE | TGGATATCTCCTGGCCAGACTGTTATGTGTGATACG | 25 |
| | GCAAACTTCGTGATGCCTCTACGGGTCCG | |
| Fe(II)-H5-rS | CGGACCCGTATCAATCTCACGTATrAGGATATCCA | 26 |

Fe³⁺ In vitro Selection

Buffers: Fe²⁺ buffer contains 50 mM bis-tris and 400 mM NaCl at pH 7.0 (adjusted with HCl). Fe³⁺ buffer contains 40 mM sodium acetate, 5 mM bis-tris, and 200 mM NaCl at pH 5.5 (adjusted with HNO₃). Universal Fe buffer contains 20 mM sodium acetate, 5 mM bis-tris, and 200 mM NaCl at pH 6.0.

In vitro selection of Fe²⁺-dependent DNAzymes: The initial pool used for all four different selection conditions was identical, using a randomized N50 region flanked by two primer-binding regions, of which one contained the single riboadenosine to serve as a cleavage site. A P2-iSp primer contained a hexaethylene glycol spacer (Spacer-C18, from IDT) modification, which stops Taq polymerization reaction from further extension, was used during the polymerase chain reaction (PCR) amplification step. The internal C18 spacer is followed by AACAACAACAACAAC (SEQ ID NO: 27), which results in production of the antisense strand with 15 nucleotides longer than the sense strand (DNA random pool). Therefore, single-stranded DNA random pools were separated from the antisense strand using denaturing polyacrylamide gel electrophoresis (PAGE).

The initial random pool for the first selection round was generated in two steps using a PCR thermocycler (C1000 Touch Thermal Cycler from Bio-Rad Laboratories Inc.). PCR1 was carried out in 96 PCR tubes with 0.1 μM IDT DNA template in three steps. In the first step, 0.1 μM primer P2-iSp was added to the PCR mixture containing 0.1 μM DNA template to undergo two cycles of extension. The second step was carried out by the addition of 0.15 μM primer P1 and two more extension cycles. Last, in the third step, 0.9 μM primer P2-iSp was added, and 10 cycles of extension were carried out. PCR2 was conducted by the addition of 1 μM primer P3 (for incorporation of the RNA base) and 0.1 μM primer P2-iSp, followed by 10 cycles of amplification. Before the PCR2 reaction was performed, 2 μl of [α-32P]-dATP (PerkinElmer) was added to label the DNA strands. The PCR products were then precipitated with 10% of a 3 M sodium acetate solution, at pH 5.2, and 2.7× volume of cold ethanol. The samples were stored at −80° C. for at least 1 hour and then centrifuged, washed, and lyophilized. Note that subsequent PCR amplifications, which were used to amplify selected DNA at the end of each selection round, were slightly different. In those reactions, PCR1 was carried out in a single step with 1 μM P2-iSp and P2 primers, followed by PCR2 using 5% of the PCR1 reaction as DNA template with 0.1 μM primer P2-iSp and 1 μM P3.

Ethanol-precipitated PCR products were dissolved in water, and an equal amount of stop buffer was added before loading samples on the gel. The stop buffer contained 8 M urea, 50 mM EDTA, and 1×TBE (tris, boric acid, and EDTA). The reaction products were purified using a 10% denaturing PAGE gel, with the use of 1×TBE as the running buffer. The PCR product was run on the PAGE gel alongside DNA size markers corresponding to the cleaved (87-mer) and intact (110-mer) pool. The gel was then covered with a plastic wrap; a radioactive triangle location marker was placed on top, and the gel was exposed to a phosphorimager cassette. After imaging the exposed film, bands that corresponded to the 110-mer marker on the gel was excised, crushed, and extracted with a solution containing 10 mM tris, 0.1 mM EDTA, and 300 mM sodium chloride (extraction buffer). Gel particles were frozen over 10 min at −80° C. and thawed in a room temperature water bath for at least 5 min to improve the extraction process. The solution was centrifuged at 10,000 g for 1 min to obtain a gel-free DNA in the extraction buffer. DNA samples were ethanol-precipitated using the aforementioned procedure.

The dried pools acquired from initial pool generation for the first selection round (and for PCR amplifications of subsequent selection rounds) were redissolved in 1× selection buffer and incubated with a desired concentration of Fe²⁺ for positive selections or a mixture of competing divalent metal ions (for counter selections) for 18 hours. Fe²⁺ concentration and incubation time at each round of the selection are indicated in TABLE 11 and TABLE 12. An initial negative selection was carried out before round 1 by incubating the DNA pool in selection buffer in the absence of divalent metal ions for 24 hours. Counter selections were carried out by incubating the DNA pools with 1 mM Mn²⁺, Cd²⁺, Zn²⁺, and Co²⁺ and 0.2 mM Pb²⁺ in selection buffer over 18 hours. Overall, four different selection conditions were carried out and each condition named with a letter (E to H; see TABLE 13). After negative and counter selection steps, uncleaved DNA pools were PAGE-purified and used for the subsequent positive selection. Cleaved DNA produced in each positive selection was PAGE-purified and used as a PCR template for a subsequent round of the selection. The stringency of positive selections was gradually increased by decreasing the reaction time and decreasing Fe²⁺ concentration as the selection progressed (TABLE 11, TABLE 12). All selection reactions were quenched by the addition of the stop buffer (50% final volume). All PAGE purifications were carried out using a 10% denaturing gel alongside the DNA size markers used earlier.

TABLE 11
Fe²⁺ dependent DNAzyme in vitro
selection condition for selections E and F.

| Selection round | Incubation time (min) | [Fe²⁺] (μM) |
|---|---|---|
| 1 | 60 | 500 |
| 2 | 60 | 500 |
| 3 | 40 | 500 |
| 4 | 30 | 250 |
| 5 | 10 | 100 |
| 6 | 5 | 50 |
| 7 | 5 | 10 |
| 8 | 5 | 10 |
| 9 | 5 | 5 |

TABLE 12
Fe²⁺ dependent DNAzyme in vitro selection condition
for selections G and H. From round 3 before each positive
selection one counter selection step was introduced. In counter selection
steps, DNA sequences that were active in a mixture of 1 mM Mn²⁺, Co²⁺,
Zn²⁺, Cd²⁺, and Pb²⁺ were removed. No counter selection
was introduced before the first two rounds of the selection.

| Selection round | Incubation time (min) | [Fe²⁺] (μM) |
|---|---|---|
| 1 | 60 | 500 |
| 2 | 60 | 500 |
| 3-R | 40 | 500 |
| 4-R | 30 | 250 |
| 5-R | 30 | 250 |
| 6-R | 10 | 100 |
| 7-R | 5 | 50 |
| 8-R | 5 | 50 |
| 9-R | 5 | 25 |

TABLE 13
Assigned letters for different Fe²⁺ selected pools.
“R” indicates incorporation of counter selection
before each positive selection, starting from round 3,
and “⁺GSH” indicates incorporation of
1 mM reduced glutathione.

| Selection Condition | Number of Sequenced Clones | Letter Code |
|---|---|---|
| Fe²⁺ | 34 | E |
| Fe²⁺⁺GSH | 40 | F |
| Fe²⁺-R | 36 | G |
| Fe²⁺⁺GSH-R | 39 | H |

In vitro selection of Fe³⁺-dependent DNAzymes: Similar to the selection of Fe²⁺-DNAzymes, in vitro selection for Fe³⁺-dependent DNAzymes was carried out using a denaturing PAGE-based purification method to separate cleavage products from uncleaved pools based on their size difference. In vitro selection was performed at pH 5.5 using rG as the cleavage site. Moreover, two different DNA pools with random region size of 35 or 50 nucleotides were used. The initial selection pools were generated through single linear PCR amplification. The PCR templates, synthesized by IDT, were designed with the goal of eliminating the need for multiple PCR amplifications to generate the initial sequence pools. DNA pools were generated by linear amplification of 360 pmol of the template mixed with 3.6 nmol of P3 primer in 90 PCR tubes (40 μl each), followed by 10 thermal cycles to complete the pool generation (40 s at 94° C., 1.25 min at 53° C., and then 1.1 min at 72° C.). In the generation of each pool, 4 μl of [α-32P]-dATP was used to internally label PCR products with 32P. Amplification of the selected pools after each positive selection round was carried out through two PCR reactions. In addition to the template and primers, each PCR reaction included Taq DNA polymerase (0.1 U/μl; NEB), 1.5 mM MgCl2, 50 mM KCl, 10 mM tris-HCl (pH 8.3 at 25° C.), and each deoxynucleoside triphosphate (dNTP) at 0.2 mM. Note that all parameters of different PCR reactions were optimized before the initiation of the in vitro selection. This optimization process was required to minimize production of side products, obtain clean products, and increase yield of the correct PCR product. DNA pools were PAGE-purified using the same protocol described above.

Dried pools were dissolved in 1× selection buffer and incubated for 24 hours (negative selection). This step was carried out before all positive selection steps unless stated. Negative selection steps were carried out to remove nonspecific cleavage that may occur in an Fe³⁺-independent manner. After the negative selection, uncleaved pools were PAGE-purified, extracted from gel, ethanol-precipitated, and dried to be used for positive selection. Dried pools were dissolved in 1× selection buffer and mixed with desired concentration of Fe³⁺ (for positive selections) for a certain period of time (TABLE 14). To initiate selection, Fe³⁺ was dissolved in selection buffer to make 2× Fe³⁺ stock solution. Then, equal volume of DNA samples were mixed with the 2× Fe³⁺ stock solution. The 2× Fe³⁺ solutions were prepared right before the positive selection. All positive selections were carried out in the dark by covering tubes with aluminum foil to prevent unwanted light-induced DNA cleavage by Fe³⁺. After round 4, each of the in vitro selection experiments carried out with the rG cleavage site at pH 5.5 were branched into two conditions by continuing or not continuing negative selection steps (see TABLE 14). Cleaved DNA obtained in each positive selection was PAGE-purified and used as template for PCR amplification reactions to generate DNA pools for the next round of the selection. Stringency of positive selections was gradually increased by decreasing both the reaction time and Fe³⁺ concentration (TABLE 14). All selection reactions were quenched with an equal volume of stop buffer. All PAGE purifications were carried out using a 10% gel alongside the DNA size markers used earlier.

TABLE 14
Fe³⁺ dependent DNAzyme in vitro selection conditions. In
one condition a negative selection step was carried out for 24
h before positive selections. In the other condition, after round
three, the negative selection discontinued (round 4NN to 9NN).

| | | |
|---|---|---|
| Selection round | Incubation time (min) | [Fe³⁺] (μM) |

Negative selection

| | | |
|---|---|---|
| 1-4 | 60 | 50 |
| 5 | 15 | 50 |
| 6 | 3 | 50 |
| 7-9 | 3 | 5 |

No negative selection

| | | |
|---|---|---|
| 4-5-NN | 60 | 50 |
| 6-NN | 10 | 50 |
| 7-NN | 3 | 50 |
| 8-9-NN | 3 | 5 |

Cloning and sequencing: On the basis of (i) the cleavage activity of the selected pools, (ii) results obtained in control experiments, and (iii) the activity assays carried out with different Fe²⁺ or Fe³⁺ concentrations, the most active pools with lowest background activity were chosen for cloning and sequencing. Cloning was carried out using PCR products with primers with no ribonucleotide cleavage site or Taq stopper. The same PCR reactions were carried out using selection primers to control activity of the species used for cloning. Negative PCR controls, with no template, were performed to assure that the observed amplifications were not caused by a contamination.

DNA sequences obtained from sequencing aligned on the basis of their sequence similarity for each individual pool. Sequence identity of the thermodynamically stable DNA tetraloop, which was engineered in the design of the random pools, remained intact in more than 96% of the obtained clones for the Fe³⁺-dependent selection. Among 149 individual sequences obtained from Fe²⁺-dependent selections, only 38 of them contain the intact tetraloop, with the rest of the clones having at least one mutation in this region. Few sequences were identified with mutations in their primer regions. Conservation of the stable tetraloop suggests that formation of this stable structure did not interfere with catalytic activity of the evolved sequences, while relatively high variation in the tetraloop region might imply that formation of this stable structure was not in favor of forming catalytically active structures. Because this region in the Fe²⁺ pool was not part of the PCR primers and considering the error rate of Taq DNA polymerase, a potential selection pressure might have caused evolution of species without the stable tetraloop over several selection rounds. The presence of the tetraloop in the active sequences can help in the prediction of active secondary structures. The sequence similarity of obtained sequences was represented using sequence similarity networks, originally used for organizing sequence similarity of protein sequences.

In vitro activity assays of Fe(II)-H5 and Fe(III)-B12 DNAzymes: In vitro activity assays were performed by incubating a solution of the DNAzymes with the indicated amount of metal solution (typically in a 1:1 volume ratio) and measuring the fluorescence change over time with a fluorometer. Metal stocks were prepared from ferric nitrate [Fe(NO3)3] and ferrous chloride (FeCl₂) dissolved in HNO₃ or HCl, respectively, which were then diluted into the relevant buffer. Both the metal solutions and the DNAzyme solutions were prepared in the relevant buffers so that there was no effect from different buffers mixing during the reaction, and their pH was checked to make sure that the solutions remained at the desired pH (especially important for the higher metal concentrations). For aerobic conditions, a SpectraMax M2 multidetection reader was used from the Roy J. Carver Biotechnology Center at the Metabolomics Center. For anaerobic conditions, a DeNovix QFX portable fluorometer was used inside of an anaerobic glove box. In addition, the metal selectivity tests were all performed with the DeNovix QFX fluorometer for consistency. For the SpectraMax M2, excitation at 633 nm and emission at 665 to 750 nm were used. For the DeNovix QFX, excitation at 470 nm and emission at 514 to 567 nm were used for the Fe(II)-H5 DNAzyme, and excitation at 635 nm and emission at 665 to 740 nm were used for the Fe(III)-B12 DNAzyme. Before the assay, the enzyme and substrate strands were annealed together using a water bath >65° C. for 5 min and then cooled to room temperature for 30 min. The fluorescence intensity in the presence of different metal ions was normalized to the fluorescent signal without addition of divalent or trivalent metal ions (i.e., just buffer) at the 30-min time point, for a direct comparison.

Cell culture, sensor delivery, and colocalization study: Iron-deficient buffer was prepared by S. McMasters of the University of Illinois School of Chemical Sciences Cell Media Facility as a standard preparation of minimum essential medium (MEM) but without fetal bovine serum (FBS) or any added iron (from Tf) to remove any potential contaminating iron species. HepG2 cells were purchased from the American Type Culture Collection (HB-8065), and was cultured in Dulbecco's modified Eagle's medium (DMEM) with adding 10% FBS, penicillin (100 U/ml), and streptomycin (100 U/ml), and in a 5% CO2, 37° C. incubator. A hemocytometer was used to determine cell density.

For delivery of DNAzyme sensors using PEI (66, 67), the corresponding Fe(II)-H5-ErS, Fe(II)-H5-iErS, Fe(III)-B12-ErS, and Fe(III)-B12-iErS (final concentration of 10 μM) were mixed in the Fe²⁺ buffer and Fe³⁺ buffer separately. The mixtures were annealed at 95° C. for 5 min and stored at room temperature to allow full hybridization. PEI (25 kDa) was dissolved in water at 1 mg/ml. For the active enzyme group, 2 μl of PEI was mixed and incubated with 2 μl of 10 μM Fe(II)-H5-ErS and Fe(III)-B12-ErS for 30 min in iron-deficient medium to allow the formation of PEI-DNA complexes (PEI-ErS) with the optimal N/P ratio (moles of amine, N, from the cationic polymer to moles of phosphate, P, from the DNA) as suggested previously (66). For the inactive enzyme group, 2 μl of PEI was mixed and incubated with 2 μl of 10 μM Fe(II)-H5-iErS and Fe(III)-B12-iErS in iron-deficient medium for 30 min to allow the formation of PEI-DNA complexes (PEI-iErS). The normal cell medium was replaced with iron-deficient medium before the PEI-ErS or PEI-iErS was added to the cells grown in the plates.

After incubating HepG2 cells with PEI-ErS or PEI-iErS complex for 4 hours, cells were washed with phosphate-buffered saline (PBS) twice to remove excess amounts of complexes in medium. Then, the cells were stained by Hoechst 33258 for 15 min. Images were obtained using a Zeiss LSM 880 confocal microscope at 63× oil objective lens with numerical aperture 1.40 and immersion medium Immersol 518 F (Zeiss) at the UIUC IGB Core Facilities. Florescence emission of Hoechst 33258 was measured over 450 to 500 nm with excitation at 401 nm. Fe(II)-H5 was excited by 488 nm and measured over 500 to 550 nm, and Fe(III)-B12 was excited at 633 nm and measured over 640 to 700 nm. A laser power of 67% and pinhole size of 1 atomic unit (AU) were used for the cellular images. Acquired images were analyzed by ImageJ using JACoP (88, 89). No background subtraction, Z-stacks, Gaussian blur filter, or change thresholds were performed for visualizing the figures. For cell fluorescence quantification, the study quantified the average fluorescence signals intensity in five cells (for TurboFect transfected cells) or five LysoTracker-labeled regions in different cells (for PEI transfected cells), per picture and three pictures per group with Image J (88), for the statistical comparison between groups.

FerroOrange was used at the final concentration of 1 μM, following the standard protocol from Sigma-Aldrich. When delivering FerroOrange with PEI, the same final concentration of FerroOrange was mixed with 2 μl of PEI and incubated at room temperature for 30 min before transfecting the cells. When delivering the DNAzymes with TurboFect, 100 pmol of DNAzyme S strand was annealed with 110 pmol of E strand in cells with 5 mM bis-tris, 40 mM sodium acetate, and 200 mM sodium chloride (pH 5.5) and then diluted with 98 μl of Opti-MEM and 1 μl of TurboFect transfection reagent. The mixture was incubated at room temperature for 20 min and incubated with cells for 4 hours. Afterward, the cells were washed with 1× Hanks' balanced salt solution (HBSS) and stained with Hoechst 33342 before imaging.

Simultaneous imaging of labile Fe²⁺ and Fe³⁺ in living cells: HepG2 or Hela cells were cultured in glass-bottom dishes until about 70% confluence. The cells were pretreated with iron-deficient MEM containing either 100 μM DFO, 100 μM ferric ammonium citrate, 5 M holo-Tf, or 50 μM Tf for 4 hours, and then, the medium was replaced with an iron-deficient MEM containing PEI-ErS or PEI-iErS complexes or Opti-MEM containing 1 μl of TurboFect transfection reagent as described above for 4 hours. Before imaging, the cells were stained by LysoTracker Red and Hoechst 33258 for 15 min. After washing with PBS, the cells were incubated in iron-deficient MEM or regular DMEM, respectively, during the imaging.

Ferroptosis-induced fluctuations of labile Fe²⁺ and Fe³⁺ pools: HepG2 cells were treated with 1 μM RSL3 in normal MEM at different time points (0, 2, 4, 6, 8, and 10 hours). Then, the cell medium was replaced by iron-deficient MEM containing PEI-ErS or PEI-iErS and incubated in a 37° C., 5% CO2 incubator for another 4 hours. Before imaging, the cells were stained by LysoTracker Red and Hoechst 33258 for 15 min. After washing with PBS, the cells were incubated in iron-deficient MEM during the imaging.

To measure the cytotoxicity of RSL3 to HepG2 cells, a standard MTT assay was used. HepG2 cells were seeded at a density of 15,000 cells per well in 96-well plates. When the cells grew to 80% confluence, the medium was replaced by the normal MEM containing 1 μM RSL3 at different time points (0, 2, 4, 6, 8, and 10 hours). Afterward, the cell medium was replaced by the iron-deficient MEM containing PEI-ErS or PEI-iErS, incubated in a 37° C., 5% CO2 incubator for another 4 hours. The absorbance at 570 nm was measured with a SpectraMax M2 microplate reader to obtain the MTT assay readings. The untreated cells were set as a blank control for normalization.

Fe²⁺ and Fe³⁺ detection in mouse brain slices: All animal studies were performed with the approval of the Institutional Animal Care and Use Committee (IACUC) of the University of Illinois at Urbana-Champaign (protocol number 22094) and the IACUC of the University of Texas at Austin (protocol number AUP-2021-00295). Eleven-month-old 5×FAD mice (RRID: MMRRC_034840-JAX) and WT mice (B6SJLF1/J) of the same sex were used and compared. To obtain the brain slices, after deep anesthesia, the mice were perfused with saline, followed by paraformaldehyde fixation. The mouse brain was isolated and fixed in 4% paraformaldehyde for 24 hours at 4° C. and then embedded in 30% sucrose in PBS for another 3 days. Coronal brain sections (section thickness, 50 μm) were obtained by microtome section and stored in cryoprotectant at −20° C. before staining. Ten brain slices from three individuals were stained with technical repeats for each group. The brain sections were rinsed with PBS three times for 5 min each and then blocked with 2% bovine serum albumin (BSA) in PBS for 10 min. To visualize Aβ (1-13), brain slices were incubated with CF350-conjugated HJ3.4 antibody (78), which was prelabeled with the Mix-n-Stain Antibody Labeling Kit (Sigma-Aldrich), with 1:500 dilution in blocking solution for 1 hour and then washed with 2% BSA in PBS for 4 min, followed by another wash in PBS for 5 min to remove extra antibody and nonspecific signaling. To image Fe²⁺ and Fe³⁺ simultaneously, 2 μM DNAzyme enzyme strand and 2.2 μM DNAzyme substrate strand for both iron oxidization states were annealed separately in 5 mM bis-tris (pH 5.5), 40 mM sodium acetate, and 200 mM NaCl buffer (bis-tris-acetate buffer) by incubation at 95° C. for 5 min and then slowly decreased to room temperature. After annealing, the Fe(II)-H5 and Fe(III)-B12 sensors were mixed in a 1:1 ratio to generate the sensor mix. The mouse brain sections prestained with HJ3.4 antibody were rinsed with the bis-tris-acetate buffer twice for 5 min each and then incubated in the sensor mix for 30 min. After incubation, the brain slices were rinsed once in the bis-tris-acetate buffer and mounted with Fluoromount-G mounting medium (SouthernBiotech, Birmingham, AL, USA). Images were taken with a Nikon spinning disk confocal or Zeiss LSM 710 confocal microscope with a 20× objective with 405-, 488-, and 640-nm channels. Afterward, images were processed and quantified with ImageJ as follows: To enable a direct comparison between groups without interference from a potential difference in background signaling between treatments, as indicated by the point-mutated inactive sensor (iErS), the fluorescent intensity of the sensor was normalized with iErS groups by subtracting the average fluorescence intensity in the imaging area or the regions of interest (APDR or non-APDR regions) in the pictures of correlated iErS group from the ErS group with the same treatment. Colocalization study and quantification of fluorescence intensity for APDR and non-APDR regions were performed by marking the region of interests on the basis of HJ3.4 staining. Five spots including four corners and the center of each image were analyzed. Three images were taken for each group.

Data processing and statistical analysis: To have a direct comparison of the iron amount between groups, the background signals were normalized by subtracting the fluorescence signals in iErS groups from the ErS groups (F-F0). No Z-stacks or change thresholds were used for statistical analysis. Graphs were plotted with GraphPad (GraphPad Software Inc.) or Origin (OriginLabs Corporation) and correspond to a single experiment. Bars represent means±SEM. Two-tailed distribution and two-sample equal variance t test were used for analyzing significance. Dixon's Q tests were performed to identify and exclude outliners. *P<0.05, **P<0.01, and ***P <0.001. All experiments have three or more biological replicates, which showed the same conclusion. The presented data described the quantification from single representative replicates from those biological replicates.

### Results

In vitro selection of Fe²⁺ and Fe³⁺-specific DNAzymes: To obtain Fe²⁺-specific DNAzymes, in vitro selection was conducted in an anaerobic glove box because Fe²⁺ is readily oxidized to Fe³⁺ in the presence of oxygen. Parallel selections were carried out in the presence or absence of 1 mM glutathione, a highly abundant cytosolic metabolite predicted to form a complex with Fe²⁺ intracellularly (63). A negative selection against the selection buffer without Fe²⁺ was incorporated before the first round, and counter selections against Mn²⁺, Co²⁺, Zn²⁺, Cd²⁺, and Pb²⁺ were introduced starting from the third round of the selections to remove DNAzymes that catalyzed the cleavage of the substrate in the presence of other components in the selection buffer or other divalent metal ions. These additional steps were necessary to improve the selectivity of the isolated DNAzymes (59). The selection was monitored by measuring the percent cleavage activity in the presence of Fe²⁺ in each round and was continued for nine rounds. Enrichment of highly Fe²⁺-specific DNAzymes and effectiveness of counter selection was confirmed by evaluating cleavage activity of the selected pools in the presence of Fe²⁺ or Fe³⁺ or several other divalent metal ions. From cloning and sequencing, a total of 149 sequences were identified from different conditions (TABLE 13). Upon testing representative individual sequences for their Fe²⁺-dependent activity, the most active and selective sequence, named Fe(II)-H5, was chosen for sensor development. First, on the basis of the predicted secondary structures of the Fe(II)-H5 cis-acting DNAzyme, the study generated six trans-acting DNAzymes including a separate enzyme (E) and substrate(S) strands by truncating sequences at different sites and testing their cleavage activity. The truncation studies resulted in an active trans-cleaving DNAzyme with minimal catalytic sequence (truncation-2b). Such a trans-cleaving DNAzyme is suitable for conjugating to fluorophore and quenchers and compatible with the catalytic beacon sensing strategy.

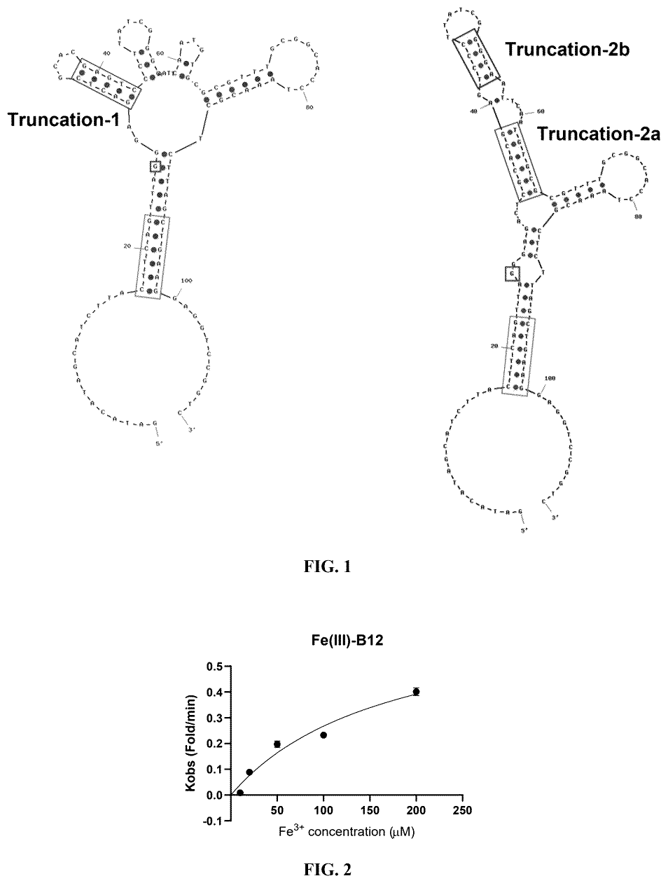

With the goal of selecting Fe³⁺-specific DNAzymes, several in vitro selection conditions and strategies were tested. It was found that the choice of in vitro selection condition including components to solubilize Fe³⁺ while keeping it accessible to the DNA molecules in the initial library is critical to the successful isolation of Fe³⁺-specific DNAzymes. As a result of these tests, in vitro selection was conducted in the presence of 5 mM bis-tris in an acetate buffer at pH 5.5, which the study found was able to stabilize Fe³⁺ in a soluble form because Fe³⁺ is known to readily form insoluble iron hydroxide complexes at pH>2.6 if not stabilized with a weakly chelating agent such as bis-tris. Inclusion of strong chelators such as citrate ions that can solubilize Fe³⁺ resulted in failure in isolating Fe³⁺-specific DNAzymes, perhaps because of the inability of DNA molecules to compete with such chelators in binding to Fe³⁺. Negative selections against the same buffer without Fe³⁺ were incorporated starting from round 1 before each positive selection to decrease the chance of isolating Fe³⁺-independent DNAzymes. The in vitro selection process was continued until the ratio of Fe³⁺-specific activity over background cleavage started to drop after round 9. The enriched pools did not show substantial cleavage activity in the presence of several other metal ions at 50 μM, such as Fe²⁺, Mg²⁺, Ca²⁺, Sr²⁺, Ba²⁺, Mn²⁺, Co²⁺, Ni²⁺, Cu²⁺, Zn²⁺, Cd²⁺, Pb²⁺, Eu³⁺, Sm³⁺, In³⁺, Tb³⁺, and Yb³⁺. From cloning and sequencing, a total of 157 DNAzymes were isolated from different selection conditions (TABLE 15). The resulting sequences were aligned on the basis of their primary sequence similarities. The activity of several representative DNAzymes were tested, and one of the most active DNAzymes, called Fe(III)-B12, was converted into trans-cleaving DNAzymes, by truncation studies. The truncation study was based on predicted secondary structures of the cis-acting Fe(III)-B12 DNAzyme (FIG. 1). The cleavage activities of three trans-acting DNAzymes generated by truncating B12 sequence at different sites were tested. The secondary structure of trans-acting Fe(III)-B12 DNAzyme with minimal catalytic region (FIG. 1) was used to design the Fe³⁺-dependent DNAzyme-based fluorescent sensor. To confirm the selectivity of the trans-cleaving iron DNAzymes, the study evaluated the cleavage activity of the Fe(II)-H5 and Fe(III)-B12 DNAzymes in the presence of different metal ions at 100 μM including Fe²⁺, Fe³⁺, Co²⁺, Mn²⁺, Zn²⁺, Pb²⁺, Cd²⁺, Ni²⁺, Cu²⁺, Mg²⁺, Ca²⁺, Sr²⁺, Ba²⁺, Eu³⁺, Sm³⁺, In³⁺, Tb³⁺, and Yb³⁺. Both DNAzymes exhibit excellent selectivity for their respective iron oxidation state.

TABLE 15
Assigned letters to different Fe³⁺ selected pools. Random
regions of 35 or 50 nucleotides are indicated, and “NN”
denotes selections carried out without negative selection.

| Selection Condition | Number of Sequenced Clones | Letter Code |
|---|---|---|
| N35 | 39 | A |
| N50 | 40 | B |
| N35 (NN) | 39 | C |
| N50 (NN) | 39 | D |

Conversion and characterization of fluorescent DNAzyme sensors: To convert the Fe²⁺- and Fe³⁺-specific DNAzymes into fluorescent sensors, the study applied the catalytic beacon design by incorporating a fluorophore (F) on one end of the substrate strand and an intermolecular quencher (Q1 or Q3) on opposite termini of the enzyme strand. The enzyme strand can bind to the substrate strand through DNA-DNA hybridization of the two binding arms. In addition, the substrate strand, which contains a ribonucleotide cleavage site, is labeled with an intramolecular quencher (Q2 or Q4) to suppress background fluorescence without substrate strand being cleaved (39). The binding arms are designed to be long enough that the melting temperature of the entire complex is higher than the desired ambient temperature (e.g., 37° C. for cellular studies). Therefore, the enzyme and uncleaved substrate strands can stably hybridize to each other under assay conditions. Upon metal-induced cleavage of the substrate at the internal ribonucleotide site, the resulting fluorophore-labeled fragment has a much lower melting temperature (<20° C.). This decrease in melting temperature allows the fluorophore-labeled fragment to dissociate from the complex, resulting in the release of the fluorophore from both inter- and intramolecular quenchers, followed by a substantial increase in fluorescent signal. Because this design decouples the fluorophores from metal-dependent DNAzyme-based cleavage of the substrate, any fluorophore and quencher pairs can be readily adapted to the sensors to achieve sensing with different excitation and emission wavelengths. Moreover, both Fe²⁺ and Fe³⁺ are known to quench fluorescence of fluorophores nonspecifically. Separating the Fe²⁺- and Fe³⁺-binding site away from the fluorophore helps minimize this issue, allowing to build Fe²⁺- and Fe³⁺-specific DNAzyme sensors with turn-on signals.

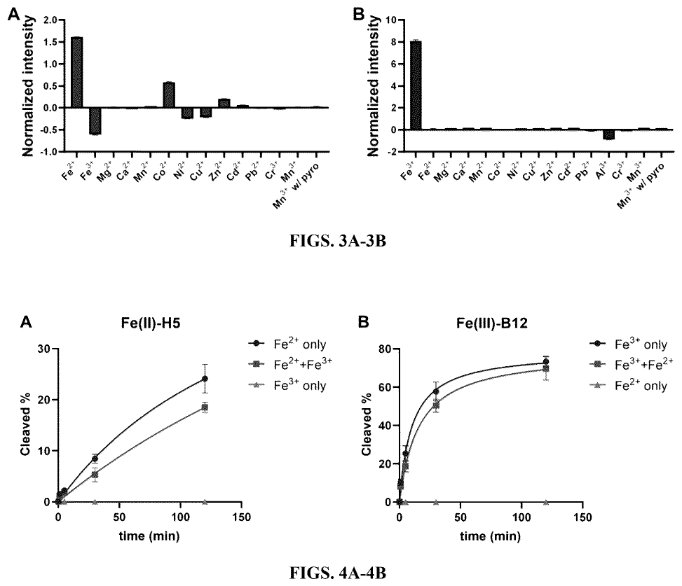

Taking advantage of the above design, the study incorporated Alexa Fluor 488 in the Fe²⁺-selective DNAzyme [Fe(II)-H5] and Alexa Fluor 647 in the Fe³⁺-selective DNAzyme [Fe(III)-B12] for simultaneous detection of both oxidation states of iron using two different fluorescent channels. Furthermore, to eliminate the artifact of fluorescent signals due to anything other than Fe²⁺- and Fe³⁺-specific activity of the DNAzymes (e.g., sensor degradation under intracellular conditions), inactive DNAzymes (iErS) that contain single-nucleotide mutations that abolish DNAzyme cleavage activity were used. Increase in the fluorescent signal of the Fe(II)-H5 and the Fe(III)-B12 DNAzyme sensors were measured in the presence of different concentrations of Fe²⁺ or Fe³⁺, respectively. Both sensors showed linear signal increase in response to increasing concentrations of their respective metal ions in a physiologically relevant range (FIG. 2). In addition, the DNAzyme-based sensors show excellent selectivity for their cognate metal ion target over the different oxidation state of the same metal ion, as well as other biologically relevant metal ions (FIGS. 3A-3B, FIGS. 4A-4B).

DNAzyme sensors monitor Fe²⁺ and Fe³⁺ simultaneously in living cells: Having demonstrated high selectivity for either Fe²⁺ or Fe³⁺ by the DNAzyme sensors under physiologically relevant concentrations, the study explored imaging Fe²⁺ and Fe³⁺ in the endosomal-lysosomal system within living cells. This system is the entry point of the iron transport protein, Tf, which is the major iron import pathway in mammalian cells by binding to and importing extracellular Fe³⁺. In addition, the endosomal-lysosomal system is known to contain both Fe²⁺ and Fe³⁺ labile pools due to the presence of metalloreductase enzymes such as six-transmembrane epithelial antigen of the prostate 3 (Metalloreductase STEAP3), which reduces Fe³⁺ to Fe²⁺, thus enabling its further transport into the cytosol (64, 65). To deliver the DNAzyme sensors into the endosomal-lysosomal system, the study used polyethylenimine (PEI), which has previously been used to deliver nanosensors into the endosomal-lysosomal system successfully (66-68). The colocalization of the signal from DNAzyme sensors and LysoTracker Red, a lysosome marker (69), confirms efficient delivery of the sensors into lysosomes of the cells. The Pearson's correlation coefficient between the iron pools and LysoTracker was around 50 to 60% (TABLE 16, TABLE 17), suggesting that although most of the iron signal was localized in lysosome, it was not evenly distributed, as not all the LysoTracker-positive regions showed iron signals. It is also possible that a small portion of Fe²⁺ or Fe³⁺ may be present in other parts of the cells. To introduce different iron concentrations in endo-lysosomes, the cells were incubated in an iron-deficient medium with increasing concentrations of Tf (61) or with DFO as an iron chelator to decrease the level of labile iron (62). Both the Fe(II)-H5 and Fe(III)-B12 DNAzyme sensors showed increase in fluorescent signal within lysosomes in response to increasing concentrations of Tf. This observation is expected even for Fe²⁺ because of the reduction of Fe³⁺ to Fe²⁺ by endogenous metalloreductases in the endosomal-lysosomal system (64, 65). However, the increase in the 5 μM Tf group was only 1.9-fold for Fe²⁺ and 1.6-fold for Fe³⁺, which may be due to the limited iron source in iron-deficient medium. To support this explanation, cells were also incubated in normal cell media, and around 4-fold increase was observed for both Fe³⁺ and Fe²⁺. In contrast, treating cells with DFO resulted in a 5.7- to 5.8-fold decrease of iron, as indicated by both DNAzyme sensors in iron-deficient media but only a 1.4- to 1.8-fold decrease of iron in normal media. Together, these results demonstrate that the Fe(II)-H5 and Fe(III)-B12 DNAzyme sensors can be used to image labile Fe²⁺ and Fe³⁺ in endo-lysosomes simultaneously. This is the first time that simultaneous imaging of Fe²⁺ and Fe³⁺ within the same location of a live cell has been demonstrated.

TABLE 16
Pearson's correlation coefficient analysis for the
colocalization between Fe²⁺ and Fe³⁺ signals,
and LysoTracker, respectively. ErS: active DNAzyme sensors.

| ErS | Fe(II)-H5 | Fe(III)-B12 |
|---|---|---|
| Endo | 61.8% | 58.8% |
| Tf 5 μM | 65.4% | 58.7% |
| Tf 50 μM | 76.0% | 69.5% |
| DFO | 54.7% | 43.7% |

TABLE 17
Pearson's correlation coefficient analysis for the colocalization
between Fe²⁺ and Fe³⁺ signals, and LysoTracker,
respectively. iErS: point mutated inactive DNAzyme sensors.

| iErS | Fe(II)-H5 | Fe(III)-B12 |
|---|---|---|
| Endo | 54.8% | 56.7% |
| Tf 5 μM | 50.1% | 55.7% |
| Tf 50 μM | 55.5% | 54.4% |
| DFO | 75.1% | 60.7% |

To quantify iron redox changes using the sensors, the Fe³⁺/Fe²⁺ ratio when treating the cells with Tf or DFO was calculated. No significant change of the Fe³⁺/Fe²⁺ ratio was observed. This is probably because the Fe³⁺ imported by Tf is readily converted into Fe²⁺ because of the reducing environment inside the endosomal-lysosomal system (65). Moreover, when treating the cells with DFO, the Fe³⁺/Fe²⁺ ratio decreased 2.4-fold. This observation is consistent with previous report that DFO is a Fe³⁺ chelator (62), which can bind to Fe³⁺ and decrease its concentration.

The above results illustrated that Fe²⁺ and Fe³⁺ detection in an endo-lysosomal system is a result of guided delivery of the sensors using PEI. When the sensors were delivered using TurboFect, a delivery agent that has less subcellular localization effect, the sensor distributed more evenly inside of the cell and showed the presence of Fe²⁺ and Fe³⁺ in other parts of the cell, such as nucleus and cytoplasm. Thus, the delivery agents used for the sensors could influence the subcellular localization of the sensors. To calibrate the sensors with other iron sensors, the Fe(II)-H5 DNAzyme sensor was costained with FerroOrange, a commercially available Fe²⁺ sensor, and observed a similar increase in fluorescence intensity when adding excess amounts of iron (100 μM ferric ammonium citrate). However, less change in fluorescence intensity was observed in the presence of the iron chelator DFO. This difference suggests that FerroOrange is probably less sensitive in the presence of low concentrations of iron. The study also found that the pattern of FerroOrange was similar but not the same as the signal from the Fe²⁺ sensor. Specifically, both sensors showed similar fluorescence distribution in cytoplasm in general, but the sensors showed an increased intensity for some of the cytoplasm regions. In addition, the sensor detected iron signal in the nucleus, which was not the case for FerroOrange. These differences are expected, as each sensor has its own cellular delivery efficiency, subcellular localization preference, sensitivity, and selectivity toward Fe²⁺. For example, FerroOrange was known to favor a localization in the Golgi and endoplasmic reticulum (70). To find out whether the difference in subcellular localization is responsible for the observed differences, the FerroOrange was delivered with PEI to help concentrate the sensor in the endosomal-lysosomal system and observed an enriched FerroOrange signal in the endosomal-lysosomal system. These results indicate that the sensor can show a similar trend as FerroOrange, yet it is more sensitive to iron.

Increase in both Fe³⁺ and Fe²⁺ levels but decrease in Fe³⁺/Fe²⁺ ratio in ferroptotic cells: Next, the study investigated whether the DNAzyme-based sensors can detect changes in iron redox states by using the Fe(II)-H5 and Fe(III)-B12 sensors to monitor Fe²⁺ and Fe³⁺ and their conversion during ferroptosis, which can be triggered by lipid peroxidation related to excess amounts of iron and its resulting Fenton reaction (5, 71). To induce ferroptosis in the model system, HepG2 cells were incubated with RAS-selective lethal 3 (RSL3) (72, 73), which is a well-known ferroptosis inducer. RSL3 inhibits glutathione peroxidase 4 (GPX4), which eliminates phospholipid peroxides, and thus allows lipid peroxides to accumulate because of iron-mediated Fenton chemistry, triggering ferroptosis-induced cell death without directly modulating total iron levels (74). The 3-(4,5-dimethylthiazol-2-yl)-2,5-diphenyl-2H-tetrazolium bromide (MTT) assay (75) showed RSL3-induced cell death over 8 hours. By monitoring the cells in this 8-hour window of ferroptosis, a rapid increase of both Fe²⁺ and Fe³⁺ levels was observed in the LysoTracker-labeled region within the first 4 hours of ferroptosis. These results indicate a rapid generation of labile Fe²⁺ and Fe³⁺ within lysosomes during the initiation stage of the ferroptosis. A larger increase of Fe²⁺ (2.3-fold increase from 0 to 2 hours and 4.3-fold increase from 2 to 4 hours) than that of Fe³⁺ (2.8-fold increase of from 0 to 4 hours) was observed. As a result, a continuous decrease of the Fe³⁺/Fe²⁺ ratio was observed during ferroptosis, which indicates that the reduction of Fe³⁺ to Fe²⁺ could serve as a source for labile Fe²⁺ in endo-lysosomes. After 4 hours, both iron levels decreased, indicating that, in the later stage of ferroptosis, the labile iron pools are being depleted from the endo-lysosome. These results demonstrate that simultaneous monitoring of Fe²⁺ and Fe³⁺ levels by the DNAzyme-based sensors can provide insights into unexplored roles of labile Fe²⁺ and Fe³⁺ during ferroptosis.

Elevated iron redox levels accumulate in amyloid β plaque regions in AD mouse brain: The study next investigated the ability of the DNAzyme-based sensors to monitor labile iron levels in a more complicated biological model. Increasing evidence has linked ferroptosis to AD because elevated iron levels were also observed in AD patients' brains, and thus, excessive accumulation of iron is considered as a risk factor in the development of AD (76). However, limited information on spatial distribution of Fe²⁺ and Fe³⁺ and their redox dynamics in AD pathogenesis is available, partly because of the limitations of existing iron sensors. To address this unmet need, the study further applied the sensors to image labile iron in brain slices of AD mice to gain insights into the relationship between iron redox activity and AD progression. Fe²⁺ and Fe³⁺ were detected simultaneously in the cortex region and compared between 11-month-old wild-type (WT) mice and 5×FAD mice that express mutant humanized amyloid precursor protein and mutant human presenilin 1 (PSEN1) transgenes, which are commonly used as an AD model (77). A 2.3-fold increase in the Fe²⁺ level and a 7.9-fold increase in the Fe³⁺ level was observed in whole-brain slices from 5×FAD mice when compared to the WT controls, indicating that Fe³⁺ accumulates in the AD mouse brains more than Fe²⁺. To understand the involvement of iron redox in the AD pathogenesis, the study further investigated the distribution of iron oxidization states in relation to amyloid β (Aβ) formation, which accumulates in AD brain and contributes to neurotoxicity (77), by comparing the fluorescence intensity of the Fe²⁺ and Fe³⁺ DNAzyme-based sensors in the cortex regions that have either Aβ plaques or not. Both iron sensors were costained with HJ3.4 antibody, which labels immunoreactive Aβ plaques (78). Fe²⁺ was increased by 2.1-fold in and surrounding the cortex regions that have Aβ plaque deposition [called Aβ plaque deposition region (APDR)], while Fe²⁺ was elevated by 1.7-fold in the surrounding cortex regions that did not show Aβ plaque deposition (called non-APDR). The statistical analysis showed that the increase in the level of Fe²⁺ in both APDR and non-APDR was significant when compared with the WT controls. However, there was no significant difference between APDR and non-APDR Fe²⁺ levels, which suggests a similar elevation of Fe²⁺ levels in the cortex regions of AD mouse. In contrast, a 2.6-fold and an 8.7-fold increase in the levels of Fe³⁺ in non-APDR and APDR, respectively, was observed when compared with their WT controls. These observations suggest that the accumulation of the oxidized state of iron in AD brain is preferentially enriched in APDR. Because of the nonhomogeneous distribution, such a closer observation and analysis instead of a brief comparison on whole-brain slices is necessary to understand how iron redox correlates with AD models.

To understand the role of iron redox cycling in AD progression, the study further used the sensors to compare the relative Fe³⁺/Fe²⁺ ratios based on the fluorescence difference between 5×FAD mice and WT mouse brains. When performing the comparison with whole-brain slices, a 2.4-fold increase of Fe³⁺/Fe²⁺ ratio in AD mouse cortex was observed when compared with its WT controls. The study then analyzed the spatial information of these redox changes in detail and found a 1.6-fold increase in the relative Fe³⁺/Fe²⁺ ratio in non-APDR and a 3.1-fold elevation in the relative Fe³⁺/Fe²⁺ in APDR of 5×FAD mouse brains when compared with WT mouse brains. This observation reveals that oxidative stress in AD brains is associated with aggregation of Aβ. Although Fe³⁺ aggregates with Aβ plaques and could reduce to Fe²⁺ to serve as one of the sources for generating reactive oxygen species (79-81), the Fe³⁺/Fe²⁺ level is still high around Aβ plaque regions, suggesting the potential existence of a continuous source of Fe³⁺ diffusing out from surrounding cells and/or proteins, or a different mechanism of Aβ plaque/ferric ion-related reactive oxygen species (ROS) generation. From these results, the study validated that both the Fe(II)-H5 and Fe(III)-B12 DNAzyme sensors can monitor the dynamic of Fe²⁺ and Fe³⁺ interconversion and provide spatiotemporal information that can gain deeper insights into iron-related diseases such as AD.

### DISCUSSION

Visualizing different oxidation states of redox-active metals provides valuable information for understanding the role of metal redox in regulating biological processes and human health. Despite this importance, current imaging tools are limited, and simultaneous imaging of different oxidation states of the same metal ion has yet to have been reported. To overcome these limitations, the study isolated DNAzymes that are highly specific for either Fe²⁺ or Fe³⁺ through in vitro selection. Upon further characterization and conversion into fluorescent sensors using the catalytic beacon strategy, this study demonstrated the ability to use these DNAzyme-based turn-on fluorescent sensors for simultaneous detection of two oxidation states of iron in living cells and in brain slices of transgenic AD mice.

Recently, it has been demonstrated that ferroptosis increases the total labile iron pool that contains both Fe²⁺ and Fe³⁺ due to degradation of the Fe³⁺-storage protein ferritin in lysosomes, causing labile iron overload (82, 83). The study monitored the iron distribution during ferroptosis and observed a similar increase of total iron. The sensors provided the first piece of information for iron redox changes during ferroptosis. An increase in the overall labile iron pool was observed within 4 hours when inducing ferroptosis with RSL3. After 4 hours of ferroptosis, a decrease of both Fe²⁺ and Fe³⁺ was observed, which suggests a depletion of labile iron from the endosomal-lysosomal system in the later stage of ferroptosis. This reduction could be induced by STEAP3 or other endogenous metalloreductases, while the initial increase of Fe³⁺ is possibly from the release of ferritin-bound Fe³⁺ (65). In addition, a decrease in the Fe³⁺/Fe²⁺ ratio was observed over time, and such an observation reveals a potential role of iron redox cycling, which may serve as a source of oxidative stress, during ferroptosis. This conclusion is consistent with oxidative stress accumulation during ferroptosis, an iron-dependent cell death pathway (84).

As a key player in redox biology, iron is also involved in the generation of reactive oxygen species from Aβ (79). By using the DNAzyme-based sensors, the study observed elevated iron signals that colocalized with aggregated Aβ plaques. The observations on both total iron increase and the distribution patterns are consistent with earlier reports demonstrating increase in the level of iron in AD brain slices stained with potassium ferricyanide/ferrocyanide or as detected by magnetic resonance imaging (MRI) (18, 22, 85). Although these previous works reported similar increases in cerebral iron accumulation around Aβ, here, the study was able to visualize both Fe²⁺ and Fe³⁺ simultaneously in single brain slices, allowing observation of the spatial relationship between the two oxidation states. In addition, the iron-specific DNAzyme-based sensors allows for spatial information about iron redox ratios, revealing a significant increase in the Fe³⁺/Fe²⁺ ratio surrounding amyloid plaque regions but not in other brain regions. The data suggest that not only total iron but also iron redox cycling is involved in the progression of AD. Combining these data with the observation that both Fe²⁺ and Fe³⁺ levels increased around Aβ plaque regions and suggests a potential role of Aβ plaques in accumulating Fe³⁺ over Fe²⁺ from surrounding cells and/or proteins in AD mouse brains. The elevated levels of iron surrounding Aβ plaques might be derived from labile iron pools that contribute to the formation of iron-Aβ adducts or transchelation of iron from ferritin by Aβ plaques during AD progression due to the potential interaction between iron and Aβ plaques, instead of simply changing the oxidation states between different forms of iron (86, 87). However, it is unknown whether the dysregulated iron is involved in amyloid plaque formation, or this is a secondary effect of amyloid plaque formation in this mouse model. Overall, the data demonstrate that the DNAzyme-based iron sensors can provide unique and powerful tools for studying the intracellular dynamics of iron redox states and have the potential to open new avenues to further investigate different biological processes that involve redox metal ions and understand their roles in several neurodegenerative diseases such as AD.

### Example 3: Deciphering Iron Redox Cycles in Ferroptosis-Based Cancer Therapy

Ferroptosis is a promising alternative approach to cancer therapy: Ferroptosis, defined in 2012,¹ is a type of regulated cell death driven by iron (Fe)-dependent lipid peroxidation.¹⁻⁵ While many cancer therapies aim to induce apoptosis in cancer cells, genetic alterations in apoptotic pathways often render drugs ineffective, leading to therapy resistance and tumor recurrence.⁶⁻¹⁰ Ferroptosis has emerged as an alternative approach to cancer therapies,^{1-5,11,12} especially for some therapy-resistant cancers (e.g., triple-negative breast cancer), as they are susceptible to ferroptosis owing to their metabolic features.¹²⁻¹⁵ Also, Ferroptosis has also been recognized as a critical cell death response triggered by a variety of conventional therapies (e.g., radio-, chemo- and immunotherapies).¹⁶⁻¹⁹ Therefore, ferroptosis inducers (FINs) hold great potential in cancer therapy, especially in combination with conventional therapies.^{12,20,21}

The molecular mechanism of ferroptosis is not fully understood, which is a key barrier to selective cancer therapies with minimal side effects: Despite a decade of intense research, the intricate molecular mechanism of ferroptosis remains to be fully deciphered,² primarily due to its complexity. For example, the reason why certain types of cancer display a higher susceptibility to ferroptosis compared to other types is not fully understood. Ferroptosis has been described as a double-edged sword,^{12,22} capable of inducing cancer cell death, while also playing a role in antitumor immunity.²³ Moreover, ferroptosis is implicated in many other diseases, including neurodegenerative diseases. However, its similarities and differences in cancer cell death, antitumor immunity, and other diseases are unclear. Consequently, designing FINs that selectively target cancers without causing undesirable side effects remains a major challenge. Therefore, a comprehensive understanding of the molecular mechanism of ferroptosis is a basic research endeavor that has a major translational impact on cancer therapy, such as designing cancer-specific FINs that do not cause other side effects or diseases.

The role of Fe in ferroptosis is a much less explored area: Ferroptosis research lies at the intersection of lipid metabolism, reactive oxygen species (ROS) biology, and Fe regulation.² Most research in the field has focused on understanding 1) ROS biology that has identified peroxidation of polyunsaturated fatty acid phospholipids (PUFA-PLs) as the most important hallmark of ferroptosis;²⁴⁻²⁶ and 2) lipid metabolism, which has led to developing many FINs and inhibitors of PUFA-PLs pathways. Despite the acknowledged central role of Fe-driven reactions in “ferroptosis”, the role of Fe in this process is much less explored. Iron manipulation has been done through Fe supplementation using carriers like transferrin or Fe depletion by Fe chelators, but experts caution against these approaches due to potential side effects beyond ferroptosis.^{3,27} Cellular Fe transport and storage are tightly regulated, and certain FINs induce ferroptosis with minimal or no alteration of the overall Fe content. However, the mechanism by which Fe triggers ferroptosis with minimal overall Fe level change are not understood.

Fe redox cycling is a key driver of ferroptosis: PUFA-PLs peroxidation, the most important ferroptosis hallmark, 1,4,25 underscores the essential role of redox reactions in ferroptosis. Redox reactions involving reactive oxygen species (ROS) are crucial in regulated cell death pathways such as apoptosis, necroptosis, and ferroptosis, where lipid peroxidation plays a key role.²⁸ For example, oxygenation of cardiolipins is vital for apoptosis.²⁹⁻³¹ While ROS is common among these cell death pathways, what sets ferroptosis apart is the unique initiation mechanism of ROS production and Fenton reaction through Fe redox cycles.⁵ The labile iron pool (LIP), including weakly bound Fe²⁺ and Fe³⁺ with molecules like glutathione, plays a major role in ferroptosis.³²⁻³⁴ Importantly, while Fe²⁺/Fe³⁺ redox cycles have been known to be essential in LIP storage, release, and transport,³⁵ studies have shown that lipid peroxidation experiences a significant lag phase when all the Fe in the reaction exists solely in the Fe²⁺ or Fe³⁺ state,³⁶ and the ratio of Fe³⁺ to Fe²⁺ has been identified as the primary determining factor for the initiation of lipid peroxidation, with 1:1 as the optimal ratio for the most effective oxidation.³⁷⁻⁴⁸ Modulating Fe²⁺/Fe³⁺ ratios has been shown to initiate lipid peroxidation even under low H₂O₂ conditions.⁴⁹⁻⁵⁴ Recent evidence demonstrates that FINO2, a class IV FIN, induces ferroptosis by oxidizing Fe²⁺ to Fe³⁺ without requiring additional Fe. It also does not react with PUFA in the absence of Fe,⁵⁵ highlighting the essential role of Fe redox cycles in a FIN. Together, these studies emphasize the significance of Fe's oxidation states and redox cycling in ferroptosis, providing insights for designing potent FINs in cancer therapy.

Understanding Fe redox cycles requires simultaneous imaging of spatial distributions of Fe²⁺ and Fe³⁺ in cancer cells: Subcellularly localized lipid peroxidation contributes differently to ferroptosis at different sites.^{2,56} While the endoplasmic reticulum (ER) serves as a central hub that drives the lipid peroxidation, other organelles, such as mitochondria and lysosomes, can initiate or enhance the susceptibility of cells to ferroptosis.³ Therefore, investigating subcellular localization of Fe and its redox cycling is crucial in understanding how they contribute to lipid peroxidation, and eventual ferroptosis, because ROS (e.g., HO·) reacts with lipids at diffusion-controlled rates and thus cannot “travel” far from the site of their generation.^{28,57,58} In contrast, Fe²⁺ and Fe³⁺ in the LIP can be transported through different parts of the cell, weakly bound to glutathione and other molecules in cells.⁵⁹⁻⁶² Until a recent report,⁶³ simultaneous imaging of spatial distributions of labile Fe²⁺ and Fe³⁺ in cancer cells, which is crucial for understanding the Fe redox cycles in ferroptosis-based cancer therapy, had not been achieved. This represents a significant technical challenge.

A study was conducted to fill a critical knowledge gap in the molecular mechanism of ferroptosis about Fe redox cycles by developing selective sensors to overcome the technical challenge of simultaneously imaging of spatial distributions of Fe²⁺ and Fe³⁺ in cancer cells. Using these sensing probes, the study aims to answer: 1) What are the spatial distributions of Fe²⁺, Fe³⁺ and Fe²⁺/Fe³⁺ ratios in cancer cells that have different resistance to ferroptosis? 2) How is Fe redox regulated by known ferroptosis-related genes and FINs? 3) Are there any other genes involved in Fe redox regulation that either promote or inhibit ferroptosis?

Compared to the extensive understanding of apoptosis in cancer, knowledge of ferroptosis in cancer is limited. Despite its potential as a therapeutic alternative, the molecular mechanism of ferroptosis remains unclear. The development of selective FINs with minimal side effects is hindered by the lack of mechanistic insights, particularly regarding the role of labile Fe in ferroptosis. This is despite the recognition of the central role of Fe, which distinguishes ferroptosis from other regulated cell death pathways. Strong evidence has shown that the ratios of Fe²⁺/Fe³⁺ in the LIP are determinative in initiating lipid peroxidation, the most important hallmark of ferroptosis.³⁷⁻⁴⁸ Despite the importance and evidence that lipid peroxidation at different cellular locations contribute to ferroptosis differently, how the distribution of Fe²⁺ and Fe³⁺ in cancer cells is related to ferroptosis has not been studied in detail. This study can overcome a major technical challenge by simultaneously imaging Fe²⁺ and Fe³⁺ with high spatial resolution, making it possible to measure Fe²⁺/Fe³⁺ ratios in different organelles of cancer cells with different degrees of ferroptosis resistance and Fe redox in response to known ferroptosis genes and FINs. With this information, this study can provide new insights into the molecular mechanism of ferroptosis-based cancer therapy. The sensors can also be used as a read out for a CRISPR-based whole genome screening to identify new genes that are related to Fe redox changes. The insights gained from this research can fill a major knowledge gap in the molecular mechanism of ferroptosis, enhance the knowledge of current FINs, facilitate the design of more selective FINs for cancer therapy, and unlock the full potential of ferroptosis as an effective therapeutic approach for cancer.

State-of-the-art methods for imaging Fe redox in cancer cells and their limitations: To study Fe redox in biological systems, different methods have been explored, but with limited success. For instance, techniques like inductively coupled plasma mass spectrometry and electron paramagnetic resonance spectroscopy⁶⁴ measure Fe concentrations and speciation but lack the ability to observe spatiotemporal distributions of Fe redox states in vivo. On the other hand, X-ray fluorescence,⁶⁵ X-ray absorption, magnetic resonance imaging,⁶⁶⁻⁶⁸ and histochemical methods using potassium ferricyanide/ferrocyanide staining offer spatial information in brain tissues.^{33,69-74} However most existing methods measure total Fe content rather than the LIP involved in ferroptosis.^{33,69,71,75,76} Of the available methods for imaging the LIP in cells, fluorescent sensors are the preferred choice, due to their high signal intensity and fast response time. Therefore fluorescent sensors based on small organic molecules that can detect Fe²⁺ or Fe³⁺ in living cells have been reported.⁷⁷⁻⁸⁴ However, many of these sensors do not display enough selectivity against either other metal ions or the different oxidation states of Fe (i.e., Fe²⁺ vs. Fe³⁺). Additionally, imaging Fe²⁺ and Fe³⁺ simultaneously requires different fluorophores. It is often difficult to transform the binding of either Fe²⁺ or Fe³⁺ into a fluorescent signal that is independent of the choice of fluorophore and achieving the simultaneous imaging often necessitates redesigning the sensors and can affect their properties such as selectivity.^{61,81} They also often encounter non-specific quenching of fluorophores by Fe²⁺ or Fe³⁺ that have unpaired electrons.^{85,86} It is also a challenge to tune the dynamic range of the detection in order to match the Fe²⁺ and Fe³⁺ concentrations in cells. Moreover, most of these sensors work in organic solvents and thus can be difficult to be adapted for in vivo imaging. Finally, it is difficult to control the timing of detection after the sensors are delivered into the cells. Therefore, there are currently no reported methods available to simultaneously image and study the ratio of Fe²⁺ and Fe³⁺ in cancer cells.

An approach of DNAzyme-based “catalytic beacon” sensors for redox-active Fe: In 1994, DNA molecules with enzymatic activity were discovered.⁸⁷ These deoxyribozymes (DNAzymes)⁸⁸⁻⁹⁰ have shown catalytic efficiency similar to other enzymes (e.g., the kobs for the “10⁻²³” DNAzyme of 109 M⁻¹ min⁻¹ rivals that of ribonucleases).⁹¹ Since most DNAzymes require metal ions for activity,^{87,88,90} the PI's group pioneered the development of DNAzymes as highly selective sensors for many metal ions including Pb²⁺,^{87,92-96} Cu²⁺,^{97,98} Zn²⁺,^{87,88,90} UO₂²⁺,⁹⁹ Na⁺,¹⁰⁰ Li⁺,¹⁰¹ and Mn²⁺,¹⁰² using the process of in vitro selection, a combinatorial selection method for obtaining DNAzymes from a large DNA library (up to 1015 sequences).^{94,95} To enhance selectivity, a counter selection step was employed to remove DNA sequences competing with the target metal ion,^{100,101,103} resulting in up to >1 million fold of selectivity against competing metal ions.⁹⁹ To transform the binding of metal ions into a fluorescent signal independent of the choice of fluorophore, previous work has described a “catalytic beacon” approach to convert these DNAzymes into turn-on sensors, by taking advantage of the difference between the melting temperatures of one of the two binding arms before and after metal-dependent cleavage.^{63,104,105}

To image metal ions in living cells, this study has overcome challenges by a) mitigating the intrinsic non-specific quenching of fluorophore by Fe²⁺ or Fe³⁺ by using the “catalytic beacon” approach that spatially separates the metal-binding site in the DNAzyme from the fluorophore; such an approach also allows free choice of a different fluorophore for Fe²⁺ and Fe³⁺ for simultaneous imaging of both ions; b) screening different rounds of in vitro selection that perform at varying concentrations of Fe²⁺ or Fe³⁺ to obtain DNAzymes with different affinities for Fe²⁺ or Fe³⁺ so that the dynamic detection ranges can match those of Fe²⁺ or Fe³⁺ in cells;⁶³ and c) using a photocaged ribonucleotide¹⁰⁸ and HIFU¹⁰⁹ to control the timing of sensor activation. Recently, DNAzymes (Fe(II)-H5) and (Fe(III)-B12) that are highly selective for Fe²⁺ and Fe³⁺ ions, respectively, were obtained.⁶³ With these advancements, these methods can be applied to provide new insights into the roles of redox-active Fe in cancer ferroptosis.

By developing the approach of DNAzyme-based catalytic beacon sensors for imaging the spatial distributions of Fe²⁺ and Fe³⁺ and their ratios, this study can overcome a major technical challenge in understanding redox cycles in ferroptosis, which is extremely complicated and difficult to understand. The proposed aims shift the paradigm of ferroptosis research from the major focus on lipid metabolism and ROS biology toward the central role of Fe as the key to differentiating ferroptosis from other regulated cell death pathways, and explores a relatively new concept that the Fe²⁺/Fe³⁺ ratio, rather than total Fe, is the primary determining factor in lipid peroxidation, which eventually results in ferroptosis.

Visualizing Spatial Distributions of Fe²⁺ and Fe³⁺ in Cancer Cells and Mouse Models

The subcellular localization of the lipid peroxidation in different organelles (e.g., ER, mitochondria, and lysosomes) was found to contribute differently to ferroptosis,^{2,56} but the reason for such differences are unclear. Since the Fe²⁺/Fe³⁺ ratio is the primary determining factor in lipid peroxidation,³⁷-39 it is hypothesized that the spatial distribution of Fe²⁺ and Fe³⁺ in different organelles plays a crucial role in contributing to these differences. To test this hypothesis, the study can use recently reported DNAzyme sensors⁶³ to visualize Fe²⁺ and Fe³⁺ in different organelles by a) employing delivery reagents that have been shown to deliver the DNAzymes to different cellular compartments, and b) conjugating different tags that allow delivering the sensors to specific subcellular organelles.¹¹⁰⁻¹¹⁶ Furthermore, some cancer cells are more resistant to ferroptosis than others, but the reason for such differences is not fully understood.¹² It is hypothesized that the Fe redox cycles in these different cell types may be different, which may contribute to the different resistance. To test this hypothesis, the study can use the DNAzyme sensors to image spatial distributions of Fe²⁺ and Fe³⁺ in cancer cells that display different degrees of ferroptosis resistance. These methods were demonstrated in HT-1080 cells first because it is a ferroptosis-sensitive cell line that has been well studied in many laboratories in the field. In this way, the sensors can be calibrated while offering additional insights into Fe redox. After demonstrating that Fe²⁺ or Fe³⁺ can be delivered and imaged in different organelles of HT-1080 cells, the study can apply the methods to different cancer cell types that are either more (COV318 cells)¹¹⁷ or less (HEY cells)^{117,118} resistant to ferroptosis to identify similarities and differences in the contributions of Fe²⁺ and Fe³⁺ to ferroptosis resistance. These two cell lines were tested as they are both derived from human ovary tissues, which have similar tissue background but different ferroptosis resistance, for comparative studies.¹¹⁷ Finally, the study can extend the methods to xenograft mouse models to determine Fe²⁺ and Fe³⁺ distributions in a physiologically relevant system.

Visualizing Fe²⁺ and Fe³⁺ in different subcellular organelles in HT-1080 cells: To achieve this aim, the study can first use delivery reagents that have been observed to preferentially deliver DNAzymes in different subcellular localizations. For example, the Fe²⁺ and Fe³⁺ DNAzyme sensors have been delivered into endosomal-lysosomal system with polyethylenimine (PEI),⁶³ and other DNA-based sensors into mitochondria with DQAsomes,^{115,119} and into cytosol with Lipofectamine 3000 or thiol mediated uptake.¹²⁰ If these delivery agents do not work well for some organelles (e.g., ER) or may cause interference from the delivery agents, the DNAzymes can be conjugated with organelle-specific tags,^{112,121,122} such as pardaxin for ER,^{112,116} dequalinium chloride for mitochondria,^{114,115} and gold nanoparticles for lysosomes,^{113,123} DNA nanomachines for Golgi.¹¹⁰ Given previous successes with delivering DNA sensors into subcellular compartments, the study can achieve this aim by following similar protocols. Commercially available stains that label ER, mitochondria, and lysosomes can be used to evaluate the DNAzyme subcellular internalization rates¹¹⁵ Flow cytometry can be used to assess cell uptake and internalization efficiency on the large population level. Confocal laser scanning microscopy can be used to assess sensor delivery and distribution in cells via co-staining with organelle-specific markers. These protocols in have been used in a recent paper⁶³.

Imaging Fe²⁺ and Fe³⁺ in different cancer cells that display different ferroptosis resistance: To demonstrate that the methods can be generally applied to different cancer cell types and to elucidate similarities and differences among these cell types, the study can further apply the sensors to compare Fe²⁺ and Fe³⁺ distributions between HEY cells, which can readily undergo ferroptosis, and COV318 cells, which are resistant to erastin-induced ferroptosis.¹¹⁷ The transfection conditions can be optimized with different cell lines, to make sure they are comparable between different cell lines. If the conditions described above cannot achieve effective targeted delivery in these cell lines, transfection conditions, including the ratio between transfection agent and sensor, buffer conditions, cell density, and transfection time, can be optimized. After the sensor delivery is optimized, the spatial distributions of Fe²⁺ and Fe³⁺ in these cells can be imaged using the same protocols as described above and the compared to elucidate similarities and differences of their spatial distributions in different organelles.

Imaging Fe²⁺ and Fe³⁺ in mouse tumor models: Understanding Fe redox roles in cancer requires moving beyond cell lines to physiologically relevant animal models. The study can perform the in vivo studies with proliferated HT-1080 cells in a nude mouse flank.¹²⁴ The mouse can be obtained from Jackson Laboratories. To facilitate optimal in vivo tumor penetration, the DNAzyme sensors can be modified with 8 to 10 phosphorothioate backbone modifications to achieve high in vivo tumor delivery efficiency, similar to antisense oligonucleotides.¹²⁷ After DNAzyme injections to the tumor site, High-Intensity Focused Ultrasound (HIFU), an FDA approved method,¹²⁸ can be used for activating the sensors in vivo. The sensor activity in live mice can be evaluated through live mice imaging with the In vivo Imaging System (IVIS) and post-injection excised tumors, in which tumors can be analyzed for DNAzyme penetration depth and Fe location. Because tumors display complex characteristics compared to 2D cell culture models, such as cell-cell interactions and variable nutrient variability, live tissue models account for realistic factors that may influence Fe's spatial distribution in tumors. Fluorescence microscopy can be used due to its sensitivity, deep tissue penetration with near-IR fluorophores, and low background.¹²⁹ This method has been successfully used by many groups in vivo using DNAzyme sensors.¹³⁰⁻¹³² Although HIFU could ablate tissue, the treatment is based on thermal increase and a relatively long-time exposure. A recent study from the PI's lab has shown that a controlled scale of exposure can take advantage of the thermal change and achieve DNAzyme sensor-based metal detection in live mice.¹⁰⁹ By replacing the Zn²⁺ DNAzyme in this published work with Fe²⁺ or Fe³⁺ DNAzyme, by changing DNA sequences, the study can achieve HIFU-controlled Fe redox imaging in the xenograft mouse model.

Preliminary results: To test and evaluate the feasibility, Fe^{2+/3+}-specific DNAzymes were delivered in HepG2 cells to detect Fe in mammalian cells and showed elevated Fe levels but a decrease in Fe redox states during RSL3-induced ferroptosis.⁶³

### Studying the Fe Redox in Relationship to Known Ferroptosis-Responsive Genes and FINs

Ferroptosis is controlled by different signaling pathways involving different genes.¹³³ Discovering these genes played a key role in not only understanding the mechanism of ferroptosis, but also finding FINs toward cancer therapy. Despite the recognized central role of Fe in ferroptosis, the link between Fe redox states, their distributions, and how they are regulated by these genes and FINs, is weak. It is hypothesized that the spatial distributions of Fe²⁺ and Fe³⁺ and their ratios change in response to these genes and their corresponding FINs during ferroptosis. To test this hypothesis, the study can use the imaging techniques described above to first visualize the pattern of Fe²⁺ and Fe³⁺, as well as their redox ratio when the expression levels of the genes are modulated or when FINs are added. For the gene-regulated ferroptosis, the study chose the NCOA4 gene as an initial focus¹³⁴ because it regulates the major process of ferritinophagy, which contributes to Fe homeostasis. Previous reports have shown that NCOA4 depletion decreased oxidative stress and thus inhibited ferroptosis, while increasing NCOA4 expression increased cell's sensitivity to ferroptosis.³⁴ However, little is known about the mechanism of how NCOA4 regulates the Fe redox state distributions that contribute to ferroptosis. These results can fill this knowledge gap. Based on literature reviews, there are more than 20 FINs and more than 15 small molecule inhibitors.¹³⁵ The study chose Sulfasalazine, an FDA approved antirheumatic drug that has been shown to be a FIN, and ferrostain-1, which can inhibit Sulfasalazine-induced ferroptosis,^{136,137} to study Fe redox changes in response to FIN and inhibitors, primarily because they have been well studied in many laboratories in the field. Together, the results from this will provide stronger links between the Fe redox and known ferroptosis-responsive genes and FINS, allowing for a better understanding of the molecular mechanisms and better design of FINs.

Investigating Fe redox changes associated with NCOA4 expression levels: The study can use NCOA4 as an example to determine the relationship between ferroptosis-related gene expression and Fe redox distributions. Specifically, the study can utilize the DNAzyme sensors to study the concentration, distribution, and related ratio of Fe²⁺/Fe³⁺ when modulating the expression levels of the NCOA4 gene. Specifically, the study can apply CRISPR interference (CRISPRi) or RNAi to downregulate NCOA4. To upregulate the NCOA4 gene, the study can deliver plasmids to overexpress NCOA4 gene or employ CRISPR activation (CRISPRa) approach to enhance the expression of NCOA4 in cells if the gene knock-in efficiency would be low. The study achieved preliminary results by incubating the cancer cells with CoCl₂ which up-regulates NCOA4 mRNA by stabilizing hypoxia inducible factors through different mechanisms.¹³⁸ To validate successful regulation of gene expression, the study can perform genome sequencing, qPCR, and western blot. After establishing this system in HT-1080 cell line, the study can further test the DNAzyme sensors in induced mice tumor models by xenograft injection of the transgenic or mutant cancer cells into nude mice obtained from Jackson Laboratories.

Once the relationship between Fe redox and NCOA4 expression is determined in cell culture, the study can test and compare the Fe distribution in the xenograft mouse tumor with transgene or mutated NCOA4 cancer cell lines. During tumor growth, the study can treat tumors with ferroptosis inducers or other therapies that induce ferroptosis (such as radiation). The DNAzyme sensors can be delivered to mice, directly into the tumor. An IVIS can be used to obtain a spatial distribution of the Fe²⁺/Fe³⁺ in vivo. The study can further analyze the Fe distribution in tumor specific areas and organs by isolating tissue and imaging tissue slices. Post-mortem tissue analysis can be performed by confocal microscopy to detect the spatial distribution of Fe²⁺ and Fe³⁺. Since the system for detecting metal ions and small molecules in live mice¹⁰⁹ and tissue slices⁶³ has already been established, these methods can be readily adopted to the proposed aim here. Overall, these results can determine the correlation between NCOA4 gene expression and redox-active Fe in cancer cells.

Detecting Fe redox state changes in response to FINs: Using the method and DNAzyme sensors described above, the study can measure the Fe²⁺/Fe³⁺ ratios in different organelles when 1) stimulating ferroptosis with Sulfasalazine, an FDA approved antirheumatic drug that initiates ferroptosis, and 2) inhibiting Sulfasalazine-induced ferroptosis using ferrostain-1^{136,137} to observe any Fe redox change in different organelles. In a recent report, a lower ratio of Fe³⁺/Fe²⁺ was observed in the lysosomal system of RSL-3, another FIN.⁶³ These preliminary results are encouraging, suggesting that the Fe²⁺/Fe³⁺ ratios may be different between normal cells and the Sulfasalazine-induced ferroptotic cells, at least in a few organelles. Encouraged by these results, the study can image spatial distributions of Fe²⁺ and Fe³⁺ between cell lines that respond to ferroptosis, such as fibrosarcoma (HT-1080) and ovarian cancer (HEY). With these assays, the study can compare the differential subcellular distribution of Fe²⁺ and Fe³⁺ between different cell types when inducing ferroptosis. By studying the labile Fe ratiometrically in four different cancer cell lines when treated with ferroptosis inducer Sulfasalazine or with ferroptosis inhibitor ferrostain-1, the study can obtain a comprehensive understanding of the role of labile Fe in ferroptosis for a wide range of cancers. These results can either demonstrate the similarity between different cancer types and draw conclusions about which organelle is the key for regulating ferroptosis or identify differences between cancer types. Either result can give evidence about how universal the Fe redox is in the ferroptosis of different cancer types. More importantly, the differences or similarities between cancer cells with and without resistance to ferroptosis could help in understanding whether Fe redox changes are involved in the resistance of ferroptosis and direct further studies on how to induce cell death in those cells that are resistant to ferroptosis.

Preliminary results: The study tested cells that knocked down NCOA4 expression with RNAi or upregulated NCOA4 with DMOG and observed Fe redox increase with DMOG and Fe redox decrease with NCOA4 RNAi. Moreover, the study observed an Fe redox decrease in the lysosomal system of RSL3 induced ferroptotic cells.⁶³

Identifying New Genes Associated with Fe Redox Distributions

To meet the high demands for Fe during proliferation, cancer cells use efficient and tightly regulated mechanisms for Fe trafficking and metabolism, both of which involve Fe²⁺ and Fe³⁺ and its redox cycles.¹³⁹⁻¹⁴¹ A deeper understanding of such Fe²⁺/Fe³⁺-based mechanisms can provide further insights to kill cancer cells by ferroptosis therapeutically. Throughout the past decade of ferroptosis research, identifying the genes that play key roles in promoting or inhibiting ferroptosis has been a crucial step in finding FINs for cancer therapy.¹⁴² In addition to known genes that have been studied so far, it is hypothesized that there may be other uncharacterized genes that may play equal, if not more prominent roles in cancer ferroptosis through Fe redox regulation. To test this hypothesis, the study can use the DNAzyme sensors for Fe²⁺ and Fe³⁺ as a readout to guide genome-wide loss-of-function screens by the CRISPR-Cas9 system in cancer cells to identify genes that regulate Fe redox. The study chose the HT1080 cell to perform the screen for the same reason it is used as a model cell in experiments described above. Moreover, it is a standard cell line that can stably express CRISPR Cas9 nuclease for the screening.¹⁴³⁻¹⁴⁵ By combining this screening with next generation sequencing (NGS) and computational analysis, the study can identify new gene regulators to provide further mechanistic insights into cancer ferroptosis and potential new targets for cancer therapy.

Identifying new Fe redox regulatory genes based on CRISPR screen using DNAzyme sensors as the readout: To identify new genes that may mediate Fe redox metabolism, the study can perform genome-wide CRISPR-Cas9 loss-of-function screens to identify genes involved in Fe-redox regulation. To this end, human genome-wide CRISPR-Cas9 library TKOv3 (Addgene #90294), can be used to infect a Cas9 stably expressed HT1080 cell line (GeneCopoeia #SL512) by following the manufacturer's instructions.¹⁴⁶ Specifically, a multiplicity of infection of 1 TU/cell can be applied for infection (the number of virus particles can be optimized with positive control lentivirus with GFP, #A32060). The following day, the cells can be split into two biological replicates and selected with 3 μg/mL puromycin for 3 days to generate a heterogeneous knockout cell pool. The study can then transfect the cells with Fe²⁺ and Fe³⁺ DNAzyme sensors. Those cells that display changed steady state Fe²⁺/Fe³⁺ ratios (with elevated Fe²⁺, Fe³⁺, or both, which indicates the potential Fe ratio changes) can be enriched by FACS separately.^{147,148} The sorted cells can be further enriched in another round with FACS after culturing 7 days to obtain cells with higher fluorescence intensity. After 3 rounds of enrichment, the enriched cells can be collected and separated into two groups. One group can go through genomic DNA sequencing, and the other group can go through RNA-seq and barcoded gRNA sequencing.¹⁴⁹ As a control, the original HT1080 cell line pool infected gRNA, and transfected with the Fe sensors can be sequenced and serve as negative control.

With the above NGS data, both the genome indels and the enriched sgRNA sequences can be obtained and analyzed following the protocol of Brunello CRISPR/Cas9 screens and MAGeCK.¹⁵³ With these established analytical pipelines, the sgRNA fold change can then be calculated by comparing the enriched and control groups to determine genes involved in maintaining normal Fe redox ratios. The indels generated endogenously in the genome can be identified to confirm and support the analysis. To identify high-confidence hits, the study can use a stringent false discovery rate (FDR) threshold of 1%. To retrieve overlapping pathways among different comparisons the study can perform Gene Set Enrichment Analysis (GSEA). After the whole genome CRISPR-Cas9 loss-of-function screens, it is expected that a set of genes capable of balancing Fe redox ratios can be obtained. Later, the study can perform further analysis to map the genes in ferroptosis pathways and exclude known genes that regulate Fe metabolism, such as the Fe transporters and storage proteins, and focus on newly identified genes that are associated with ferroptosis and the regulation of Fe levels.

Establishing and validating a robust signature for Fe redox changes: During the whole-genome CRISPR screening, the study can perform parallel RNA-seq to compare RNA levels between enriched knockout cells and those without gene knockouts. To establish a Fe redox signature, the study can use RNA-seq to analyze genome knockouts that alter Fe redox ratios and genes that are differentially expressed in cells with higher Fe²⁺ or Fe³⁺ levels. To identify key genes that are linked to both Fe redox ratios and ferroptosis, gene ontology (GO) analysis can be performed for gene annotation. Gene dependencies can be analyzed from screen data and single candidate gene-based RNA-seq results to identify high-confidence candidate genes that regulate Fe redox.¹⁵⁴ The study can divide the NGS data randomly into two groups: 90% for gene signature discovery, and 10% for validation. For 90% of the data, the study can use gene expression profiling to systematically measure cellular transcriptome reprogramming in cells with or without a Fe redox change, using isogenic conditions for the analysis. The study can then perform further analyses to search for genes differentially expressed between control cells and other cells under different experimental conditions. The study can select genes whose expression fold changes differ by a factor of 2 or greater (P<0.001) between the experimental group versus control sets. To validate the Fe redox signature, the study can apply a supervised clustering approach to analyze the remaining 10% of the NGS data, which can be well characterized for their Fe redox ratio changes. Using the established signature described above, the study can predict Fe redox in 10% of samples and compare with true Fe redox data obtained from experiments. Statistical significance can be computed by comparing against ˜10,000 random labels of samples. By combining the RNA-seq and genomic analysis, the study aims to establish gene-Fe redox response signatures by connecting regulatory genes both upstream and downstream of the Fe redox cycle in ferroptosis. This unique signature derived from an integrative analyses process can be a powerful predictive tool that could guide future therapies for cancer.

For further experimental follow-up, the study can select top-ranked, statistically significant gene candidates and consider whether candidates satisfy the following criteria: 1) Fe redox regulation according to the CRISPR-Cas9 knockout screens; 2) involvement in ferroptosis according to GO annotation; 3) belonging to the same pathways as differentially expressed genes. Using the aforementioned criteria, a few candidates can be identified to study their regulatory function in changing Fe redox and Fe²⁺/Fe³⁺ distributions in cell lines with HT1080 cells as a proof of concept. The top 3-5 genes can then be selected for further validation. The study can use RNAi to knock down candidate gene expression and sensors can then be delivered into knock-down cell lines to observe Fe changes by the methods described above. Confocal microscopy can be used to study spatial localization and colocalization with other important proteins, such as ferritin, 155 GPX4,^{124,156} and ferroptosis suppressor protein 1.^{142,157} Candidate genes that show iron redox or distribution changes can be carried over for further studies.

Studying the ferroptosis vulnerability by using Fe redox-related gene KO cell lines and xenograft mouse model: It is hypothesized that the regulatory genes for Fe redox can trigger or silence ferroptosis by regulation of Fe redox activity and distributions, and thus may be used as potential targets for cancer therapy in the future. Thus, the study can evaluate the cell or xenograft tumor vulnerability to FINs upon gene manipulation. The study can regulate ferroptosis using sulfasalazine and lipostatin-1 in the cells with knocked down candidate genes and showed changes in Fe redox distribution and observe the Fe redox changes with the DNAzyme sensors. Cell viability, lipid peroxidation levels, and intracellular Fe redox can be evaluated by propidium iodide staining, BODIPY C11 staining, and DNAzyme sensors, respectively. Fluorescent imaging can be used to visualize the result. The top 1-2 candidates with the largest changes in Fe redox and/or distribution pattern and can regulate cell viability or alter lipid peroxidation levels can be identified for further validations. To further verify the regulatory behavior, the study can generate the mutated cell line with the gRNA of the 1-2 top candidates in HT1080-Cas9 cells and confirm earlier observations with the gene knock out (KO) cell lines.

Furthermore, in vivo studies with xenograft mouse models can also be utilized for the KO cell models with the protocols described above. The study can establish the xenograft tumor models by injecting 5×10⁶ WT HT-1080 or KO HT-1080 cells. When the tumor reaches 50-100 mm³, ferroptosis inducer sulfasalazine (dissolve in DMSO and dilute in PBS, inject intraperitoneally at a dose of 100 mg/kg daily) and ferroptosis inhibitor liproxstatin-1 (dissolve in PBS and intraperitoneally inject at a dose of 10 mg/kg every day) can be administrated, and the tumor volume can be measured every 3 days using established procedures.^{125,158,159} By comparing the tumor volume changes before and after the drug injection, the study can focus on 1) gene-KO-induced tumor growth differences before drug injection; 2) KO-tumor total Fe accumulation (by ICP-MS) and Fe redox states before drug injection (by DNAzyme-based imaging); 3) The tumor vulnerability to ferroptosis inducers; 4) Ferroptosis inducer-induced Fe redox changes by in vivo DNAzyme imaging; 5) whether the potential tumor inhibiting could be rescued by ferroptosis inhibitor. Based on systematically analyzing the above factors, the study aims to gain a more comprehensive understanding role that Fe redox regulation machinery in ferroptosis therapies. Additionally, if there are available small molecular inhibitors for the candidate genes, the study can also test if inhibiting the candidate gene with small molecule inhibitors can help with sulfasalazine-induced ferroptosis in ferroptosis-resistant ovarian cancer cells and show the potential of developing a combined therapy in the future.

Preliminary results: The study detected the Fe signal changes with DNAzyme sensors and flow cytometry. More than 20% cells showed elevated fluorescence intensity, which moved from Q4 to Q2, when these cells were treated with Transferrin, a Fe transporter that increase Fe influx, indicating that the method can identify Fe increased cells in CRISPR screen.

### Potential Modifications

Sensor stability: DNA sensors' nonspecific degradation can potentially create artifacts during detection. DNAzyme 3D structures display global folding properties like proteins or tRNA,^{160,161} enabling more resistance to nuclease degradation while remaining less immunogenic than proteins.^{162,163} To improve their stability, DNAzymes can be protected with functional groups at both ends to minimize degradation by exonucleases. Hydrolysis at the RNA cleavage site can be addressed by 2′-caging modification, allowing caged DNAzymes to remain stable in human serum for nearly two days.^{108,164} Moreover, the study can use an inactive DNAzyme in which a nucleotide critical to DNAzyme activity is substituted to generate as negative controls for normalizing the non-specific degradation caused artificial signals.^{100,101,109}

Perturbation of cellular functions by DNAzyme sensors: Sensor delivery into cells may potentially perturb cell metabolism. Despite this limitation, many sensors, including small molecule and fluorescent protein-based sensors, have provided invaluable information that is otherwise difficult to obtain. The study can take advantage of the sensors' high sensitivity to allow for the use of low quantities of DNAzyme sensor relative to the ion pool-just enough to observe the Fe redox. In addition, the DNAzyme does not compete with other biomolecules; upon binding to the Fe, the DNAzyme can cleave the substrate strand and then release Fe.^{165,166} With the small amount of DNAzyme and release of Fe following cleavage, the DNAzyme sensors should have minimal influence on the cellular metal concentration. Additionally, endogenous nucleic acid immune factors are sensitive to long cytosolic dsDNAs (>45 bp, cGAS; >80 bp, AIM2) and free 5′-/3′-ends (retinoic acid-inducible gene I, RIG-1). 167-169 DNAzymes are not typical dsDNA, and are composed of a tightly folded ssDNA catalytic core (˜20nt) and two dsDNA binding arms (˜20 bp). The dsDNA regions in DNAzymes are below 45 bp to prevent the activation of cGAS and AIM2.168 In addition, both ends of DNAzymes are protected by modification groups (fluorophores and quenchers) to avoid binding from RIG-1. The study can optimize the DNAzyme length by monitoring interferon levels using QPCR and western blot. The study can also set up controls and compare the relative expression change between groups. Importantly, the study can use loss-of-function DNAzymes as references to identify background noise that do not depend on Fe²⁺ or Fe³⁺.⁶³ This inactivated DNAzyme has almost the same sequence to the active DNAzyme, except it contains a single-point mutation in the catalytic core that eliminate Fe²⁺- or Fe³⁺-dependent activity. The signals from the inactive DNAzymes can be compared with and, if needed, subtracted from those of the active DNAzymes to account for the background noise.⁶³ Also, the differences between with/without FINs can be compared; with the same procedure, but different drug treatments, the sensors can likely display signal from the drug treatments instead of other artifacts.

HIFU may perturb cell metabolisms: While HIFU has been approved by FDA for clinical applications,¹²⁸ the study can rule out artifacts by using the controls mentioned in 4b, including using inactive DNAzymes to rule out any effect that are not dependent on Fe²⁺ or Fe³⁺. Since this study is mainly interested in Fe²⁺/Fe³⁺ ratios, artifacts can be minimized by comparing Fe²⁺ and Fe³⁺ signals relative to each other.

Insufficient sensitivity: Fe²⁺ and Fe³⁺-DNAzyme show significant fluorescent turn-on in the presence of Fe²⁺ (0-250 μM) and Fe³⁺ (0-40 μM). LIP is weakly bound to small anions, polypeptides, and membrane surface components.¹⁷⁰ Labile Fe is present at 2-5 μM, with most free Fe present in the Fe²⁺ state in the cytoplasm (90% of total Fe is stored as Fe³⁺, bound to proteins, like ferritin). Therefore, DNAzymes are suitable for detection. If needed, the study can perform re-selection for higher sensitivity¹⁷¹ or introduce modified nucleotides with functional groups (e.g., carboxylate) for stronger Fe binding.

Different subcellular localizations: The DNAzyme sensor may preferentially go to a specific subcellular location,¹⁷² such as the lysosome. To rule out this possibility, the study can use a ssDNA of the same length but with a different sequence that does not form the DNAzyme structures that can sense Fe²⁺ or Fe³⁺ as a control in all the experiments. The study can also conjugate the DNAzyme with a different fluorophore to rule out the possibility of the fluorophore directing the sensor to a specific location.

False positive results of CRISPR-based whole genome screen and gene manipulation: CRISPR-based whole genome screen and gene manipulation may suffer from off-target effects. To eliminate false positive results, the study can use a sgRNA library well designed by algorithms to maximize on-target specificity and activity.^{173,174} For each targeted gene, multiple sgRNAs can be designed and these sgRNAs results can be analyzed to minimize the influence from off-target effect. Also, the gRNA sequencing data can be compared with the genome DNA sequencing data to confirm the edits were from the corelated gRNA. Top 3-5 candidate genes can be validated with RNAi or small molecule based gene down-regulation.

### Example 4: Iron Redox Changes in Diseased Models

A study was conducted to detect iron redox changes in multiple diseased models, including cancer cell ferroptosis, iron redox in normal brain aging, and neurodegenerative diseases including Alzheimer's disease and the cuprizone model of Multiple Sclerosis.

For studying the redox changes in cancer ferroptosis, the sensors were delivered to the cultured cell lines with transfection reagents, and ferroptosis was induced with different ferroptosis inducers. During RSL3-induced ferroptosis, both Fe²⁺ and Fe³⁺ increased, while the Fe³⁺/Fe²⁺ ratio decreased. This decrease indicates that iron could be a source of oxidative stress during cell death. Interestingly, when ferroptosis was induced by Erastin, the Fe³⁺/Fe²⁺ ratio increased instead of decreased. To this end, the study observed different trends with ferroptosis inducers, which stimulate ferroptosis with different signaling pathways. This indicates that the sensors can be used to understand the role of iron redox changes, and when it is involved in the ferroptosis process in the future.

Moreover, to study how ferroptosis-related gene regulates iron redox, the study stimulated the expression of NCOA4 by incubating the cells with CoCl₂ or DMOG, and used RNAi to reduce the expression of NCOA4, and detected the expression of iron levels with the DNAzyme sensors. An increase in the Fe³⁺/Fe²⁺ ratio was observed with an elevation of NCOA4, while a decrease in the Fe³⁺/Fe²⁺ ratio was observed with a reduction of NCOA4. These observations indicate that NCOA4 can not only regulate the library of labile iron but also the ratio between different iron forms.

In addition to studying ferroptosis in cancer cell lines, the study also applied iron sensors to understand the iron redox changes in mice brains. The iron was stained with the sensors in both young and old mice's brains to compare how iron redox changes over their aging. During the staining, the study found that the background fluorescence was significantly different between young and old mice and can cover the differences in iron signal. To reduce the background, the study compared two different reagents that reduce the autofluorescence background, TrueBlack and Autofluorescence Eliminator. Both have two different protocols, treating the brains with them before staining, or after staining. The study compared both protocols for both dyes and found a 30 s TrueBlack treatment (1:100 diluted in 70% ethanol) worked best in reducing background but not causing non-specific signaling interference in the channel of Alexa 647. With these conditions, the study observed a decrease in labile iron, but an increase of iron redox in the hippocampal dentate gyrus region of aged mice brains. On the other hand, the iron redox did not change much in the CA1 region of the hippocampus of aged mice brains. This information suggests that the iron redox change may relate to cognitive aging in some of the brain regions and is worth in-depth investigation.

To apply the iron sensors with other antibodies and other sensors, such as GFP staining in transgenic mice with GFP expressions, the study changed the fluorophore and quencher pairs of the Fe²⁺ sensor.

TABLE 18
Sequence of the Fe²⁺ sensor used.

| | | SEQ |
|---|---|---|
| Strand | Sequence | ID NO |
| Enzyme Strand | /5IAbRQ/TGGATATCTCCTAGCCAGACTGTTATGTGT | 28 |
| | GATACGGCAAACTTCGTGATGCCTCTACGGGTCCG | |
| Mutated Enzyme | /5IAbRQ/TGGATATCTCCTAGTCAGACTGTTATGTGT | 29 |
| Strand | GATACGGCAAACTTCGTGATGCCTCTACGGGTCCG | |
| Substrate Strand | /5IAbRQ/CGGACCCGTATCAATCTCACGTATrAGGAT | 30 |
| | ATCCA/3AlexF546N/ | |
| Enzyme Strand | TGGATATCTCCTAGCCAGACTGTTATGTGTGATAC | 24 |
| (without | GGCAAACTTCGTGATGCCTCTACGGGTCCG | |
| fluorophores/ | | |
| quenchers) | | |
| Mutated Enzyme | TGGATATCTCCTAGTCAGACTGTTATGTGTGATACG | 31 |
| Strand (without | GCAAACTTCGTGATGCCTCTACGGGTCCG | |
| fluorophores/ | | |
| quenchers) | | |
| Substrate Strand | CGGACCCGTATCAATCTCACGTATrAGGATATCCA | 26 |
| (without | | |
| fluorophores/ | | |
| quenchers) | | |

The change in sensing output did not influence the sensing with the iron DNAzymes. By changing the fluorophore and quencher pair, the study achieved co-staining with GFP antibody and DAPI, which labels the nucleus, with the mice's brain slices. The study further compared how iron changes in different brain regions in transgenic mice with Flt1 gene overexpression and wild-type mice. When the Flt1 gene was expressed in young mice's brains, the iron levels decreased, and the Fe³⁺/Fe²⁺ ratio increased in the hippocampal dentate gyrus region, which mimicked the aged mice's brains. On the contrary, when the Flt1 gene was knocked down in old mice brains, the iron level increases, and the Fe³⁺/Fe²⁺ level decreases in the hippocampal dentate gyrus region of old mice brains. These data reveal that the Flt1 gene is regulating iron redox in the hippocampal dentate gyrus region and may be important for cognitive aging.

These sensors were further delivered into primary neuron cells to visualize how iron redox changes when regulating the gene expressions. The primary neurons were cultured for 7 days before 3-day infection with viruses to regulate their gene expression and then transfected with the iron sensors with TurboFect.

By changing the fluorophore and co-staining with copper DNAzymes, the study was able to achieve simultaneous imaging of Fe²⁺, Fe³⁺, Cut, and Cu²⁺ on mice brain slices.

The study also co-stained the sensors with other antibodies to identify cell identities in neuron degenerative diseases, such as Alzheimer's disease. The antibody staining was performed before staining iron sensors. After traditional immunochemistry staining (need to avoid antigen exposures that require H2O₂ or other oxidative reagents), brain slices were changed into the BisTris buffer for iron sensors and incubated with iron sensors for 30 minutes. Then the brain slices were mounted onto slices and imaged immediately to minimize the background cleavage or degradation of the sensors. The iron redox changes in specific cell types were evaluated by taking images with a confocal microscope and analyzing the iron signal that colocalized with specific antibodies. With these tools, the study observed elevated iron redox in the Aβ region of 5×FAD mice brains. The study also observed a decrease in iron levels in microglial cells of wildtype mice brains in the hippocampal region, but an increase in iron levels in 3×Tg mice brains. These results suggest that the iron levels in the microglial cells may be involved in the pathology of 3×Tg mice over their aging. More detailed investigations are still going on to test how they are correlated with this disease process.

With a similar technique, the iron changes were studied with the cuprizone model of Multiple Sclerosis as well. By imaging the iron changes in the corpus callosum brain region, which is mostly impacted by the disease status, the study observed an elevated iron redox over the course of cuprizone treatment. The Fe³⁺/Fe²⁺ elevation was back to normal upon the removal of the chemical, and remyelination of the mice brains.

To sum up, this study has demonstrated different applications of the iron sensors and optimized their conditions for the applications. As the ongoing research, the sensors can be used for further investigation of the role of iron redox changes in both normal and disease aging processes.

### Example 5: Additional Modifications of Iron Sensors

Point mutations were intentionally introduced into the Fe³⁺ B12 DNAzyme catalytic core, and subsequently assessed in comparison to the unaltered Fe³⁺ B12 substrate strand under controlled reaction conditions. These conditions included a solution comprising 5 mM Bis Tris, 40 mM Sodium Acetate, 200 mM NaCl at pH 5.5, with concentrations of 250 nM for both the mutated B12 DNAzyme Strand and the FAM-labeled Original B12 Substrate Strand, along with 100 μM FeCl₃. The reaction proceeded overnight at ambient temperature, followed by analysis via electrophoresis on a 15% denaturing gel.

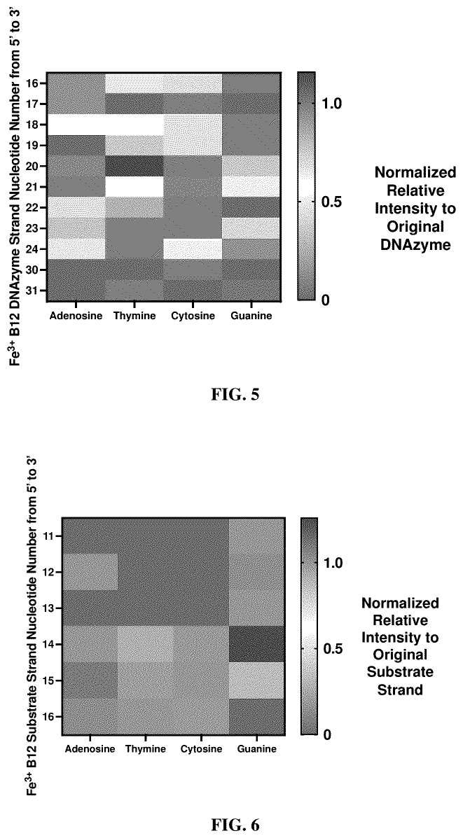

FIG. 5 presents a heat map derived from gel electrophoresis analysis, depicting Fe³⁺ B12 DNAzyme mutations activity that were normalized to the relative intensity of the original Fe³⁺ B12 DNAzyme. The heat map reveals regions of elevated intensity, notably illustrated by dark red shading, particularly evident at position 20 and within the thymine box. These observations suggest heightened activity in these specific regions compared to the baseline DNAzyme activity. Thus, these findings indicate the potential for increased enzymatic activity associated with this DNAzyme variant. TABLE 19 shows the Fe³⁺ B12 DNAzyme original and Fe³⁺ B12 DNAzyme C20T sequence.

TABLE 19
Sequences for the original Fe³⁺ B12 DNAzyme
and the variant with the C20T mutation.

| | | SEQ |
|---|---|---|
| | | ID |
| Name | Sequence 5′ to 3′ | NO |
| Fe³⁺ B12 | GCGGCATGCGCGTTTGCGG | 21 |
| DNAzyme | CACCTAAACGCTCCTAATA | |
| Original | GAG | |
| Fe³⁺ B12 | GCGGCATGCGCGTTTGCGG | 32 |
| DNAzyme | TACCTAAACGCTCCTAATA | |
| C20T | GAG | |

Point mutations were intentionally introduced into the Fe³⁺ B12 Substrate Strand's catalytic core, and subsequently assessed in comparison to the unaltered Fe³⁺ B12 DNAzyme strand under controlled reaction conditions. These conditions included a solution comprising 5 mM Bis Tris, 40 mM Sodium Acetate, 200 mM NaCl at pH 5.5, with concentrations of 250 nM for both the Original B12 DNAzyme Strand and the FAM-labeled mutated B12 Substrate Strand, along with 100 μM FeCl₃. The reaction proceeded overnight at ambient temperature, followed by analysis via electrophoresis on a 15% denaturing gel.

FIG. 6 presents a heat map derived from gel electrophoresis analysis, depicting Fe³⁺ B12 Substrate mutations activity that were normalized to the relative intensity of the original Fe³⁺ B12 Substrate. The heat map reveals regions of elevated intensity, notably illustrated by dark red shading, particularly evident at position 14 and within the guanine box and at position 15 and within the adenosine box. These observations suggest heightened activity in these specific regions compared to the baseline substrate activity. Thus, these findings indicate the potential for increased enzymatic activity associated with this DNAzyme variant. TABLE 20 shows the Fe³⁺ B12 Substrate strand original, Fe³⁺ B12 Substrate A14G, and Fe³⁺ B12 Substrate C15A.

TABLE 20
Sequences for the original Fe³⁺ B12
Substrate and the variants with the
A14G or C15A mutations.

| | | SEQ |
|---|---|---|
| | Sequence | ID |
| Name | 5′ to 3′ | NO |
| Fe³⁺ B12 Substrate | CTCTATTArGGGAG | 23 |
| Original | ACTCGCATGCCGC | |
| Fe³⁺ B12 Substrate | CTCTATTArGGGAG | 33 |
| A14G | GCTCGCATGCCGC | |
| Fe³⁺ B12 Substrate | CTCTATTArGGGAG | 34 |
| C15A | AATCGCATGCCGC | |

Point mutations were intentionally introduced into the Fe²⁺ H5 DNAzyme, and subsequently assessed in comparison to the unaltered Fe²⁺ H5 Substrate under controlled reaction conditions. These conditions include a solution containing 25 mM Bis Tris, 200 mM NaCl at pH 6.5, with concentrations set at 250 nM for both the mutated H5 DNAzyme Strand and the FAM-labeled Original H5 Substrate Strand, alongside 100 μM FeCl₂. The reaction proceeded overnight at room temperature under anaerobic conditions, followed by analysis via electrophoresis on a 15% denaturing gel.

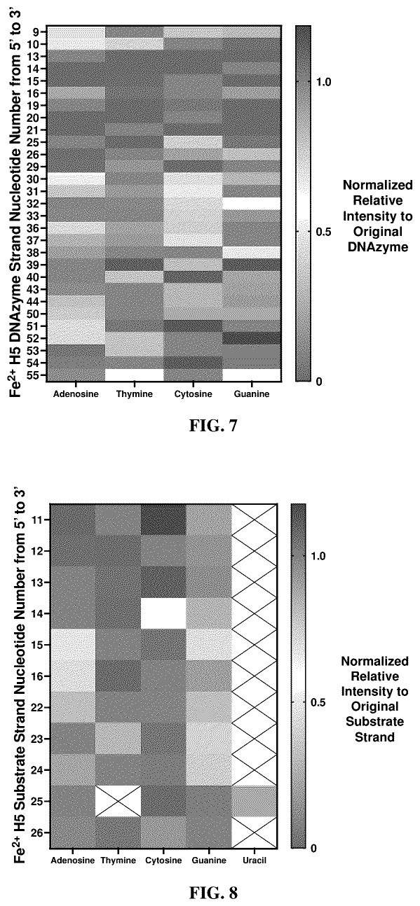

FIG. 7 presents a heat map derived from gel electrophoresis analysis, depicting Fe²⁺ H5 DNAzyme mutations activity that were normalized to the relative intensity of the original Fe²⁺ H5 DNAzyme. The heat map reveals regions of elevated intensity, notably illustrated by dark red shading, particularly evident at position 39 and within the thymine box, at position 39 and within the guanine box, at position 40 and within the cytosine box, at position 51 and within the cytosine box, at position 52 and within the guanine box, and at position 54 and within the cytosine box. These observations suggest heightened activity in these specific regions compared to the baseline DNAzyme activity. Thus, these findings indicate the potential for increased enzymatic activity associated with this DNAzyme variant. TABLE 21 shows the Fe²⁺ H5 DNAzyme original sequence, Fe²⁺ H5 DNAzyme A39T sequence, Fe²⁺ H5 DNAzyme A39G sequence, Fe²⁺ H5 DNAzyme A40C sequence, Fe²⁺ H5 DNAzyme G51C sequence, Fe²⁺ H5 DNAzyme C52G sequence, and Fe²⁺ H5 DNAzyme T54C sequence.

TABLE 21
Sequences for the original Fe²⁺ H5
DNAzyme and the improved variants.

| | | SEQ |
|---|---|---|
| | | ID |
| Name | Sequence 5′ to 3′ | NO |
| Fe²⁺ H5 | TGGATATCTCCTAGCCAGACTGTTATGTGTGATACGG | 24 |
| DNAzyme | CAAACTTCGTGATGCCTCTACGGGTCCG | |
| Original | | |
| Fe²⁺ H5 | TGGATATCTCCTAGCCAGACTGTTATGTGTGATACGG | 35 |
| DNAzyme A39T | CTAACTTCGTGATGCCTCTACGGGTCCG | |
| Fe²⁺ H5 | TGGATATCTCCTAGCCAGACTGTTATGTGTGATACGG | 36 |
| DNAzyme A39G | CGAACTTCGTGATGCCTCTACGGGTCCG | |
| Fe²⁺ H5 | TGGATATCTCCTAGCCAGACTGTTATGTGTGATACGG | 37 |
| DNAzyme A40C | CACACTTCGTGATGCCTCTACGGGTCCG | |
| Fe²⁺ H5 | TGGATATCTCCTAGCCAGACTGTTATGTGTGATACGG | 38 |
| DNAzyme G51C | CAAACTTCGTGATCCCTCTACGGGTCCG | |
| Fe²⁺ H5 | TGGATATCTCCTAGCCAGACTGTTATGTGTGATACGG | 39 |
| DNAzyme C52G | CAAACTTCGTGATGGCTCTACGGGTCCG | |
| Fe²⁺ H5 | TGGATATCTCCTAGCCAGACTGTTATGTGTGATACGG | 40 |
| DNAzyme T54C | CAAACTTCGTGATGCCCCTACGGGTCCG | |

Point mutations were intentionally introduced into the Fe²⁺ H5 Substrate Strand, and subsequently assessed in comparison to the unaltered Fe²⁺ H5 DNAzyme under controlled reaction conditions. These conditions include a solution containing 25 mM Bis Tris, 200 mM NaCl at pH 6.5, with concentrations set at 250 nM for both the Original H5 DNAzyme Strand and the FAM-labeled mutated H5 Substrate Strand, alongside 100 μM FeCl₂. The reaction proceeded overnight at room temperature under anaerobic conditions, followed by analysis via electrophoresis on a 15% denaturing gel.

FIG. 8 presents a heat map derived from gel electrophoresis analysis, depicting Fe²⁺ H5 Substrate mutations activity that were normalized to the relative intensity of the original Fe²⁺ H5 Substrate. The heat map reveals regions of elevated intensity, notably illustrated by dark red shading, particularly evident at position 11 and within the cytosine box, at position 13 and within the cytosine box, and at position 14 and within the thymine box. These observations suggest heightened activity in these specific regions compared to the baseline substrate activity. Thus, these findings indicate the potential for increased enzymatic activity associated with this DNAzyme variant. TABLE 22 shows the Fe²⁺ H5 Substrate strand original, Fe²⁺ H5 Substrate T11C, Fe²⁺ H5 Substrate A13C, and Fe²⁺ H5 Substrate A14T.

TABLE 22
Sequences for the original Fe²⁺ H5 Substrate
and the variants with the T11C or A13C
or A14T mutations.

| | | SEQ |
|---|---|---|
| | | ID |
| Name | Sequence 5′ to 3′ | NO |
| Fe²⁺ H5 Substrate | CGGACCCGTATCAATCTC | 26 |
| Original | ACGTATrAGGATATCCA | |
| Fe²⁺ H5 Substrate | CGGACCCGTACCAATCTC | 41 |
| T11C | ACGTATrAGGATATCCA | |
| Fe²⁺ H5 Substrate | CGGACCCGTATCCATCTC | 42 |
| A13C | ACGTATrAGGATATCCA | |
| Fe²⁺ H5 Substrate | CGGACCCGTATCATTCTC | 43 |
| A14T | ACGTATrAGGATATCCA | |

### Example 6: Copper Sensors

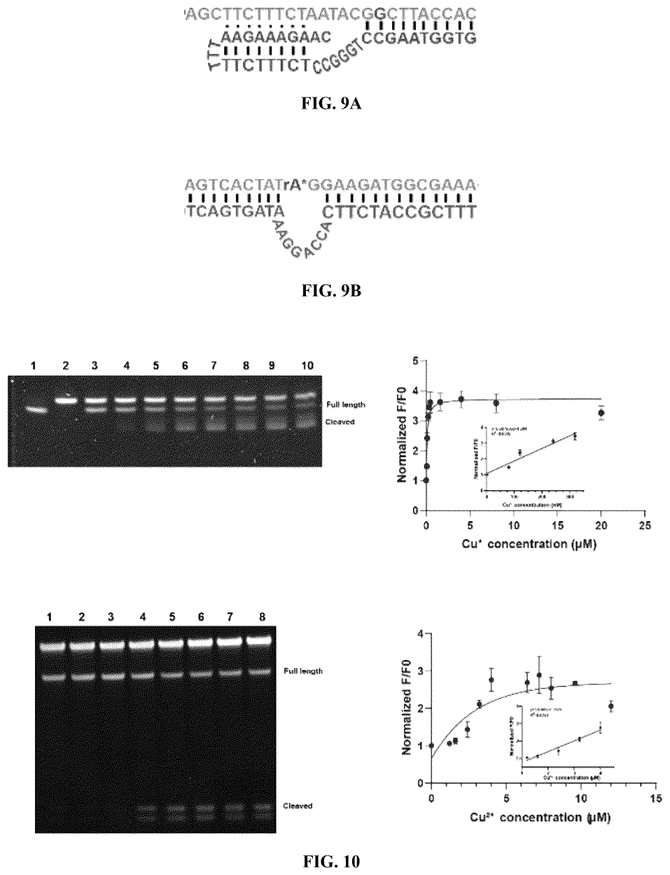

Two Cu sensors selective for Cu⁺ and Cu²⁺ were published previously (J. Am. Chem. Soc. 2007, 129, 32, 9838-9839, doi:10.1021/ja0717358) (Anal. Chem. 2016, 88, 6, 3341-3347, doi:10.1021/acs.analchem.5b04904) and can be considered as the starting point. However, neither of these two sensors were designed to be suitable for Cu imaging in living cells. A study was conducted which performed a series of reengineering of the sensors hybridizing regions for optimal performance in living cells for quantitative imaging. The new version of Cu sensor sequences is shown in FIGS. 9A-9B.

The performance of these two new sensors are highly effective with nM level detection range for Cu⁺ and low μM level detection range for Cu²⁺ as shown in FIG. 10.

The study then converted these two DNAzymes into fluorescent sensors for the detection of Cu ions in solution.

Since the DNAzyme sensors are exposed to various nucleic acid enzymes in biological samples during imaging, it is common to have some extent of sensor degradation and dehybridization, which can increase the background signal. Although the study applied modifications to the end of DNA strands to minimize the exonuclease activities, the background could not be fully removed. Thus, it is critical to estimate the background signal levels for accurate background subtraction and more precise signal quantification. Therefore, inactive copper sensors were designed to achieve these goals, with the catalytic core sequence being changed to other sequences that do not have Cu binding and catalytic activities.

In this iE design, the catalytic core sequence are converted into complement sequence of the original active sequences. Surprisingly, although the Cu⁺ inactive DNAzyme have very low background, the study found that the Cu²⁺ inactive DNAzyme has relatively strong background cleavage signal. However, when looking at the DNAzyme sequences without fluorophore and quencher labeling, the inactive sequences did not show much background cleavage on denaturing gel.

After more careful tests, it was realized that the fluorophore and quencher labels lead to unspecific substrate degradation and signal turn-on in the inactive DNAzyme groups. After realizing this issue, the study performed a screening of Cu²⁺ DNAzyme inactive sequences with fluorophore labels aiming to identify the best sequence with minimum background signal when fluorophores are present.

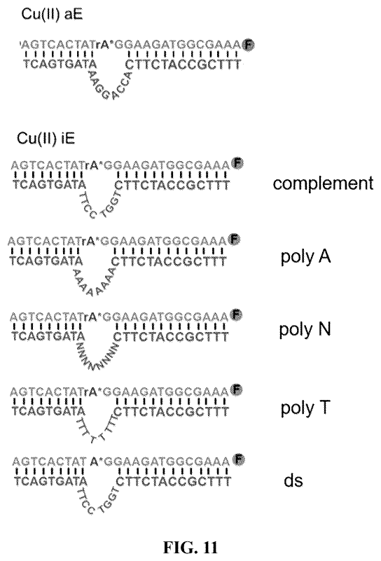

After screening, the study identified two inactive sequences that have the minimum activity to fluorophore labeled substrates, the complement sequence, and the poly T sequence. These sequences are shown in FIG. 11.

Next, the study further evaluated these two inactive sequences with both fluorophore and quencher labels. The study found that the poly T inactive sequence has the minimum background signal and can be suitable for cellular imaging control.

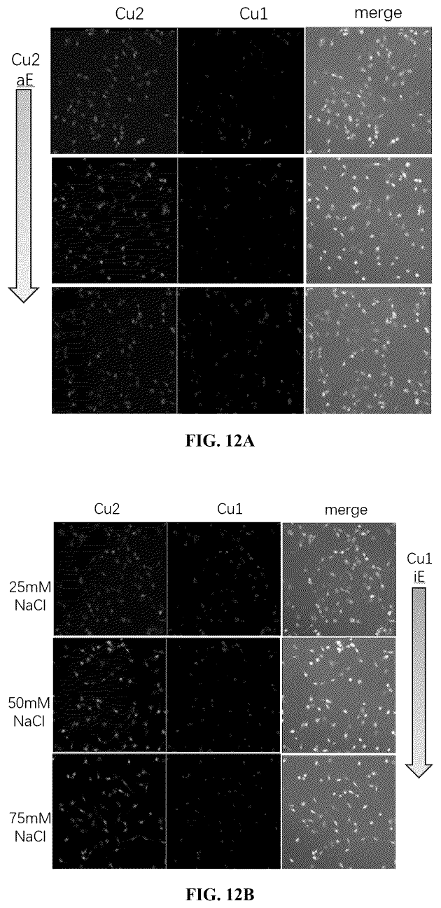

When the study applied the Cu DNAzyme sensors in Hela cells, it was noticed that different Cu sensors perform optimally at different buffer and ionic strength conditions as demonstrated in FIGS. 12A-12B. When Cu²⁺ DNAzymes perform best at low metal ion concentrations, Cu⁺ DNAzymes need to anneal at high metal ions concentrations for better detections.

These decoupling of annealing conditions for different Cu sensors made these sensors hard to be used at the same time for cellular imaging. When the sensors were tested in cells individually, the Cu sensors could differentiate endogenous Cu levels with weak turn-on. However, when two sensors are co-delivered and used in cells, the background signals are high, leading to obstacles in quantitative Cu imaging.

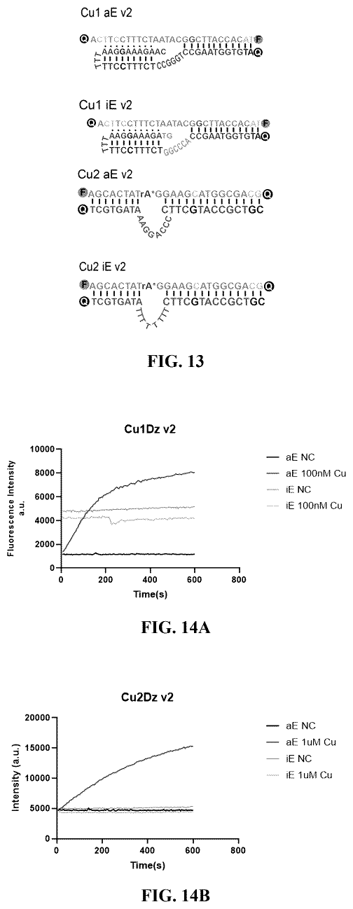

To overcome this issue, the study developed a further upgraded version of the sensor for better tolerance at cellular ionic conditions. The sequences of upgraded version are shown in FIG. 13.

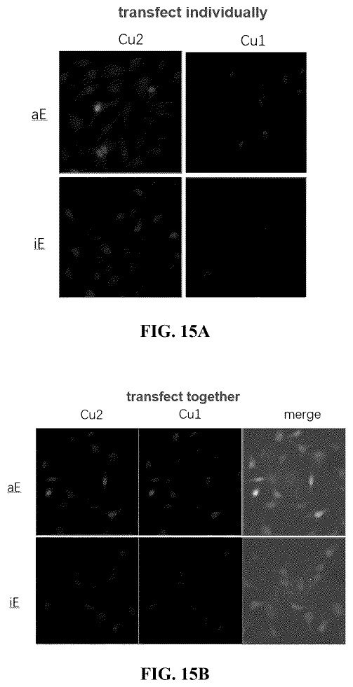

Both new version of Cu sensors are proved to have strong signal turn-on and low background as shown in FIGS. 14A-14B. The study then applied these sensors for Cu quantitative imaging in live cells. With these upgraded sensors, endogenous Cu could be detected in live cells both individually and simultaneously with minimum cross interference as shown in FIGS. 15A-15B. TABLE 23 summarizes the copper sensors explored in this study.

TABLE 23
Copper sensors.

| | | SEQ |
|---|---|---|
| | | ID NO |
| Version 1 unlabeled DNAzymes | | |
| Cu1 aS Cy5 | AGCTTCTTTCTAATACGGCTTACCAC | 44 |
| Cu1 aE IBRQ | GTGGTAAGCCTGGGCCTCTTTCTTTTTAAGAAAGAAC | 45 |
| Cu1 iE IBRQ | GTGGTAAGCCACCCGGTCTTTCTTTTTAAGAAAGATG | 46 |
| Cu2 PSrS | AGTCACTATrA*GGAAGATGGCGAAA | 47 |
| Cu2 aE | TTTCGCCATCTTCACCAGGAAATAGTGACT | 48 |
| Cu2 iE | TTTCGCCATCTTCTGGTCCTTATAGTGACT | 49 |
| complement | | |
| Cu2 iE polyA | TTTCGCCATCTTCAAAAAAAAATAGTGACT | 50 |
| Cu2 iE polyT | TTTCGCCATCTTCTTTTTTTTATAGTGACT | 51 |
| Cu2 iE polyN | TTTCGCCATCTTCNNNNNNNNATAGTGACT | 52 |
| Cu2 iE ds | TTTCGCCATCTTCCTATAGTGACT | 53 |

Version 1 Cu sensors

| Cu1 aS Cy5 | AGCTTCTTTCTAATACGGCTTACCAC/3Cy5Sp/ | 54 |
|---|---|---|
| Cu1 aS Cy5 | /5IAbRQ/AGCTTCTTTCTAATACGGCTTACCAC/3Cy5Sp/ | 55 |
| IBRQ | | |
| Cu1 aE IBRQ | /5IAbRQ/GTGGTAAGCCTGGGCCTCTTTCTTTTTAAGAAA | 56 |
| | GAAC | |
| Cu1 iE IBRQ | /5IAbRQ/GTGGTAAGCCACCCGGTCTTTCTTTTTAAGAAA | 57 |
| | GATG | |
| Cu2 PSrS 5FAM | /56-FAM/AGTCACTATrA*GGAAGATGGCGAAA | 58 |
| Cu2 PSrS 5FAM | /56-FAM/AGTCACTATrA*GGAAGATGGCGAAA/3IABkFQ/ | 59 |
| 3IBFQ | | |
| Cu2 aE 3IBFQ | TTTCGCCATCTTCACCAGGAAATAGTGACT/3IABkFQ/ | 60 |
| Cu2 iE polyT | TTTCGCCATCTTCTTTTTTTTATAGTGACT/3IABkFQ/ | 61 |
| 3IBFQ | | |
| Cu2 iE | TTTCGCCATCTTCTGGTCCTTATAGTGACT/3IABkFQ/ | 62 |
| complement | | |
| 3IBFQ | | |
| Version 2 Cu sensors | | |
| Cu1 aS 3Cy5 | /5IAbRQ/ACTTCCTTTCTAATACGGCTTACCACAT/3Cy5Sp/ | 63 |
| 5IBRQ v2 | | |
| Cu1 aE 5IBRQ | /5IAbRQ/ATGTGGTAAGCCTGGGCCTCTTTCCTTTTTAAG | 64 |
| v2 | GAAAGAAC | |
| Cu1 iE 5IBRQ | /5IAbRQ/ATGTGGTAAGCCACCCGGTCTTTCCTTTTTAAG | 65 |
| v2 | GAAAGATG | |
| Cu2 PSrS 5FAM | /56-FAM/AGCACTATrA*GGAAGCATGGCGACG/3IABkFQ/ | 66 |
| 3IBFQ v2 | | |
| Cu2 aE 3IBFQ | CGTCGCCATGCTTCACCAGGAAATAGTGCT/3IABkFQ/ | 67 |
| v2 | | |
| Cu2 polyT iE | CGTCGCCATGCTTCTTTTTTTTATAGTGCT/3IABkFQ/ | 68 |
| 3IBFQ v2 | | |

In summary, this study reengineered different versions of Cu⁺ and Cu²⁺ DNAzyme sensors from published DNAzyme sequences and overcame limitations in designing inactive controls for quantitative imaging, high background signals from unspecific degradation of sensors, and inconsistency in optimal imaging conditions. Using these sensors, the study achieved live cell imaging of endogenous Cu ions and Cu⁺/Cu²⁺ redox ratio calculations.

### Example 7: Simultaneous Cu⁺/Cu²⁺ Imaging in Brain Cells Using DNAzyme

Copper serves as an indispensable trace element crucial for the biological functions of living organisms. Within physiological conditions, it transitions between Cu²⁺ and Cut, with the latter prevailing as the primary oxidation state. In cellular environments, copper ions exhibit coordination with various nitrogen and sulfur-based ligands [1]. This versatility allows copper to participate in single-electron transfer reactions, thus playing pivotal roles in diverse biological processes such as respiration, free radical scavenging, iron metabolism, connective tissue biogenesis, and neuropeptide synthesis [2-4]. Intracellular chaperones and transporters, including CTR-1, ATOX-1, and GSH, [5] rigorously regulate copper levels to prevent the accumulation of free ions. Cellular copper ions exist predominantly in two forms: a static pool, tightly bound to proteins and macromolecules, and a labile pool, weakly bound to cellular ligands, rendering it mobile [6]. Mismanagement of these cellular copper pools can lead to oxidative stress, instigating the generation of reactive oxygen species (ROS) via Fenton-type reactions [7].

Given copper's vital biological functions, dysregulation of copper homeostasis can result in cellular dysfunctions stemming from abnormal increases in reactive oxygen species (ROS), which in turn cause oxidative damage to proteins, lipids, and DNA/RNA [8, 9]. These stress responses are implicated in various diseases, including cancer [10], neurodegenerative diseases such as Alzheimer's, Parkinson's, and Huntington's diseases [11-14], and genetic disorders such as Menkes and Wilson's diseases [15-17]. While these associations are intriguing, the precise causal links between copper homeostasis, brain function, and disease pathology remain inadequately understood.

Both too much and too little copper are stress to cell survival. Cuproptosis, a term emerging from recent research, refers to a unique form of cell death triggered by dysregulated copper homeostasis. Unlike other programmed cell death, cuproptosis specifically involves the disruption of cellular processes due to abnormal copper accumulation within the mitochondria. This aberrant accumulation prompts the aggregation of lipoylated dihydrolipoamide S-acetyltransferase (DLAT), an enzyme intricately linked to the mitochondrial tricarboxylic acid (TCA) cycle, thereby inducing proteotoxic stress and ultimately culminating in cell death. Predominantly observed in cells reliant on oxidative phosphorylation (OXPHOS) for energy production, cuproptosis is characterized by the concomitant aggregation of lipoylated mitochondrial enzymes DLAT and the depletion of iron-sulfur cluster (Fe—S) proteins. Cuproplasia, a recently identified phenomenon, encompasses the regulated growth and proliferation of cells dependent on copper. This term encapsulates the diverse effects of copper on cellular processes, including signaling pathways, enzymatic activities, and non-enzymatic functions. Importantly, copper signaling can be suppressed using selective chelators or enhanced through metal ionophores that redistribute copper stores within cells. Additionally, manipulation of cell signaling governing copper homeostasis, by genetic or pharmacological means, can modulate cuproplasia based cellular proliferation and growth. Thus, both cuproptosis and cuproplasia offers avenues for pharmacological intervention of human health.

Copper's biological relevance has motivated the development of techniques to measure its cellular levels. These include inductively coupled plasma mass spectrometry (ICP-MS) [18], X-ray fluorescence microscopy (XFM) and nanosecondary ion mass spectrometry (Nano-SIMS) [20], amongst others. While these techniques have provided important insights into the biology of copper, they are not capable of measuring and tracking it in living cells. Therefore, there is significant interest in the development of sensors that can selectively respond to copper ions, enabling its direct visualization in live cells. Over the last two decades several fluorescent probes for Cu⁺ [5, 21-26] and Cu²⁺ [23, 27-29] have been reported in the literature. However, most of the probes have low selectivity to Cu⁺ and Cu²⁺ for imaging copper Cu⁺ and Cu²⁺ at the same time. Therefore, there is significant interest in the development of sensors that can selectively respond to Cu⁺ and Cu²⁺, enabling its direct visualization in live cells. To meet this challenge, some have taken advantage of DNAzymes (deoxyribozymes or catalytic DNA) as a general platform for developing metal ion sensors [30]. First discovered in 1994, DNAzymes are metalloenzymes that recruit metal ions as a cofactor for catalysis [31-33]. Unlike screening the rationally designed small molecule or genetically encoded protein sensors, in vitro selection allows selection of DNAzymes with desired sensitivity and specificity for a metal ion of interest using a much larger library of DNA molecules containing up to 10¹⁵ different sequences. DNA synthesis is cost-effective with a variety of useful modifications, and its biocompatibility makes DNAzyme-based sensors excellent tools for live-cell imaging of metal ions.

In this study, Cu⁺ and Cu²⁺ dependent DNAzyme were applied to characterize labile copper pools in living cells. The study also established the sensing system for comparative metal ion visualization in the three major types of cells in the brain: neurons, astrocytes, and microglia with the goal of determining whether there is copper homeostasis in living neurons from AD patients and healthy individuals. The work reported here opens a new avenue for the selective detection of Li⁺ in living BD neurons and thus provides the opportunity for further studies that uncover the physiological response to lithium therapy.

Copper DNAzyme can detect changes of labile Cu⁺ and Cu²⁺ levels in SH-SY5Y cells and three major types of cells in the brain. In parallel, Cu⁺ and Cu²⁺ DNAzyme can be used in the future to monitor changes in intracellular copper homeostasis associated with AD. Taken together, this work provides a unique approach to DNAzyme-based imaging of Cu⁺ and Cu²⁺ that related to neurodegenerative diseases. In addition, the foundational information on cell type-specific changes in labile copper offers a starting point for further investigations of copper biology in the brain and beyond to advance understanding of transition metal signaling.

### Materials and Methods

Materials: Oligonucleotides used in this study (TABLE 24) were all ordered from Integrated DNA Technologies (Coralville, IA). DNA stock solutions were prepared with molecular biology grade water (Corning). TurboFect transfection reagent was purchased from Thermo Fisher Scientific. Amyloid-β (1-40) peptide (Aβ40) was synthesized by Cayman Chemical. 40% Acrylamide/Bis Solution (29:1) was from Bio-Rad. All the other chemicals were obtained from Sigma-Aldrich.

TABLE 24
DNA sequences used in this study. The asterisk (*) represents a
phosphorothioate (PS) modification

| | | SEQ ID |
|---|---|---|
| Name | Sequence from 5′ to 3′ | NO |
| Cu1 aS Cy5 | /5IAbRQ/AGCTTCTTTCTAATACGGCTTACCACT/3Cy59p/ | 69 |
| Cu1 aE Q | /5IAbRQ/GTGGTAAGCCTGGGCCTCTTTCTTTTTAAGAAA | 56 |
| | GAAC | |
| Cu1 iE Q | /5IAbRQ/GTGGTAAGCCACCCGGTCTTTCTTTTTAAGAAA | 70 |
| | GAAC | |
| Cu2 PSrS | /56-FAM/AGTCACTATrA*GGAAGATGGCGAAA/3Dab/ | 71 |
| FAM | | |
| Cu2 dS | /56-FAM/AGTCACTATAGGAAGATGGCGAAA/3Dab/ | 72 |
| FAM | | |
| Cu2 aE Q | TTTCGCCATCTTCACCAGGAAATAGTGACT/3Dab/ | 73 |

Instrumentations: Gels imaging were captured by Bio-Rad Gel Doc 2000 imaging system. Fluorescence spectra were collected by Hitachi fluorimeter. Cell images were obtained using a Zeiss LSM 880 confocal microscope at 63× magnification.

Gel based activity assays: For Cu⁺ DNAzyme, 1 μM of Cu1 aS and 2 μM of Cu1 aE were added in 50 mM Bis-Tris, 150 mM NaCl, pH 7.5. The solution was heated at 90° C. for 5 min and slowly cooled down to room temperature for more than 1 h to anneal the DNAzyme. After annealing, each sample contains a final of 5 μL of 0.25 μM DNAzyme complex. 2 ul of 50 μM of freshly prepared ascorbate was mixed with DNAzyme complex. After adding 2 μl different concentration of Cu²⁺, incubated at room temperature for 60 min. The samples were separated by 15% polyacrylamide gel electrophoresis (PAGE) gels. For Cu²⁺ DNAzyme, the DNAzyme complex was annealed with 5 μM of Cu2 PSrS and 7.5 μM Cu2 aE in 50 mM MES, 25 mM NaCl, pH 6.0. 2 μL different concentration of Cu²⁺ was added to a final of 5 μL of 0.7 μM DNAzyme complex, incubated at room temperature for 60 min. The reaction was quenched by stop-solution (50 mM EDTA, 8 M Urea, 1×TBE). The products were then loaded into 15% denaturing PAGE (dPAGE) gels. The gels were analyzed with Bio-Rad Gel Doc 2000 imaging system.

Fluorescence based activity assay: For Cu⁺ sensor, 5 M of Cu1 aS Cy5 and 9 UM of Cu1 aE Q were annealed in a buffer of 50 mM Bis-Tris, 150 mM NaCl, pH 7.5. For each test, 2 μL of the annealed sensor was diluted into 194 μL of buffer (50 mM Bis-Tris, 150 mM NaCl, pH 7.5), and 2 μL of 5 mM ascorbate was added. Then 2 μL of 2 μl different concentration of Cu²⁺ were added. The signaling kinetics were monitored (Ex=635 nm; Em=666 nm) using Hitachi fluorimeter. For Cu²⁺ sensor, the DNAzyme complex was formed by annealing the 5 μM of Cu2 PSrS FAM and 7.5 μM Cu2 aE Q in 50 mM MES, 25 mM NaCl, pH 6.0. For each well, 2 μL of the annealed sensor was diluted into 196 μL of buffer (50 mM MES, 25 mM NaCl, pH 6.0). 2 μL different concentration of Cu²⁺ were added to initiate cleavage reaction, and the signaling kinetics were monitored (Ex=488 nm; Em=520 nm) using Hitachi fluorimeter.

Aβ preparation: For Aβ monomer, 1 mg of commercial Aβ40 monomer powder was dissolved in 1 ml 1,1,1,3,3,3-hexafluoro-2-propanol (HFIP), incubated at room temperature for 2 hours. The resulting solution was aliquoted into multiple tubes. Then evaporated in vacuum desiccator overnight and dried by vacuum centrifuge for 15 min to generate monomeric films. The monomeric films can be resuspended by DMSO and then aliquoted into multiple tubes and store in-20 freezer. For Aβ oligomers, 100 μM Aβ42 oligomers solution was prepared by dissolving the Y mg monomeric films in (Y/4514.1)×10⁷ μL PBS buffer and incubated at 4° C. overnight. The resulting solution was then centrifuged at 3000 rpm for 15 mins to remove the insoluble aggregates. For Aβ fibrils, 100 μM Aβ42 fibrils solution was prepared by dissolving the Y mg monomeric films in (Y/4514.1)×10⁷ μL PBS buffer and string at 37° C. for 3 days.

Cell lines culture: The HeLa cells and SH-SY5Y cells (from ATCC) were cultured in DMEM supplemented with 10% fetal bovine serum (FBS), 100 U/mL penicillin, and 100 μg/mL streptomycin, and incubated in a humidified 5% CO₂ incubator at 37° C. After cells reached 80% confluency in culture flask, 0.25% trypsin/EDTA was added to detach it from the surface of the flask, then the cells were seeded in optical dishes before transfection.

Human neuronal progenitor cells (NPCs) culture and neuronal differentiation: The NPCs cells were cultured in geltrex (Thermos Fisher)-coated plates in NPC maintaining medium (Neurobasal Medium), 1×B27 minus vitamin A supplement, 2 mM GlutaMAX, 1% NEAA, 100 U/mL penicillin, 100 μg/mL streptomycin, and 20 ng/ml FGF2. Dissociated with 25% accutase (Corning) into single NPCs, and reseeded on 20 ng/ml laminin and poly-d-lysine-coated plates in NPC culture medium for 1 day. Then, half-medium change with neuronal differentiation medium composed of BrainPhys medium (Stem Cell), 1×SM1 supplement (Stem Cell), 1×N2 supplement (Stem Cell), 20 ng/mL BDNF (GeminiBio), 20 ng/mL GDNF (GeminiBio), and 200 nM l-ascorbic acid. The cells were fed by half-change of the medium every 3 days. For the first culture change only, 200 nM compound E (Calbiochem) was added. In the first 2 weeks of differentiation, 1 mM dibutyryl cyclic AMP (dbcAMP, Cayman) was added. Once a week, 1 μg/mL laminin was added when doing the half-change. Thereafter, the cells were fed by performing half-media change until Day 28 for future experiments.

Cell imaging: After 24 h transfection, the cells were washed with 1×HBSS, then 100 μL of Opti-MEM (Invitrogen) containing annealed Cu⁺ DNAzyme (final 400 nM) and Cu²⁺ DNAzyme (final 400 nM) with 2 μL of TurboFect Transfection Reagent were added to transfect DNAzyme. After 4 hours transfection, cells were washed with 1×HBSS and the medium was replaced by Opti-MEM for confocal microscopy images. Images were taken using a Zeiss LSM 880 confocal microscope at 60× oil. Quantification of the images were analyzed by ZEN Blue software.

### Results and Discussion

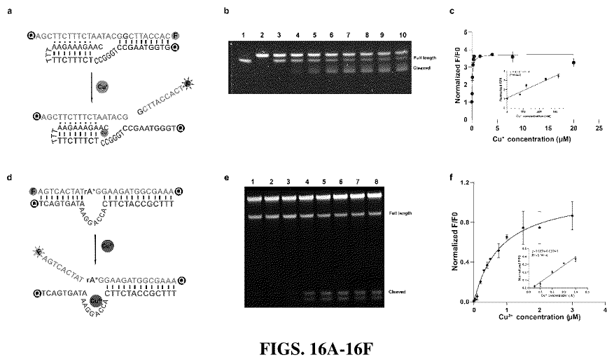

In vitro Activity Assay for Cu⁺ and Cu²⁺ DNAzyme: FIG. 16A and FIG. 16D are the secondary structure of Cu⁺ DNAzyme and Cu²⁺ DNAzyme, respectively. The quenching group Q were labeled at both the end of the substrate strand and the enzyme strand, to ensure the low fluorescence background signal of DNAzyme before being activated. The cleavage activity of Cu⁺ DNAzyme and Cu²⁺ DNAzyme were first verified by PAGE gels and dPAGE gels, respectively. As shown in FIG. 16B and FIG. 16E, the copper DNAzymes show Cu⁺/Cu²⁺ concentration dependent cleavage. And Cu⁺ DNAzymes shows ascorbic concentration dependent cleavage, there was no cleavage products at low concentrations of ascorbic, while Cu²⁺ DNAzyme has no activity with high ascorbic, indicating the Cu⁺ and Cu²⁺ DNAzyme have excellent selectivity to Cu²⁺ and Cu⁺, respectively.

To test sensitivity of Cu⁺ and Cu²⁺ DNAzyme, the kinetics of fluorescence increase at 666 nm and 520 nm in the presence of varying concentrations of Cu⁺ and Cu²⁺ were monitored. Fluorescence enhancement rates were higher with increasing levels of Cu⁺ and Cu²⁺. The fluorescence ratio of Cu⁺ and Cu²⁺ DNAzyme in the time window of 30 min were plotted in FIG. 16C and FIG. 16F, respectively. At low Cu⁺ concentrations, the response was linear (inset of FIG. 16C; linear regression equation: y=0.007935x⁺1.084). For Cu⁺ DNAzyme, a detection limit of 53.92 nM was determined based on 36/slope, where σ is the background variation of the sensor in the absence of Cut. For Cu²⁺ DNAzyme, the linear regression equation is y=1.062x−0.03241 (inset of FIG. 16F). The detection limit was calculated to be 35.16 nM Cu²⁺ based on 36/slope.

Fluorescence Imaging of Cu⁺ and Cu²⁺ in Live Cells: After establishing that Cu⁺ and Cu²⁺ DNAzyme are sensitive to Cu⁺ and Cu²⁺, respectively, and with high selectivity, the study next tested the ability of copper DNAzyme to respond to changes in labile copper levels in living cells. Specifically, Cu⁺ and Cu²⁺ DNAzyme were applied to live SH-SY5Y cells and cellular copper levels were perturbed by incubating with pyrrolidine dithiocarba-mate (PDTC) to increase intracellular copper levels and bathocuproine disulphonate (BCS) to decrease intracellular copper levels [23]. The study also explored the intracellular labile copper levels influenced by Amyloid-β (Aβ) peptides, a historic hallmark of Alzheimer's disease (AD) disease [35, 36].

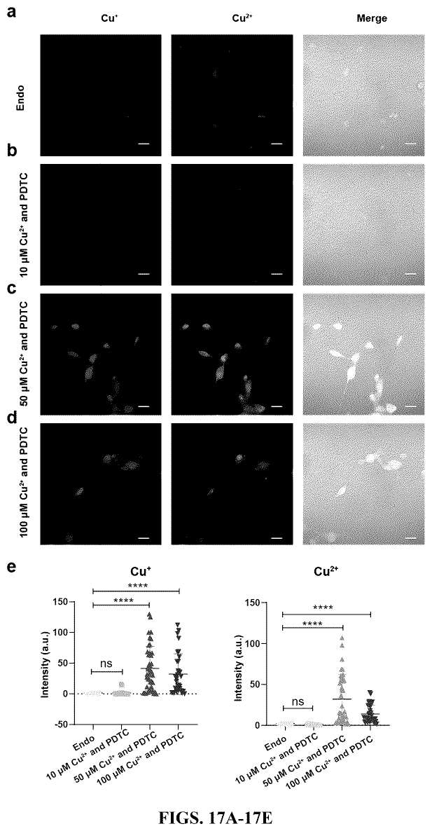

Fluorescence Imaging of Cu⁺ and Cu²⁺ in Live Cells Under Situations of Copper Elevation or Depletion: To confirm the ability of copper DNAzyme to image intracellular copper ions, the study artificially increased the copper levels using PDTC as copper supplement. As shown in FIGS. 17A-17E, comparing the images of cells before and after the exogenous copper source treatment, the fluorescence signal of both Cu⁺ and Cu²⁺ sensor was increased following the addition of PDTC in a dose-dependent manner. A significant increase in both Cu⁺ and Cu²⁺ sensor fluorescence signal was observed following treatment with 50 PM PDTC (FIG. 17C). The fluorescence signal was quantified in the graph of FIG. 17E and was more clearly observed from the separated channels of Cu⁺ and Cu²⁺ DNAzyme based sensors. Confirming that copper DNAzyme can be used to monitor small changes of copper ions level in live cells.

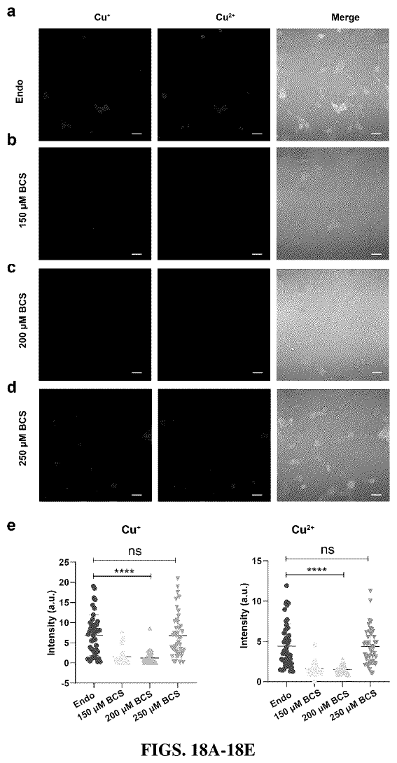

To confirm further the ability of copper DNAzyme to image intracellular copper ions, BCS was used for intracellular Cu deficiency to decrease intracellular copper levels. As shown in FIGS. 18A-18E, as compared to control cells (FIG. 18A) the pretreated the SH-SY5Y cells with BCS for overnight inducing copper depletion displayed a significant decrease in fluorescence intensity following with 150 μM or 200 μM BCS treatment (FIGS. 18B-18C and FIG. 18E), establishing that copper DNAzyme can monitor changes in labile copper levels in live cells. These data confirm that copper DNAzyme can detect changes of intracellular labile Cu⁺ and Cu²⁺ levels with pharmacological manipulation.

Fluorescence Imaging of Cu⁺ and Cu²⁺ Under Situations of Amyloid-β in SH-SY5Y cells: AD is the most common form of dementia, the amyloid cascade hypothesis implicates the pathological accumulation of Aβ and its aggregation from monomers into oligomers and fibrils, as a key event in the development of AD [37]. Many pieces of evidence have subsequently shown that the oligomeric form of Aβ is the most toxic species, resulting in a reformulated amyloid cascade hypothesis in which Aβ oligomers are proposed to be central to AD pathogenesis. Indeed, accumulated evidence shows that Aβ oligomers disrupt cellular function in cultured cells and animal models [38-41]. After verifying that copper DNAzyme can detect the changes of intracellular labile Cu⁺ and Cu²⁺ levels in neuronal SH-SY5Y cells, pretreated SH-SY5Y cells with Aβ40 monomers, oligomers and fibrils for 24 h to further explore the imaging of Aβ40-induced changes in intracellular Cu⁺ and Cu²⁺ homeostasis in live cells based on copper DNAzyme.

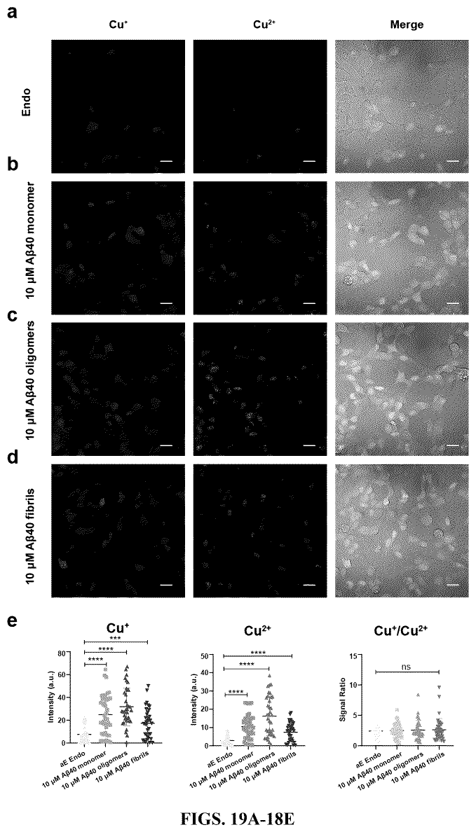

As shown in FIG. 19A, the endogenous fluorescence signal of Cu⁺ and Cu²⁺ DNAzyme is low. But after treatment with Aβ40, the fluorescence signals of both intracellular Cu⁺ and Cu²⁺ DNAzyme were enhanced (FIGS. 19B-19D). It should be noted that the fluorescence intensity significantly increased following 10 μM Aβ40 oligomers treatment (FIG. 19C and FIG. 19E), this may be because Aβ40 oligomers are the most toxic species to cells, generate more ROS, and cause the most serious disruption of intracellular copper homeostasis [42]. As controls, treatment of cells with inactive Cu⁺ and Cu²⁺ DNAzyme as well as the preincubating of the cells with Aβ40 did not lead to significant changes in fluorescence signal compared to cells treated with active copper DNAzyme. Therefore, Cu⁺ and Cu²⁺ DNAzyme could be used in the future to monitor changes in intracellular copper homeostasis associated with AD.

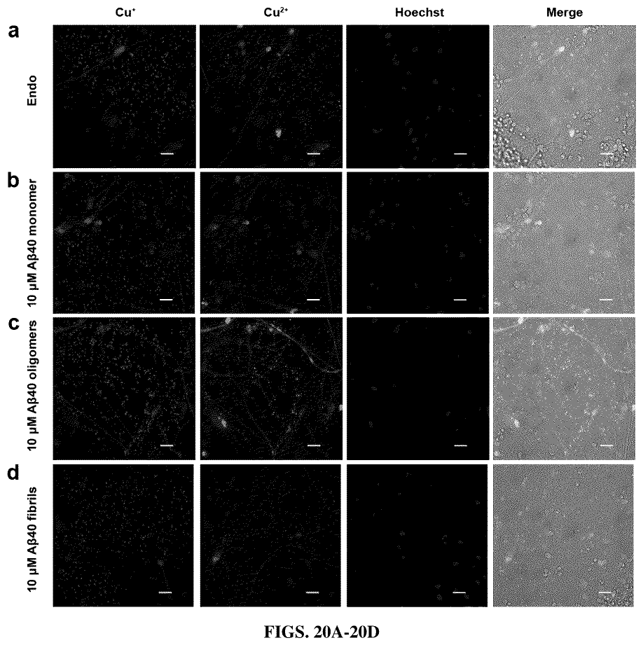

Imaging of Cu⁺ and Cu²⁺ with DNAzyme in Human-Induced Pluripotent Stem Cell (iPSC)-Derived Neurons: The study investigated the simultaneous Cu⁺/Cu²⁺ imaging in an iPSCs-based model, a powerful tool for investigating the pathogenesis of neuropsychological disorders. These results are shown in FIGS. 20A-20D.

In conclusion, taking advantage of the selectivity of DNAzyme, this study has developed a catalytic fluorescent sensor for labile copper pools (Cu⁺ and Cu²⁺) imaging in multiple cell lines including human cell lines (HeLa and SH-SY5Y), neuronal progenitor cells, astrocytes, and microglia. Such crosstalk could serve as a neuroprotective mechanism in neurodegenerative disease. These studies are consistent with direct detection of an increase/decrease of copper ions in live cells upon treatment with copper elevation (PDTC) or depletion (BCS). The DNAzyme based method can also be used to monitor changes in intracellular copper homeostasis associated with AD. This work provides a unique approach to DNAzyme-based imaging of Cu⁺ and Cu²⁺ that related to neurodegenerative diseases. It is anticipated that such DNAzyme based sensor will be of value in providing foundational information on the continuum between metal signaling and metal metabolism.

### Example 8: Further Optimization of Iron DNAzymes

RNA Cleavage Dependence of Fe²⁺ H5 and Fe³⁺ B12 DNAzymes: To investigate the Fe²⁺ H5 and Fe³⁺ B12 DNAzymes, various cleavage site modifications were incorporated into the substrate strands. The original Fe²⁺ H5 DNAzyme was combined with Fe²⁺ H5 substrate strands containing either a 2′-hydroxyl (2′OH), 2′-hydrogen (2′H), or 2′O-methyl (2′OMe) at the cleavage site. The original Fe²⁺ H5 DNAzyme cleaves the Fe²⁺ H5 substrate strand containing 2′OH at the cleavage site in the presence of Fe²⁺ under anaerobic conditions. However, the DNAzyme does not cleave the substrate strands containing 2′H or 2′OMe in the presence of Fe²⁺. Similarly, the original Fe³⁺ B12 DNAzyme was combined with Fe³⁺ B12 substrate strands containing either 2′OH, 2′H, or 2′OMe at the cleavage site. The original Fe³⁺ B12 DNAzyme cleaves the Fe³⁺ B12 substrate strand containing 2′OH at the cleavage site in the presence of Fe³⁺. The DNAzyme does not cleave the substrate strands containing 2′H or 2′OMe at the cleavage site in the presence of Fe³⁺. These findings highlight the critical role of the 2′OH group at the cleavage site in enabling DNAzyme activity for both Fe²⁺ H5 and Fe³⁺ B12 systems, as substitutions with 2′H or 2′OMe impede cleavage.

Truncations of Fe²⁺ H5 and Fe³⁺ B12 DNAzymes' Binding Arms: To further investigate the Fe²⁺ H5 and Fe³⁺ B12 DNAzymes, the DNAzymes were truncated at the 5′ and 3′ ends and tested using the FAM-labeled original substrate strand through activity assays assessed by PAGE gel. The activity of the Fe²⁺ H5 DNAzyme with the original substrate strand remains relatively unchanged when 1 or 2 nucleotides are truncated from either the 3′ or 5′ end. However, a significant decrease in relative intensity is observed when 3 or 4 nucleotides are truncated from the 3′ end. The activity of the Fe³⁺ B12 DNAzyme with the original substrate strand is maintained when 1 or 2 nucleotides are truncated from the 3′ end. A notable reduction in relative intensity occurs when 3, 4, or 5 nucleotides are truncated from the 5′ end. Additionally, truncation of 1, 2, or 3 nucleotides from the 3′ end also results in a decrease in relative intensity. These findings suggest that truncation of nucleotides in the binding arm leads to reduced cleavage of the substrate strand, potentially due to lower melting temperatures of the binding arms causing instability in the overall structure for both Fe²⁺ H5 and Fe³⁺ B12 DNAzymes.

pH Dependency of Fe²⁺ H5 and Fe³⁺ B12 DNAzymes: To further investigate the impact of reaction conditions on catalytic activity, the study evaluated the influence of pH system. For the Fe²⁺ H5 DNAzyme, the study assessed cleavage efficiency across a pH range of 6.0 to 7.5 in MOPS, MES, Bis-Tris, or Tris under anaerobic conditions with 100 μM FeCl₂. Highest activity was observed at pH 7.5, with significant reductions in activity at both lower pH levels, indicating a narrow optimal pH window for catalytic performance. This suggests that the 2′-hydroxyl group at the cleavage site plays a crucial role in facilitating catalysis.

Similarly, the pH dependency of Fe³⁺ B12 was tested across a range of pH 4.0 to 6.0 with 100 μM FeCl₃. The highest observed activity occurs at pH 5.5 in the presence of Bis-Tris and sodium acetate, with marked reductions in activity at pH values outside this optimal range. Moreover, no activity was detected in conditions utilizing either Bis-Tris or sodium acetate alone. The cleavage reaction and kinetics of Fe³⁺ B12 were further investigated under varying buffer conditions. Fluorescence intensity was monitored over time using a 5′ Alexa Fluor™ 647-labeled substrate and a 3′ Iowa Black™ RQ quencher-labeled DNAzyme at an enzyme-to-substrate ratio of 1:1. In the presence of 100 μM Fe³⁺, increasing concentrations of Bis-Tris, NaCl, or sodium acetate influenced the DNAzyme's cleavage activity, with higher buffer concentrations generally enhancing the reaction rate. Additionally, the analysis of relative intensities under buffer replacement conditions confirmed these findings. In the Bis-Tris replacement study, the Fe³⁺ B12 original FAM-substrate strand cleaved effectively only in the presence of 5 mM Bis-Tris, 40 mM sodium acetate, and 200 mM NaCl at pH 5.5. Conversely, the sodium acetate replacement study employed 40 mM of various ligands with 5 mM Bis-Tris and 200 mM NaCl, also demonstrating the relative intensities. The DNAzyme cleaved the substrate strand when sodium acetate was replaced with formate.

To further elucidate the dependency of Fe³⁺ B12 substrate cleavage on Bis-Tris and sodium acetate a variety of additional conditions were tested, including different concentrations of BSA, human serum, and cell lysate, both with and without Bis-Tris. The results revealed that Fe³⁺ B12 effectively cleaved the substrate strand only in the presence of both Fe³⁺ and Bis-Tris, underscoring the critical role of Bis-Tris in facilitating this catalytic activity. Overall, these findings suggest that Fe³⁺-dependent catalysis dedicatedly requires both Bis-Tris and sodium acetate in a slightly more acidic environment compared to the Fe²⁺ H5 system, likely due to differences in their binding interactions with metal ions and solubility.

Combination of Fe²⁺ H5 DNAzyme strand Improvements: Given the enhanced activity observed with several point mutations in the Fe²⁺ H5 DNAzyme strand, the study investigated whether combinations of these point mutations would further improve the DNAzyme's activity. The study designed 41 combinations of the 6-point mutations located at positions 39 (A39T and A39G), 40 (A40C), 51 (G51C), 52 (C52G), and 54 (T54C), along with the original Fe²⁺ H5. Each sequence was tested with the original FAM-labeled Fe²⁺ substrate strand in the presence of 100 μM FeCl₂ for 2 hours at room temperature. The relative intensities of the cleavage of the original FAM-labeled Fe²⁺ H5 DNAzyme strand when combined with different Fe²⁺ H5 point mutation combinations in the presence of FeCl₂ showed that some improved point mutations, when combined, decreased the relative activity, including Combos 1-8, 15-20, and 31. Conversely, other combinations increased the relative activity, including Combos 12-14, 29, 30, 36-39, and 41. These results suggest that the effects of nucleotide point mutations on Fe²⁺ H5 activity are complex and context dependent. Certain combinations of mutations enhance activity, while others diminish it, indicating interactions that are not merely additive. The specific positions of the mutations play a critical role in determining the overall activity, with some combinations inducing beneficial conformational changes and others disrupting key structural elements. These findings highlight the importance of understanding the contextual interplay of mutations for optimizing DNAzyme performance. The identified combinations that increase activity offer valuable insights for the future engineering of more efficient DNAzyme variants.

Combining Enhanced Fe²⁺ H5 and Fe³⁺ B12 DNAzymes Single Point Mutations: To assess the potential additive effects of combining enhanced Fe²⁺ H5 DNAzyme strands with substrate strands, the study evaluated the relative intensities of point mutations within these strands. Reactions were conducted in a buffer solution containing 25 mM Bis-Tris, 200 mM NaCl at pH 6.5, with 250 nM each of the mutated H5 DNAzyme strand and the FAM-labeled mutated H5 substrate strand, and 100 μM FeCl₂. The reactions were incubated for 30 minutes at room temperature under anaerobic conditions and were subsequently analyzed by denaturing PAGE gels. Several combinations of the improved Fe²⁺ H5 DNAzymes with the enhanced substrate strands, including Fe²⁺ H5 DNAzyme A39T with substrate A13C, Fe²⁺ H5 DNAzyme A39T with substrate A14T, Fe²⁺ H5 DNAzyme G51C with substrate A14T, Fe²⁺ H5 DNAzyme C52G with substrate A13C, and Fe²⁺ H5 DNAzyme C52G with substrate A14T, exhibited reduced relative activity in substrate strand cleavage compared to the original Fe²⁺ H5 DNAzyme-substrate pairs. In contrast, other combinations showed increased cleavage activity. Notable examples include Fe²⁺ H5 DNAzyme A39G with substrate T11C, Fe²⁺ H5 DNAzyme A39T with substrate T11C, Fe²⁺ H5 DNAzyme A40C with substrate T11C, and Fe²⁺ H5 DNAzyme T54C with substrate T11C. These findings indicate that specific mutations can modulate the catalytic efficiency of the Fe²⁺ H5 DNAzyme-substrate complex, either enhancing or diminishing its cleavage activity.

Furthermore, the selectivity of the improved Fe²⁺ H5 FAM-substrates with DNAzymes was investigated under physiological metal ion concentrations using an activity assay assessed through a PAGE gel. All competing metal ions exhibited negligible changes in their normalized fluorescence intensity compared to Fe²⁺ for all combinations of Fe²⁺ H5 FAM-substrates with DNAzymes, indicating excellent Fe²⁺ selectivity. The fidelity of selectivity was consistent across all combinations of the improved DNAzyme and substrate strands. Furthermore, point mutations that enhance activity did not compromise selectivity.

To evaluate the potential additive effects of combining the improved Fe³⁺ B12 DNAzyme with various substrate strands, the study analyzed the relative intensities of point mutations in these strands. Reactions were conducted in a solution containing 5 mM Bis-Tris, 40 mM sodium acetate, and 200 mM NaCl at pH 5.5, with concentrations of 250 nM for both the mutated B12 DNAzyme strand and the FAM-labeled mutated B12 substrate strand, along with 100 μM FeCl₃ for 30 minutes at room temperature and subsequently analyzed by PAGE gels Some combinations of the improved Fe³⁺ B12 DNAzymes in combination with the improved substrate strands, such as Fe³⁺ B12 Original DNAzyme with substrate C15A and Fe³⁺ B12 Original DNAzyme with substrate C15T, exhibited reduced relative activity in substrate strand cleavage compared to the original Fe³⁺ B12 DNAzyme-substrate combination. In contrast, other combinations, including Fe³⁺ B12 DNAzyme C20T with the original substrate, Fe³⁺ B12 DNAzyme C20T with substrate A14G, and Fe³⁺ B12 DNAzyme C20T with substrate C15A, showed increased cleavage activity. These findings indicate that specific mutations can either enhance or diminish the catalytic efficiency of the Fe³⁺ B12 DNAzyme-substrate complex.

In addition, the selectivity of the enhanced Fe³⁺ B12 FAM-substrates with DNAzymes was evaluated under physiological metal ion concentrations using an activity assay, which was analyzed through PAGE gels. The normalized fluorescence intensity of all competing metal ions showed negligible variations compared to Fe³⁺ for all combinations of Fe³⁺ B12 FAM-substrates with DNAzymes. This result indicates that the Fe³⁺ B12 FAM-substrate with DNAzyme exhibits superior selectivity for Fe³⁺. The selectivity was consistent across all combinations of the enhanced DNAzyme and substrate strands. Additionally, point mutations that enhance activity did not compromise the selectivity.

Kinetic Assessment of Enhanced Fe²⁺ H5 and Fe³⁺ B12 DNAzymes Single Point Mutations: To better understand how the point mutations improving turn-on fluorescence signaling in the Fe²⁺ H5 and Fe³⁺ B12 DNAzymes affected kinetic behavior, kinetic assays were performed at two enzyme-to-substrate (E:S) ratios: 10:1 and 1:1. Notably, some Fe²⁺ H5 DNAzyme and substrate point combinations, such as Fe²⁺ H5 DNAzyme A39G and substrate A13C, exhibited 1.5 fold increased fluorescence compared to the original Fe²⁺ H5 DNAzyme and substrate. The original Fe²⁺ H5 DNAzyme and substrate combination at a 10:1 ratio had a higher catalytic efficiency (0.0001159 min⁻¹·μM⁻¹) compared to other improved combinations, which had slightly lower catalytic efficiency values. The binding affinity decreased as FeCl₂ concentration increased for all improved Fe²⁺ H5 DNAzymes and substrates at a 10:1 E:S ratio. The Michaelis constant (Kₘ) for the original Fe²⁺ H5 DNAzyme and substrate was slightly higher or slightly lower than the improved combinations at a 10:1 E:S ratio.

Similarly, kinetic assays were performed at a E:S 1:1 ratio to evaluate how the mutations for the Fe²⁺ H5 DNAzymes affected their kinetics over time. As observed with the 10:1 assay, some Fe²⁺ H5 DNAzyme and substrate point combinations, such as Fe²⁺ H5 DNAzyme A39G and original substrate, exhibited increased fluorescence compared to the original Fe²⁺ H5 DNAzyme and substrate. The original Fe²⁺ H5 DNAzyme and substrate combination at a 1:1 ratio had a catalytic efficiency (0.0001304 min⁻¹·μM⁻¹), which was slightly higher or lower than the improved combinations. The Michaelis constant (Kₘ) for the original Fe²⁺ H5 DNAzyme and substrate was slightly lower than the improved combinations at a 1:1 E:S ratio.

Notably, some Fe³⁺ B12 DNAzyme and substrate point combinations, such as Fe³⁺ B12 DNAzyme C20T and substrate C15A, exhibited a 4.9 fold increased fluorescence compared to the original Fe³⁺ B12 DNAzyme and substrate. There was a linear signal increase in response to rising FeCl₃ concentrations for all improved Fe³⁺ B12 DNAzymes and substrates at a 10:1 E:S ratio. The original Fe³⁺ B12 DNAzyme and substrate combination at a 10:1 ratio had a higher catalytic efficiency (0.01258 min⁻¹·μM⁻¹) compared to other improved combinations, which had lower catalytic efficiency values. The binding affinity decreased as FeCl₃ concentration increased for all improved Fe³⁺ B12 DNAzymes and substrates at a 10:1 E:S ratio. The Michaelis constant (Kₘ) for the original Fe³⁺ B12 DNAzyme and substrate was lower than the improved combinations at a 10:1 E:S ratio.

Kinetic assays were also performed at a E:S 1:1 ratio to assess the effect of mutations on Fe³⁺ B12 DNAzymes over time. Like the E:S 10:1 assay, some Fe³⁺ B12 DNAzyme and substrate point combinations, such as Fe³⁺ B12 Original DNAzyme and substrate A14G, exhibited increased fluorescence compared to the original Fe³⁺ B12 DNAzyme and substrate. The original Fe³⁺ B12 DNAzyme and substrate combination at a 1:1 ratio had a catalytic efficiency (0.01324 min⁻¹·μM⁻¹), which was slightly higher or lower than the improved combinations. The Michaelis constant (Kₘ) for the original Fe³⁺ B12 DNAzyme and substrate was lower than the improved combinations at a 1:1 E:S ratio.

Sequences used in this study are provided in TABLE 25.

TABLE 25
List of all sequences used in this study. The IDT codes are presented for fluorophore,
quenchers, and modified nucleobases.

| | | |
|---|---|---|
| | | SEQ ID |
| NAME | Sequence (5′ to 3′) | NO |

Fe²⁺ H5 Substrate Cleavage Site Study

| | | |
|---|---|---|
| Fe²⁺ H5 Original | CGGACCCGTATCAATCTCACGTATrAGGATATCCA | 26 |
| Substrate (2′OH) | | |
| Fe²⁺ H5 Original | CGGACCCGTATCAATCTCACGTATAGGATATCCA | 74 |
| Substrate (2′H) | | |
| Fe²⁺ H5 Original | CGGACCCGTATCAATCTCACGTATmAGGATATCCA | 75 |
| Substrate | | |
| (2′OMe) | | |

Fe³⁺ B12 Substrate Cleavage Site Study

| | | |
|---|---|---|
| Fe³⁺ B12 Original | CTCTATTArGGGAGACTCGCATGCCGC | 23 |
| Substrate (2′OH) | | |
| Fe³⁺ B12 Original | CTCTATTAGGGAGACTCGCATGCCGC | 76 |
| Substrate (2′H) | | |
| Fe³⁺ B12 Original | CTCTATTAmGGGAGACTCGCATGCCGC | 77 |
| Substrate | | |
| (2′OMe) | | |

Fe²⁺ H5 Substrate Point Mutations

| | | |
|---|---|---|
| Fe²⁺ H5 Original | /56- | 78 |
| Substrate FAM | FAM/CGGACCCGTATCAATCTCACGTATrAGGATATCCA | |
| Fe²⁺ H5 Substrate | /56- | 79 |
| T11A FAM | FAM/CGGACCCGTAACAATCTCACGTATrAGGATATCCA | |
| Fe²⁺ H5 Substrate | /56- | 80 |
| T11C FAM | FAM/CGGACCCGTACCAATCTCACGTATrAGGATATCCA | |
| Fe²⁺ H5 Substrate | /56- | 81 |
| T11G FAM | FAM/CGGACCCGTAGCAATCTCACGTATrAGGATATCCA | |
| Fe²⁺ H5 Substrate | /56- | 82 |
| C12A FAM | FAM/CGGACCCGTATAAATCTCACGTATrAGGATATCCA | |
| Fe²⁺ H5 Substrate | /56- | 83 |
| C12T FAM | FAM/CGGACCCGTATTAATCTCACGTATrAGGATATCCA | |
| Fe²⁺ H5 Substrate | /56- | 84 |
| C12G FAM | FAM/CGGACCCGTATGAATCTCACGTATrAGGATATCCA | |
| Fe²⁺ H5 Substrate | /56- | 85 |
| A13T FAM | FAM/CGGACCCGTATCTATCTCACGTATrAGGATATCCA | |
| Fe²⁺ H5 Substrate | /56- | 86 |
| A13C FAM | FAM/CGGACCCGTATCCATCTCACGTATrAGGATATCCA | |
| Fe²⁺ H5 Substrate | /56- | 87 |
| A13G FAM | FAM/CGGACCCGTATCGATCTCACGTATrAGGATATCCA | |
| Fe²⁺ H5 Substrate | /56- | 88 |
| A14T FAM | FAM/CGGACCCGTATCATTCTCACGTATrAGGATATCCA | |
| Fe²⁺ H5 Substrate | /56- | 89 |
| A14C FAM | FAM/CGGACCCGTATCACTCTCACGTATrAGGATATCCA | |
| Fe²⁺ H5 Substrate | /56- | 90 |
| A14G FAM | FAM/CGGACCCGTATCAGTCTCACGTATrAGGATATCCA | |
| Fe²⁺ H5 Substrate | /56- | 91 |
| T15A FAM | FAM/CGGACCCGTATCAAACTCACGTATrAGGATATCCA | |
| Fe²⁺ H5 Substrate | /56- | 92 |
| T15C FAM | FAM/CGGACCCGTATCAACCTCACGTATrAGGATATCCA | |
| Fe²⁺ H5 Substrate | /56- | 93 |
| T15G FAM | FAM/CGGACCCGTATCAAGCTCACGTATrAGGATATCCA | |
| Fe²⁺ H5 Substrate | /56- | 94 |
| C16A FAM | FAM/CGGACCCGTATCAATATCACGTATrAGGATATCCA | |
| Fe²⁺ H5 Substrate | /56- | 95 |
| C16T FAM | FAM/CGGACCCGTATCAATTTCACGTATrAGGATATCCA | |
| Fe²⁺ H5 Substrate | /56- | 96 |
| C16G FAM | FAM/CGGACCCGTATCAATGTCACGTATrAGGATATCCA | |
| Fe²⁺ H5 Substrate | /56- | 97 |
| T22A FAM | FAM/CGGACCCGTATCAATCTCACGAATrAGGATATCCA | |
| Fe²⁺ H5 Substrate | /56- | 98 |
| T22C FAM | FAM/CGGACCCGTATCAATCTCACGCATrAGGATATCCA | |
| Fe²⁺ H5 Substrate | /56- | 99 |
| T22G FAM | FAM/CGGACCCGTATCAATCTCACGGATrAGGATATCCA | |
| Fe²⁺ H5 Substrate | /56- | 100 |
| A23T FAM | FAM/CGGACCCGTATCAATCTCACGTTTrAGGATATCCA | |
| Fe²⁺ H5 Substrate | /56- | 101 |
| A23C FAM | FAM/CGGACCCGTATCAATCTCACGTCTrAGGATATCCA | |
| Fe²⁺ H5 Substrate | /56- | 102 |
| A23G FAM | FAM/CGGACCCGTATCAATCTCACGTGTrAGGATATCCA | |
| Fe²⁺ H5 Substrate | /56- | 103 |
| T24A FAM | FAM/CGGACCCGTATCAATCTCACGTAArAGGATATCCA | |
| Fe²⁺ H5 Substrate | /56- | 104 |
| T24C FAM | FAM/CGGACCCGTATCAATCTCACGTACrAGGATATCCA | |
| Fe²⁺ H5 Substrate | /56- | 105 |
| T24G FAM | FAM/CGGACCCGTATCAATCTCACGTAGrAGGATATCCA | |
| Fe²⁺ H5 Substrate | /56- | 106 |
| rA25rU FAM | FAM/CGGACCCGTATCAATCTCACGTATrUGGATATCCA | |
| Fe²⁺ H5 Substrate | /56- | 107 |
| rA25rC FAM | FAM/CGGACCCGTATCAATCTCACGTATrCGGATATCCA | |
| Fe²⁺ H5 Substrate | /56- | 108 |
| rA25rG FAM | FAM/CGGACCCGTATCAATCTCACGTATrGGGATATCCA | |
| Fe²⁺ H5 Substrate | /56- | 109 |
| G26A FAM | FAM/CGGACCCGTATCAATCTCACGTATrAAGATATCCA | |
| Fe²⁺ H5 Substrate | /56- | 110 |
| G26T FAM | FAM/CGGACCCGTATCAATCTCACGTATrATGATATCCA | |
| Fe²⁺ H5 Substrate | /56- | 111 |
| G26C FAM | FAM/CGGACCCGTATCAATCTCACGTATrACGATATCCA | |

Fe²⁺ H5 DNAzyme Point Mutations

| | | |
|---|---|---|
| Fe²⁺ H5 Original | TGGATATCTCCTAGCCAGACTGTTATGTGTGATACGGC | 24 |
| DNAzyme | AAACTTCGTGATGCCTCTACGGGTCCG | |
| Fe²⁺ H5 | TGGATATCACCTAGCCAGACTGTTATGTGTGATACGGC | 112 |
| DNAzyme T9A | AAACTTCGTGATGCCTCTACGGGTCCG | |
| Fe²⁺ H5 | TGGATATCCCCTAGCCAGACTGTTATGTGTGATACGGC | 113 |
| DNAzyme T9C | AAACTTCGTGATGCCTCTACGGGTCCG | |
| Fe²⁺ H5 | TGGATATCGCCTAGCCAGACTGTTATGTGTGATACGGC | 114 |
| DNAzyme T9G | AAACTTCGTGATGCCTCTACGGGTCCG | |
| Fe²⁺ H5 | TGGATATCTGCTAGCCAGACTGTTATGTGTGATACGGC | 115 |
| DNAzyme C10G | AAACTTCGTGATGCCTCTACGGGTCCG | |
| Fe²⁺ H5 | TGGATATCTTCTAGCCAGACTGTTATGTGTGATACGGC | 116 |
| DNAzyme C10T | AAACTTCGTGATGCCTCTACGGGTCCG | |
| Fe²⁺ H5 | TGGATATCTACTAGCCAGACTGTTATGTGTGATACGGC | 117 |
| DNAzyme C10A | AAACTTCGTGATGCCTCTACGGGTCCG | |
| Fe²⁺ H5 | TGGATATCTCCTGGCCAGACTGTTATGTGTGATACGGC | 25 |
| DNAzyme A13G | AAACTTCGTGATGCCTCTACGGGTCCG | |
| Fe²⁺ H5 | TGGATATCTCCTTGCCAGACTGTTATGTGTGATACGGC | 118 |
| DNAzyme A13T | AAACTTCGTGATGCCTCTACGGGTCCG | |
| Fe²⁺ H5 | TGGATATCTCCTCGCCAGACTGTTATGTGTGATACGGC | 119 |
| DNAzyme A13C | AAACTTCGTGATGCCTCTACGGGTCCG | |
| Fe²⁺ H5 | TGGATATCTCCTAACCAGACTGTTATGTGTGATACGGC | 120 |
| DNAzyme G14A | AAACTTCGTGATGCCTCTACGGGTCCG | |
| Fe²⁺ H5 | TGGATATCTCCTACCCAGACTGTTATGTGTGATACGGC | 121 |
| DNAzyme G14C | AAACTTCGTGATGCCTCTACGGGTCCG | |
| Fe²⁺ H5 | TGGATATCTCCTATCCAGACTGTTATGTGTGATACGGC | 122 |
| DNAzyme G14T | AAACTTCGTGATGCCTCTACGGGTCCG | |
| Fe²⁺ H5 | TGGATATCTCCTAGGCAGACTGTTATGTGTGATACGGC | 123 |
| DNAzyme C15G | AAACTTCGTGATGCCTCTACGGGTCCG | |
| Fe²⁺ H5 | TGGATATCTCCTAGTCAGACTGTTATGTGTGATACGGC | 124 |
| DNAzyme C15T | AAACTTCGTGATGCCTCTACGGGTCCG | |
| Fe²⁺ H5 | TGGATATCTCCTAGACAGACTGTTATGTGTGATACGGC | 125 |
| DNAzyme C15A | AAACTTCGTGATGCCTCTACGGGTCCG | |
| Fe²⁺ H5 | TGGATATCTCCTAGCGAGACTGTTATGTGTGATACGGC | 126 |
| DNAzyme C16G | AAACTTCGTGATGCCTCTACGGGTCCG | |
| Fe²⁺ H5 | TGGATATCTCCTAGCTAGACTGTTATGTGTGATACGGC | 127 |
| DNAzyme C16T | AAACTTCGTGATGCCTCTACGGGTCCG | |
| Fe²⁺ H5 | TGGATATCTCCTAGCAAGACTGTTATGTGTGATACGGC | 128 |
| DNAzyme C16A | AAACTTCGTGATGCCTCTACGGGTCCG | |
| Fe²⁺ H5 | TGGATATCTCCTAGCCAGGCTGTTATGTGTGATACGGC | 129 |
| DNAzyme A19G | AAACTTCGTGATGCCTCTACGGGTCCG | |
| Fe²⁺ H5 | TGGATATCTCCTAGCCAGTCTGTTATGTGTGATACGGC | 130 |
| DNAzyme A19T | AAACTTCGTGATGCCTCTACGGGTCCG | |
| Fe²⁺ H5 | TGGATATCTCCTAGCCAGCCTGTTATGTGTGATACGGC | 131 |
| DNAzyme A19C | AAACTTCGTGATGCCTCTACGGGTCCG | |
| Fe²⁺ H5 | TGGATATCTCCTAGCCAGAGTGTTATGTGTGATACGGC | 132 |
| DNAzyme C20G | AAACTTCGTGATGCCTCTACGGGTCCG | |
| Fe²⁺ H5 | TGGATATCTCCTAGCCAGATTGTTATGTGTGATACGGC | 133 |
| DNAzyme C20T | AAACTTCGTGATGCCTCTACGGGTCCG | |
| Fe²⁺ H5 | TGGATATCTCCTAGCCAGAATGTTATGTGTGATACGGC | 134 |
| DNAzyme C20A | AAACTTCGTGATGCCTCTACGGGTCCG | |
| Fe²⁺ H5 | TGGATATCTCCTAGCCAGACAGTTATGTGTGATACGGC | 135 |
| DNAzyme T21A | AAACTTCGTGATGCCTCTACGGGTCCG | |
| Fe²⁺ H5 | TGGATATCTCCTAGCCAGACCGTTATGTGTGATACGGC | 136 |
| DNAzyme T21C | AAACTTCGTGATGCCTCTACGGGTCCG | |
| Fe²⁺ H5 | TGGATATCTCCTAGCCAGACGGTTATGTGTGATACGGC | 137 |
| DNAzyme T21G | AAACTTCGTGATGCCTCTACGGGTCCG | |
| Fe²⁺ H5 | TGGATATCTCCTAGCCAGACTGTTGTGTGTGATACGGC | 138 |
| DNAzyme A25G | AAACTTCGTGATGCCTCTACGGGTCCG | |
| Fe²⁺ H5 | TGGATATCTCCTAGCCAGACTGTTTTGTGTGATACGGC | 139 |
| DNAzyme A25T | AAACTTCGTGATGCCTCTACGGGTCCG | |
| Fe²⁺ H5 | TGGATATCTCCTAGCCAGACTGTTCTGTGTGATACGGC | 140 |
| DNAzyme A25C | AAACTTCGTGATGCCTCTACGGGTCCG | |
| Fe²⁺ H5 | TGGATATCTCCTAGCCAGACTGTTAAGTGTGATACGGC | 141 |
| DNAzyme T26A | AAACTTCGTGATGCCTCTACGGGTCCG | |
| Fe²⁺ H5 | TGGATATCTCCTAGCCAGACTGTTACGTGTGATACGGC | 142 |
| DNAzyme T26C | AAACTTCGTGATGCCTCTACGGGTCCG | |
| Fe²⁺ H5 | TGGATATCTCCTAGCCAGACTGTTAGGTGTGATACGGC | 143 |
| DNAzyme T26G | AAACTTCGTGATGCCTCTACGGGTCCG | |
| Fe²⁺ H5 | TGGATATCTCCTAGCCAGACTGTTATGTATGATACGGC | 144 |
| DNAzyme G29A | AAACTTCGTGATGCCTCTACGGGTCCG | |
| Fe²⁺ H5 | TGGATATCTCCTAGCCAGACTGTTATGTCTGATACGGC | 145 |
| DNAzyme G29C | AAACTTCGTGATGCCTCTACGGGTCCG | |
| Fe²⁺ H5 | TGGATATCTCCTAGCCAGACTGTTATGTTTGATACGGC | 146 |
| DNAzyme G29T | AAACTTCGTGATGCCTCTACGGGTCCG | |
| Fe²⁺ H5 | TGGATATCTCCTAGCCAGACTGTTATGTGAGATACGGC | 147 |
| DNAzyme T30A | AAACTTCGTGATGCCTCTACGGGTCCG | |
| Fe²⁺ H5 | TGGATATCTCCTAGCCAGACTGTTATGTGCGATACGGC | 148 |
| DNAzyme T30C | AAACTTCGTGATGCCTCTACGGGTCCG | |
| Fe²⁺ H5 | TGGATATCTCCTAGCCAGACTGTTATGTGGGATACGGC | 149 |
| DNAzyme T30G | AAACTTCGTGATGCCTCTACGGGTCCG | |
| Fe²⁺ H5 | TGGATATCTCCTAGCCAGACTGTTATGTGTAATACGGC | 150 |
| DNAzyme G31A | AAACTTCGTGATGCCTCTACGGGTCCG | |
| Fe²⁺ H5 | TGGATATCTCCTAGCCAGACTGTTATGTGTCATACGGC | 151 |
| DNAzyme G31C | AAACTTCGTGATGCCTCTACGGGTCCG | |
| Fe²⁺ H5 | TGGATATCTCCTAGCCAGACTGTTATGTGTTATACGGC | 152 |
| DNAzyme G31T | AAACTTCGTGATGCCTCTACGGGTCCG | |
| Fe²⁺ H5 | TGGATATCTCCTAGCCAGACTGTTATGTGTGGTACGGC | 153 |
| DNAzyme A32G | AAACTTCGTGATGCCTCTACGGGTCCG | |
| Fe²⁺ H5 | TGGATATCTCCTAGCCAGACTGTTATGTGTGTTACGGC | 154 |
| DNAzyme A32T | AAACTTCGTGATGCCTCTACGGGTCCG | |
| Fe²⁺ H5 | TGGATATCTCCTAGCCAGACTGTTATGTGTGCTACGGC | 155 |
| DNAzyme A32C | AAACTTCGTGATGCCTCTACGGGTCCG | |
| Fe²⁺ H5 | TGGATATCTCCTAGCCAGACTGTTATGTGTGAAACGGC | 156 |
| DNAzyme T33A | AAACTTCGTGATGCCTCTACGGGTCCG | |
| Fe²⁺ H5 | TGGATATCTCCTAGCCAGACTGTTATGTGTGACACGGC | 157 |
| DNAzyme T33C | AAACTTCGTGATGCCTCTACGGGTCCG | |
| Fe²⁺ H5 | TGGATATCTCCTAGCCAGACTGTTATGTGTGAGACGGC | 158 |
| DNAzyme T33G | AAACTTCGTGATGCCTCTACGGGTCCG | |
| Fe²⁺ H5 | TGGATATCTCCTAGCCAGACTGTTATGTGTGATACAGC | 159 |
| DNAzyme G36A | AAACTTCGTGATGCCTCTACGGGTCCG | |
| Fe²⁺ H5 | TGGATATCTCCTAGCCAGACTGTTATGTGTGATACCGC | 160 |
| DNAzyme G36C | AAACTTCGTGATGCCTCTACGGGTCCG | |
| Fe²⁺ H5 | TGGATATCTCCTAGCCAGACTGTTATGTGTGATACTGC | 161 |
| DNAzyme G36T | AAACTTCGTGATGCCTCTACGGGTCCG | |
| Fe²⁺ H5 | TGGATATCTCCTAGCCAGACTGTTATGTGTGATACGAC | 162 |
| DNAzyme G37A | AAACTTCGTGATGCCTCTACGGGTCCG | |
| Fe²⁺ H5 | TGGATATCTCCTAGCCAGACTGTTATGTGTGATACGCC | 163 |
| DNAzyme G37C | AAACTTCGTGATGCCTCTACGGGTCCG | |
| Fe²⁺ H5 | TGGATATCTCCTAGCCAGACTGTTATGTGTGATACGTC | 164 |
| DNAzyme G37T | AAACTTCGTGATGCCTCTACGGGTCCG | |
| Fe²⁺ H5 | TGGATATCTCCTAGCCAGACTGTTATGTGTGATACGGG | 165 |
| DNAzyme C38G | AAACTTCGTGATGCCTCTACGGGTCCG | |
| Fe²⁺ H5 | TGGATATCTCCTAGCCAGACTGTTATGTGTGATACGGT | 166 |
| DNAzyme C38T | AAACTTCGTGATGCCTCTACGGGTCCG | |
| Fe²⁺ H5 | TGGATATCTCCTAGCCAGACTGTTATGTGTGATACGGA | 167 |
| DNAzyme C38A | AAACTTCGTGATGCCTCTACGGGTCCG | |
| Fe²⁺ H5 | TGGATATCTCCTAGCCAGACTGTTATGTGTGATACGGC | 36 |
| DNAzyme A39G | GAACTTCGTGATGCCTCTACGGGTCCG | |
| Fe²⁺ H5 | TGGATATCTCCTAGCCAGACTGTTATGTGTGATACGGC | 35 |
| DNAzyme A39T | TAACTTCGTGATGCCTCTACGGGTCCG | |
| Fe²⁺ H5 | TGGATATCTCCTAGCCAGACTGTTATGTGTGATACGGC | 168 |
| DNAzyme A39C | CAACTTCGTGATGCCTCTACGGGTCCG | |
| Fe²⁺ H5 | TGGATATCTCCTAGCCAGACTGTTATGTGTGATACGGC | 169 |
| DNAzyme A40G | AGACTTCGTGATGCCTCTACGGGTCCG | |
| Fe²⁺ H5 | TGGATATCTCCTAGCCAGACTGTTATGTGTGATACGGC | 170 |
| DNAzyme A40T | ATACTTCGTGATGCCTCTACGGGTCCG | |
| Fe²⁺ H5 | TGGATATCTCCTAGCCAGACTGTTATGTGTGATACGGC | 37 |
| DNAzyme A40C | ACACTTCGTGATGCCTCTACGGGTCCG | |
| Fe²⁺ H5 | TGGATATCTCCTAGCCAGACTGTTATGTGTGATACGGC | 171 |
| DNAzyme T43A | AAACATCGTGATGCCTCTACGGGTCCG | |
| Fe²⁺ H5 | TGGATATCTCCTAGCCAGACTGTTATGTGTGATACGGC | 172 |
| DNAzyme T43C | AAACCTCGTGATGCCTCTACGGGTCCG | |
| Fe²⁺ H5 | TGGATATCTCCTAGCCAGACTGTTATGTGTGATACGGC | 173 |
| DNAzyme T43G | AAACGTCGTGATGCCTCTACGGGTCCG | |
| Fe²⁺ H5 | TGGATATCTCCTAGCCAGACTGTTATGTGTGATACGGC | 174 |
| DNAzyme T44A | AAACTACGTGATGCCTCTACGGGTCCG | |
| Fe²⁺ H5 | TGGATATCTCCTAGCCAGACTGTTATGTGTGATACGGC | 175 |
| DNAzyme T44C | AAACTCCGTGATGCCTCTACGGGTCCG | |
| Fe²⁺ H5 | TGGATATCTCCTAGCCAGACTGTTATGTGTGATACGGC | 176 |
| DNAzyme T44G | AAACTGCGTGATGCCTCTACGGGTCCG | |
| Fe²⁺ H5 | TGGATATCTCCTAGCCAGACTGTTATGTGTGATACGGC | 177 |
| DNAzyme T50A | AAACTTCGTGAAGCCTCTACGGGTCCG | |
| Fe²⁺ H5 | TGGATATCTCCTAGCCAGACTGTTATGTGTGATACGGC | 178 |
| DNAzyme T50C | AAACTTCGTGACGCCTCTACGGGTCCG | |
| Fe²⁺ H5 | TGGATATCTCCTAGCCAGACTGTTATGTGTGATACGGC | 179 |
| DNAzyme T50G | AAACTTCGTGAGGCCTCTACGGGTCCG | |
| Fe²⁺ H5 | TGGATATCTCCTAGCCAGACTGTTATGTGTGATACGGC | 180 |
| DNAzyme G51A | AAACTTCGTGATACCTCTACGGGTCCG | |
| Fe²⁺ H5 | TGGATATCTCCTAGCCAGACTGTTATGTGTGATACGGC | 38 |
| DNAzyme G51C | AAACTTCGTGATCCCTCTACGGGTCCG | |
| Fe²⁺ H5 | TGGATATCTCCTAGCCAGACTGTTATGTGTGATACGGC | 181 |
| DNAzyme G51T | AAACTTCGTGATTCCTCTACGGGTCCG | |
| Fe²⁺ H5 | TGGATATCTCCTAGCCAGACTGTTATGTGTGATACGGC | 39 |
| DNAzyme C52G | AAACTTCGTGATGGCTCTACGGGTCCG | |
| Fe²⁺ H5 | TGGATATCTCCTAGCCAGACTGTTATGTGTGATACGGC | 182 |
| DNAzyme C52T | AAACTTCGTGATGTCTCTACGGGTCCG | |
| Fe²⁺ H5 | TGGATATCTCCTAGCCAGACTGTTATGTGTGATACGGC | 183 |
| DNAzyme C52A | AAACTTCGTGATGACTCTACGGGTCCG | |
| Fe²⁺ H5 | TGGATATCTCCTAGCCAGACTGTTATGTGTGATACGGC | 184 |
| DNAzyme C53G | AAACTTCGTGATGCGTCTACGGGTCCG | |
| Fe²⁺ H5 | TGGATATCTCCTAGCCAGACTGTTATGTGTGATACGGC | 185 |
| DNAzyme C53T | AAACTTCGTGATGCTTCTACGGGTCCG | |
| Fe²⁺ H5 | TGGATATCTCCTAGCCAGACTGTTATGTGTGATACGGC | 186 |
| DNAzyme C53A | AAACTTCGTGATGCATCTACGGGTCCG | |
| Fe²⁺ H5 | TGGATATCTCCTAGCCAGACTGTTATGTGTGATACGGC | 187 |
| DNAzyme T54G | AAACTTCGTGATGCCGCTACGGGTCCG | |
| Fe²⁺ H5 | TGGATATCTCCTAGCCAGACTGTTATGTGTGATACGGC | 40 |
| DNAzyme T54C | AAACTTCGTGATGCCCCTACGGGTCCG | |
| Fe²⁺ H5 | TGGATATCTCCTAGCCAGACTGTTATGTGTGATACGGC | 188 |
| DNAzyme T54A | AAACTTCGTGATGCCACTACGGGTCCG | |
| Fe²⁺ H5 | TGGATATCTCCTAGCCAGACTGTTATGTGTGATACGGC | 189 |
| DNAzyme C55G | AAACTTCGTGATGCCTGTACGGGTCCG | |
| Fe²⁺ H5 | TGGATATCTCCTAGCCAGACTGTTATGTGTGATACGGC | 190 |
| DNAzyme C55T | AAACTTCGTGATGCCTTTACGGGTCCG | |
| Fe²⁺ H5 | TGGATATCTCCTAGCCAGACTGTTATGTGTGATACGGC | 191 |
| DNAzyme C55A | AAACTTCGTGATGCCTATACGGGTCCG | |

Fe³⁺ B12 Substrate Point Mutations

| | | |
|---|---|---|
| Fe³⁺ B12 Original | /56-FAM/CTCTATTArGGGAGACTCGCATGCCGC | 192 |
| Substrate FAM | | |
| Fe³⁺ B12 | /56-FAM/CTCTATTArGGAAGACTCGCATGCCGC | 193 |
| Substrate G11A | | |
| FAM | | |
| Fe³⁺ B12 | /56-FAM/CTCTATTArGGTAGACTCGCATGCCGC | 194 |
| Substrate G11T | | |
| FAM | | |
| Fe³⁺ B12 | /56-FAM/CTCTATTArGGCAGACTCGCATGCCGC | 195 |
| Substrate G11C | | |
| FAM | | |
| Fe³⁺ B12 | /56-FAM/CTCTATTArGGGTGACTCGCATGCCGC | 196 |
| Substrate A12T | | |
| FAM | | |
| Fe³⁺ B12 | /56-FAM/CTCTATTArGGGCGACTCGCATGCCGC | 197 |
| Substrate A12C | | |
| FAM | | |
| Fe³⁺ B12 | /56-FAM/CTCTATTArGGGGGACTCGCATGCCGC | 198 |
| Substrate A12G | | |
| FAM | | |
| Fe³⁺ B12 | /56-FAM/CTCTATTArGGGAAACTCGCATGCCGC | 199 |
| Substrate G13A | | |
| FAM | | |
| Fe³⁺ B12 | /56-FAM/CTCTATTArGGGATACTCGCATGCCGC | 200 |
| Substrate G13T | | |
| FAM | | |
| Fe³⁺ B12 | /56-FAM/CTCTATTArGGGACACTCGCATGCCGC | 201 |
| Substrate G13C | | |
| FAM | | |
| Fe³⁺ B12 | /56-FAM/CTCTATTArGGGAGTCTCGCATGCCGC | 202 |
| Substrate A14T | | |
| FAM | | |
| Fe³⁺ B12 | /56-FAM/CTCTATTArGGGAGCCTCGCATGCCGC | 203 |
| Substrate A14C | | |
| FAM | | |
| Fe³⁺ B12 | /56-FAM/CTCTATTArGGGAGGCTCGCATGCCGC | 204 |
| Substrate A14G | | |
| FAM | | |
| Fe³⁺ B12 | /56-FAM/CTCTATTArGGGAGAATCGCATGCCGC | 205 |
| Substrate C15A | | |
| FAM | | |
| Fe³⁺ B12 | /56-FAM/CTCTATTArGGGAGATTCGCATGCCGC | 206 |
| Substrate C15T | | |
| FAM | | |
| Fe³⁺ B12 | /56-FAM/CTCTATTArGGGAGAGTCGCATGCCGC | 207 |
| Substrate C15G | | |
| FAM | | |
| Fe³⁺ B12 | /56-FAM/CTCTATTArGGGAGACACGCATGCCGC | 208 |
| Substrate T16A | | |
| FAM | | |
| Fe³⁺ B12 | /56-FAM/CTCTATTArGGGAGACCCGCATGCCGC | 209 |
| Substrate T16C | | |
| FAM | | |
| Fe³⁺ B12 | /56-FAM/CTCTATTArGGGAGACGCGCATGCCGC | 210 |
| Substrate T16G | | |
| FAM | | |

Fe³⁺ B12 DNAzyme Point Mutations

| | | |
|---|---|---|
| Fe³⁺ B12 Original | GCGGCATGCGCGTTTGCGGCACCTAAACGCTCCTAATA | 21 |
| DNAzyme | GAG | |
| Fe³⁺ B12 | GCGGCATGCGCGTTTACGGCACCTAAACGCTCCTAATA | 211 |
| DNAzyme G16A | GAG | |
| Fe³⁺ B12 | GCGGCATGCGCGTTTCCGGCACCTAAACGCTCCTAATA | 212 |
| DNAzyme G16C | GAG | |
| Fe³⁺ B12 | GCGGCATGCGCGTTTTCGGCACCTAAACGCTCCTAATA | 213 |
| DNAzyme G16T | GAG | |
| Fe³⁺ B12 | GCGGCATGCGCGTTTGGGGCACCTAAACGCTCCTAATA | 214 |
| DNAzyme C17G | GAG | |
| Fe³⁺ B12 | GCGGCATGCGCGTTTGTGGCACCTAAACGCTCCTAATA | 215 |
| DNAzyme C17T | GAG | |
| Fe³⁺ B12 | GCGGCATGCGCGTTTGAGGCACCTAAACGCTCCTAATA | 216 |
| DNAzyme C17A | GAG | |
| Fe³⁺ B12 | GCGGCATGCGCGTTTGCAGCACCTAAACGCTCCTAATA | 217 |
| DNAzyme G18A | GAG | |
| Fe³⁺ B12 | GCGGCATGCGCGTTTGCTGCACCTAAACGCTCCTAATA | 218 |
| DNAzyme G18T | GAG | |
| Fe³⁺ B12 | GCGGCATGCGCGTTTGCCGCACCTAAACGCTCCTAATA | 219 |
| DNAzyme G18C | GAG | |
| Fe³⁺ B12 | GCGGCATGCGCGTTTGCGACACCTAAACGCTCCTAATA | 220 |
| DNAzyme G19A | GAG | |
| Fe³⁺ B12 | GCGGCATGCGCGTTTGCGCCACCTAAACGCTCCTAATA | 221 |
| DNAzyme G19C | GAG | |
| Fe³⁺ B12 | GCGGCATGCGCGTTTGCGTCACCTAAACGCTCCTAATA | 222 |
| DNAzyme G19T | GAG | |
| Fe³⁺ B12 | GCGGCATGCGCGTTTGCGGGACCTAAACGCTCCTAATA | 223 |
| DNAzyme C20G | GAG | |
| Fe³⁺ B12 | GCGGCATGCGCGTTTGCGGTACCTAAACGCTCCTAATA | 32 |
| DNAzyme C20T | GAG | |
| Fe³⁺ B12 | GCGGCATGCGCGTTTGCGGAACCTAAACGCTCCTAATA | 224 |
| DNAzyme C20A | GAG | |
| Fe³⁺ B12 | GCGGCATGCGCGTTTGCGGCGCCTAAACGCTCCTAATA | 225 |
| DNAzyme A21G | GAG | |
| Fe³⁺ B12 | GCGGCATGCGCGTTTGCGGCCCCTAAACGCTCCTAATA | 226 |
| DNAzyme A21C | GAG | |
| Fe³⁺ B12 | GCGGCATGCGCGTTTGCGGCTCCTAAACGCTCCTAATA | 227 |
| DNAzyme A21T | GAG | |
| Fe³⁺ B12 | GCGGCATGCGCGTTTGCGGCAGCTAAACGCTCCTAATA | 228 |
| DNAzyme C22G | GAG | |
| Fe³⁺ B12 | GCGGCATGCGCGTTTGCGGCATCTAAACGCTCCTAATA | 229 |
| DNAzyme C22T | GAG | |
| Fe³⁺ B12 | GCGGCATGCGCGTTTGCGGCAACTAAACGCTCCTAATA | 230 |
| DNAzyme C22A | GAG | |
| Fe³⁺ B12 | GCGGCATGCGCGTTTGCGGCACGTAAACGCTCCTAATA | 231 |
| DNAzyme C23G | GAG | |
| Fe³⁺ B12 | GCGGCATGCGCGTTTGCGGCACTTAAACGCTCCTAATA | 232 |
| DNAzyme C23T | GAG | |
| Fe³⁺ B12 | GCGGCATGCGCGTTTGCGGCACATAAACGCTCCTAATA | 233 |
| DNAzyme C23A | GAG | |
| Fe³⁺ B12 | GCGGCATGCGCGTTTGCGGCACCGAAACGCTCCTAATA | 234 |
| DNAzyme T24G | GAG | |
| Fe³⁺ B12 | GCGGCATGCGCGTTTGCGGCACCCAAACGCTCCTAATA | 235 |
| DNAzyme T24C | GAG | |
| Fe³⁺ B12 | GCGGCATGCGCGTTTGCGGCACCAAAACGCTCCTAATA | 236 |
| DNAzyme T24A | GAG | |
| Fe³⁺ B12 | GCGGCATGCGCGTTTGCGGCACCTAAACGGTCCTAATA | 237 |
| DNAzyme C30G | GAG | |
| Fe³⁺ B12 | GCGGCATGCGCGTTTGCGGCACCTAAACGTTCCTAATA | 238 |
| DNAzyme C30T | GAG | |
| Fe³⁺ B12 | GCGGCATGCGCGTTTGCGGCACCTAAACGATCCTAATA | 239 |
| DNAzyme C30A | GAG | |
| Fe³⁺ B12 | GCGGCATGCGCGTTTGCGGCACCTAAACGCACCTAATA | 240 |
| DNAzyme T31A | GAG | |
| Fe³⁺ B12 | GCGGCATGCGCGTTTGCGGCACCTAAACGCCCCTAATA | 22 |
| DNAzyme T31C | GAG | |
| Fe³⁺ B12 | GCGGCATGCGCGTTTGCGGCACCTAAACGCGCCTAATA | 241 |
| DNAzyme T31G | GAG | |

Fe²⁺ H5 DNAzyme Truncations

| | | |
|---|---|---|
| Fe²⁺ H5 | GGATATCTCCTAGCCAGACTGTTATGTGTGATACGGCA | 242 |
| DNAzyme | AACTTCGTGATGCCTCTACGGGTCCG | |
| Truncation 51 | | |
| Fe²⁺ H5 | GATATCTCCTAGCCAGACTGTTATGTGTGATACGGCAA | 243 |
| DNAzyme | ACTTCGTGATGCCTCTACGGGTCCG | |
| Truncation 52 | | |
| Fe²⁺ H5 | TGGATATCTCCTAGCCAGACTGTTATGTGTGATACGGC | 244 |
| DNAzyme | AAACTTCGTGATGCCTCTACGGGTCC | |
| Truncation 31 | | |
| Fe²⁺ H5 | TGGATATCTCCTAGCCAGACTGTTATGTGTGATACGGC | 245 |
| DNAzyme | AAACTTC GTG ATG CCTCTA CGGTC | |
| Truncation 32 | | |
| Fe²⁺ H5 | TGGATATCTCCTAGCCAGACTGTTATGTGTGATACG | 246 |
| DNAzyme | GCAAACTTCGTGATGCCTCTACGGGT | |
| Truncation 33 | | |
| Fe²⁺ H5 | TGGATATCTCCTAGCCAGACTGTTATGTGTGATACGGC | 247 |
| DNAzyme | AAACTTCGTGATGCCTCTACGGG | |
| Truncation 34 | | |

Fe³⁺ B12 DNAzyme Truncations

| | | |
|---|---|---|
| Fe³⁺ B12 | CGGCATGCGCGTTTGCGGCACCTAAACGCTCCTAATAG | 248 |
| DNAzyme | AG | |
| Trunctation 51 | | |
| Fe³⁺ B12 | GGCATGCGCGTTTGCGGCACCTAAACGCTCCTAATAGA | 249 |
| DNAzyme | G | |
| Trunctation 52 | | |
| Fe³⁺ B12 | GCATGCGCGTTTGCGGCACCTAAACGCTCCTAATAGAG | 250 |
| DNAzyme | | |
| Trunctation 53 | | |
| Fe³⁺ B12 | CATGCGCGTTTGCGGCACCTAAACGCTCCTAATAGAG | 251 |
| DNAzyme | | |
| Trunctation 54 | | |
| Fe³⁺ B12 | ATGCGCGTTTGCGGCACCTAAACGCTCCTAATAGAG | 252 |
| DNAzyme | | |
| Trunctation 55 | | |
| Fe³⁺ B12 | GCGGCATGCGCGTTTGCGGCACCTAAACGCTCCTAATA | 253 |
| DNAzyme | GA | |
| Trunctation 31 | | |
| Fe³⁺ B12 | GCGGCATGCGCGTTTGCGGCACCTAAACGCTCCTAATA | 254 |
| DNAzyme | G | |
| Trunctation 32 | | |
| Fe³⁺ B12 | GCGGCATGCGCGTTTGCGGCACCTAAACGCTCCTAATA | 255 |
| DNAzyme | | |
| Trunctation 33 | | |

Fe²⁺ H5 DNAzyme Combinations

| | | |
|---|---|---|
| | TGGATATCTCCTAGCCAGACTGTTATGTGTGATACGGC | 24 |
| | AAACTTCGTGATGCCTCTACGGGTCCG | |
| Fe²⁺ H5 | TGGATATCTCCTAGCCAGACTGTTATGTGTGATACGGC | 256 |
| DNAzyme | TAACTTCGTGATGGCTCTACGGGTCCG | |
| Combo 1 | | |
| Fe²⁺ H5 | TGGATATCTCCTAGCCAGACTGTTATGTGTGATACGGC | 257 |
| DNAzyme | ACACTTCGTGATGGCTCTACGGGTCCG | |
| Combo 2 | | |
| Fe²⁺ H5 | TGGATATCTCCTAGCCAGACTGTTATGTGTGATACGGC | 258 |
| DNAzyme | ACACTTCGTGATGCCCCTACGGGTCCG | |
| Combo 3 | | |
| Fe²⁺ H5 | TGGATATCTCCTAGCCAGACTGTTATGTGTGATACGGC | 259 |
| DNAzyme | TAACTTCGTGATCCCTCTACGGGTCCG | |
| Combo 4 | | |
| Fe²⁺ H5 | TGGATATCTCCTAGCCAGACTGTTATGTGTGATACGGC | 260 |
| DNAzyme | AAACTTCGTGATCCCCCTACGGGTCCG | |
| Combo 5 | | |
| Fe²⁺ H5 | TGGATATCTCCTAGCCAGACTGTTATGTGTGATACGGC | 261 |
| DNAzyme | | |
| Combo 6 | AAACTTCGTGATGGCCCTACGGGTCCG | |
| Fe²⁺ H5 | TGGATATCTCCTAGCCAGACTGTTATGTGTGATACGGC | 262 |
| DNAzyme | AAACTTCGTGATCGCTCTACGGGTCCG | |
| Combo 7 | | |
| Fe²⁺ H5 | TGGATATCTCCTAGCCAGACTGTTATGTGTGATACGGC | 263 |
| DNAzyme | ACACTTCGTGATCCCTCTACGGGTCCG | |
| Combo 8 | | |
| Fe²⁺ H5 | TGGATATCTCCTAGCCAGACTGTTATGTGTGATACGGC | 264 |
| DNAzyme | GAACTTCGTGATGGCTCTACGGGTCCG | |
| Combo 9 | | |
| Fe²⁺ H5 | TGGATATCTCCTAGCCAGACTGTTATGTGTGATACGGC | 265 |
| DNAzyme | TAACTTCGTGATGCCCCTACGGGTCCG | |
| Combo 10 | | |
| Fe²⁺ H5 | TGGATATCTCCTAGCCAGACTGTTATGTGTGATACGGC | 266 |
| DNAzyme | GAACTTCGTGATGCCCCTACGGGTCCG | |
| Combo 11 | | |
| Fe²⁺ H5 | TGGATATCTCCTAGCCAGACTGTTATGTGTGATACGGC | 267 |
| DNAzyme | TCACTTCGTGATGCCTCTACGGGTCCG | |
| Combo 12 | | |
| Fe²⁺ H5 | TGGATATCTCCTAGCCAGACTGTTATGTGTGATACGGC | 268 |
| DNAzyme | GCACTTCGTGATGCCTCTACGGGTCCG | |
| Combo 13 | | |
| Fe²⁺ H5 | TGGATATCTCCTAGCCAGACTGTTATGTGTGATACGGC | 269 |
| DNAzyme | GAACTTCGTGATCCCTCTACGGGTCCG | |
| Combo 14 | | |
| Fe²⁺ H5 | TGGATATCTCCTAGCCAGACTGTTATGTGTGATACGGC | 270 |
| DNAzyme | TAACTTCGTGATCGCTCTACGGGTCCG | |
| Combo 15 | | |
| Fe²⁺ H5 | TGGATATCTCCTAGCCAGACTGTTATGTGTGATACGGC | 271 |
| DNAzyme | TAACTTCGTGATCCCCCTACGGGTCCG | |
| Combo 16 | | |
| Fe²⁺ H5 | TGGATATCTCCTAGCCAGACTGTTATGTGTGATACGGC | 272 |
| DNAzyme | ACACTTCGTGATCGCTCTACGGGTCCG | |
| Combo 17 | | |
| Fe²⁺ H5 | TGGATATCTCCTAGCCAGACTGTTATGTGTGATACGGC | 273 |
| DNAzyme | TAACTTCGTGATGGCCCTACGGGTCCG | |
| Combo 18 | | |
| Fe²⁺ H5 | TGGATATCTCCTAGCCAGACTGTTATGTGTGATACGGC | 274 |
| DNAzyme | GAACTTCGTGATCCCCCTACGGGTCCG | |
| Combo 19 | | |
| Fe²⁺ H5 | TGGATATCTCCTAGCCAGACTGTTATGTGTGATACGGC | 275 |
| DNAzyme | ACACTTCGTGATCCCCCTACGGGTCCG | |
| Combo 20 | | |
| Fe²⁺ H5 | TGGATATCTCCTAGCCAGACTGTTATGTGTGATACGGC | 276 |
| DNAzyme | ACACTTCGTGATGGCCCTACGGGTCCG | |
| Combo 21 | | |
| Fe²⁺ H5 | TGGATATCTCCTAGCCAGACTGTTATGTGTGATACGGC | 277 |
| DNAzyme | GCACTTCGTGATGGCTCTACGGGTCCG | |
| Combo 22 | | |
| Fe²⁺ H5 | TGGATATCTCCTAGCCAGACTGTTATGTGTGATACGGC | 278 |
| DNAzyme | TCACTTCGTGATGCCCCTACGGGTCCG | |
| Combo 23 | | |
| Fe²⁺ H5 | TGGATATCTCCTAGCCAGACTGTTATGTGTGATACGGC | 279 |
| DNAzyme | GCACTTCGTGATCCCTCTACGGGTCCG | |
| Combo 24 | | |
| Fe²⁺ H5 | TGGATATCTCCTAGCCAGACTGTTATGTGTGATACGGC | 280 |
| DNAzyme | TCACTTCGTGATCCCTCTACGGGTCCG | |
| Combo 25 | | |
| Fe²⁺ H5 | TGGATATCTCCTAGCCAGACTGTTATGTGTGATACGGC | 281 |
| DNAzyme | GAACTTCGTGATGGCCCTACGGGTCCG | |
| Combo 26 | | |
| Fe²⁺ H5 | TGGATATCTCCTAGCCAGACTGTTATGTGTGATACGGC | 282 |
| DNAzyme | TCACTTCGTGATGGCTCTACGGGTCCG | |
| Combo 27 | | |
| Fe²⁺ H5 | TGGATATCTCCTAGCCAGACTGTTATGTGTGATACGGC | 283 |
| DNAzyme | AAACTTCGTGATCGCCCTACGGGTCCG | |
| Combo 28 | | |
| Fe²⁺ H5 | TGGATATCTCCTAGCCAGACTGTTATGTGTGATACGGC | 284 |
| DNAzyme | GAACTTCGTGATCGCTCTACGGGTCCG | |
| Combo 29 | | |
| Fe²⁺ H5 | TGGATATCTCCTAGCCAGACTGTTATGTGTGATACGGC | 285 |
| DNAzyme | GCACTTCGTGATGCCCCTACGGGTCCG | |
| Combo 30 | | |
| Fe²⁺ H5 | TGGATATCTCCTAGCCAGACTGTTATGTGTGATACGGC | 286 |
| DNAzyme | GCACTTCGTGATCGCTCTACGGGTCCG | |
| Combo 31 | | |
| Fe²⁺ H5 | TGGATATCTCCTAGCCAGACTGTTATGTGTGATACGGC | 287 |
| DNAzyme | TAACTTCGTGATCGCCCTACGGGTCCG | |
| Combo 32 | | |
| Fe²⁺ H5 | TGGATATCTCCTAGCCAGACTGTTATGTGTGATACGGC | 288 |
| DNAzyme | TCACTTCGTGATCCCCCTACGGGTCCG | |
| Combo 33 | | |
| Fe²⁺ H5 | TGGATATCTCCTAGCCAGACTGTTATGTGTGATACGGC | 289 |
| DNAzyme | TCACTTCGTGATCGCTCTACGGGTCCG | |
| Combo 34 | | |
| Fe²⁺ H5 | TGGATATCTCCTAGCCAGACTGTTATGTGTGATACGGC | 290 |
| DNAzyme | TCACTTCGTGATGGCCCTACGGGTCCG | |
| Combo 35 | | |
| Fe²⁺ H5 | TGGATATCTCCTAGCCAGACTGTTATGTGTGATACGGC | 291 |
| DNAzyme | ACACTTCGTGATCGCCCTACGGGTCCG | |
| Combo 36 | | |
| Fe²⁺ H5 | TGGATATCTCCTAGCCAGACTGTTATGTGTGATACGGC | 292 |
| DNAzyme | GCACTTCGTGATCCCCCTACGGGTCCG | |
| Combo 37 | | |
| Fe²⁺ H5 | TGGATATCTCCTAGCCAGACTGTTATGTGTGATACGGC | 293 |
| DNAzyme | GAACTTCGTGATCGCCCTACGGGTCCG | |
| Combo 38 | | |
| Fe²⁺ H5 | TGGATATCTCCTAGCCAGACTGTTATGTGTGATACGGC | 294 |
| DNAzyme | GCACTTCGTGATGGCCCTACGGGTCCG | |
| Combo 39 | | |
| Fe²⁺ H5 | TGGATATCTCCTAGCCAGACTGTTATGTGTGATACGGC | 295 |
| DNAzyme | TCACTTCGTGATCGCCCTACGGGTCCG | |
| Combo 40 | | |
| Fe²⁺ H5 | TGGATATCTCCTAGCCAGACTGTTATGTGTGATACGGC | 296 |
| DNAzyme | GCACTTCGTGATCGCCCTACGGGTCCG | |
| Combo 41 | | |

Fe²⁺ H5 DNAzymes and Substrates for Kinetics

| | | |
|---|---|---|
| Fe²⁺ H5 Original | /5IABkFQ/TGGATATCTCCTAGCCAGACTGTTATGTGTG | 18 |
| DNAzyme 5′ | ATACGGCAAACTTCGTGATGCCTCTACGGGTCCG | |
| Iowa Black ™ FQ | | |
| Fe²⁺ H5 | /5IABkFQ/TGGATATCTCCTAGCCAGACTGTTATGTGTG | 297 |
| DNAzyme A39G | ATACGGCGAACTTCGTGATGCCTCTACGGGTCCG | |
| 5′ Iowa Black ™ | | |
| FQ | | |
| Fe²⁺ H5 | /5IABkFQ/TGGATATCTCCTAGCCAGACTGTTATGTGTG | 298 |
| DNAzyme A40C | ATACGGCACACTTCGTGATGCCTCTACGGGTCCG | |
| 5′ Iowa Black ™ | | |
| FQ | | |
| Fe²⁺ H5 | /5IABkFQ/TGGATATCTCCTAGCCAGACTGTTATGTGTG | 299 |
| DNAzyme T54C | ATACGGCAAACTTCGTGATGCCCCTACGGGTCCG | |
| 5′ Iowa Black ™ | | |
| FQ | | |
| Fe²⁺ H5 Original | /5IABkFQ/CGGACCCGTATCAATCTCACGTATrAGGATAT | 20 |
| Substrate 3′ Alexa | CCA/3AlexF488N/ | |
| Fluor ™ 488 | | |
| (NHS Ester) | | |
| Fe²⁺ H5 Substrate | /5IABkFQ/CGGACCCGTACCAATCTCACGTATrAGGATAT | 300 |
| T11C 3′ Alexa | CCA/3AlexF488N/ | |
| Fluor ™ 488 | | |
| (NHS Ester) | | |
| Fe²⁺ H5 Substrate | /5IABkFQ/CGGACCCGTATCCATCTCACGTATrAGGATAT | 301 |
| A13C 3′ Alexa | CCA/3AlexF488N/ | |
| Fluor ™ 488 | | |
| (NHS Ester) | | |

Fe³⁺ B12 DNAzymes and Substrates for Kinetics

| | | |
|---|---|---|
| F^{e3+} B12 Original | GCGGCATGCGCGTTTGCGGCACCTAAACGCTCCTAATA | 15 |
| DNAzyme 3′ | GAG/3IAbRQSp/ | |
| Iowa Black ™ RQ | | |
| Fe³⁺ B12 | GCGGCATGCGCGTTTGTGGTACCTAAACGCTCCTAATA | 302 |
| DNAzyme C20T | GAG/3IAbRQSp/ | |
| 3′ Iowa Black ™ | | |
| RQ | | |
| Fe³⁺ B12 Original | /5Alex647N/CTCTATTArGGGAGACTCGCATGCCGC/ | 17 |
| Substrate 5′ Alexa | 3IAbRQSp/ | |
| Fluor ™ 647 | | |
| (NHS Ester) | | |
| Fe³⁺ B12 | /5Alex647N/CTCTATTArGGGAGGCTCGCATGCCGC/ | 303 |
| Substrate A14G | 3IAbRQSp/ | |
| 5′ Alexa Fluor ™ | | |
| 647 (NHS Ester) | | |
| Fe³⁺ B12 | /5Alex647N/CTCTATTArGGGAGAATCGCATGCCGC/ | 304 |
| Substrate C15A 5′ | 3IAbRQSp/ | |
| Alexa Fluor ™ | | |
| 647 (NHS Ester) | | |
| Fe³⁺ B12 | /5Alex647N/CTCTATTArGGGAGATTCGCATGCCGC/ | 305 |
| Substrate C15T 5′ | 3IAbRQSp/ | |
| Alexa Fluor ™ | | |
| 647 (NHS Ester) | | |

### Example 9: Additional Applications of Iron DNAzymes

Given the observed dependence of the Fe³⁺ sensor on Bis-Tris in in vitro assays, a study was conducted which evaluated the sensor's performance when prepared in Bis-Tris and Tris buffers and subsequently delivered into HeLa cells for comparison. The study compared the cells treated with 100 μM FeCl₃ in DMEM, or only DMEM after the transfection of the iron DNAzyme sensors. While Fe²⁺ exhibited distinct fluorescence responses in both buffers, Fe³⁺ showed a significant fluorescence increase only in the presence of Bis-Tris but not Tris buffer. These findings confirm that Bis-Tris is a co-factor for the Fe³⁺ DNAzyme sensor.

To assess and compare the performance of the mutated Fe³⁺ and Fe²⁺ sensors with their original counterparts, the study evaluated the Fe³⁺ DNAzyme mutant (C20T with substrate C15A) and the Fe²⁺ DNAzyme mutant (C15T) alongside the original DNAzymes. These constructs were delivered into HeLa cells, and fluorescence signals were analyzed by comparing the active DNAzyme sensors (aE) and their inactive controls (iE) following a 2-hour incubation in DMEM supplemented with either 100 μM FeCl₃ or an equal amount of water. The mutated Fe²⁺ control demonstrated a lower background signal, whereas the mutated Fe³⁺ sensor exhibited increased fluorescence intensity. With the mutations, the improved sensor pairs showed better performance when sensing intrinsic iron.

To assess the performance of the mutated sensors on tissue sections, the study applied the sensors on brain slices and compared the iron between wild-type and 5×FAD mice. The Mutated Fe³⁺ sensor showed brighter signaling than the original sensor.

Ferroptosis is a Fe-related cell death. To understand how Fe²⁺ and Fe³⁺ have a role in this process, the cells were incubated with Sulfasalazine (SAS), which inhibits System Xc- and induces ferroptosis. Elevated Fe²⁺ and Fe³⁺ levels, as well as an elevated Fe³⁺/Fe²⁺ ratio over time, were observed when compared with nontreated controls. This elevation is inhibited by Ferrostatin-1 (Fer-1), an antioxidant that inhibits lipid peroxidation by scavenging alkoxyl radicals. More interestingly, treating these cells with Fer-1 alone slightly reduced the Fe levels in the cells. This observation indicates the association between Fe levels and lipid peroxidation.

To understand how labile iron and iron redox changes are associated with cell ferroptosis, the study focused on the regulatory effects of NCOA4 on Fe²⁺ and Fe³⁺. NCOA4 is a key gene that regulates iron homeostasis through ferritinophagy, degrading iron storage protein, and releasing iron into the cell. To understand how NCOA4 influences Fe²⁺ and Fe³⁺, the study used the CRISPR/Cas technique to knock down NCOA4 expressions. As shown with the western blot, single guide RNAs knocked down the expression of NCOA4 in the HT1080 cells. In these cells, reduced Fe²⁺ and Fe³⁺ signals were detected as indicated by the DNAzyme sensors. In the meanwhile, the redox ratio of Fe³⁺/Fe²⁺ elevated, indicating the NCOA4 may not only regulate the labile iron amount but also the Fe redox states.

To further understand how NCOA4 is involved in regulating labile Fe during ferroptosis, the study induced ferroptosis in normal cells and NCOA4 down-regulated cells. The control cells that were treated with control guide RNA instead of NCOA4 guide RNA, showed elevated Fe levels and Fe³⁺/Fe²⁺ levels when treated with SAS. On the contrary, when inhibiting ferroptosis with Fer-1, reduced Fe²⁺ and Fe³⁺ levels were observed. More interestingly, adding Fer-1 to SAS-treated cells not only revoked the ferroptosis but also reduced the Fe²⁺ and Fe³⁺ levels. These results indicate that the elevation of Fe²⁺ and Fe³⁺ levels is associated with ferroptosis and lipid peroxidation. Since NCOA4 is a key regulator in labile Fe levels, the study tested similar treatments in NCOA4 downregulated cells and observed a smaller increase in Fe²⁺ and Fe³⁺ levels. This observation indicates that the increase of labile Fe levels during induced ferroptosis was partially through the NCOA4-mediated ferritinophagy.

### Example 10: Copper Homeostasis and Cuproptosis in Neuron Cells

With extensive research on regulatory cell death in AD, increasing evidence has revealed that copper dyshomeostasis and neurotoxicity could play a contributing role in AD neurodegeneration by mediating cuproptosis, oxidative stress, Aβ plaque deposition, neuronal death, and synaptic damage. Zhang et al. reported that cuproptosis in neurons is contributed by the oxidative stress process caused by the conversion of Cu⁺ to Cu²⁺, and subsequent proteotoxic stress. A study demonstrated that DNAzyme was well suited for detecting Cu⁺ and Cu²⁺ homeostasis in neurons cells and cuproptosis within cancer cells. The study tried to knock out FDX1 by CRISPR-Cas9 technology, but the SH-SY5Y cells can barely survival. To investigate whether DNAzyme can be used for detecting Cu⁺ and Cu²⁺ homeostasis in cuproptosis within neurons cells, the study constructed SH-SY5Y cells with FDX1 (nontarget as a control) knocked down using RNAi technology. Depletion of FDX1 by RNAi in SH-SY5Y cells caused a strong decrease of Cu⁺ fluorescence signal and significant increase of Cu²⁺ fluorescence signal, while that Cu²⁺/Cu⁺ ratios significantly increased compared with the nontarget group. The FDX1 knockdown efficiency was determined through qPCR, the result shows that the expression of FDX1 was inhibited by the siRNAs. This data demonstrated that DNAzyme can be used for imaging Cu⁺ and Cu²⁺ homeostasis in cuproptosis within neurons cells.

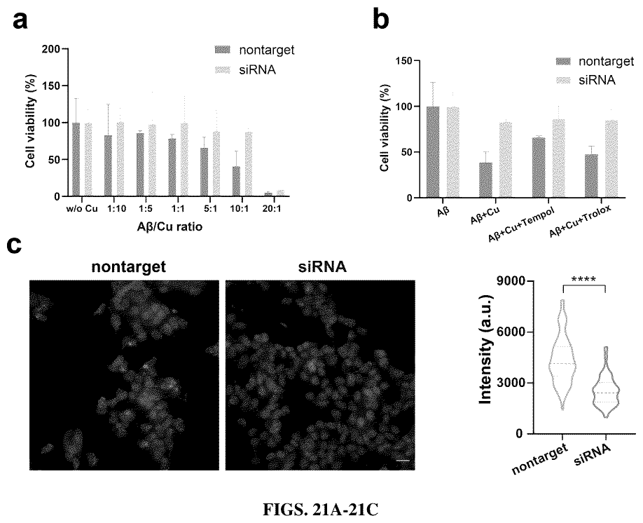

Both oxidative stresses and proteotoxic stresses mediated cuproptosis were also observed in neurons during copper overload by elesclomol (ES), which is a copper ionophore, contributing to neuronal death. The high redox properties of copper can promote the Aβ-induced oxidative stress and subsequent neuronal death. Imaging data shows that with Aβ treatment, the intracellular Cu level increased. The hypothesis is that the property of Aβ can be acted as a copper ionophore is part of what contributes to the pathological effects of cuproptosis in AD. To explore more about whether cuproptosis is related to AD neurodegeneration and understand the role of Aβ-Cu in neuronal death, the viability of SH-SY5Y cells with FDX1 knocked down by RNAi were monitored by stimulating with different CuCl₂ concentration and 10 μM Aβ for overnight. Aβ-Cu complex exhibited significantly higher cytotoxicity to SH-SY5Y cells with nontarget control group than the FDX1 gene knocked out cells, especially in higher Aβ/Cu ratio group. Study also showed that the degree of Cu-Aβ cytotoxicity correlates with the levels of Cu²⁺ ions that accelerate fiber formation. Cu²⁺ ions bound to Aβ are consistently more toxic to neuronal cells than Aβ in the absence of Cu²⁺ ions. To further test the hypothesis that Aβ-Cu can induce cuproptosis, SH-SY5Y cells with FDX1 knocked down was treated with ROS scavengers Tempol or Trolox, and Tempol or Trolox fail to fully rescue this cell death. The fluorescence intensity of ROS in nontarget control group is higher than the Fdx1 gene knocked out cells, which means oxidative stress decreased with lower Cu1 generated FDX1 knocked out of SH-SY5Y cells Together, the data showed that Aβ-Cu involved in both oxidative stress and proteotoxic stress mediated cuproptosis during neuronal death. Study show that oxidative stress primarily contributed to neuron death at the early stage of cuproptosis, which exacerbated proteotoxic stress-dominated neuron death at the later stage of cuproptosis. Example results are shown in FIGS. 21A-21C.

### Example Aspects

Example 1: A composition for simultaneously detecting a target ion in multiple oxidation states, the composition comprising: i) a first DNAzyme sensor comprising: a first substrate strand comprising a first cleavage site, wherein the first cleavage site is uncleaved when the target ion in a first oxidation state is not present; and a first enzyme strand at least partially complementary to the first substrate strand and comprising a first catalytic loop; wherein the first catalytic loop is capable of cleaving the first substrate strand at the first cleavage site in the presence of the target ion in the first oxidation state, wherein said cleavage provides a first detectable signal; and ii) a second DNAzyme sensor comprising: a second substrate strand comprising a second cleavage site, wherein the second cleavage site is uncleaved when the target ion in a second oxidation state is not present; and a second enzyme strand at least partially complementary to the second substrate strand and comprising a second catalytic loop; wherein the second catalytic loop is capable of cleaving the second substrate strand at the second cleavage site in the presence of the target ion in the second oxidation state, wherein said cleavage provides a second detectable signal.

Example 2: The composition of any examples herein, particularly Example 1, wherein each cleavage site comprises 1 to 5 RNA bases.

Example 3: The composition of any examples herein, particularly Example 1, wherein each cleavage site is interspersed between two segments of DNA each comprising 3 to 30 DNA bases.

Example 4: The composition of any examples herein, particularly Example 1, wherein each substrate strand further comprises at least one non-natural nucleic acid.

Example 5: The composition of any examples herein, particularly Example 4, wherein the at least one non-natural nucleic acid is a locked nucleic acid (LNA).

Example 6: The composition of any examples herein, particularly Example 1, wherein the detectable signal is a fluorophore or fluorescent dye.

Example 7: The composition of any examples herein, particularly Example 1, wherein the detectable signal is a photoacoustic dye; and wherein, when the substrate strand is cleaved, the detectable signal is activated upon exposure to an acoustic signal.

Example 8: The composition of any examples herein, particularly Example 1, wherein the detectable signal is conjugated to a first 8 of the substrate strand; and wherein a quencher is conjugated to a complementary end of the enzyme strand or to a second end of the substrate strand.

Example 9: The composition of any examples herein, particularly Example 1, wherein the target ion is iron; wherein the first cleavage site is uncleaved when Fe²⁺ is not present and the first catalytic loop comprises SEQ ID NO: 306 or a variant thereof; and wherein the second cleavage site is uncleaved when Fe³⁺ is not present and the second catalytic loop comprises SEQ ID NO: 307 or a variant thereof.

Example 10: The composition of any examples herein, particularly Example 9, wherein the first substrate strand comprises 80% similarity or more to any one of the sequences in TABLE 1; wherein the first enzyme strand comprises 80% similarity or more to any one of the sequences in TABLE 2; wherein the second substrate strand comprises 80% similarity or more to any one of the sequences in TABLE 3; and wherein the second enzyme strand comprises 80% similarity or more to any one of the sequences in TABLE 4.

Example 11: The composition of any examples herein, particularly Example 1, wherein the target ion is copper; wherein the first cleavage site is uncleaved when Cu⁺ is not present and the first catalytic loop comprises SEQ ID NO: 308 or a variant thereof; and wherein the second cleavage site is uncleaved when Cu²⁺ is not present and the second catalytic loop comprises SEQ ID NO: 309 or a variant thereof.

Example 12: The composition of any examples herein, particularly Example 11, wherein the first substrate strand comprises 80% similarity or more to any one of the sequences in TABLE 5; wherein the first enzyme strand comprises 80% similarity or more to any one of SEQ ID NOs: 45, 56, 64; wherein the second substrate strand comprises 80% similarity or more to any one of the sequences in TABLE 7; and wherein the second enzyme strand comprises 80% similarity or more to any one of the sequences in TABLE 8.

Example 13: The composition of any examples herein, particularly Example 1, wherein the first substrate strand, the first enzyme strand, the second substrate strand, and/or the second enzyme strand comprises at least one point mutation.

Example 14: The composition of any examples herein, particularly Example 1, wherein the first substrate strand and the first enzyme strand are linked together and/or the second substrate strand and the second enzyme strand are linked together.

Example 15: A method of using the composition of any examples herein, particularly Example 1 to simultaneously detect the target ion in the first oxidation state and the second oxidation state in a cell or tissue, the method comprising: a) providing the composition to the cell or tissue; and b) detecting the first detectable signal and the second detectable signal.

Example 16: The method of any examples herein, particularly Example 15, further comprising, before step a), providing a reference level of the detectable signal by: i) providing to the cell or tissue a first inactive DNAzyme sensor and a second inactive DNAzyme sensor; wherein the first inactive DNAzyme sensor comprises: the first substrate strand; and a first inactive enzyme strand at least partially complementary to the first substrate strand and comprising at least one mutation, wherein the at least one mutation prevents the first inactive enzyme strand from cleaving the first substrate strand; wherein the second inactive DNAzyme sensor comprises: the second substrate strand; and a second inactive enzyme strand at least partially complementary to the second substrate strand and comprising at least one mutation, wherein the at least one mutation prevents the second inactive enzyme strand from cleaving the second substrate strand; ii) detecting the first detectable signal and the second detectable signal, thereby providing a first reference level of the first detectable signal and a second reference level of the second detectable signal; wherein the first and second reference levels are used to eliminate background noise in step b).

Example 17: The method of any examples herein, particularly Example 16, wherein the target ion is iron; wherein the first substrate strand comprises 80% similarity or more to any one of the sequences in TABLE 1; wherein the first inactive enzyme strand comprises 80% similarity or more to any one of SEQ ID NOs: 25 or 31; wherein the second substrate strand comprises 80% similarity or more to any one of the sequences in TABLE 3; and wherein the second inactive enzyme strand comprises 80% similarity or more to SEQ ID NO: 22.

Example 18: The method of any examples herein, particularly Example 16, wherein the target ion is copper; wherein the first substrate strand comprises 80% similarity or more to any one of the sequences in TABLE 5; wherein the first inactive enzyme strand comprises 80% similarity or more to any one of SEQ ID NOs: 46, 57, 65, or 70; wherein the second substrate strand comprises 80% similarity or more to any one of the sequences in TABLE 7; and wherein the second inactive enzyme strand comprises 80% similarity or more to any one of SEQ ID NOs: 49-53, 61-62, or 68.

Example 19: The method of any examples herein, particularly Example 15, wherein step b) comprises imaging the cell or tissue.

Example 20: The method of any examples herein, particularly Example 15, wherein the cell or tissue is cancerous, exhibits qualities of or is derived from a patient having a neurogenerative disease (e.g., Alzheimer's disease) or multiple sclerosis, or exhibits qualities of or is derived from a patient experiencing old age or an age-related disease or disorder.

Example 21: A DNAzyme sensor comprising: a substrate strand comprising a cleavage site, wherein the cleavage site is uncleaved when Fe²⁺ is not present; and an enzyme strand at least partially complementary to the substrate strand and comprising a catalytic loop; wherein the catalytic loop is capable of cleaving the substrate strand at the cleavage site in the presence of Fe²⁺, wherein said cleavage provides a detectable signal; and wherein the catalytic loop comprises SEQ ID NO: 306 or a variant thereof.

Example 22: The DNAzyme sensor of any examples herein, particularly Example 21, wherein the cleavage site comprises 1 to 5 RNA bases.

Example 23: The DNAzyme sensor of any examples herein, particularly Example 21, wherein the cleavage site is interspersed between two segments of DNA each comprising 3 to 30 DNA bases.

Example 24: The DNAzyme sensor of any examples herein, particularly Example 21, wherein the substrate strand further comprises at least one non-natural nucleic acid.

Example 25: The DNAzyme sensor of any examples herein, particularly Example 24, wherein the at least one non-natural nucleic acid is a locked nucleic acid (LNA).

Example 26: The DNAzyme sensor of any examples herein, particularly Example 21, wherein the detectable signal is a fluorophore or fluorescent dye.

Example 27: The DNAzyme sensor of any examples herein, particularly Example 26, wherein the detectable signal is Alexa Fluor 647 or Alexa Fluor 488.

Example 28: The DNAzyme sensor of any examples herein, particularly Example 21, wherein the detectable signal is a photoacoustic dye; and wherein, when the substrate strand is cleaved, the detectable signal is activated upon exposure to an acoustic signal.

Example 29: The DNAzyme sensor of any examples herein, particularly Example 28, wherein the detectable signal is indocyanine green, methylene blue, or Evans blue.

Example 30: The DNAzyme sensor of any examples herein, particularly Example 21, wherein the detectable signal is conjugated to a first end of the substrate strand; and wherein a quencher is conjugated to a complementary end of the enzyme strand or to a second end of the substrate strand.

Example 31: The DNAzyme sensor of any examples herein, particularly Example 30, wherein the quencher is Iowa Black RQ or Iowa Black FQ.

Example 32: The DNAzyme sensor of any examples herein, particularly Example 21, wherein the substrate strand comprises 80% similarity or more to any one of the sequences in TABLE 1; and wherein the enzyme strand comprises 80% similarity or more to any one of the sequences in TABLE 2.

Example 33: The DNAzyme sensor of any examples herein, particularly Example 21, wherein the substrate strand and/or enzyme strand comprises at least one point mutation.

Example 34: The DNAzyme sensor of any examples herein, particularly Example 21, wherein the substrate strand and the enzyme strand are linked together.

Example 35: The DNAzyme sensor of any examples herein, particularly Example 21, wherein the catalytic loop has a Michaelis constant (Kₘ) for Fe²⁺ or from about 1×10⁻⁴ min⁻¹ μM⁻¹ to about 1.5×10⁻⁴ min⁻¹ μM⁻¹.

Example 36: A method of using the DNAzyme sensor of any examples herein, particularly Example 21 to detect Fe²⁺ in a cell or tissue, the method comprising: a) providing the DNAzyme sensor to the cell or tissue; and b) detecting the detectable signal.

Example 37: The method of any examples herein, particularly Example 36, further comprising, before step a), providing a reference level of the detectable signal by: i) providing to the cell or tissue an inactive DNAzyme sensor comprising: the substrate strand; and an inactive enzyme strand at least partially complementary to the substrate strand and comprising at least one mutation, wherein the at least one mutation prevents the inactive enzyme strand from cleaving the substrate strand; and ii) detecting the detectable signal, thereby providing a reference level of the detectable signal; wherein the reference level is used to eliminate background noise in step b).

Example 38: The method of any examples herein, particularly Example 37, wherein the substrate strand comprises 80% similarity or more to any one of the sequences in TABLE 1; and wherein the inactive enzyme strand comprises 80% similarity or more to any one of SEQ ID NOs: 25 or 31.

Example 39: The method of any examples herein, particularly Example 36, wherein step b) comprises imaging the cell or tissue.

Example 40: The method of any examples herein, particularly Example 36, wherein the cell or tissue is cancerous, exhibits qualities of or is derived from a patient having a neurogenerative disease (e.g., Alzheimer's disease) or multiple sclerosis, or exhibits qualities of or is derived from a patient experiencing old age or an age-related disease or disorder.

Example 41: A DNAzyme sensor comprising: a substrate strand comprising a cleavage site, wherein the cleavage site is uncleaved when Fe³⁺ is not present; and an enzyme strand at least partially complementary to the substrate strand and comprising a catalytic loop; wherein the catalytic loop is capable of cleaving the substrate strand at the cleavage site in the presence of Fe³⁺, wherein said cleavage provides a detectable signal; and wherein the catalytic loop comprises SEQ ID NO: 307 or a variant thereof.

Example 42: The DNAzyme sensor of any examples herein, particularly Example 41, wherein the cleavage site comprises 1 to 5 RNA bases.

Example 43: The DNAzyme sensor of any examples herein, particularly Example 41, wherein the cleavage site is interspersed between two segments of DNA each comprising 3 to 30 DNA bases.

Example 44: The DNAzyme sensor of any examples herein, particularly Example 41, wherein the substrate strand further comprises at least one non-natural nucleic acid.

Example 45: The DNAzyme sensor of any examples herein, particularly Example 44, wherein the at least one non-natural nucleic acid is a locked nucleic acid (LNA).

Example 46: The DNAzyme sensor of any examples herein, particularly Example 41, wherein the detectable signal is a fluorophore or fluorescent dye.

Example 47: The DNAzyme sensor of any examples herein, particularly Example 46, wherein the detectable signal is Alexa Fluor 647 or Alexa Fluor 488.

Example 48: The DNAzyme sensor of any examples herein, particularly Example 41, wherein the detectable signal is a photoacoustic dye; and wherein, when the substrate strand is cleaved, the detectable signal is activated upon exposure to an acoustic signal.

Example 49: The DNAzyme sensor of any examples herein, particularly Example 48, wherein the detectable signal is indocyanine green, methylene blue, or Evans blue.

Example 50: The DNAzyme sensor of any examples herein, particularly Example 41, wherein the detectable signal is conjugated to a first end of the substrate strand; and wherein a quencher is conjugated to a complementary end of the enzyme strand or to a second end of the substrate strand.

Example 51: The DNAzyme sensor of any examples herein, particularly Example 50, wherein the quencher is Iowa Black RQ or Iowa Black FQ.

Example 52: The DNAzyme sensor of any examples herein, particularly Example 41, wherein the substrate strand comprises 80% similarity or more to any one of the sequences in TABLE 3; and wherein the enzyme strand comprises 80% similarity or more to any one of the sequences in TABLE 4.

Example 53: The DNAzyme sensor of any examples herein, particularly Example 41, wherein the substrate strand and/or enzyme strand comprises at least one point mutation.

Example 54: The DNAzyme sensor of any examples herein, particularly Example 41, wherein the substrate strand and the enzyme strand are linked together.

Example 55: The DNAzyme sensor of any examples herein, particularly Example 41, wherein the catalytic loop has a Michaelis constant (Kₘ) for Fe³⁺ or from about 1×10⁻² min⁻¹ μM⁻¹ to about 1.5×10⁻² min⁻¹ μM⁻¹.

Example 56: A method of using the DNAzyme sensor of any examples herein, particularly Example 41 to detect Fe³⁺ in a cell or tissue, the method comprising: a) providing the DNAzyme sensor to the cell or tissue; and b) detecting the detectable signal.

Example 57: The method of any examples herein, particularly Example 56, further comprising, before step a), providing a reference level of the detectable signal by: i) providing to the cell or tissue an inactive DNAzyme sensor comprising: the substrate strand; and an inactive enzyme strand at least partially complementary to the substrate strand and comprising at least one mutation, wherein the at least one mutation prevents the inactive enzyme strand from cleaving the substrate strand; and ii) detecting the detectable signal, thereby providing a reference level of the detectable signal; wherein the reference level is used to eliminate background noise in step b).

Example 58: The method of any examples herein, particularly Example 57, wherein the substrate strand comprises 80% similarity or more to any one of the sequences in TABLE 3; and wherein the inactive enzyme strand comprises 80% similarity or more to SEQ ID NO: 22.

Example 59: The method of any examples herein, particularly Example 56, wherein step b) comprises imaging the cell or tissue.

Example 60: The method of any examples herein, particularly Example 56, wherein the cell or tissue is cancerous, exhibits qualities of or is derived from a patient having a neurogenerative disease (e.g., Alzheimer's disease) or multiple sclerosis, or exhibits qualities of or is derived from a patient experiencing old age or an age-related disease or disorder.

Example 61: A kit comprising: i) a DNAzyme sensor comprising: a substrate strand comprising a cleavage site, wherein the cleavage site is uncleaved when a target molecule is not present; and an enzyme strand at least partially complementary to the substrate strand and comprising a catalytic loop; wherein the catalytic loop is capable of cleaving the substrate strand at the cleavage site in the presence of the target molecule, wherein said cleavage provides a detectable signal; and ii) an inactive DNAzyme sensor comprising: the substrate strand; and an inactive enzyme strand at least partially complementary to the substrate strand and comprising at least one mutation, wherein the at least one mutation prevents the inactive enzyme strand from cleaving the substrate strand.

Example 62: The kit of any examples herein, particularly Example 61, wherein the cleavage site comprises 1 to 5 RNA bases.

Example 63: The kit of any examples herein, particularly Example 61, wherein the cleavage site is interspersed between two segments of DNA each comprising 3 to 30 DNA bases.

Example 64: The kit of any examples herein, particularly Example 61, wherein the substrate strand further comprises at least one non-natural nucleic acid.

Example 65: The kit of any examples herein, particularly Example 64, wherein the at least one non-natural nucleic acid is a locked nucleic acid (LNA).

Example 66: The kit of any examples herein, particularly Example 61, wherein the detectable signal is a fluorophore or fluorescent dye.

Example 67: The kit of any examples herein, particularly Example 66, wherein the detectable signal is Alexa Fluor 647 or Alexa Fluor 488.

Example 68: The kit of any examples herein, particularly Example 61, wherein the detectable signal is a photoacoustic dye; and wherein, when the substrate strand is cleaved, the detectable signal is activated upon exposure to an acoustic signal.

Example 69: The kit of any examples herein, particularly Example 68, wherein the detectable signal is indocyanine green, methylene blue, or Evans blue.

Example 70: The kit of any examples herein, particularly Example 61, wherein the detectable signal is conjugated to a first end of the substrate strand; and wherein a quencher is conjugated to a complementary end of the enzyme strand or to a second end of the substrate strand.

Example 71: The kit of any examples herein, particularly Example 70, wherein the quencher is Iowa Black RQ or Iowa Black FQ.

Example 72: The kit of any examples herein, particularly Example 61, wherein the target molecule is Fe²⁺; wherein the substrate strand comprises 80% similarity or more to any one of the sequences in TABLE 1; wherein the enzyme strand comprises 80% similarity or more to any one of the sequences in TABLE 2; and wherein the inactive enzyme strand comprises 80% similarity or more to any one of SEQ ID NOs: 25 or 31.

Example 73: The kit of any examples herein, particularly Example 61, wherein the target molecule is Fe³⁺; wherein the substrate strand comprises 80% similarity or more to any one of the sequences in TABLE 3; wherein the enzyme strand comprises 80% similarity or more to any one of the sequences in TABLE 4; and wherein the inactive enzyme strand comprises 80% similarity or more to SEQ ID NO: 22.

Example 74: The kit of any examples herein, particularly Example 61, wherein the target molecule is Cu⁺; wherein the substrate strand comprises 80% similarity or more to any one of the sequences in TABLE 5; wherein the enzyme strand comprises 80% similarity or more to any one of the sequences in TABLE 6; and wherein the inactive enzyme strand comprises 80% similarity or more to any one of SEQ ID NOs: 46, 57, 65, or 70.

Example 75: The kit of any examples herein, particularly Example 61, wherein the target molecule is Cu²⁺; wherein the substrate strand comprises 80% similarity or more to any one of the sequences in TABLE 7; wherein the enzyme strand comprises 80% similarity or more to any one of the sequences in TABLE 8; and wherein the inactive enzyme strand comprises 80% similarity or more to any one of SEQ ID NOs: 49-53, 61-62, or 68.

Example 76: The kit of any examples herein, particularly Example 61, wherein the substrate strand and/or enzyme strand comprises at least one point mutation.

Example 77: The kit of any examples herein, particularly Example 61, wherein the substrate strand and the enzyme strand are linked together.

Example 78: A method of using the kit of any examples herein, particularly Example 61 to detect a target molecule in a cell or tissue by: a) providing the inactive DNAzyme sensor to the cell or tissue; b) detecting the detectable signal, thereby providing a reference level of the detectable signal; c) providing the DNAzyme sensor to the cell or tissue; and d) detecting the detectable signal; wherein the reference level is used to eliminate background noise in step d).

Example 79: The method of any examples herein, particularly Example 78, wherein step b) comprises imaging the cell or tissue.

Example 80: The method of any examples herein, particularly Example 78, wherein the cell or tissue is cancerous, exhibits qualities of or is derived from a patient having a neurogenerative disease (e.g., Alzheimer's disease) or multiple sclerosis, or exhibits qualities of or is derived from a patient experiencing old age or an age-related disease or disorder.

Example 81: A method of determining an effect of a therapeutic agent on a target molecule, the method comprising: a) administering the therapeutic agent to a cell or tissue; b) exposing the cell or tissue to a DNAzyme sensor comprising: a substrate strand comprising a cleavage site, wherein the cleavage site is uncleaved when the target molecule is not present; and an enzyme strand at least partially complementary to the substrate strand and comprising a catalytic loop; wherein the catalytic loop is capable of cleaving the substrate strand at the cleavage site in the presence of the target molecule, wherein said cleavage provides a detectable signal; and c) detecting the detectable signal; and d) using said detectable signal to determine the effect of the therapeutic agent on iron.

Example 82: The method of any examples herein, particularly Example 81, wherein the effect of the therapeutic agent on iron comprises a change in target molecule amount, concentration, activity, or spatial distribution within the cell or tissue compared to a reference signal produced by a control cell or tissue not exposed to the therapeutic agent.

Example 83: The method of any examples herein, particularly Example 81, wherein step c) comprises imaging the cell or tissue.

Example 84: The method of any examples herein, particularly Example 81, wherein the cell or tissue is cancerous.

Example 85: The method of any examples herein, particularly Example 81, wherein the cell or tissue exhibits qualities of or is derived from a patient having a neurogenerative disease (e.g., Alzheimer's disease) or multiple sclerosis.

Example 86: The method of any examples herein, particularly Example 81, wherein the cell or tissue exhibits qualities of or is derived from a patient experiencing old age or an age-related disease or disorder.

Example 87: The method of any examples herein, particularly Example 81, wherein the method is used to determine the effect of the therapeutic agent on Fe²⁺ and the catalytic loop comprises SEQ ID NO: 306 or a variant thereof; and/or wherein the method is used to determine the effect of the therapeutic agent on Fe³⁺ and the catalytic loop comprises SEQ ID NO: 307 or a variant thereof.

Example 88: The method of any examples herein, particularly Example 87, wherein the method is used to determine the effect of the therapeutic agent on Fe²⁺; wherein the substrate strand comprises 80% similarity or more to any one of the sequences in TABLE 1; and wherein the enzyme strand comprises 80% similarity or more to any one of the sequences in TABLE 2.

Example 89: The method of any examples herein, particularly Example 87, wherein the method is used to determine the effect of the therapeutic agent on Fe³⁺; wherein the substrate strand comprises 80% similarity or more to any one of the sequences in TABLE 3; and wherein the enzyme strand comprises 80% similarity or more to any one of the sequences in TABLE 4.

Example 90: The method of any examples herein, particularly Example 87, wherein the method is used to simultaneously determine the effect of the therapeutic agent on Fe²⁺ and Fe³⁺; wherein step b) further comprises exposing the cell or tissue to a first DNAzyme sensor and a second DNAzyme sensor; wherein the first DNAzyme sensor detects Fe²⁺ and comprises a first substrate strand and a first enzyme strand; wherein the second DNAzyme sensor detects Fe³⁺ and comprises a second substrate strand and a second enzyme strand; wherein the first substrate strand comprises 80% similarity or more to any one of the sequences in TABLE 1; wherein the first enzyme strand comprises 80% similarity or more to any one of the sequences in TABLE 2; wherein the second substrate strand comprises 80% similarity or more to any one of the sequences in TABLE 3; and wherein the second enzyme strand comprises 80% similarity or more to any one of the sequences in TABLE 4.

Example 91: The method of any examples herein, particularly Example 87, wherein the therapeutic agent induces ferroptosis.

Example 92: The method of any examples herein, particularly Example 81, wherein the method is used to determine the effect of the therapeutic agent on Cu⁺ and the catalytic loop comprises SEQ ID NO: 308 or a variant thereof; and/or wherein the method is used to determine the effect of the therapeutic agent on Cu²⁺ and the catalytic loop comprises SEQ ID NO: 309 or a variant thereof.

Example 93: The method of any examples herein, particularly Example 92, wherein the method is used to determine the effect of the therapeutic agent on Cut; wherein the substrate strand comprises 80% similarity or more to any one of the sequences in TABLE 5; and wherein the enzyme strand comprises 80% similarity or more to any one of the sequences in TABLE 6.

Example 94: The method of any examples herein, particularly Example 92, wherein the method is used to determine the effect of the therapeutic agent on Cu²⁺; wherein the substrate strand comprises 80% similarity or more to any one of the sequences in TABLE 7; and wherein the enzyme strand comprises 80% similarity or more to any one of the sequences in TABLE 8.

Example 95: The method of any examples herein, particularly Example 92, wherein the method is used to simultaneously determine the effect of the therapeutic agent on Cu⁺ and Cu²⁺ wherein step b) further comprises exposing the cell or tissue to a first DNAzyme sensor and a second DNAzyme sensor; wherein the first DNAzyme sensor detects Cu⁺ and comprises a first substrate strand and a first enzyme strand; wherein the second DNAzyme sensor detects Cu²⁺ and comprises a second substrate strand and a second enzyme strand; wherein the first substrate strand comprises 80% similarity or more to any one of the sequences in TABLE 5; wherein the first enzyme strand comprises 80% similarity or more to any one of the sequences in TABLE 6; wherein the second substrate strand comprises 80% similarity or more to any one of the sequences in TABLE 7; and wherein the second enzyme strand comprises 80% similarity or more to any one of the sequences in TABLE 8.

Example 96: The method of any examples herein, particularly Example 92, wherein the therapeutic agent induces cuproptosis.

Example 97: The method of any examples herein, particularly Example 81, further comprising, before step a), providing a reference level of the detectable signal by: i) providing to the cell or tissue an inactive DNAzyme sensor comprising: the substrate strand; and an inactive enzyme strand at least partially complementary to the substrate strand and comprising at least one mutation, wherein the at least one mutation prevents the inactive enzyme strand from cleaving the substrate strand; and ii) detecting the detectable signal, thereby providing a reference level of the detectable signal; wherein the reference level is used to eliminate background noise in step c).

Example 98: The method of any examples herein, particularly Example 81, wherein the therapeutic agent is an anti-cancer agent.

Example 99: The method of any examples herein, particularly Example 81, wherein the therapeutic agent is used to treat a neurogenerative disease (e.g., Alzheimer's disease) or multiple sclerosis

Example 100: The method of any examples herein, particularly Example 81, wherein the therapeutic agent is used to treat an age-related disease or disorder.

The following patents, applications and publications as listed below and throughout this document are hereby incorporated by reference in their entirety herein.

### REFERENCE LIST FOR EXAMPLE 1

Jo, T. G. et al., Colorimetric detection of Fe³⁺ and Fe²⁺ and sequential fluorescent detection of A13+ and pyrophosphate by an imidazole-based chemosensor in a near-perfect aqueous solution. Dyes and Pigments. 2017 Apr. 1; 139:136-47.
Rattanopas, S. et al., Indole-based fluorescent sensors for selective sensing of Fe²⁺ and Fe³⁺ in aqueous buffer systems and their applications in living cells. Journal of Photochemistry and Photobiology A: Chemistry. 2019 May 15; 377:138-48.
Sen, S. et al., A ratiometric fluorescent chemosensor for iron: discrimination of Fe²⁺ and Fe³⁺ and living cell application. Analyst. 2012; 137 (14): 3335-42.
Mati, S. S. et al., Exploring selective recognition between Fe²⁺, Fe³⁺ and their implementation in bio-imaging: A combined spectroscopic and theoretical investigation. Journal of Molecular Structure. 2019 May 15; 1184:102-9.
Au-Yeung, H. Y. et al., Molecular imaging of labile iron (II) pools in living cells with a turn-on fluorescent probe. Journal of the American Chemical Society. 2013 Oct. 9; 135 (40): 15165-73.
Aron, A. T. et al., In vivo bioluminescence imaging of labile iron accumulation in a murine model of Acinetobacter baumannii infection. Proceedings of the National Academy of Sciences. 2017 Nov. 28; 114 (48): 12669-74.

### REFERENCE LIST FOR EXAMPLE 2

1. D. Galaris, A. et al., Iron homeostasis and oxidative stress: An intimate relationship. Biochim. Biophys. Acta Mol. Cell Res. 1866, 118535 (2019).
2. N. Kim, et al., Redox-active metal ions and amyloid-degrading enzymes in Alzheimer's disease. Int. J. Mol. Sci. 22, 7697 (2021).
3. E. L. Que, et al., Metals in neurobiology: Probing their chemistry and biology with molecular imaging. Chem. Rev. 108, 1517-1549 (2008).
4. K. P. Carter, et al., Fluorescent sensors for measuring metal ions in living systems. Chem. Rev. 114, 4564 4601 (2014).
5. J. M. Braughler, et al., The involvement of iron in lipid peroxidation. Importance of ferric to ferrous ratios in initiation. J. Biol. Chem. 261, 10282-10289 (1986).
6. S. J. Dixon, et al., Ferroptosis: An iron-dependent form of nonapoptotic cell death. Cell 149, 1060-1072 (2012).
7. B. R. Stockwell, et al., Ferroptosis: A regulated cell death nexus linking metabolism, redox biology, and disease. Cell 171, 273-285 (2017).
8. A. Ashraf, et al., Iron dyshomeostasis, lipid peroxidation and perturbed expression of cystine/glutamate antiporter in Alzheimer's disease: Evidence of ferroptosis. Redox Biol. 32, 101494 (2020).
9. N. Yan, et al., Iron metabolism, ferroptosis, and the links with Alzheimer's disease. Front. Neurosci. 13, 1443 (2020).
10. Z. Shen, et al., Emerging strategies of cancer therapy based on ferroptosis. Adv. Mater. 30, e1704007 (2018).
11. T. Xu, et al., Molecular mechanisms of ferroptosis and its role in cancer therapy. J. Cell. Mol. Med. 23, 4900-4912 (2019).
12. A. Spolaor, et al., Determination of Fe²⁺ and Fe³⁺ species by FIA-CRC-ICP-MS in Antarctic ice samples. J. Anal. At. Spectrom. 27, 310-317 (2012).
13. Q. Hu, Simultaneous separation and quantification of iron and transition species using LC-ICP-MS. Am. J. Anal. Chem. 2, 675-682 (2011).
14. N. Mironova-Ulmane, et al., “The Optical And Epr Spectra of Fe²⁺ and Fe³⁺ Ions in the Blood of the Chernobyl Clean-Up Worker” in World Congress on Medical Physics and Biomedical Engineering 2006, R. Magjarevic, J. H. Nagel, Eds. (Springer, 2007), IFMBE Proceedings, pp. 2096-2098.
15. S. Sasaki, Fe²⁺ and Fe³⁺ ions distinguishable by x-ray anomalous scattering: Method and its application to magnetite. Rev. Sci. Instrum. 66, 1573 (1995).
16. R. J. Ogg, et al., The correlation between phase shifts in gradient-echo MR images and regional brain iron concentration. Magn. Reson. Imaging 17, 1141-1148 (1999).
17. O. Dietrich, et al., MR imaging differentiation of Fe²⁺ and Fe³⁺ based on relaxation and magnetic susceptibility properties. Neuroradiology 59, 403-409 (2017).
18. L. H. P. Vroegindeweij, et al., Quantification of different iron forms in the aceruloplasminemia brain to explore iron-related neurodegeneration. NeuroImage Clin. 30, 102657 (2021).
19. W. Breuer, et al., Iron acquired from transferrin by K562 cells is delivered into a cytoplasmic pool of chelatable iron (II). J. Biol. Chem. 270, 24209-24215 (1995).
20. O. Kakhlon, et al., The labile iron pool: Characterization, measurement, and participation in cellular processes1 1This article is part of a series of reviews on “Iron and Cellular Redox Status.” The full list of papers may be found on the homepage of the journal. Free Radic. Biol. Med. 33, 1037-1046 (2002).
21. M. Kruszewski, Labile iron pool: The main determinant of cellular response to oxidative stress. Mutat. Res. 531, 81-92 (2003).
22. M. A. Smith, et al., Iron accumulation in Alzheimer disease is a source of redox-generated free radicals. Proc. Natl. Acad. Sci. U.S.A. 94, 9866-9868 (1997).
23. H. Y. Au-Yeung, J et al., Molecular imaging of labile iron (II) pools in living cells with a turn-on fluorescent probe. J. Am. Chem. Soc. 135, 15165-15173 (2013).
24. A. T. Aron, et al., An endoperoxide reactivity-based FRET probe for ratiometric fluorescence imaging of labile iron pools in living cells. J. Am. Chem. Soc. 138, 14338-14346 (2016).
25. T. Hirayama, et al., A universal fluorogenic switch for Fe(II) ion based on N-oxide chemistry permits the visualization of intracellular redox equilibrium shift towards labile iron in hypoxic tumor cells. Chem. Sci. 8, 4858-4866 (2017).
26. T. Hirayama, et al., High-throughput screening for the discovery of iron homeostasis modulators using an extremely sensitive fluorescent probe. ACS Sens. 5, 2950-2958 (2020).
27. A. T. Aron, et al., In vivo bioluminescence imaging of labile iron accumulation in a murine model of Acinetobacter baumannii infection. Proc. Natl. Acad. Sci. U.S.A. 114, 12669-12674 (2017).
28. R. K. Muir, N et al., Measuring dynamic changes in the labile iron pool in vivo with a reactivity-based probe for positron emission tomography. ACS Cent. Sci. 5, 727-736 (2019).
29. T. Hirayama, et al., Organelle-specific analysis of labile Fe(II) during ferroptosis by using a cocktail of various colour organelle-targeted fluorescent probes. Metallomics 11, 111-117 (2019).
30. R. R. Breaker, et al., A DNA enzyme that cleaves RNA. Chem. Biol. 1, 223-229 (1994).
31. Y. Li, et al., Deoxyribozymes: New players in the ancient game of biocatalysis. Curr. Opin. Struct. Biol. 9, 315-323 (1999).
32. J. Li, et al., In vitro selection and characterization of a highly efficient Zn(II)-dependent RNA-cleaving deoxyribozyme. Nucleic Acids Res. 28, 481-488 (2000).
33. Y. Lu, New transition metal-dependent DNAzymes as efficient endonucleases and as selective metal biosensors. Chemistry 8, 4588-4596 (2002).
34. M. Cepeda-Plaza, et al., Metal ion as both a cofactor and a probe of metal-binding sites in a uranyl-specific DNAzyme: A uranyl photocleavage study. Nucleic Acids Res. 41, 9361-9370 (2013).
35. K. Hwang, et al., Biochemical and biophysical understanding of metal ion selectivity of DNAzymes. Inorganica Chim. Acta. 452, 12-24 (2016).
36. R. J. Lake, et al., DNAzymes as activity-based sensors for metal ions: Recent applications, demonstrated advantages, current challenges, and future directions. Acc. Chem. Res. 52, 3275-3286 (2019).
37. H. E. Ihms, et al., “In vitro Selection of Metal Ion-Selective DNAzymes” in Ribozymes. Methods in Molecular Biology, J. S. Hartig, Ed. (Humana Press, 2012), vol. 848, pp. 297-316.
38. J. Li, et al., A highly sensitive and selective catalytic DNA biosensor for lead ions. J. Am. Chem. Soc. 122, 10466-10467 (2000).
39. J. Liu, et al., Improving fluorescent DNAzyme biosensors by combining inter- and intramolecular quenchers. Anal. Chem. 75, 6666-6672 (2003).
40. J. Liu, et al., A catalytic beacon sensor for uranium with parts-per-trillion sensitivity and millionfold selectivity. Proc. Natl. Acad. Sci. U.S.A. 104, 2056-2061 (2007).
41. J. Liu, et al., A DNAzyme catalytic beacon sensor for paramagnetic Cu²⁺ ions in aqueous solution with high sensitivity and selectivity. J. Am. Chem. Soc. 129, 9838-9839 (2007).
42. J. Liu, et al., Rational design of “turn-on” allosteric DNAzyme catalytic beacons for aqueous mercury ions with ultrahigh sensitivity and selectivity. Angew. Chem. Int. Ed. Engl. 46, 7587-7590 (2007).
43. T. Lan, et al., A highly selective lead sensor based on a classic lead DNAzyme. Chem. Commun. (Camb) 46, 3896-3898 (2010).
44. C. E. McGhee, et al., DNAzyme sensors for detection of metal ions in the environment and imaging them in living cells. Curr. Opin. Biotechnol. 45, 191-201 (2017).
45. Z. Yang, et al., Optical control of metal ion probes in cells and zebrafish using highly selective DNAzymes conjugated to upconversion nanoparticles. J. Am. Chem. Soc. 140, 17656-17665 (2018).
46. Y. Lin, et al., Enzyme-mediated endogenous and bioorthogonal control of a DNAzyme fluorescent sensor for imaging metal ions in living cells. Angew. Chem. Int. Ed. Engl. 58, 17061-17067 (2019).
47. E. M. McConnell, et al., Biosensing with DNAzymes. Chem. Soc. Rev. 50, 8954-8994 (2021).
48. S. Xing, et al., Detection and quantification of tightly bound Zn²⁺ in blood serum using a photocaged chelator and a DNAzyme fluorescent sensor. Anal. Chem. 93, 5856-5861 (2021).
49. W. Zhou, et al., In vitro selection of chromium-dependent DNAzymes for sensing chromium (III) and chromium (VI). Chemistry 22, 9835-9840 (2016).
50. W. Zhou, et al., An exceptionally selective DNA cooperatively binding two Ca²⁺ ions. Chembiochem. 18, 518-522 (2017).
51. P. J. J. Huang, et al., Rational evolution of Cd²⁺-specific DNAzymes with phosphorothioate modified cleavage junction and Cd²⁺ sensing. Nucleic Acids Res. 43, 6125-6133 (2015).
52. P. Bruesehoff, et al., Improving metal ion specificity during in vitro selection of catalytic DNA. Comb. Chem. High Throughput Screen. 5, 327-335 (2002).
53. N. Carmi et al., In vitro selection of self-cleaving DNAs. Chem. Biol. 3, 1039-1046 (1996).
54. S. W. Santoro, et al., A general purpose RNA-cleaving DNA enzyme. Proc. Natl. Acad. Sci. U.S.A. 94, 4262-4266 (1997).
55. S. W. Santoro, et al., RNA cleavage by a DNA enzyme with extended chemical functionality. J. Am. Chem. Soc. 122, 2433-2439 (2000).
56. K. E. Nelson, et al., In vitro selection of high temperature Zn²⁺-dependent DNAzymes. J. Mol. Evol. 61, 216-225 (2005).
57. R. Saran, et al., A silver DNAzyme. Anal. Chem. 88, 4014-4020 (2016).
58. C. E. McGhee, et al., DNAzyme-based lithium-selective imaging reveals higher lithium accumulation in bipolar disorder patient-derived neurons. ACS Cent. Sci. 7, 1809-1820 (2021).
59. S.-F. Torabi, et al., In vitro selection of a sodium-specific DNAzyme and its application in intracellular sensing. Proc. Natl. Acad. Sci. U.S.A. 112, 5903-5908 (2015).
60. S.-F. Torabi, et al., Identification of the same Na⁺-specific DNAzyme motif from two in vitro selections under different conditions. J. Mol. Evol. 81, 225-234 (2015).
61. D. J. R. Lane, et al., Cellular iron uptake, trafficking and metabolism: Key molecules and mechanisms and their roles in disease. Biochim. Biophys. Acta 1853, 1130-1144 (2015).
62. D. Richardson, et al., The effect of the iron (III) chelator, desferrioxamine, on iron and transferrin uptake by the human malignant melanoma cell. Cancer Res. 54, 685-689 (1994).
63. R. C. Hider, et al., Iron speciation in the cytosol: An overview. Dalton Trans. 42, 3220-3229 (2013).
64. R. S. Ohgami, D et al., The Steap proteins are metalloreductases. Blood 108, 1388-1394 (2006).
65. K. Pantopoulos, et al., Mechanisms of mammalian iron homeostasis. Biochemistry 51, 5705-5724 (2012).
66. Q.-Q. Zhao, et al., N/P ratio significantly influences the transfection efficiency and cytotoxicity of a polyethylenimine/chitosan/DNA complex. Biol. Pharm. Bull. 32, 706-710 (2009).
67. P. A. Longo, et al., Transient mammalian cell transfection with polyethylenimine (PEI). Methods Enzymol. 529, 227-240 (2013).
68. R. V. Benjaminsen, et al., The possible “proton sponge” effect of polyethylenimine (PEI) does not include change in lysosomal pH. Mol. Ther. 21, 149-157 (2013).
69. B. Chazotte, Labeling lysosomes in live cells with LysoTracker. Cold Spring Harb. Protoc. 2011, pdb.prot5571 (2011).
70. FerroOrange Labile ferrous ion detecting probe | Goryo Chemical Inc. FerroOrange Labile Ferr. Ion Detect. Probe Goryo Chem. Inc.; https://goryochemical.com/en/research-reagent/4703/.
71. H. Feng, et al., Unsolved mysteries: How does lipid peroxidation cause ferroptosis? PLOS Biol. 16, e2006203 (2018).
72. W. S. Yang, et al., Synthetic lethal screening identifies compounds activating iron-dependent, nonapoptotic cell death in oncogenic-RAS-harboring cancer cells. Chem. Biol. 15, 234-245 (2008).
73. R. Shintoku, et al., Lipoxygenase-mediated generation of lipid peroxides enhances ferroptosis induced by erastin and RSL3. Cancer Sci. 108, 2187-2194 (2017).
74. X. Sui, et al., RSL3 drives ferroptosis through GPX4 inactivation and ROS production in colorectal cancer. Front. Pharmacol. 9, 1371 (2018).
75. T. Mosmann, Rapid colorimetric assay for cellular growth and survival: Application to proliferation and cytotoxicity assays. J. Immunol. Methods 65, 55-63 (1983).
76. J.-L. Liu, et al., Iron and Alzheimer's disease: From pathogenesis to therapeutic implications. Front. Neurosci. 12, 632 (2018).
77. H. Oakley, et al., Intraneuronal β-amyloid aggregates, neurodegeneration, and neuron loss in transgenic mice with five familial Alzheimer's disease mutations: potential factors in amyloid plaque formation. J. Neurosci. 26, 10129-10140 (2006).
78. J. H. Roh, et al., Sleep-wake cycle and diurnal fluctuation of amyloid-β as biomarkers of brain amyloid pathology. Sci. Transl. Med. 4, 150ra122 (2012).
79. D. G. Smith, et al., The redox chemistry of the Alzheimer's disease amyloid β peptide. Biochim. Biophys. Acta 1768, 1976-1990 (2007).
80. J. Everett, et al., Evidence of redox-active iron formation following aggregation of ferrihydrite and the Alzheimer's disease peptide β-amyloid. Inorg. Chem. 53, 2803-2809 (2014).
81. J. Everett, et al., Ferrous iron formation following the co-aggregation of ferric iron and the Alzheimer's disease peptide β-amyloid (1-42). J. R. Soc. Interface 11, 20140165 (2014).
82. M. Gao, et al., Ferroptosis is an autophagic cell death process. Cell Res. 26, 1021-1032 (2016).
83. G. O. Latunde-Dada, Ferroptosis: Role of lipid peroxidation, iron and ferritinophagy. Biochim. Biophys. Acta Gen. Subj. 1861, 1893-1900 (2017).
84. F. Kuang, et al., Oxidative damage and antioxidant defense in ferroptosis. Front. Cell Dev. Biol. 8, 586578 (2020).
85. J. M. G. van Bergen, et al., Colocalization of cerebral iron with amyloid β in mild cognitive impairment. Sci. Rep. 6, 35514 (2016).
86. N. D. Telling, et al., Iron biochemistry is correlated with amyloid plaque morphology in an established mouse model of Alzheimer's disease. Cell Chem. Biol. 24, 1205-1215.e3 (2017).
87. J. Everett, et al., Iron stored in ferritin is chemically reduced in the presence of aggregating Aβ (1-42). Sci. Rep. 10, 10332 (2020).
88. C. A. Schneider, et al., NIH Image to ImageJ: 25 years of image analysis. Nat. Methods 9, 671-675 (2012).
89. S. Bolte, et al., A guided tour into subcellular colocalization analysis in light microscopy. J. Microsc. 224, 213-232 (2006).
90. N. R. Markham, et al., UNAFold: Software for nucleic acid folding and hybridization. Methods Mol. Biol. 453, 3-31 (2008).
91. S. W. Taylor, et al., Ferric ion complexes of a DOPA-containing adhesive protein from Mytilus edulis. Inorg. Chem. 35, 7572-7577 (1996).
92. D. S. Hwang, et al., Protein- and metal-dependent interactions of a prominent protein in mussel adhesive plaques. J. Biol. Chem. 285, 25850-25858 (2010).
93. H. Zeng, et al., Strong reversible Fe³⁺-mediated bridging between dopa-containing protein films in water. Proc. Natl. Acad. Sci. U.S.A. 107, 12850-12853 (2010).

### REFERENCE LIST FOR EXAMPLE 3

1 Dixon, S. J., et al., Ferroptosis: an iron-dependent form of nonapoptotic cell death. Cell 149, 1060, doi:10.1016/j.cell.2012.03.042 (2012).
2 Stockwell, B. R. Ferroptosis turns 10: Emerging mechanisms, physiological functions, and therapeutic applications. Cell 185, 2401, doi:10.1016/j.cell.2022.06.003 (2022).
3 Jiang, X., et al., Ferroptosis: mechanisms, biology and role in disease. Nat. Rev. Mol. Cell Biol. 22, 266, doi:10.1038/s41580-020-00324-8 (2021).
4 Dixon, S. J. & Stockwell, B. R. The Hallmarks of Ferroptosis. 3, 35, doi:10.1146/annurev-cancerbio-030518-055844 (2019).
5 Chen, X., et al., Iron metabolism in ferroptosis. Front Cell Dev. Biol. 8, 590226, doi:10.3389/fcell.2020.590226 (2020).
6 Igney, F. H. & Krammer, P. H. Death and anti-death: tumour resistance to apoptosis. Nat. Rev. Cancer 2, 277, doi:10.1038/nrc776 (2002).
7 Sweeny, L., et al., A novel extracellular drug conjugate significantly inhibits head and neck squamous cell carcinoma. Oral Oncol. 49, 991, doi:10.1016/j.oraloncology.2013.07.006 (2013).
8 Pelaz, B., et al., Diverse applications of nanomedicine. ACS Nano 11, 2313, doi:10.1021/acsnano.6b06040 (2017).
9 Luo, D., et al., Nanoparticles yield increased drug uptake and therapeutic efficacy upon sequential near-infrared irradiation. ACS Nano 14, 15193, doi:10.1021/acsnano.0c05425 (2020).
10 Jiang, D. W., et al., Nanomedicines for renal management: From imaging to treatment. Acc. Chem. Res. 53, 1869, doi:10.1021/acs.accounts.0c00323 (2020).
11 Doll, S., et al., ACSL4 dictates ferroptosis sensitivity by shaping cellular lipid composition. Nat. Chem. Biol. 13, 91, doi:10.1038/nchembio.2239 (2017).
12 Lei, G., et al., Targeting ferroptosis as a vulnerability in cancer. Nat. Rev. Cancer, doi:10.1038/s41568-022-00459-0 (2022).
13 Roshanzamir, F., et al., Metastatic triple negative breast cancer adapts its metabolism to destination tissues while retaining key metabolic signatures. Proc. Natl. Acad. Sci. U.S.A. 119, e2205456119, doi:10.1073/pnas.2205456119 (2022).
14 Hangauer, M. J., et al., Drug-tolerant persister cancer cells are vulnerable to GPX4 inhibition. Nature 551, 247, doi:10.1038/nature24297 (2017).
15 Viswanathan, V. S., et al., Dependency of a therapy-resistant state of cancer cells on a lipid peroxidase pathway. Nature 547, 453, doi:10.1038/nature23007 (2017).
16 Lei, G., et al., The role of ferroptosis in ionizing radiation-induced cell death and tumor suppression. Cell Res. 30, 146, doi:10.1038/s41422-019-0263-3 (2020).
17 Wang, W., et al., CD8⁺ T cells regulate tumour ferroptosis during cancer immunotherapy. Nature 569, 270, doi:10.1038/s41586-019-1170-y (2019).
18 Guo, J., et al., Ferroptosis: A Novel Anti-tumor Action for Cisplatin. Cancer research and treatment 50, 445, doi:10.4143/crt.2016.572 (2018).
19 Sun, X., et al., Activation of the p62-Keap1-NRF2 pathway protects against ferroptosis in hepatocellular carcinoma cells. Hepatology 63, 173, doi:10.1002/hep.28251 (2016).
20 Jiang, Z., et al., TYRO3 induces anti-PD-1/PD-L1 therapy resistance by limiting innate immunity and tumoral ferroptosis. J. Clin. Invest. 131, doi:10.1172/JCI139434 (2021).
21 Sun, X., et al., Metallothionein-1G facilitates sorafenib resistance through inhibition of ferroptosis. Hepatology 64 (2016).
22 Dang, Q., et al., Ferroptosis: a double-edged sword mediating immune tolerance of cancer. Cell Death Dis. 13, 925, doi:10.1038/s41419-022-05384-6 (2022).
23 Xu, C., et al., The glutathione peroxidase Gpx4 prevents lipid peroxidation and ferroptosis to sustain Treg cell activation and suppression of antitumor immunity. Cell Reports 35, doi:10.1016/j.celrep.2021.109235 (2021).
24 Dixon, S. J. & Stockwell, B. R. The Hallmarks of Ferroptosis. Annual Review of Cancer Biology 3, 35, doi:10.1146/annurev-cancerbio-030518-055844 (2019).
25 Chen, X., et al., Characteristics and Biomarkers of Ferroptosis. Front Cell Dev Biol 9, 637162, doi:10.3389/fcell.2021.637162 (2021).
26 Dixon, Scott J., et al., Ferroptosis: An Iron-Dependent Form of Nonapoptotic Cell Death. Cell 149, 1060, doi:10.1016/j.cell.2012.03.042 (2012).
27 Al-Khabori, M., et al., Side effects of Deferasirox Iron Chelation in Patients with Beta Thalassemia Major or Intermedia. Oman medical journal 28, 121, doi:10.5001/omj.2013.31 (2013).
28 Stoyanovsky, D. A., et al., Iron catalysis of lipid peroxidation in ferroptosis: Regulated enzymatic or random free radical reaction? Free Radic. Biol. Med. 133, 153, doi:10.1016/j.freeradbiomed.2018.09.008 (2019).
29 Kagan, V. E., et al., Cytochrome c/cardiolipin relations in mitochondria: a kiss of death. Free Radic. Biol. Med. 46, 1439, doi:10.1016/j.freeradbiomed.2009.03.004 (2009).
30 Kim, Y.-M., et al., Cellular Non-heme Iron Content Is a Determinant of Nitric Oxide-mediated Apoptosis, Necrosis, and Caspase Inhibition *. J. Biol. Chem. 275, 10954, doi:https://doi.org/10.1074/jbc.275.15.10954 (2000).
31 Morgan, M. J., et al., TNFα and reactive oxygen species in necrotic cell death. Cell Res. 18, 343, doi:10.1038/cr.2008.31 (2008).
32 Doll, S. & Conrad, M. Iron and ferroptosis: A still ill-defined liaison. IUBMB Life 69, 423, doi:10.1002/iub.1616 (2017).
33 Kakhlon, O. & Cabantchik, Z. I. The labile iron pool: characterization, measurement, and participation in cellular processes1 1This article is part of a series of reviews on “Iron and cellular redox status.” The full list of papers may be found on the homepage of the journal. Free Radic. Biol. Med. 33, 1037, doi:10.1016/s0891-5849 (02) 01006-7 (2002).
34 Mancias, J. D., et al., Quantitative proteomics identifies NCOA4 as the cargo receptor mediating ferritinophagy. Nature 509, 105, doi:10.1038/nature13148 (2014).
35 Lane, D. J. R., et al., Cellular iron uptake, trafficking and metabolism: Key molecules and mechanisms and their roles in disease. Biochimica et Biophysica Acta (BBA)—Molecular Cell Research 1853, 1130, doi:https://doi.org/10.1016/j.bbamcr.2015.01.021 (2015).
36 Braughler, J. M., et al., The involvement of iron in lipid peroxidation. Importance of ferric to ferrous ratios in initiation. J. Biol. Chem. 261, 10282, doi:https://doi.org/10.1016/S0021-9258 (18) 67521-0 (1986).
37 Braughler, J. M., et al., The involvement of iron in lipid peroxidation. Importance of ferric to ferrous ratios in initiation. J. Biol. Chem. 261, 10282, doi:https://doi.org/10.1016/S0021-9258 (18) 67521-0 (1986).
38 Wang, Q., et al., Degradation mechanism of methylene blue in a heterogeneous Fenton-like reaction catalyzed by ferrocene. Ind. Eng. Chem. Res. 53, 643, doi:10.1021/ie403402q (2014).
39 Wang, S., et al., Iron and magnetic: new research direction of the ferroptosis-based cancer therapy. Am J Cancer Res 8, 1933 (2018).
40 Minotti, G. & Aust, S. D. Redox cycling of iron and lipid peroxidation. Lipids 27, 219, doi:10.1007/BF02536182 (1992).
41 Morini, S., et al., 283 edn 207 (International Society for Horticultural Science (ISHS), Leuven, Belgium).
42 Kukiełka, E. & Cederbaum, A. I. NADPH- and NADH-dependent oxygen radical generation by rat liver nuclei in the presence of redox cycling agents and iron. Arch. Biochem. Biophys. 283, 326, doi:https://doi.org/10.1016/0003-9861 (90) 90650-N (1990).
43 Bucher, J. R., et al., The requirement for ferric in the initiation of lipid peroxidation by chelated ferrous iron. Biochem. Biophys. Res. Commun. 111, 777, doi:https://doi.org/10.1016/0006-291X (83) 91366-9 (1983). 44 Braughler, J. M., et al., Oxidation of ferrous iron during peroxidation of lipid substrates. Biochimica et Biophysica Acta (BBA)—Lipids and Lipid Metabolism 921, 457, doi:https://doi.org/10.1016/0005-2760 (87) 90072-5 (1987).
45 Minotti, G. & Aust, S. D. An investigation into the mechanism of citrate· FE²⁺-dependent lipid peroxidation. Free Radic. Biol. Med. 3, 379, doi:https://doi.org/10.1016/0891-5849 (87) 90016-5 (1987).
46 Braughler, J. M., et al., Novel 21-amino steroids as potent inhibitors of iron-dependent lipid peroxidation. J. Biol. Chem. 262, 10438, doi:10.1016/S0021-9258 (18) 60979-2 (1987).
47 Minotti, G. & Aust, S. D. The requirement for iron (III) in the initiation of lipid peroxidation by iron (II) and hydrogen peroxide. J. Biol. Chem. 262, 1098, doi:https://doi.org/10.1016/S0021-9258 (19) 75755-X (1987).
48 Ming Ko, K. & Godin, D. V. Ferric ion-induced lipid peroxidation in erythrocyte membranes: effects of phytic acid and butylated hydroxytoluene. Mol. Cell. Biochem. 95, 125, doi:10.1007/BF00219970 (1990).
49 Minotti, G. & Aust, S. D. The role of iron in the initiation of lipid peroxidation. Chem. Phys. Lipids 44, 191, doi:10.1016/0009-3084 (87) 90050-8 (1987).
50 Mozuraityte, R., et al., The role of iron in peroxidation of polyunsaturated fatty acids in liposomes. J. Agric. Food Chem. 56, 537, doi:10.1021/jf0716073 (2008).
51 Zhang, L., et al., An adenosine triphosphate-responsive autocatalytic Fenton nanoparticle for tumor Ablation with Self-Supplied H₂O₂ and Acceleration of Fe(III)/Fe(II) Conversion. Nano Lett. 18, 7609, doi:10.1021/acs.nanolett.8b03178 (2018).
52 Chen, L., et al., Fe (2+)/Fe (3+) ions chelated with ultrasmall polydopamine nanoparticles induce ferroptosis for cancer therapy. ACS Biomater. Sci. Eng. 5, 4861, doi:10.1021/acsbiomaterials.9b00461 (2019).
53 He, Y. J., et al., Fenton reaction-independent ferroptosis therapy via glutathione and iron redox couple sequentially triggered lipid peroxide generator. Biomaterials 241, 119911, doi:10.1016/j.biomaterials.2020.119911 (2020).
54 Yang, Y., et al., Iron-doxorubicin prodrug loaded liposome nanogenerator programs multimodal ferroptosis for efficient cancer therapy. Asian J. Pharm. Sci. 16, 784, doi:10.1016/j.ajps.2021.05.001 (2021).
55 Gaschler, M. M., et al., FINO2 initiates ferroptosis through GPX4 inactivation and iron oxidation. Nat. Chem. Biol. 14, 507, doi:10.1038/s41589-018-0031-6 (2018).
56 von Krusenstiern, A. N., et al., Identification of essential sites of lipid peroxidation in ferroptosis. Nat. Chem. Biol. 19, 719, doi:10.1038/s41589-022-01249-3 (2023).
57 Minotti, G. & Aust, S. D. Redox cycling of iron and lipid peroxidation. Lipids 27, 219, doi:10.1007/BF02536182 (1992).
58 Dorfman, L. M. & Adams, G. E. Reactivity of the Hydroxyl Radical in Aqueous Solutions. (1973).
59 Armenta, D. A., et al., Ferroptosis inhibition by lysosome-dependent catabolism of extracellular protein. Cell Chemical Biology 29, 1588, doi:10.1016/j.chembiol.2022.10.006 (2022).
60 Shvartsman, M. & Ioav Cabantchik, Z. Intracellular iron trafficking: role of cytosolic ligands. Biometals 25, 711, doi:10.1007/s10534-012-9529-7 (2012).
61 Hirayama, T., et al., Organelle-specific analysis of labile Fe (ii) during ferroptosis by using a cocktail of various colour organelle-targeted fluorescent probes. Metallomics 11, 111, doi:10.1039/C8MT00212F (2019).
62 Paradkar, P. N., et al., Regulation of Mitochondrial Iron Import through Differential Turnover of Mitoferrin 1 and Mitoferrin 2. Mol. Cell. Biol. 29, 1007, doi:10.1128/MCB.01685-08 (2009).
63 Wu, Y., et al., Simultaneous Fe (2+)/Fe (3+) imaging shows Fe (3+) over Fe (2+) enrichment in Alzheimer's disease mouse brain. Sci Adv 9, eade7622, doi:10.1126/sciadv.ade7622 (2023).
64 Mironova-Ulmane, N., et al. in World Congress on Medical Physics and Biomedical Engineering 2006. (eds R. Magjarevic & J. H. Nagel) 2096 (Springer Berlin Heidelberg).
65 Sasaki, S. J. R. o. S. I. Fe²⁺ and Fe³⁺ ions distinguishable by x-ray anomalous scattering: Method and its application to magnetite. 66, 1573 (1995).
66 Ogg, R. J., et al., The correlation between phase shifts in gradient-echo MR images and regional brain iron concentration. Magn. Reson. Imaging 17, 1141, doi: Doi 10.1016/S0730-725x (99) 00017-X (1999).
67 Dietrich, O., et al., MR imaging differentiation of Fe²⁺ and Fe³⁺ based on relaxation and magnetic susceptibility properties. Neuroradiology 59, 403, doi:10.1007/s00234-017-1813-3 (2017).
68 Vroegindeweij, L. H. P., et al., Quantification of different iron forms in the aceruloplasminemia brain to explore iron-related neurodegeneration. NeuroImage: Clinical 30, 102657, doi:https://doi.org/10.1016/j.nicl.2021.102657 (2021).
69 Kruszewski, M. Labile iron pool: the main determinant of cellular response to oxidative stress. Mutat. Res. 531, 81, doi:10.1016/j.mrfmmm.2003.08.004 (2003).
70 van Rooden, S., et al., Cortical phase changes in Alzheimer's disease at 7T MRI: a novel imaging marker. Alzheimers Dement 10, e19, doi:10.1016/j.jalz.2013.02.002 (2014).
71 Cabantchik, Z. I. Labile iron in cells and body fluids: physiology, pathology, and pharmacology. Front Pharmacol 5, 45, doi:10.3389/fphar.2014.00045 (2014).
72 Zeineh, M. M., et al., Activated iron-containing microglia in the human hippocampus identified by magnetic resonance imaging in Alzheimer disease. Neurobiol. Aging 36, 2483, doi:10.1016/j.neurobiolaging.2015.05.022 (2015).
73 Krummel, M., et al., Visualizing Cancer. Cancer Cell 38, 753, doi:10.1016/j.ccell.2020.11.014 (2020).
74 Rowe, S. P. & Pomper, M. G. Molecular imaging in oncology: Current impact and future directions. Ca-a Cancer Journal for Clinicians, doi:10.3322/caac.21713 (2021).
75 Epsztejn, S., et al., Fluorescence analysis of the labile iron pool of mammalian cells. Anal. Biochem. 248, 31, doi:10.1006/abio. 1997.2126 (1997).
76 Gao, M., et al., Ferroptosis is an autophagic cell death process. Cell Res. 26, 1021, doi:10.1038/cr.2016.95 (2016).
77 Wei, Y., et al., A turn-on fluorescent sensor for imaging labile Fe (3+) in live neuronal cells at subcellular resolution. Chembiochem 13, 1569, doi:10.1002/cbic.201200202 (2012).
78 OuYang, H., et al., A highly selective rhodamine-based optical-electrochemical multichannel chemosensor for Fe³⁺. Tetrahedron Lett. 54, 2964, doi:10.1016/j.tetlet.2013.03.112 (2013).
79 Au-Yeung, H. Y., et al., Molecular imaging of labile iron (II) pools in living cells with a turn-on fluorescent probe. J. Am. Chem. Soc. 135, 15165, doi:10.1021/ja4072964 (2013).
80 Spangler, B., et al., A reactivity-based probe of the intracellular labile ferrous iron pool. Nat. Chem. Biol. 12, 680, doi:10.1038/nchembio.2116 (2016).
81 Hirayama, T., et al., A universal fluorogenic switch for Fe (ii) ion based on N-oxide chemistry permits the visualization of intracellular redox equilibrium shift towards labile iron in hypoxic tumor cells. Chem. Sci. 8, 4858, doi:10.1039/c6sc05457a (2017).
82 Aron, A. T., et al., Activity-based sensing fluorescent probes for iron in biological systems. Curr. Opin. Chem. Biol. 43, 113, doi:10.1016/j.cbpa.2017.12.010 (2018).
83 Sahoo, S. K. & Crisponi, G. Recent advances on iron (III) selective fluorescent probes with possible applications in bioimaging. Molecules 24, doi:10.3390/molecules24183267 (2019).
84 Hirayama, T., et al., High-throughput screening for the discovery of iron homeostasis modulators using an extremely sensitive fluorescent probe. ACS Sens. 5, 2950, doi:10.1021/acssensors.0c01445 (2020).
85 Kemlo, J. A. & Shepherd, T. M. Quenching of excited singlet states by metal ions. Chem. Phys. Lett. 47, 158, doi:https://doi.org/10.1016/0009-2614 (77) 85329-3 (1977).
86 Varnes, A. W., et al., Interactions of transition-metal ions with photoexcited states of flavines. Fluorescence quenching studies. J. Am. Chem. Soc. 94, 946, doi:10.1021/ja00758a037 (1972).
87 Breaker, R. R. & Joyce, G. F. A DNA enzyme that cleaves RNA. Chem. Biol. 1, 223, doi:10.1016/1074-5521 (94) 90014-0 (1994).
88 Li, Y. & Breaker, R. R. Deoxyribozymes: new players in the ancient game of biocatalysis. Curr. Opin. Struct. Biol. 9, 315, doi:10.1016/S0959-440X (99) 80042-6 (1999).
89 Lu, Y. New Transition-Metal-Dependent DNAzymes as Efficient Endonucleases and as Selective Metal Biosensors. Chemistry-A European Journal 8, 4588, doi:10.1002/1521-3765 (20021018) 8:20<4588::Aid-chem4588>3.0.Co; 2-q (2002).
90 Baum, D. A. & Silverman, S. K. Deoxyribozymes: useful DNA catalysts in vitro and in vivo. Cell. Mol. Life Sci. 65, 2156, doi:10.1007/s00018-008-8029-y (2008).
91 Santoro, S. W. & Joyce, G. F. A general purpose RNA-cleaving DNA enzyme. Proc. Natl Acad. Sci. USA 94, 4262, doi:10.1073/pnas.94.9.4262 (1997).
92 Pan, T. & Uhlenbeck, O. C. In vitro selection of RNAs that undergo autolytic cleavage with Pb²⁺. Biochemistry 31, 3887, doi:10.1021/bi00131a001 (1992).
93 Geyer, C. R. & Sen, D. Evidence for the metal-cofactor independence of an RNA phosphodiester-cleaving DNA enzyme. Chem. Biol. 4, 579, doi:10.1016/s1074-5521 (97) 90244-1 (1997).
94 Roth, A. & Breaker, R. R. An amino acid as a cofactor for a catalytic polynucleotide. Proc. Natl Acad. Sci. USA 95, 6027, doi:10.1073/pnas.95.11.6027 (1998).
95 May, J. P., et al., Covalent Schiff base catalysis and turnover by a DNAzyme: a M²⁺—independent AP-endonuclease mimic. J. Am. Chem. Soc. 126, 4145, doi:10.1021/ja037625s (2004).
96 Sidorov, A. V., et al., Sequence-specific cleavage of RNA in the absence of divalent metal ions by a DNAzyme incorporating imidazolyl and amino functionalities. Nucleic Acids Res. 32, 1591, doi:10.1093/nar/gkh326 (2004).
97 Cuenoud, B. & Szostak, J. W. A DNA metalloenzyme with DNA ligase activity. Nature 375, 611, doi:10.1038/375611a0 (1995).
98 Carmi, N., et al., In vitro selection of self-cleaving DNAs. Chem. Biol. 3, 1039, doi:10.1016/s1074-5521 (96) 90170-2 (1996).
99 Liu, J., et al., A catalytic beacon sensor for uranium with parts-per-trillion sensitivity and millionfold selectivity. Proc. Natl Acad. Sci. USA 104, 2056, doi:10.1073/pnas.0607875104 (2007).
100 Torabi, S. F., et al., In vitro selection of a sodium-specific DNAzyme and its application in intracellular sensing. Proc. Natl Acad. Sci. USA 112, 5903, doi:10.1073/pnas. 1420361112 (2015).
101 McGhee, C. E., et al., DNAzyme-based lithium-selective imaging reveals higher lithium accumulation in bipolar disorder patient-derived neurons. ACS Cent. Sci. 7, 1809, doi:10.1021/acscentsci.1c00843 (2021).
102 Fan, H., et al., A Highly Selective Mn(II)-Specific DNAzyme and Its Application in Intracellular Sensing. JACS Au, doi:10.1021/jacsau.3c00062 (2023).
103 Bruesehoff, P. J., et al., Improving metal ion specificity during in vitro selection of catalytic DNA. Comb. Chem. High Throughput Screen. 5, 327, doi:10.2174/1386207023330264 (2002).
104 Li, J. & Lu, Y. A highly sensitive and selective catalytic DNA biosensor for lead ions. J. Am. Chem. Soc. 122, 10466, doi:10.1021/ja0021316 (2000).
105 Liu, J. & Lu, Y. Improving fluorescent DNAzyme biosensors by combining inter- and intramolecular quenchers. Anal. Chem. 75, 6666, doi:10.1021/ac034924r (2003).
106 Chaloux, E.; Altmann, E., Lead in Water: St. Paul Schools Delayed Fixes. 2016. https://youtu.be/UdWxFsLsKOc.
107 ANDalyze, Inc. http://andalyze.com/.
108 Hwang, K., et al., Photocaged DNAzymes as a general method for sensing metal ions in living cells. Angew. Chem. Int. Ed. Engl. 53, 13798, doi:10.1002/anie.201408333 (2014).
109 Wang, X., et al., Noninvasive and spatiotemporal control of DNAzyme-based imaging of metal ions in vivo using high-intensity focused ultrasound. J. Am. Chem. Soc. 144, 5812, doi:10.1021/jacs.1c11543 (2022).
110 Modi, S., et al., Two DNA nanomachines map pH changes along intersecting endocytic pathways inside the same cell. Nat. Nanotechnol. 8, 459, doi:10.1038/nnano.2013.92 (2013).
111 Chan, M. S. & Lo, P. K. Nanoneedle-Assisted Delivery of Site-Selective Peptide-Functionalized DNA Nanocages for Targeting Mitochondria and Nuclei. Small 10, 1255, doi:https://doi.org/10.1002/smll.201302993 (2014).
112 Qin, B., et al., Targeting DNA to the endoplasmic reticulum efficiently enhances gene delivery and therapy. Nanoscale 12, 18249, doi:10.1039/DONR03156A (2020).
113 Fujiwara, Y., et al., Direct uptake and degradation of DNA by lysosomes. Autophagy 9, 1167, doi:10.4161/auto.24880 (2013).
114 Chan, M. S. & Lo, P. K. Nanoneedle-Assisted Delivery of Site-Selective Peptide-Functionalized DNA Nanocages for Targeting Mitochondria and Nuclei. 10, 1255, doi:https://doi.org/10.1002/smll.201302993 (2014).
115 Hong, S., et al., A photo-regulated aptamer sensor for spatiotemporally controlled monitoring of ATP in the mitochondria of living cells. Chem Sci 11, 713, doi:10.1039/c9sc04773e (2019).
116 Yuan, X., et al., Virus-like Nonvirus Cationic Liposome for Efficient Gene Delivery via Endoplasmic Reticulum Pathway. ACS Cent Sci 6, 174, doi:10.1021/acscentsci.9b01052 (2020).
117 Battaglia, A. M., et al., Iron Administration Overcomes Resistance to Erastin-Mediated Ferroptosis in Ovarian Cancer Cells. Front Oncol 12, 868351, doi:10.3389/fonc.2022.868351 (2022).
118 Lee, H., et al., Energy-stress-mediated AMPK activation inhibits ferroptosis. Nat. Cell Biol. 22, 225, doi:10.1038/s41556-020-0461-8 (2020).
119 D'Souza, G. G., et al., Mitochondrial leader sequence—plasmid DNA conjugates delivered into mammalian cells by DQAsomes co-localize with mitochondria. Mitochondrion 5, 352, doi:10.1016/j.mito.2005.07.001 (2005).
120 Shu, Z., et al., Disulfide-unit conjugation enables ultrafast cytosolic internalization of antisense DNA and siRNA. Angew. Chem. Int. Ed. Engl. 58, 6611, doi:10.1002/anie.201900993 (2019).
121 Biswas, S. & Torchilin, V. P. Nanopreparations for organelle-specific delivery in cancer. Adv. Drug Deliv. Rev. 66, 26, doi:10.1016/j.addr.2013.11.004 (2014).
122 Saminathan, A., et al., Organelle-level precision with next-generation targeting technologies. Nat. Rev. Mater. 7, 355, doi:10.1038/s41578-021-00396-8 (2021).
123 Wu, P., et al., A DNAzyme-gold nanoparticle probe for uranyl ion in living cells. J. Am. Chem. Soc. 135, 5254, doi:10.1021/ja400150v (2013).
124 Yang, W. S., et al., Regulation of ferroptotic cancer cell death by GPX4. Cell 156, 317, doi:10.1016/j.cell.2013.12.010 (2014).
125 Mao, C., et al., DHODH-mediated ferroptosis defence is a targetable vulnerability in cancer. Nature 593, 586, doi:10.1038/s41586-021-03539-7 (2021).
126 https://www.jax.org/strain/002019.
127 Hong, D., et al., AZD9150, a next-generation antisense oligonucleotide inhibitor of STAT3 with early evidence of clinical activity in lymphoma and lung cancer. Sci. Transl. Med. 7, 314ra185, doi:10.1126/scitranslmed.aac5272 (2015).
128<https://www.fda.gov/drugs/resources-information-approved-drugs/fda-approves-device-treatment-osteoid-osteoma-extremities> (2023).
129 Bouvier-Muller, A. & Duconge, F. Application of aptamers for in vivo molecular imaging and theranostics. Adv Drug Deliv Rev 134, 94, doi:10.1016/j.addr.2018.08.004 (2018).
130 Park, J. Y., et al., Hybridization-based aptamer labeling using complementary oligonucleotide platform for PET and optical imaging. Biomaterials 100, 143, doi:10.1016/j.biomaterials.2016.05.035 (2016).
131 Nakatsuka, M. A., et al., In vivo ultrasound visualization of non-occlusive blood clots with thrombin-sensitive contrast agents. Biomaterials 34, 9559, doi:10.1016/j.biomaterials.2013.08.040 (2013).
132 Hu, H., et al., Aptamer-conjugated Mn3O4@SiO2 core-shell nanoprobes for targeted magnetic resonance imaging. Nanoscale 5, 10447, doi:10.1039/c3nr03490a (2013).
133 Tang, D., et al., Ferroptosis: molecular mechanisms and health implications. Cell Res. 31, 107, doi:10.1038/s41422-020-00441-1 (2021).
134 Dixon, S. J. & Pratt, D. A. Ferroptosis: A flexible constellation of related biochemical mechanisms. Mol. Cell 83, 1030, doi:10.1016/j.molcel.2023.03.005 (2023).
135 Du, Y. & Guo, Z. Recent progress in ferroptosis: inducers and inhibitors. Cell Death Discov 8, 501, doi:10.1038/s41420-022-01297-7 (2022).
136 Skouta, R., et al., Ferrostatins inhibit oxidative lipid damage and cell death in diverse disease models. J. Am. Chem. Soc. 136, 4551, doi:10.1021/ja411006a (2014).
137 Zhao, Y., et al., The role of erastin in ferroptosis and its prospects in cancer therapy. Onco Targets Ther 13, 5429, doi:10.2147/OTT.S254995 (2020).
138 Li, X. Q., et al., NCOA4 is regulated by HIF and mediates mobilization of murine hepatic iron stores after blood loss. Blood 136, 2691, doi:10.1182/blood.2020006321 (2020).
139 Steegmann-Olmedillas, J. L. The role of iron in tumour cell proliferation. Clin. Transl. Oncol. 13, 71, doi:10.1007/s12094-011-0621-1 (2011).
140 Zhang, S., et al., Disordered hepcidin-ferroportin signaling promotes breast cancer growth. Cell. Signal. 26, 2539, doi:10.1016/j.cellsig.2014.07.029 (2014).
141 Greene, C. J., et al., Transferrin receptor 1 upregulation in primary tumor and downregulation in benign kidney is associated with progression and mortality in renal cell carcinoma patients. Oncotarget 8, 107052, doi:10.18632/oncotarget.22323 (2017).
142 Bersuker, K., et al., The CoQ oxidoreductase FSP1 acts parallel to GPX4 to inhibit ferroptosis. Nature 575, 688, doi:10.1038/s41586-019-1705-2 (2019).
143 Joung, J., et al., Genome-scale CRISPR-Cas9 knockout and transcriptional activation screening. Nat. Protoc. 12, 828, doi:10.1038/nprot.2017.016 (2017).
144 Liu, J., et al., Identification of the transcription factor Miz1 as an essential regulator of diphthamide biosynthesis using a CRISPR-mediated genome-wide screen. PLOS Genet. 16, e1009068, doi:10.1371/journal.pgen. 1009068 (2020).
145 Tsvetkov, P., et al., Copper induces cell death by targeting lipoylated TCA cycle proteins. Science 375, 1254, doi:10.1126/science.abf0529 (2022).
146 Hart, T., et al., Evaluation and Design of Genome-Wide CRISPR/SpCas9 Knockout Screens. G3 (Bethesda) 7, 2719, doi:10.1534/g3.117.041277 (2017).
147 Menasche, B. L., et al., Fluorescence activated cell sorting (FACS) in genome-wide genetic screening of membrane trafficking. Curr Protoc Cell Biol 82, e68, doi:10.1002/cpcb.68 (2019).
148 Condon, K. J., et al., Genome-wide CRISPR screens reveal multitiered mechanisms through which mTORC1 senses mitochondrial dysfunction. Proc. Natl Acad. Sci. USA 118, e2022120118, doi:10.1073/pnas.2022120118 (2021).
149 Wang, T., et al., Genetic screens in human cells using the CRISPR-Cas9 system. Science 343, 80, doi:10.1126/science. 1246981 (2014).
150 Yi, S., et al., Functional variomics and network perturbation: connecting genotype to phenotype in cancer. Nat. Rev. Genet. 18, 395, doi:10.1038/nrg.2017.8 (2017).
151 Hou, J., et al., Single-cell CRISPR immune screens reveal immunological roles of tumor intrinsic factors. NAR cancer 4, zcac038, doi:10.1093/narcan/zcac038 (2022).
152 Shin, W., et al., Single-cell transcriptomic mapping of intestinal epithelium that undergoes 3D morphogenesis and mechanodynamic stimulation in a gut-on-a-chip. iScience 25, 105521, doi:10.1016/j.isci.2022.105521 (2022).
153 Li, W., et al., MAGeCK enables robust identification of essential genes from genome-scale CRISPR/Cas9 knockout screens. Genome Biol. 15, 554, doi:10.1186/s13059-014-0554-4 (2014).
154 Tsherniak, A., et al., Defining a cancer dependency map. Cell 170, 564, doi:https://doi.org/10.1016/j.cell.2017.06.010 (2017).
155 Fujimaki, M., et al., Iron supply via NCOA4-mediated ferritin degradation maintains mitochondrial functions. Mol. Cell. Biol. 39, e00010, doi:10.1128/MCB.00010⁻¹⁹ (2019).
156 Friedmann Angeli, J. P., et al., Inactivation of the ferroptosis regulator Gpx4 triggers acute renal failure in mice. Nat. Cell Biol. 16, 1180, doi:10.1038/ncb3064 (2014).
157 Doll, S., et al., FSP1 is a glutathione-independent ferroptosis suppressor. Nature 575, 693, doi:10.1038/s41586-019-1707-0 (2019).
158 Zilka, O., et al., On the mechanism of cytoprotection by ferrostatin-1 and liproxstatin-1 and the role of lipid peroxidation in ferroptotic cell death. ACS Cent. Sci. 3, 232, doi:10.1021/acscentsci.7b00028 (2017).
159 Kim, R., et al., Ferroptosis of tumour neutrophils causes immune suppression in cancer. Nature 612, 338, doi:10.1038/s41586-022-05443-0 (2022).
160 Ponce-Salvatierra, A., et al., Crystal structure of a DNA catalyst. Nature 529, 231, doi:10.1038/nature16471 (2016).
161 Liu, H., et al., Crystal structure of an RNA-cleaving DNAzyme. Nat. Commun. 8, 2006, doi:10.1038/s41467-017-02203-x (2017).
162 Keefe, A. D., et al., Aptamers as therapeutics. Nat. Rev. Drug Discov. 9, 537, doi:10.1038/nrd3141 (2010).
163 Zhou, J. & Rossi, J. Aptamers as targeted therapeutics: current potential and challenges. Nat. Rev. Drug Discov. 16, 181, doi:10.1038/nrd.2016.199 (2017).
164 Hwang, K., et al., Metal-dependent DNAzymes for the quantitative detection of metal ions in living cells: Cecent progress, current challenges, and latest results on FRET ratiometric sensors. Inorg. Chem. 58, 13696, doi:10.1021/acs.inorgchem.9b01280 (2019).
165 Brown, A. K., et al., A lead-dependent DNAzyme with a two-step mechanism. Biochemistry 42, 7152, doi:10.1021/bi027332w (2003).
166 Brown, A. K., et al., Biochemical characterization of a uranyl ion-specific DNAzyme. Chembiochem 10, 486, doi:10.1002/cbic.200800632 (2009).
167 Luecke, S., et al., cGAS is activated by DNA in a length-dependent manner. EMBO Rep. 18, 1707, doi:10.15252/embr.201744017 (2017).
168 Andreeva, L., et al., cGAS senses long and HMGB/TFAM-bound U-turn DNA by forming protein-DNA ladders. Nature 549, 394, doi:10.1038/nature23890 (2017).
169 Zhou, W., et al., Structure of the human cGAS-DNA complex reveals enhanced control of immune surveillance. Cell 174, 300, doi:10.1016/j.cell.2018.06.026 (2018).
170 Que, E. L., et al., Metals in neurobiology: probing their chemistry and biology with molecular imaging. Chem. Rev. 108, 1517, doi:10.1021/cr078203u (2008).
171 Chan, L., et al., Sequence mutation and structural alteration transform a noncatalytic DNA sequence into an eficient RNA-ceaving DNAzyme. J. Mol. Evol. 81, 245, doi:10.1007/s00239-015-9712-x (2015).
172 Lacroix, A., et al., Uptake and Fate of Fluorescently Labeled DNA Nanostructures in Cellular Environments: A Cautionary Tale. ACS Cent Sci 5, 882, doi:10.1021/acscentsci.9b00174 (2019).
173 Doench, J. G., et al., Optimized sgRNA design to maximize activity and minimize off-target effects of CRISPR-Cas9. Nat. Biotechnol. 34, 184, doi:10.1038/nbt.3437 (2016). 174 Aquino-Jarquin, G. Current advances in overcoming obstacles of CRISPR/Cas9 off-target genome editing. Mol. Genet. Metab. 134, 77, doi:10.1016/j.ymgme.2021.08.002 (2021).

### REFERENCE LIST FOR EXAMPLE 7

1. Crichton, R. and J.-L. Pierre, Old iron, young copper: from Mars to Venus. Biometals, 2001. 14 (2): p. 99-112.
2. Camakaris, J., I. Voskoboinik, and J. Mercer, Molecular mechanisms of copper homeostasis. Biochemical and biophysical research communications, 1999. 261 (2): p. 225-232.
3. Nevitt, T., H. Öhrvik, and D. J. Thiele, Charting the travels of copper in eukaryotes from yeast to mammals. Biochimica et Biophysica Acta (BBA)-Molecular Cell Research, 2012. 1823 (9): p. 1580-1593.
4. Wang, Y., et al., Advances in the understanding of mammalian copper transporters. Advances in nutrition, 2011. 2 (2): p. 129-137.
5. Cotruvo Jr, J. A., et al., Synthetic fluorescent probes for studying copper in biological systems. Chemical Society Reviews, 2015. 44 (13): p. 4400-4414.
6. Aron, A. T., et al., Recognition- and reactivity-based fluorescent probes for studying transition metal signaling in living systems. Accounts of chemical research, 2015. 48 (8): p. 2434-2442.
7. Valko, M., H. Morris, and M. Cronin, Metals, toxicity and oxidative stress. Current medicinal chemistry, 2005. 12 (10): p. 1161-1208.
8. Halliwell, B. and J. M. Gutteridge, [1] Role of free radicals and catalytic metal ions in human disease: an overview. Methods in enzymology, 1990. 186: p. 1-85.
9. Gaetke, L. M. and C. K. Chow, Copper toxicity, oxidative stress, and antioxidant nutrients. Toxicology, 2003. 189 (1-2): p. 147-163.
10. Tsang, T., et al., Copper is an essential regulator of the autophagic kinases ULK1/2 to drive lung adenocarcinoma. Nature cell biology, 2020. 22 (4): p. 412-424.
11. Fox, J. H., et al., Mechanisms of copper ion mediated Huntington's disease progression. PloS one, 2007. 2 (3): p. e334.
12. Desai, V. and S. G. Kaler, Role of copper in human neurological disorders. The American journal of clinical nutrition, 2008. 88 (3): p. 855S-858S.
13. Xiao, G., et al., Huntington disease arises from a combinatory toxicity of polyglutamine and copper binding. Proceedings of the National Academy of Sciences, 2013. 110 (37): p. 14995-15000.
14. Hare, D. J., et al., Imaging metals in biology: balancing sensitivity, selectivity and spatial resolution. Chemical Society Reviews, 2015. 44 (17): p. 5941-5958.
15. Barnes, N., et al., The copper-transporting ATPases, Menkes and Wilson disease proteins, have distinct roles in adult and developing cerebellum. Journal of Biological Chemistry, 2005. 280 (10): p. 9640-9645.
16. Kaler, S. G., ATP7A-related copper transport diseases-emerging concepts and future trends. Nature reviews Neurology, 2011. 7 (1): p. 15-29.
17. Fieten, H., et al., The Menkes and Wilson disease genes counteract in copper toxicosis in Labrador retrievers: a new canine model for copper-metabolism disorders. Disease models & mechanisms, 2016. 9 (1): p. 25-38.
18. Outten, C. E., O'Halloran, and T. V, Femtomolar sensitivity of metalloregulatory proteins controlling zinc homeostasis. Science, 2001. 292 (5526): p. 2488-2492.
19. De Jonge, M. D. and S. Vogt, Hard X-ray fluorescence tomography—an emerging tool for structural visualization. Current opinion in structural biology, 2010. 20 (5): p. 606-614.
20. Hong-Hermesdorf, A., et al., Subcellular metal imaging identifies dynamic sites of Cu accumulation in Chlamydomonas. Nature chemical biology, 2014. 10 (12): p. 1034-1042.
21. Fahrni, C. J., Synthetic fluorescent probes for monovalent copper. Current opinion in chemical biology, 2013. 17 (4): p. 656-662.
22. Morgan, M. T., et al., Ratiometric two-photon microscopy reveals attomolar copper buffering in normal and Menkes mutant cells. Proceedings of the National Academy of Sciences, 2019. 116 (25): p. 12167-12172.
23. Lee, S., et al., Activity-based sensing with a metal-directed acyl imidazole strategy reveals cell type-dependent pools of labile brain copper. Journal of the American Chemical Society, 2020. 142 (35): p. 14993-15003.
24. Chung, C. Y.-S., et al., Activity-based ratiometric FRET probe reveals oncogene-driven changes in labile copper pools induced by altered glutathione metabolism. Proceedings of the National Academy of Sciences, 2019. 116 (37): p. 18285-18294.
25. Yi, X.-Q., et al., Porphyrinic probe for fluorescence “turn-on” monitoring of Cu⁺ in aqueous buffer and mitochondria. ACS sensors, 2019. 4 (4): p. 856-864.
26. Priessner, M., et al., Selective detection of Cu⁺ ions in live cells via fluorescence lifetime imaging microscopy. Angewandte Chemie, 2021. 133 (43): p. 23332-23337.
27. Zhu, A., et al., Carbon-dot-based dual-emission nanohybrid produces a ratiometric fluorescent sensor for in vivo imaging of cellular copper ions. Angewandte Chemie International Edition, 2012. 51 (29): p. 7185-7189.
28. Wang, S., et al., Activatable Small-Molecule Photoacoustic Probes that Cross the Blood-Brain Barrier for Visualization of Copper (II) in Mice with Alzheimer's Disease. Angewandte Chemie International Edition, 2019. 58 (36): p. 12415-12419.
29. Que, E. L., D. W. Domaille, and C. J. Chang, Metals in neurobiology: probing their chemistry and biology with molecular imaging. Chemical reviews, 2008. 108 (5): p. 1517-1549.
30. McConnell, E. M., et al., Biosensing with DNAzymes. Chemical Society Reviews, 2021.
31. Breaker, R. R. and G. F. Joyce, A DNA enzyme that cleaves RNA. Chemistry & biology, 1994. 1 (4): p. 223-229.
32. Li, Y. and R. R. Breaker, Deoxyribozymes: new players in the ancient game of biocatalysis. Current opinion in structural biology, 1999. 9 (3): p. 315-323.
33. Lyu, M., et al., PNA-assisted DNAzymes to cleave double-stranded DNA for genetic engineering with high sequence fidelity. Journal of the American Chemical Society, 2021. 143 (26): p. 9724-9728.
34. Verhaegh, G., M.]. Richard, and P. Hainaut, Regulation of p53 by metal ions and by antioxidants: dithiocarbamate down-regulates p53 DNA-binding activity by increasing the intracellular level of copper. Mol. Cell. Biol, 1997. 17: p. 5699-5706.
35. He, Y., et al., Amyloid β oligomers suppress excitatory transmitter release via presynaptic depletion of phosphatidylinositol-4, 5-bisphosphate. Nature communications, 2019. 10 (1): p. 1-18.
36. Zott, B., et al., A vicious cycle of β amyloid-dependent neuronal hyperactivation. Science, 2019. 365 (6453): p. 559-565.
37. Hardy, J. A. and G. A. Higgins, Alzheimer's disease: the amyloid cascade hypothesis. Science, 1992. 256 (5054): p. 184-185.
38. Marshall, K. E., et al., A critical role for the self-assembly of Amyloid-β1-42 in neurodegeneration. Scientific Reports, 2016. 6: p. 30182.
39. Lambert, M. P., et al., Diffusible, nonfibrillar ligands derived from Aβ1-42 are potent central nervous system neurotoxins. Proceedings of the National Academy of ences of the United States of America, 1998. 95 (11): p. 6448-6453.
40. Lacor, P. N., et al., Aβ Oligomer-Induced Aberrations in Synapse Composition, Shape, and Density Provide a Molecular Basis for Loss of Connectivity in Alzheimer's Disease. Journal of Neuroscience, 2007. 27 (4): p. 796-807.
41. Yichi, Z., et al., A Lifespan Observation of a Novel Mouse Model: In vivo Evidence Supports Aβ Oligomer Hypothesis. Plos One, 2014. 9 (1): p. e85885.
42. Savelieff, M. G., et al., Untangling Amyloid-β, Tau, and Metals in Alzheimer's Disease. Acs Chemical Biology, 2013. 8 (5).

What is claimed is:

1. A composition for simultaneously detecting a target ion in multiple oxidation states, the composition comprising:
i) a first DNAzyme sensor comprising:
a first substrate strand comprising a first cleavage site, wherein the first cleavage site is uncleaved when the target ion in a first oxidation state is not present; and
a first enzyme strand at least partially complementary to the first substrate strand and comprising a first catalytic loop;
wherein the first catalytic loop is capable of cleaving the first substrate strand at the first cleavage site in the presence of the target ion in the first oxidation state, wherein said cleavage provides a first detectable signal; and
ii) a second DNAzyme sensor comprising:
a second substrate strand comprising a second cleavage site, wherein the second cleavage site is uncleaved when the target ion in a second oxidation state is not present; and
a second enzyme strand at least partially complementary to the second substrate strand and comprising a second catalytic loop;
wherein the second catalytic loop is capable of cleaving the second substrate strand at the second cleavage site in the presence of the target ion in the second oxidation state, wherein said cleavage provides a second detectable signal.

2. The composition of claim 1, wherein the target ion is iron;
wherein the first cleavage site is uncleaved when Fe²⁺ is not present and the first catalytic loop comprises SEQ ID NO: 306 or a variant thereof; and
wherein the second cleavage site is uncleaved when Fe³⁺ is not present and the second catalytic loop comprises SEQ ID NO: 307 or a variant thereof.

3. The composition of claim 2, wherein the first substrate strand comprises 80% similarity or more to any one of the sequences in TABLE 1;
wherein the first enzyme strand comprises 80% similarity or more to any one of the sequences in TABLE 2;
wherein the second substrate strand comprises 80% similarity or more to any one of the sequences in TABLE 3; and
wherein the second enzyme strand comprises 80% similarity or more to any one of the sequences in TABLE 4.

4. The composition of claim 1, wherein the target ion is copper;
wherein the first cleavage site is uncleaved when Cu⁺ is not present and the first catalytic loop comprises SEQ ID NO: 308 or a variant thereof; and
wherein the second cleavage site is uncleaved when Cu²⁺ is not present and the second catalytic loop comprises SEQ ID NO: 309 or a variant thereof.

5. The composition of claim 4, wherein the first substrate strand comprises 80% similarity or more to any one of the sequences in TABLE 5;
wherein the first enzyme strand comprises 80% similarity or more to any one of the sequences in TABLE 6;
wherein the second substrate strand comprises 80% similarity or more to any one of the sequences in TABLE 7; and
wherein the second enzyme strand comprises 80% similarity or more to any one of the sequences in TABLE 8.

6. A method of using the composition of claim 1 to simultaneously detect the target ion in the first oxidation state and the second oxidation state in a cell or tissue, the method comprising:
a) providing the composition to the cell or tissue; and
b) detecting the first detectable signal and the second detectable signal.

7. A DNAzyme sensor comprising:
a substrate strand comprising a cleavage site, wherein the cleavage site is uncleaved when Fe²⁺ is not present; and
an enzyme strand at least partially complementary to the substrate strand and comprising a catalytic loop;
wherein the catalytic loop is capable of cleaving the substrate strand at the cleavage site in the presence of Fe²⁺, wherein said cleavage provides a detectable signal; and
wherein the catalytic loop comprises SEQ ID NO: 306 or a variant thereof.

8. The DNAzyme sensor of claim 7, wherein the cleavage site comprises from 1 RNA base to 5 RNA bases.

9. The DNAzyme sensor of claim 7, wherein the substrate strand further comprises at least one non-natural nucleic acid.

10. The DNAzyme sensor of claim 7, wherein the detectable signal is a fluorophore, fluorescent dye, or photoacoustic dye.

11. The DNAzyme sensor of claim 7, wherein the detectable signal is conjugated to a first end of the substrate strand; and
wherein a quencher is conjugated to a complementary end of the enzyme strand or to a second end of the substrate strand.

12. The DNAzyme sensor of claim 7, wherein the substrate strand comprises 80% similarity or more to any one of the sequences in TABLE 1; and
wherein the enzyme strand comprises 80% similarity or more to any one of the sequences in TABLE 2.

13. A method of using the DNAzyme sensor of claim 7 to detect Fe²⁺ in a cell or tissue, the method comprising:
a) providing the DNAzyme sensor to the cell or tissue; and
b) detecting the detectable signal.

14. A DNAzyme sensor comprising:
a substrate strand comprising a cleavage site, wherein the cleavage site is uncleaved when Fe³⁺ is not present; and
an enzyme strand at least partially complementary to the substrate strand and comprising a catalytic loop;
wherein the catalytic loop is capable of cleaving the substrate strand at the cleavage site in the presence of Fe³⁺, wherein said cleavage provides a detectable signal; and
wherein the catalytic loop comprises SEQ ID NO: 307 or a variant thereof.

15. The DNAzyme sensor of claim 14, wherein the cleavage site comprises from 1 RNA base to 5 RNA bases.

16. The DNAzyme sensor of claim 14, wherein the substrate strand further comprises at least one non-natural nucleic acid.

17. The DNAzyme sensor of claim 14, wherein the detectable signal is a fluorophore, fluorescent dye, or photoacoustic dye.

18. The DNAzyme sensor of claim 14, wherein the detectable signal is conjugated to a first end of the substrate strand; and
wherein a quencher is conjugated to a complementary end of the enzyme strand or to a second end of the substrate strand.

19. The DNAzyme sensor of claim 14, wherein the substrate strand comprises 80% similarity or more to any one of the sequences in TABLE 3; and
wherein the enzyme strand comprises 80% similarity or more to any one of the sequences in TABLE 4.

20. A method of using the DNAzyme sensor of claim 14 to detect Fe³⁺ in a cell or tissue, the method comprising:
a) providing the DNAzyme sensor to the cell or tissue; and
b) detecting the detectable signal.
